(19) United States
(12) Patent Application Publication
(10) Pub. No.: US 2004/0060081 A1
(43) Pub. Date: Mar. 25, 2004

(54) Plant genes that confer resistance to strains of magnaporthe grisea having avri co39 cultivar specificity gene

(76) Inventors: Sally A. Leong, Stoneham, MA (US); Mark L. Farman, Lexington, KY (US); Rajinder S. Chauhan, Madison, WI (US); Timothy J. Durfee, Madison, WI (US)

Correspondence Address: USDA-ARS-OFFICE OF TECHNOLOGY TRANSFER, NATIONAL CTR FOR AGRICULTURAL UTILIZATION RESEARCH, 1815 N. UNIVERSITY STREET, PEORIA, IL, 61604, US

(21) Appl. No.: 10/415,058

(22) PCT Filed: Oct. 19, 2001
(86) PCT No.: PCT/US01/46331

(51) Int. Cl. A01H 1/00; A01H 5/00; C12Q 1/68; C07H 21/04; C12N 9/24; C12N 15/82; C12N 5/04

(52) U.S. Cl. 800/279; 800/320.2; 435/6; 435/69.1; 435/320.1; 435/200; 435/419; 536/23.2

(57) ABSTRACT

A plant pathogen resistance gene present in rice cultivar CO39 and other plant species is disclosed. The gene is referred to as Pi-CO39(t) and confers resistance to strains of the plant pathogen, Magnaporthe grisea (causal agent of rice blast and other plant diseases), having a corresponding AVR1 CO39 avirulence gene. Also disclosed are methods of using the resistance gene and its encoded products for improving resistance of plants to this pathogen.

This application claims priority under 35 U.S.C. §119(e)(1) to U.S. Provisional Application No. 60/242,313, filed Oct. 20, 2000, and to U.S. Provisional Application No. 60/303,897, filed Jul. 9, 2001, the entireties of both of which are incorporated by reference herein.

Pursuant to 35 U.S.C. §202(c), it is acknowledged that the U.S. Government has certain rights in the invention described herein, which was made in part with funds from the National Institutes of Health (Grant No. GM33716) and the United States Department of Agriculture—Agricultural Research Service (Project Nos.3655-0500-008-00D, 3655-22000-010-00D and 58-3655-7-208).

### FIELD OF THE INVENTION

This invention relates to the field of disease resistance in plants. In particular, the invention is drawn to novel resistance genes present in rice cultivar CO39 and other plant species, that confers resistance to strains of the rice blast pathogen, Magnaporthe grisea, having a corresponding avirulence gene. This invention further provides methods of using the resistance gene and its encoded products for improving resistance of plants to this pathogen.

### BACKGROUND OF THE INVENTION

Various patent and scientific publications are referred to throughout the specification to describe the state of the art to which this invention pertains. Full citations of such publications not appearing within the specification may be found at the end of the specification. Each of these publications is incorporated by reference herein in its entirety.

Rice is a major staple food for about two-thirds of the world's population More than ninety percent of the world's rice is grown and consumed in developing countries. Rice blast disease, caused by the fungus Magnaporthe grisea, threatens rice crops worldwide. The disease can cause yield losses of ten to thirty percent in infested fields. Rice blast has been an ongoing problem in rice growing areas of the southern United States. It has now become a significant problem in rice growing areas of California, as well.

The “gene-for-gene” hypothesis has been advanced to explain the very specific disease resistance/susceptibility relationship that often exists between races of a plant pathogen and cultivars of its host species. The gene-for-gene hypothesis has been found applicable to many host-pathogen interactions, including that of the rice blast fungus, Magnaporthe grisea, and its host, Oryza sativa. To be able to understand and manipulate this host-pathogen relationship is of great practical interest as M. grisea is rapidly able to overcome new disease resistance in rice soon after their deployment. Moreover, M. grisea exists as a complex genus with many subspecific groups that are sometimes infertile, but differ in their host range. How these different subspecific groups interrelate evolutionarily is of great concern to plant breeders since some of these alternate hosts are frequently found growing in close proximity to, or in rotation with rice, and M. grisea isolates infecting these alternate hosts can sometimes also infect rice.

Gene-for-gene resistance (also known as hypersensitive resistance (KR) or race-specific resistance) depends for its activation on specific recognition of the invading pathogen by the plant. Many individual plant genes have been identified that control gene-for-gene resistance. These genes are referred to as resistance (R) genes. The function of a particular R gene depends on the genotype of the pathogen. A pathogen gene is referred to as an Avr gene if its expression causes the pathogen to produce a signal that triggers a strong defense response in a plant having a corresponding R gene. This response is not observed in the absence of either the Avr gene in the pathogen or the corresponding R gene in the plant. It should be noted that a single plant may have many R genes, and a single pathogen may have many Avr genes. However, strong resistance occurs only when an Avr gene (which is usually a dominant allele) and its corresponding specific R gene (also usually a dominant allele) are matched in a host-pathogen interaction. In this instance, resistance generally occurs as activation of a HR response, in which the cells in the immediate vicinity of the infection undergo programmed necrosis in order to prevent the further advance of the pathogen into living plant tissue. Other features of the resistance response may also include synthesis of antimicrobial metabolites or pathogen-inhibiting enzymes, reinforcement of plant cell walls in the infected area, and induction of signal transduction pathways leading to systemic acquired resistance (SAR) in the plant.

The molecular basis of host-cultivar specificity and pathogenic variability in M. grisea has been partly elucidated with the identification, mapping and, in some instances, cloning of specific Avr genes from pathogenic isolates of M. grisea. For instance, AVR2-YAMO (more recently named Avr-Pita)(cultivar specificity) and PWL2 (host specificity) (Valent & Chumley, pp. 3.113-3.134 in Rice Blast Disease (R. Zeigler, S. A. Leong, P. Teng, Eds.), Wallingford: CAB International, 1994) both function as classic avirulence genes by preventing infection of a specific cultivar or host. AVR2-YAMO encodes a 223-amino acid protein with homology to proteases, while PWL2 encodes a 145-amino acid polypeptide which is glycine-rich. Based on the predicted amino acid sequences of the proteins, both may be secreted (Sweigard et al., Plant Cell 7:1221-1233, 1995).

Homologs of both AVR2-YAMO and PWL2 appear to be widely distributed in rice and in other grass-infecting isolates of M. grisea, thereby confirming that M. grisea isolates which do not infect rice still may carry host or cultivar specificity genes for rice. In some cases, homologs of AVR2-YAMO and PWL2 have been shown to be functional and to exhibit the same host or cultivar specificity as AVR2-YAMO or PWL2 (Kang et al., Molecular Plant-Microbe Interactions. 8:939-498, 1995; Orbach et al., Plant Cell. 12:2019-2032, 2000;.Jia et al., EMBO J 12:4004-4014, 2000).

As another example of a potentially useful Avr gene, the cultivar specificity gene AVR1-CO39, which determines avirulence on rice cultivar CO39, has been identified (Valent et al., Genetics 127: 87-101, 1991) and mapped to a position on M. grisea chromosome 1 (Smith & Leong, Theor. Appl. Genet. 88: 901-908, 1994). The avirulence gene in M. grisea (AVR1-CO39) has been cloned and sequenced (Farman & Leong, 1998; see also PCT US99/04047 and commonly-owned co-pending U.S. application Ser. No. 09/257,585).

Indica rice cultivar CO39 was originally bred for blast resistance and agronomic value and has been lately used as a tester for blast pathogenicity assays as well as a recurrent parent for developing near-isogenic-lines. Genetic analysis of blast resistance in CO39 to M. grisea progeny, 6082, which carries AVR1-CO39, as well as to the Guy 11(AVR1-CO39) transformant has shown that resistance is controlled by a single dominant locus. The resistance phenotype is uniform and consistently of reaction type 1 and is inherited as a simple Mendelian trait among different segregating populations (F1/F2/F3/F4). The resistance gene(s), designated as Pi-CO39(t), has been mapped to the short arm of rice chromosome 11. However, neither fine mapping nor cloning of the Pi-CO39(t) locus has been reported.

In addition to cloned cultivar and host specificity genes from M. grisea, the availability of the corresponding R genes from rice would provide usefuil tools for manipulating and augmenting resistance to this pathogen in the field. Accordingly, it is an object of the present invention to provide a new cloned rice R gene that confers resistance to strains of Magnaporthe grisea carrying the AVR1-CO39 avirulence gene. It is a further object of the present invention to provide methods for using the R gene and the corresponding fungal avirulence gene to confer or improve resistance of other cultivars and plant species to rice blast and other plant diseases.

### SUMMARY OF THE INVENTION

One aspect of the invention features an isolated nucleic acid segment from chromosome 11 of Indica rice cultivar CO39, which comprises one or more genes that confer resistance to strains of the rice blast pathogen, Magnaporthe grisea, that have the avirulence gene AVR1-CO39. The locus comprising the gene(s) co-segregates with one or more of the following markers: (1) RGA8 (GenBank Accession No. AF074889; Mago et al., 1999); (2) RGA38 (GenBank Accession No. AF074895; Mago et al., 1999); and (3) G320 (GenBank Accession No. RICG320A Fukuoka et al., 1994). The gene (or, if more than one, the plurality of genes) is referred to herein as Pi-CO39(t). It should be noted that the term Pi-CO39(t) gene, if used in the singular herein, also refers to any plurality of genes associated with resistance to AVR1-CO39expressing strains of M. grisea.

According to another aspect of the invention, a transgenic plant comprising the Pi-CO39(t) resistance gene is provided. Expression of the gene in the transgenic plants confers a resistance response upon challenge with the gene product of AVR1-CO39 or microorganisms expressing the AVR1-CO39 gene. In a preferred embodiment, the plant is rice. In another embodiment, the plant is a monocot other than rice, which is susceptible to diseases caused by Magnaporthe. Such plants include, for example, turf grasses such as Lolium perenne. In another embodiment, the plant is a dicotyledenous species.

According to another aspect of the invention, a method of enhancing pathogen resistance in a plant is provided. The method comprises the following steps: (1) transforming the plant with the Pi-CO39(t) gene; and (2) pre-treating the transformed plant with either the AVR1-CO39 gene product or a non-pathogenic organism (e.g., an epiphytic bacterium or a non-pathogenic fungus) that expresses a portion of an AVR1-CO39 gene effective to trigger expression of a CO39-specific R gene in the plants. Triggering expression of the R gene in this manner will confer upon the plant increased resistance not only to Magnaporthe grisea, but also to other plant pathogens whose infective ability is reduced or prevented by the R gene product and its associated activity.

These and other features and advantages of the present invention will be described in greater detail in the description and examples set forth below.

### BRIEF DESCRIPTION OF THE DRAWINGS

FIG. 1. Schematic diagrams showing a genetic map linked with physical maps of a region of rice chromosome 11-associated with Pi-CO39 (t). The genetic map displays the resistance gene(s), Pi-CO39(t) with respect to co-segregating markers. Designations to the right of the schematic diagram of chromosome 11 of rice CO39 are names of genetic markers. Numbers to the left represent distance in centiMorgans (cM). The physical map is from Japonica rice variety, Nipponbare and Indica rice variety CO39. The physical map shows minimum tiling path of BAC clones from Contig 43 of a BAC library from Japonica rice variety, Nipponbare and its relationship to a contig of BAC clones from Indica rice variety CO39.

FIG. 2. Diagram showing comparative sequence analysis of BAC clones from variety Nipponbare (Japonica, susceptible) and CO39 (Indica, resistant) at regions that cosegregate with Pi-CO39 (t). NSL (Nipponbare serpin-like genes); CSL (CO39 serpin-like genes); NBR (Nipponbare NBS-LRR disease resistance-like genes); CODR (CO39 NBS-LRR disease resistance-like genes); note NBR2 is a rice Pib-like gene; NBR3, NBR5 and CODR4 are rice Pi-ta-like genes; KIN1 and KIN2 are receptor kinases; CODR3 is a rice Xa1-like gene; another CODR gene, homologous to NBR1 or NBR2, may exist between CSL3 and CODR1 on E2P5; and the solid bars indicate retroelements.

### DETAILED DESCRIPTION OF THE INVENTION

I. Definitions

Various terms relating to the biological molecules of the present invention are used hereinabove and also throughout the specifications and claims.

The term “pathogen-inoculated” refers to the inoculation of a plant with a pathogen.

The term “disease defense response” refers to a change in metabolism, biosynthetic activity or gene expression that enhances the plant's ability to suppress the replication and spread of a microbial pathogen (i.e., to resist the microbial pathogen). Examples of plant disease defense responses include, but are not limited to, production of low molecular weight compounds with antimicrobial activity (referred to as phytoalexins) and induction of expression of defense (or defense-related) genes, whose products include, for example, peroxidases, cell wall proteins, proteinase inhibitors, hydrolytic enzymes, pathogenesis-related (PR) proteins and phytoalexin biosynthetic enzymes, such as phenylalanine ammonia lyase and chalcone synthase. Such defense responses appear to be induced in plants by several signal transduction pathways involving secondary defense signaling molecules produced in plants. Agents that induce disease defense responses in plants include, but are not limited to: (1) microbial pathogens, such as fungi, bacteria and viruses; (2) microbial components and other defense response elicitors, such as proteins and protein fragments, small peptides, β-glucans, elicitins and harpins, cryptogein and oligosaccharides; and (3) secondary defense signaling molecules produced by the plant, such as salicylic acid, H₂O₂, nitric oxide, ethylene and jasmonates.

With reference to nucleic acids of the invention, the term “isolated nucleic acid” or “polynucleotide” is sometimes used. This term, when applied to DNA, refers to a DNA molecule that is separated from sequences with which it is immediately contiguous (in the 5′ and 3′ directions) in the naturally occurring genome of the organism from which it was derived. For example, the “isolated nucleic acid” may comprise a DNA molecule inserted into a vector, such as a plasmid or virus vector, or integrated into the genomic DNA of a procaryote or eucaryote. An “isolated nucleic acid molecule” may also comprise a cDNA molecule.

With respect to RNA molecules of the invention the term “isolated nucleic acid” or “polynucleotide” primarily refers to an RNA molecule encoded by an isolated DNA molecule as defined above. Alternatively, the term may refer to an RNA molecule that has been sufficiently separated from RNA molecules with which it would be associated in its natural state (i.e., in cells or tissues), such that it exists in a “substantially pure” form (the term “substantially pure” is defined below).

With respect to protein, the term “isolated protein” or “isolated and purified protein” is sometimes used herein. This term refers primarily to a protein produced by expression of an isolated nucleic acid molecule of the invention. Alternatively, this term may refer to a protein which has been sufficiently separated from other proteins with which it would naturally be associated, so as to exist in “substantially pure” form.

With respect to antibodies of the invention, the term “immunologically specific” refers to antibodies that bind to one or more epitopes of a protein of interest, but which do not substantially recognize and bind other molecules in a sample containing a mixed population of antigenic biological molecules.

The term “substantially pure” refers to a preparation comprising at least 50-60% by weight the compound of interest (e.g., nucleic acid, oligonucleotide, protein, etc.). More preferably, the preparation comprises at least 75% by weight, and most preferably 90-99% by weight, the compound of interest Purity is measured by methods appropriate for the compound of interest (e.g. chromatographic methods, agarose or polyacrylamide gel electrophoresis, HPLC analysis, and the like).

When used herein in describing components of media or other experimental results, the term “about” means within a margin of commonly acceptable error for the determination being made, using standard methods. For plant transformation or tissue culture media in particular, persons skilled in the art would appreciate that the concentrations of various components initially added to culture media may change somewhat during use of the media, e.g., by evaporation of liquid from the medium or by condensation onto the medium. Moreover, it is understood that the precise concentrations of the macronutrients, vitamins and carbon sources are less critical to the efficacy of the media than are the micronutrient, hormone and antibiotic concentrations.

With respect to single stranded oligonucleotides and polynucleotides, the term “specifically hybridizing” refers to the association between two single-stranded nucleotide molecules of sufficiently complementary sequence to permit such hybridization under pre-determined conditions generally used in the art (sometimes termed “substantially complementary”). In particular, the term refers to hybridization of an oligonucleotide or polynucleotide with a substantially complementary sequence contained within a single-stranded DNA or RNA molecule of the invention, to the substantial exclusion of hybridization of the oligonucleotide or polynucleotide with single-stranded nucleic acids of non-complementary sequence.

The term “substantially the same” refers to nucleic acid or amino acid sequences having sequence variation that do not materially affect the nature of the protein (i.e. the structure, stability characteristics and/or biological activity of the protein). With particular reference to nucleic acid sequences, the term “substantially the same” is intended to refer to the coding region and to conserved sequences governing expression, and refers primarily to degenerate codons encoding the same amino acid, or alternate codons encoding conservative substitute amino acids in the encoded polypeptide. With reference to amino acid sequences, the term “substantially the same” refers generally to conservative substitutions and/or variations in regions of the polypeptide not involved in determination of structure or function.

Nucleic acid sequences and amino acid sequences can be compared using computer programs that align the similar sequences of the nucleic or amino acids thus define the differences. In preferred methodologies, the Blastn and Blastp 2.0 programs provided by the National Center for Biotechnology Information (at www.ncbi.nlm.nih.gov/blast/: Altschul et al., 1990, J. Mol. Biol. 215:403-410) using a gapped alignment with default parameters, may be used to determine the level of identity and similarity between nucleic acid sequences and amino acid sequences. However, equivalent alignments and similarity/identity assessments can be obtained through the use of any standard alignment software. For instance, the DNAstar system (Madison, Wis.) may be used to align sequence fragments of genomic or other DNA sequences. Alternatively, GCG Wisconsin Package version 9.1, available from the Genetics Computer Group in Madison, Wis., and the default parameters used (gap creation penalty=12, gap extension penalty=4) by that program may also be used to compare sequence identity and similarity.

The terms “percent identical” and “percent similar” are also used herein in comparisons among amino acid and nucleic acid sequences. When referring to amino acid sequences, “percent identical” refers to the percent of the amino acids of the subject amino acid sequence that have been matched to identical amino acids in the compared amino acid sequence by a sequence analysis program. “Percent similar” refers to the percent of the amino acids of the subject amino acid sequence that have been matched to identical or conserved amino acids. Conserved amino acids are those which differ in structure but are similar in physical properties such that the exchange of one for another would not appreciably change the tertary structure of the resulting protein. Conservative substitutions are defined by Taylor (1986, J. Theor. Biol. 119:205). Polypeptides having sequences greater than 70% identical, preferably greater than 80%, and more preferably greater than 90% and most preferably greater than 95% identical to the polypeptides encoded by the nucleic acid sequences described herein are considered within the scope of the invention. When referring to nucleic acid molecules, “percent identical” refers to the percent of the nucleotides of the subject nucleic acid sequence that have been matched to identical nucleotides by a sequence analysis program. Polynucleotides having sequences greater than 60% identical, preferably greater than 70% identical, more preferably preferably greater than 80% identical, and more preferably greater than 90% identical, and most preferably greater than 95% identical to the polynucleotides described herein are considered within the scope of the invention.

A “coding sequence” or “coding region” refers to a nucleic acid molecule having sequence information necessary to produce a gene product (RNA or protein), when the sequence is expressed.

The term “operably linked” or “operably inserted” means that the regulatory sequences necessary for expression of the coding sequence are placed in a nucleic acid molecule in the appropriate positions relative to the coding sequence so as to enable expression of the coding sequence. This same definition is sometimes applied to the arrangement of other transcription control elements (e.g. enhancers) in an expression vector.

Transcriptional and translational control sequences, sometimes referred to herein as “expression control” sequences or elements, or “expression regulating” sequences or elements, are DNA regulatory elements such as promoters, enhancers, ribosome binding sites, polyadenylation signals, terminators, and the like, that provide for the expression of a coding sequence in a host cell. The term “expression” is intended to include transcription of DNA and translation of the mRNA transcript.

The terms “promoter”, “promoter region” or “promoter sequence” refer generally to transcriptional regulatory regions of a gene, which may be found at the 5′ or 3′ side of the coding region, or within the coding region, or within introns. Typically, a promoter is a DNA regulatory region capable of binding RNA polymerase in a cell and initiating transcription of a downstream (3′ direction) coding sequence. The typical 5′ promoter sequence is bounded at its 3′ terminus by the transcription initiation site and extends upstream (5′ direction) to include the minimum number of bases or elements necessary to initiate transcription at levels detectable above background. Within the promoter sequence is a transcription initiation site (conveniently defined by mapping with nuclease S1), as well as protein binding domains (consensus sequences) responsible for the binding of RNA polymerase.

A “vector” is a replicon, such as plasmid, phage, cosmid, or virus to which another nucleic acid segment may be operably inserted so as to bring about the replication or expression of the segment.

The term “nucleic acid construct” or “DNA construct” is sometimes used to refer to a coding sequence or sequences operably linked to appropriate regulatory sequences and inserted into a vector for transforming a cell. This term may be used interchangeably with the term “transforming DNA”. Such a nucleic acid construct may contain a coding sequence for a gene product of interest, along with a selectable marker gene and/or a reporter gene. These constructs may be administered to plants in a viral or plasmid vector. Other methods of delivery such as Agrobacterium T-DNA mediated transformation and transformation using the biolistic process are also contemplated to be within the scope of the present invention. In addition to specific methods described herein, the transforming DNA may be prepared according to standard protocols such as those set forth in “Current Protocols in Molecular Biology”, eds. Frederick M. Ausubel et al., John Wiley & Sons, 2001. In certain embodiments, such constructs are chimeric, i.e., the coding sequence is from a different source one or more of the regulatory sequences (e.g., coding sequence from rice and promoter from maize or Arabidopsis). However, non-chimeric DNA constructs also can be used. A plant species or cultivar may be transformed with a DNA construct (chimeric or non-chimeric) that encodes a polypeptide from a different plant species or cultivar, or a non-plant species. Alternatively, a plant species or cultivar may be transformed with a DNA construct (chimeric or non-chimeric) that encodes a polypeptide from the same plant species or cultivar. The term “transgene” is sometimes used to refer to the DNA construct within the transformed cell or plant.

The term “selectable marker gene” refers to a gene encoding a product that, when expressed, confers a selectable phenotype such as antibiotic resistance on a transformed cell.

The term “reporter gene” refers to a gene that encodes a product which is easily detectable by standard methods, either directly or indirectly.

A “heterologous” region of a nucleic acid construct is an identifiable segment (or segments) of the nucleic acid molecule within a larger molecule that is not found in association with the larger molecule in nature. Thus, when the heterologous region encodes a plant gene, the gene will usually be flanked by DNA that does not flank the plant genomic DNA in the genome of the source organism. In another example, a heterologous region is a construct where the coding sequence itself is not found in nature (e.g., a cDNA where the genomic coding sequence contains introns, or synthetic sequences having codons different than the native gene). Allelic variations or naturally occurring mutational events do not give rise to a heterologous region of DNA as defined herein. The term “DNA construct”, as defined above, is also used to refer to a heterologous region, particularly one constructed for use in transformation of a cell.

A cell has been “transformed” or “transfected” by exogenous or heterologous DNA when such DNA has been introduced inside the cell. The transforming DNA may or may not be integrated (covalently linked) into the genome of the cell. In prokaryotes, yeast, and plant or animal cells for example, the transforming DNA may be maintained on an episomal element such as a plasmid. With respect to eukaryotic cells, a stably transformed cell is one in which the transforming DNA has become integrated into a chromosome so that it is inherited by daughter cells through chromosome replication. This stability is demonstrated by the ability of the eukaryotic cell to establish cell lines or clones comprised of a population of daughter cells containing the transforming DNA. A “clone” is a population of cells derived from a single cell or common ancestor by mitosis. A “cell line” is a clone of a primary cell that is capable of stable growth in vitro for many generations.

II. Description

In accordance with the present invention, a novel rice resistance gene (or genes) has been identified and localized to a specific region on chromosome 11 of the rice genome. This gene is referred to herein as Pi-CO39(t), to denote its function as a gene in rice cultivar CO39 that confers resistance to strains of the plant pathogen, Magnaporthe grisea, that contain the cultivar specificity gene AVR1-CO39.

The genetic mapping of Pi-CO39(t) in rice line CO39 and identification of large insert clones linked to resistance are described in detail in Example 1. The gene was mapped to 5.9 cM from marker RG1094 and 1.0 cM from marker S2712 on chromosome 11 in 260 F2 progenies. Three additional markers, RGA8, RGA38 and G320, were found to co-segregate with the Pi-CO39(t) gene in 400 F2 progenies among 1,100 F2 progenies phenotypically tested for resistance. These markers were used to isolate BAC clones from the resistant variety CO39 and the susceptible variety Nipponbare. Single BAC clones hybridizing to all co-segregating markers were obtained from the Nipponbare library; these are clones 82N20, 55L08, 20A20, 5M17. Several BAC clones hybridizing to co-segregating marker RGA38 were obtained from the CO39 library; these are clones 1L23, 36K6,4A14. Sequence analysis of the RGA38 and RGA8 homologs in CO39 yielded SEQ ID NO: 1 and SEQ ID NO:2, respectively. These two sequences are about 97% and 84% identical, respectively, to RGA38 and RGA8 as reported by Mago et al. (GenBank Accession Numbers set forth above).

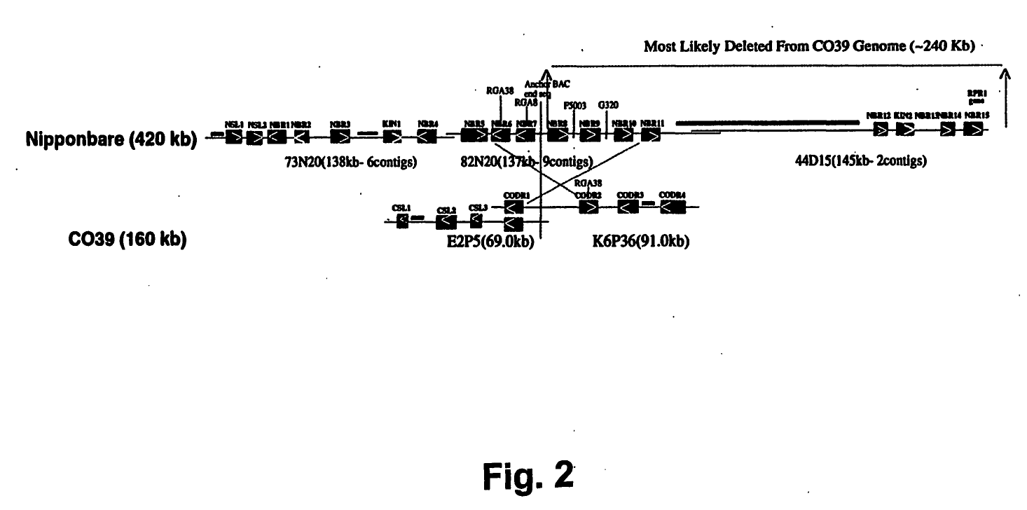

It is believed that part or all of the genomic region of rice containing Pi-CO39(t) resides on one or more of the CO39 BAC clones listed above. A contig of about 0.5 mb has been constructed in the relevant region of the Nipponbare library. Sequence information of portions of the Nipponbare contig is set forth in Example 2 as SEQ ID NOS: 3 and 4. Sequences of two BAC clones, K6P36 and E2P5, from the relevant region of CO39 chromosome 11 are set forth herein as SEQ ID NO:5 and SEQ ID NO:6. A detailed analysis of the BAC clones and the genes predicted to be located on the clones is also set forth in the examples. Sequences representing the open reading frames of BAC clones K6P36 and E2P5 are set forth as SEQ ID NOS: 7-14, and described in FIG. 2 and in the examples.

The following description sets forth the general procedures involved in practicing the present invention. To the extent that specific materials are mentioned, it is merely for purposes of illustration and is not intended to limit the invention Unless otherwise specified, general cloning procedures, such as those set forth in Sambrook et al., Molecular Cloning, Cold Spring Harbor Laboratory (1989) (hereinafter “Sambrook et al.”) or Ausubel et al. (eds) Current Protocols in Molecular Biology, John Wiley & Sons (2001) (hereinafter “Ausubel et al.”) are used.

### A. Preparation of Pi-CO39(t) Nucleic Acid M lecules, Encoded P lypeptides and Transgenic Plants

1. Nucleic Acid Molecules

Pi-CO39(t) nucleic acid molecules of the invention may be prepared by two general methods: (1) they may be synthesized from appropriate nucleotide triphosphates, or (2) they may be isolated from biological sources. Both methods utilize protocols well known in the art.

The availability of the Pi-CO39(t) nucleotide sequence information enables preparation of an isolated nucleic acid molecule of the invention by oligonucleotide synthesis. Synthetic oligonucleotides may be prepared by the phosphoramadite method employed in the Applied Biosystems 38A DNA Synthesizer or similar devices. The resultant construct may be purified according to methods known in the art, such as high performance liquid chromatography (HPLC).

Pi-CO39(t) genes also may be isolated from appropriate biological sources using methods known in the art. In one embodiment, large insert clones have been isolated from BAC libraries of a resistant and susceptible rice cultivar. In an alternative embodiment, a cDNA clone comprising the open reading frame of the genomic Pi-CO39(t) locus may be isolated.

In accordance with the present invention, nucleic acids having the appropriate level sequence homology with part or all the coding and/or regulatory regions of Pi-CO39(t) may be identified by using hybridization and washing conditions of appropriate stringency. For example, hybridizations may be performed, according to the method of Sambrook et al., using a hybridization solution comprising: 5×SSC, 5× Denhardt's reagent, 1.0% SDS, 100 μg/ml denatured, fragmented salmon sperm DNA, 0.05% sodium pyrophosphate and up to 50% formamide. Hybridization is carried out at 37-42° C. for at least six hours. Following hybridization, filters are washed as follows: (1) 5 minutes at room temperature in 2×SSC and 1% SDS; (2) 15 minutes at room temperature in 2×SSC and 0.1% SDS; (3) 30 minutes-1 hour at 37° C. in 2×SSC and 0.1% SDS; (4) 2 hours at 45-55° in 2×SSC and 0.1% SDS, changing the solution every 30 minutes. Alternatively, a modification of the Amasino hybridization protocol (Anal. Biochem. 152: 304-307) is preferred for use in the present invention and is described in greater detail in Example 1.

One common formula for calculating the stringency conditions required to achieve hybridization between nucleic acid molecules of a specified sequence homology (Sambrook et al., 1989):

Tₘ=81.5° C.+16.6 Log [Na+]+0.41(% G+C)−0.63 (% formamide)−600/#bp in duplex

As an illustration of the above formula, using [N+]=[0.368] and 50% formamide, with GC content of 42% and an average probe size of 200 bases, the Tₘ is 57° C. The Tₘ of a DNA duplex decreases by 1-1.5° C. with every 1% decrease in homology. Thus, targets with greater than about 75% sequence identity would be observed using a hybridization temperature of 42° C. In a preferred embodiment, the hybridization is at 37° C. and the final wash is at 42° C., in a more preferred embodiment the hybridization is at 42° C. and the final wash is at 50° C., and in a most preferred embodiment the hybridization is at 42° C. and final wash is at 65° C., with the above hybridization and wash solutions. Conditions of high stringency include hybridization at 42° C. in the above hybridization solution and a final wash at 65° C. in 0.1×SSC and 0.1% SDS for 10 minutes.

Nucleic acids of the present invention may be maintained as DNA in any convenient cloning vector. In a preferred embodiment, clones are maintained in plasmid cloning/expression vector, such as pGEM-T (Promega Biotech, Madison, Wis.) or pBluescript (Stratagene, La Jolla, Calif.), either of which is propagated in a suitable E. coli host cell.

Pi-CO39(t) nucleic acid molecules of the invention include cDNA, genomic DNA, RNA, and fragments thereof which may be single- or double-stranded. Thus, this invention provides oligonucleotides (sense or antisense strands of DNA or RNA) having sequences capable of hybridizing with at least one sequence of a nucleic acid molecule of the present invention. Such oligonucleotides are useful as probes for detecting Pi-CO39(t) genes or mRNA in test samples of plant tissue, e.g. by PCR amplification, or for the positive or negative regulation of expression of Pi-CO39(t) genes at or before translation of the mRNA into proteins.

2. Polypeptides

Polypeptides encoded by the Pi-CO39(t) gene may be prepared in a variety of ways, according to known methods. If produced in situ the polypeptides may be purified from appropriate sources, e.g., plant tissue.

Alternatively, the availability of nucleic acid molecules encoding the polypeptides will enable production of the proteins using in vitro expression methods known in the art. For example, a cDNA or gene may be cloned into an appropriate in vitro transcription vector, such a pSP64 or pSP65 for in vitro transcription, followed by cell-free translation in a suitable cell-free translation system, such as wheat germ or rabbit reticulocytes. In vitro transcription and translation systems are commercially available, e.g., from Promega Biotech, Madison, Wis. or BRL, Rockville, Md.

According to a preferred embodiment, larger quantities of Pi-CO39(t)-encoded polypeptides may be produced by expression in a suitable procaryotic or eucaryotic system. For example, part or all of a Pi-CO39(t) gene or cDNA may be inserted into a plasmid vector adapted for expression in a bacterial cell (such as E. coli) or a yeast cell (such as Saccharomyces cerevisiae), or into a baculovirus vector for expression in an insect cell. Such vectors comprise the regulatory elements necessary for expression of the DNA in the host cell, positioned in such a manner as to permit expression of the DNA in the host cell. Such regulatory elements required for expression include promoter sequences, transcription initiation sequences and, optionally, enhancer sequences.

The Pi-CO39(t) polypeptide(s) produced by gene expression in a recombinant procaryotic or eucyarotic system may be purified according to methods known in the art. In a preferred embodiment, a commercially available expression/secretion system can be used, whereby the recombinant protein is expressed and theater secreted from the host cell, to be easily purified from the surrounding medium. If expression/secretion vectors are not used, an alternative approach involves purifying the recombinant protein by affinity separation, such as by immunological interaction with antibodies that bind specifically to the recombinant protein. Such methods are commonly used by skilled practitioners.

The present invention also provides antibodies capable of immunospecifically binding to Pi-CO39(t)-encoded polypeptides. Polyclonal or monoclonal antibodies are prepared according to standard methods. Monoclonal antibodies may be prepared according to general methods of Köhler and Milstein, following standard protocols. Recombinant monoclonal antibodies also may be prepared in accordance with standard methods, e.g., via phage display libraries of genes encoding human or animal antibodies or fragments, which may be panned with plant proteins. In a preferred embodiment, antibodies are prepared, which react immunospecifically with various epitopes of the Pi-CO39(t)-encoded polypeptides.

Polyclonal or monoclonal antibodies that immunospecifically interact with one or more of the polypeptides encoded by Pi-CO39(t) can be utilized for identifying and purifying such proteins. For example, antibodies may be utilized for affinity separation of proteins with which they immunospecifically interact. Antibodies may also be used to immunoprecipitate proteins from a sample containing a mixture of proteins and other biological molecules.

3. Transgenic Plants

The present invention includes transgenic plants comprising one or more copies of the Pi-CO39(t) gene or genes. This is accomplished by transforming plant cells with a transgene that comprises part of all of a Pi-CO39(t) coding sequence, controlled by either native or recombinant regulatory sequences, as described below. Transgenic plants of any species are included in the invention. Preferred are monocots having susceptibility to pathogenic species of Magnaporthe; these include rice, wheat, barley, maize and other cereal crops, as well as turfgrasses such as Lolium perenne L., Lolium multiflorium Lam. and the cereal Setaria italica.

Transgenic plants can be generated using standard plant transformation methods known to those skilled in the art. These include, but are not limited to, Agrobacterium vectors, polyethylene glycol treatment of protoplasts, biolistic DNA delivery, UV laser microbeam, gemini virus vectors or other plant viral vectors, calcium phosphate treatment of protoplasts, electroporation of isolated protoplasts, agitation of cell suspensions in solution with microbeads coated with the transforming DNA, agitation of cell suspension in solution with silicon fibers coated with transforming DNA, direct DNA uptake, liposome-mediated DNA uptake, and the like. Such methods have been published in the art. See, e.g., Methods for Plant Molecular Biology (Weissbach & Weissbach, eds., 1988); Methods in Plant Molecular Biology (Schuler & Zielinski, eds., 1989); Plant Molecular Biology Manual (Gelvin, Schilperoort, Verma, eds., 1993); and Methods in Plant Molecular Biology—A Laboratorv Manual (Maliga, Klessig, Cashmore, Gruissem & Varner, eds., 1994).

The method of transformation depends upon the plant to be transformed. Agrobacterium vectors are often used to transform dicot species. Agrobacterium binary vectors include, but are not limited to, BIN19 (Bevan, 1984) and derivatives thereof, the pBI vector series (Jefferson et al., 1987), and binary vectors pGA482 and pGA492 (An, 1986) For transformation of monocot species, biolistic bombardment with particles coated with transforming DNA and silicon fibers coated with transforming DNA are often useful for nuclear transformation. Alternatively, Agrobacterium “superbinary” vectors have been used successfully for the transformation of rice, maize and various other monocot species.

DNA constructs for transforming a selected plant comprise a coding sequence of interest operably linked to appropriate 5′ (e.g., promoters and translational regulatory sequences) and 3′ regulatory sequences (e.g., terminators). In a preferred embodiment, the Pi-CO39(t) gene under control of its own 5′ and 3′ regulatory elements is utilized.

In an alternative embodiment, the coding region of the gene is placed under a powerful constitutive promoter, such as the Cauliflower Mosaic Virus (CaMV) 35S promoter or the figwort mosaic virus 35S promoter. Other constitutive promoters contemplated for use in the present invention include, but are not limited to: T-DNA mannopine synthetase, nopaline synthase (NOS) and octopine syntiase (OCS) promoters. In preferred embodiments, a strong monocot promoter is used, for example, the maize ubiquitin promoter, the rice actin promoter or the rice tubulin promoter (Jeon et al., Plant Physiology. 123:1005-14, 2000).

Transgenic plants expressing Pi-CO39(t) coding sequences under an inducible promoter are also contemplated to be within the scope of the present invention. Inducible plant promoters include the tetracycline repressor/operator controlled promoter, the heat shock gene promoters, stress (e.g., wounding)-induced promoters, defense responsive gene promoters (e.g. phenylalanine ammonia lyase genes), wound induced gene promoters (e.g. hydroxyproline rich cell wall protein genes), chemically-inducible gene promoters (e.g., nitrate reductase genes, glucanase genes, chitanase genes, etc.) and dark-inducible gene promoters (e.g., asparagine synthetase gene) to name a few. The use of pathogen- and wound-inducible promoters is described in more detail below.

Tissue specific and development-specific promoters are also contemplated for use in the present invention. Examples of these include, but are not limited to: the ribulose bisphosphate carboxylase (RuBisCo) small subunit gene promoters or chlorophyll a/b binding protein (CAB) gene promoters for expression in photosynthetic tissue; the various seed storage protein gene promoters for expression in seeds; and the root-specific glutamine synthetase gene promoters where expression in roots is desired.

The coding region is also operably linked to an appropriate 3′ regulatory sequence. In a preferred embodiment, the nopaline synthetase polyadenylation region (NOS) is used. Other useful 3′ regulatory regions include, but are not limited to the octopine (OCS) polyadenylation region.

Using an Agrobacterium binary vector system for transformation, the selected coding region, under control of appropriate regulatory elements, is linked to a nuclear drug resistance marker, such as kanamycin resistance. Other useful selectable marker systems include, but are not limited to: other genes that confer antibiotic or herbicide resistances (e.g., resistance to hygromycin or bialaphos) or herbicide resistance (e.g., resistance to sulfonylurea, phosphinothricin, or glyphosate).

Plants are transformed and thereafter screened for one or more properties, including the presence of Pi-CO39(t) protein, Pi-CO39(t) mRNA, or enhanced resistance to plant pathogens, in particular Magnaporthe grisea. It should be recognized that the amount of expression, as well as the tissue-specific pattern of expression of the transgenes in transformed plants can vary depending on the position of their insertion into the nuclear genome. Such positional effects are well known in the art. For this reason, several nuclear transformants should be regenerated and tested for expression of the transgene.

Transgenic plants that exhibit one or more of the aforementioned desirable phenotypes can be used for plant breeding, or directly in agricultural or horticultural applications. Plants containing one transgene may also be crossed with plants containing a complementary transgene in order to produce plants with enhanced or combined phenotypes.

### B. Uses of Pi-CO39(t) Nucleic Acids, Proteins and Transgenic Plants

The potential of recombinant genetic engineering methods to enhance disease resistance in agronomically important plants has received considerable attention in recent years. Protocols are currently available for the stable introduction of genes into plants (as described in detail above), as well as for augmentation of gene expression. The present invention provides nucleic acid molecules which, upon stable introduction into a recipient plant, can enhance the plant's ability to resist pathogen attack.

1. Pi-CO39(t) Nucleic Acids and Transgenic Plants

Pi-CO39(t) nucleic acids (genomic clones or cDNAs) may be used fov a variety of purposes in accordance with the present invention. The DNA, RNA, or fragments thereof may be used as probes to detect the presence of and/or expression of Pi-CO39(t) genes. Methods in which Pi-CO39(t) 19Unucleic acids may be utilized as probes for such assays include, but are not limited to: (1) in situ hybridization; (2) Southern hybridization (3) northern hybridization; and (4) assorted amplification reactions such as polymerase chain reactions (PCR). The Pi-CO39(t) nucleic acids of the invention may also be utilized as probes to identify homologs from other rice cultivars and from other plant species. As described above, Pi-CO39(t) nucleic acids are also used to advantage to produce large quantities of substantially pure Pi-CO39(t) proteins, or selected portions thereof.

Of greater significance, however, is the use of Pi-CO39(t) nucleic acidw to broaden the scope of resistance of rice cultivars and other plant species to a variety of M. grisea isolates, and even to plant pathogens other than M. grisea. For instance, in one embodiment of the invention, the Pi-CO39(t) coding region is operably linked to a heterologous promoter, preferably one that is either generally pathogen inducible (i.e. inducible upon challenge by a broad range of pathogens) or wound inducible. Such promoters include, but are not limited to:

a) promoters of genes encoding lipoxygenases (preferably from plants, most preferably from rice, e.g., Peng et al., J. Biol. Chem. 269: 3755-3761, 1994; Peng et al., Abstract presented at the general meeting of the International Program on Rice Biotechnology, Malacca, Malaysia, Sep. 15-19, 1997);

b) promoters of genes encoding peroxidases (preferably from plants, most preferably from rice, e.g., Chittoor et al., Mol. Plant-Microbe Interactions 10: 861-871, 1997);

c) promoters of genes encoding hydroxymethylglutaryl-CoA reductase (preferably from plants, most preferably from rice, e.g., Nelson et al., Plant Mol. Biol. 25: 401-412, 1994);

d) promoters of genes encoding phenylalanine ammonia lyase (preferably from rice; e.g., Lamb et al., Abstract of the general meeting of the International Program on Rice Biotechnology, Malacca, Malaysia, Sep. 15-19, 1997);

e) promoters of genes encoding glutathione-S-transferase (preferably from plants, most preferably from rice, or alternatively, the PRP1 promoter from potato);

f) promoters from pollen-specific genes, such as corn Zmg13, which has been show to be expressed in rice transgenic pollen carrying the corn gene (Aldemita et al., Abstract of the general meeting of the International Program on Rice Biotechnology, Malacca, Malaysia, Sept. 15-19, 1997);

g) promoters from genes encoding chitinases (preferably from plants, most preferably from rice; e.g., Zhu & Lamb, Mol. Gen. Genet. 226: 289-296, 1991);

h) promoters from genes induced early (within 5 hours post-inoculation) in the interaction of M. grisea and rice (e.g., Bhargava & Hamer, Abstract B-10, 8th International Congress Molecular Plant Microbe Interactions, Knoxville, Ten. July 14-19, 1996);

i) promoters from plant (preferably rice) viral genes, either contained on a bacterial plasmid or on a plant viral vector, as described by Hammond-Kosack et al., Mol. Plant-Microbe Interactions 8: 181-185 (1994);

j) promoters from genes involved in the plant (preferably rice) respiratory burst (e.g., Groom et al., Plant J. 10(3): 515-522, 1996); and

k) promoters from plant (preferably rice) anthocyanin pathway genes (e.g., Reddy, pp 341-352 in Rice Genetics III, supra; Reddy et al., Abstract of the general meeting of the International Program on Rice Biotechnology, Malacca, Malaysia, Sep. 15-19, 1997).

The chimeric gene is then used to transform the plant of interest. Upon wounding or challenge with a plant pathogen, the resulting transgenic plants would be induced to produce the Pi-CO39(t) gene product, thereby triggering the R gene defense response. In this embodiment, care must be taken to avoid using a promoter that is induced by necrosis, since use of such a promoter could result in a self-perpetuating hypersensitive response that may be lethal to the plant (see, e.g., Kim et al., Proc. Natl. Acad. Sci. USA 91: 10445-10449, 1994).

A preferred embodiment utilizes the Pi-CO39(t) gene controlled by its own regulatory sequences, rendering it either constitutively expressed or inducible by the product of the corresponding AVR1-CO39 avirulence gene that has been cloned. In this embodiment, the selected plant is transformed and a disease resistance response is generated by exposing the transformed plant to either or both of (1) the gene product of the AVR1-CO39 gene or (2) a suspension of non-pathogenic recombinant microorganisms (e.g., epiphytic or endophytic bacteria, or even a non-pathogenic stain of Magnaporthe) comprising the AVR1-CO39 gene. Upon pathogen attack, two levels of protection can occur in the transgenic plant: (1) the gene product produced by the recombinant epiphytes or endophytes triggers an interaction on the plant surface that prevents further penetration by the pathogen (e.g., the fungal conidia develop appresoria, but do not develop penetration pegs); or (2) the gene product produced by the recombinant epiphytes is carried into the plant tissue at the wound site, where it interacts with the corresponding R gene; product and induces an internal disease defense response. Thus, this pre-treatment confers resistance to Maganporthe isolates (and, presumably, other plant pathogens) which normally are virulent on those cultivars. These methods are described in detail in PCT US99/04047 and commonly-owned co-pending U.S. application Ser. No. 09/257,585.

In another embodiment, plants themselves can be co-transformed with Pi-CO39(t) and the fungal AVR1-CO39 gene. Co-expression of the genes results in an internal triggering mechanism to induce the resistance response.

It should be noted that constitutive production of the Pi-CO39(t) gene product may induce resistance without the aid of the AVR1-CO39 gene. Accordingly, it may not be necessary in all instances to use an inducible system.

2. Pi-CO39(t) Proteins and Antibodies

Purified gene products of Pi-CO39(t), or fragments thereof, may be used to produce polyclonal or monoclonal antibodies, which also may serve as sensitive detection reagents for the presence and accumulation of Pi-CO39(t) polypeptides. Polyclonal or monoclonal antibodies immunologically specific for Pi-CO39(t) polypeptides may be used in a variety of assays designed to detect and quantitate the proteins. Such assays include, but are not limited to: (1) flow cytometric analysis; (2) immunochemical localization of expressed proteins in cells or tissues; and (3) immunoblot analysis (e.g., dot blot, Western blot) of extracts from various cells and tissues. Additionally, as described above, antibodies can be used for purification of Pi-CO39(t) polypeptides (e.g., affinity column purification, inmmunoprecipitation).

The following specific examples are provided to illustrate embodiments of the invention. They are not intended to limit the scope of the invention in any way.

### EXAMPLE 1

### Genetic Mapping of Locus Comprising Blast Resistance Genes Pi-CO39(t) in Rice Line CO39 and Identification of Large Insert Clones Linked to Resistance

Materials & Methods

M. grisea strains. Isolate ‘Guy 11’—virulent on CO39 and 51583—originally collected from a diseased rice plant in French Guyana, was provided by J L Notteghem (Institute de Recherches Agronomiques Tropicales Montpellier Cedex, France). Progeny 6082, avirulent on CO39 and virulent on 51583, was produced by crossing isolate 2539 and Guy 11 as described in Smith and Leong (1994). Guy 11 transformant (G11XF18-1(0) A#6), carrying aviruilence gene, AVR1-CO39 was produced by Farman and Leong (1998). Fungal cultures were stored at −20° C. in 6-mm chromatography paper discs (Whatman) as described by Valent et al. (1991).

Seed germination, inoculation procedure and disease severity rati˜gs. Seeds of rice genotypes CO39 and 51583 were procured from different sources as described in Smith and Leong (1994). Seeds were surface sterilized in 10% bleach and germinated on petri plates lined with moist blotting paper. Individual seedlings, usually 5-6 days after germination, were transplanted to disposable plastic square cubicles. The growth medium was Bacto professional planting mix (Michigan Peat Co., Housten, Tex.). Seedlings were flooded with water continuously. Seedlings were grown for 3-4 weeks in a growth chamber equipped with full spectrum white light GROW-LOX bulbs (230 μE/m/sec) set for 16 h photoperiods. Day/night temperatures were 28° C./21° C., respectively, and percent relative humidity was 33%. Plants were inoculated at the three leaf stage, 20-25 days after transplanting.

Inoculum was prepared by growing each isolate on oatmeal agar plates under full spectrum white light bulbs (Sylvania GRO-LOX 20W) (20-55 μE/m/sec) at 22° C. for 15-20 days. Spores were detached by gently rubbing the agar surface with a bent glass rod after adding 5 ml of 0.2% gelatin solution and sprayed on seedlings at a concentration of 10⁴ spores/ml. Plants were placed in a plastic bag and tied from the top. Bags were removed after 24 hours.

Seven days after inoculation, ratings for disease severity were recorded on the youngest leaf that was expanded at the time of inoculation. We based our judgements on the ability of affected tissue to support conidiation under high humidity conditions and thus complete the disease cycle (Valent et al. 1991). To describe the interaction phenotype, a numerical system similar to that of Yu et al. (1987) has been adopted in this study: Type 0—no visible symptoms; Type 1—small dark brown, pin point-sized, non-sporulating lesions Type 2—dark brown, non-sporulating lesions 2-3 mm in length, Type 3—circular, sporulating lesions with the tan centers and dark brown margins; Type 4—large diamond-shaped, sporulating with tan centers and dark brown margins. Reaction phenotypes with lesion types 0, 1 and 2 were considered resistant while those producing reaction types 3 and 4 were considered susceptible.

Inheritance experiments. Reciprocal crosses between CO39 (resistant) and 51583 (susceptible) were made. Each F₁ seedling was tested for disease reaction and grown to maturity to produce a F₂ population. Individual plant progenies derived from a single F₂plant constituted a single F₃ family. Segregation data were tested for Mendelian inheritance using the Chi-square method. Twelve homozygous resistant F₃ lines and six homozygous susceptible F₃ lines were selected for preliminary mapping using bulked segregant analysis. Equal amounts of DNA from each line were combined to make resistant and susceptible pools of DNA for bulked segregant analysis (Michelmore et al. 1991).

Microsatellite analysis. Microsatellite primer pairs for 20 loci on rice chromosomes 4, 6, 11 and 12 were synthesized using an ABI DNA synthesizer according to the manufacturers instructions. Total genomic DNA (75-100 ng) of CO39 and 51583 as well as of pools from 10 resistant or 6 susceptible F₃ progenies was used as template for PCR amplification by initial incubation at 92° C. for 5 min followed by 35 cycles of denaturation at 92° C. for 1 min; annealing at 55° C. for 1 min and extension at 72° C. for 2 min and a final 4 min extension at 72° C. Polymorphisms between the PCR products from DNA of parents and the resistant or susceptible F₃ progeny were analysed by electrophoresing the PCR products on 40-cm-long 4.5% denaturing polyacrylamide gels (PAGE) run for 1.5 h at 75 constant watts and silver stained according to the manufacturer's instructions (Promega). The polymorphic and co-segregating marker, RM202, was tested with DNA of a large number of individual F₂ progenies. The PCR products for the RM202 marker were resolved in 3.0% MetaPhor agarose (FMC Bioproducts) prepared in 0.5×TBE and run at 10.0 V/cm for 5 h.

Plant DNA extraction, restriction digestion, electrophoresis and Southern analysis. Plant DNA was prepared from fresh or frozen leaf tissue from individual plants according to the method of McCouch et al. (1988). Total genomic DNA of CO39 and 51583 was digested with several 6 bp restriction endonucleases to detect polymorphisms. The parental DNA as well as of individual F₂ progenies was digested with EcoR1, BamH1, EcoRV, Hind111, Dra1, Nae1 for mapping. Genomic and cDNA probes as well as microsatellite primer pair sequences from the high-density e Cornell maps (Causse et al. 1994; Chen et al. 1997) and the Japanese Rice Genome Program (Harushima et al. 1998) were selected. Electrophoresis and Southern hybridization analysis were done according to Farman & Leong (1998). Hybridization methods were modified from Amasino (1986). The hybridization buffer was prepared according to the Amasino protocol, but without the PEG and NaCl and with reduced concentrations of NaHPO₄: 0.125M NaHPO₄, 7% SDS, 50% formamide, 1.0 mM EDTA, pH 7.2. High stringency conditions were used (42° C., 16 h). Post hybridization washes were: 2×SSC+0.1% SDS and in 0.1×SSC+1.0% SDS at 42° C. for 15 min each and in 0.1×SSC+0.1% SDS at 65° C. for 20 min, respectively. The final washing conditions were of greater stringency than were the hybridization conditions, giving a T^{m} of 68° C. Thus, greater than 95% homology would be required to maintain a hybrid. None of the post hybridization phosphate- containing buffers described in Amasino (1986) were employed.

Construction of Large insert DNA library. Indica rice variety CO39, which is a source of blast resistance locus in this study, was the plant material used for constructing a large insert DNA library.

The conventional binary cosmid vector pCLDO4541, designed for Agrobacterium-mediated plant transformation (Bent et al., 1994), was selected as a cloning vector. The vector has a cos site and a polylinker from pBluescript SK/KS which can facilitate cloning of foreign DNA at five restriction sites (Cal1, Hind111, EcoR1, BamH1 and Xba1). PCLDO4541 has been used for stable cloning and maintenance of large DNA inserts without any rearrangements (Tao and Zhang, 1998; Wu et al., 2000). The preparation of vector DNA, restriction digestion, and dephosphorylation were done according to Zhang et al. (1996).

Isolation of high molecular weight DNA, partial digestion and size selection. Rice seedlings were grown for 2-3 weeks, etiolated for 24-36 hrs under complete darkness and 25-30 g of fresh leaf tissue was used for nuclei isolation as per the “nuclei isolation method” described by Zhang et al (1995). Fresh leaf tissue (20 g) was ground into fine powder using a mortar and pestle in liquid nitrogen and immediately transferred into ice-cold homogenization buffer (HB: 10 mM trizma base, 80 mM KCl, 1 mM spermidine, 1 mM spermine, pH 9.4,0.5 M sucrose) plus 0.5% Triton®X-100 and 0.15% β-mercaptoethanol, mixed well and filtered through cheesecloth and Miracloth (Calbiochem-Novabiochem, La Jolla, Calif., USA) and centrifuged at 1800×g for 25 min. After washing in wash buffer the nuclear pellet was resuspended in 500 μl of HB and processed for microbead preparation according to Zhang et al. (1995). Nuclei were embedded in low melting agarose microbeads. The microbeads were incubated in lysis buffer (0.5 M EDTA, pH 9.0, 1% sodium lauryl sarcosine, 0.1 mg/ml proteinase K) at 55° C. for 36 h, followed by treatment with TE buffer (10 mM Tris-HCl, 1 mM EDTA, pH 8.0) plus 0.1 mM phenylmethylsulfonyl fluoride (PMSF) for I h, three times. The mirobeads were finally kept in TE buffer. Partial digestion of high molecular weight DNA with BamH1 was performed in the beads according to Zhang et al (1995).

Size fractionation of partially digested DNA in microbeads. Partially digested DNA was size selected using a modification of the method of Osoegawa et al. (1998). Microbeads containing the partially digested DNA were applied to the central well of a 1% low melting temperature agarose (SeaPlaque®GTG® agarose) gel and the lambda concatamer size markers in the flanking wells. The fractionation was done in BIORAD CHEF-DR®III sysytem. The DNA was separated in three different stages using a pulse direction of 120°. The first direction of the field allowed the DNA to migrate 1-1.5 cm from the $wells toward the top edge of the gel by electrophoresis at 5.0 V/cm for 6 h with a pulse time of 15 s. In the 2nd step, the CHEF running conditions were the same but current was reversed in order to bring all the fragments remaining in the gel back toward the wells. Small DNA fragments (≈50-100 kb) which moved beyond the wells were excised and discarded. Fresh 1% low melting agarose solution was poured into the excised portion of the gel. New marker DNA was loaded into the flanking wells not previously used. The high molecular weight DNA was then resolved at 6 V/cm for 16 h with an increasing pulse time of 0.1 s-to-40 s. Running buffer (0.5×TBE), CHEF chamber temperature (12° C.) and the angle (120°) were the same for all three steps. After electrophoresis, the flanking marker lanes along with peripheral portion of both the sides of high MW rice DNA lane were cut, stained with ethidium bromide, destained, washed in distilled water thoroughly, and aligned with the digested genomic DNA lane to mark the position of selected size ranges. Gel slices were cut at an interval of 0.5 cm to obtain gel slices containing DNA in the range of 150-500 kb.

Ligation, transformation, and storage of the library. Gel slices containing the size (200-300 kb) selected DNA were dialysed against TE buffer and the agarose digested with gelase (Epicentre Technologies, Madison, Wis.) as reported by Zhang et al. (1996). Insert DNA, which was BamH1 digested, was ligated to dephosphorylated vector DNA in 1:3 ratio at 16° C. overnight. The ligated DNA was transformed into Electromax DH10B E. coli cells (Gibco BRL, Grand Island, N.Y.) by electroporation using the Cell Porator and Voltage Booster system (Gibco BRL). The Cell Porator settings were the same as recommended by the manufacturer. Transformed cells were incubated at 37° C. for 1 h in 1 ml of SOC medium (2% Bacto typtopane, 0.5% Bacto Yeast Extrect, 10 mM NaCL, 2.5 mM KCL, 10 mMMgCl₂, 10 mM MgSO₄ and 20 mM glucose, pH 7.0), and then plated on LB plates containing X-gal (80 μg/ml), IPTG(0.55 mM), and tetracycline (15 μg/l). The pates were incubated at 37° C. for 18-20 hrs for blue/white color development. The white colonies were picked with toothpicks and transferred to 384-well microtitre dishes containing 70 μl LB cell freezing medium (36 mM K₂HPO₄, 13.2 mM KH₂PO₄, 1.7 mM sodium citrate, 0.4 mM MgSO₄, 6.8 mM (NH₄)₂, 4.4% glycerol LB 25 g/l). The microtitre dishes were incubated at 37° C. for 24 h. The library was replicated and stored at −80° C.

Arraying of clones on high density filters. Large DNA insert clones were arrayed onto high density filters using a 384-pin Biomeck 2000 Robotics Workstation (Beckman, Fullerton, Calif). Each arrayed filter (7.5 cm×11 cm) contained duplicate copies of each clone's DNA in a 3×3 grid with no colony in the center (1,536 clones/filter). The library consisted of 23,040 clones arrayed on 15 filters. The filters were processed according to Zhang et al. (1996) and stored at 4° C. before probing.

Identification of clones linked to blast resistance locus (Pi-CO39(t)). The library was probed with a co-segregating RFLP marker RGA38 using the following conditions for probe preparation and hybridization.

BAC filter probing and hybridization. Probes were labelled using random hexamer Oligolabelling Kit (Pharmacia) except that 1 ng uncut lambda DNA was included along with the probe DNA and hybridized at 42° C. in 50% formamide, 7% SDS, 0.125 M Na₂HPO₄ (pH7.2) and 1 mM Na EDTA overnight. The BAC filters were washed three times for 20 min each in 2×SSC+0.1% SDS at 42° C. for 1st wash and in 0.5×SSC+0.l% SDS and in 0.1×SSC+0.1% SDS at 65° C. for 2nd and 3rd washes, respectively. The filters were exposed to Phosphor screen and scanned after 30 min-1 h exposure using a Packard Cyclone Storage system. Overnight exposures were also scanned to see the background hybridization of all colonies caused by hybridization of lambda to the vector to facilitate the determination of the clone address. Mniprep DNA from recombinant BAC clones was isolated using a modification of alkaines lysis method described in Zhang et al. (1996). Large scale isolation of BAC DNA was done according to the QIAGEN® large-construct kit

Results

Genetic Analysis of resistance in CO39. Resistance phenotype of the F₁, individual F₂ progenies, and F₃ families was consistently of reaction type 1 and the reaction phenotype of the susceptible seedlings was reaction type 4 in all segregating populations. All the reciprocal F₁ tested i.e. CO39×51583 or 51583×CO39 were resistant to M. grisea strain 6082, thereby, indicating that resistance is controlled by a dominant locus in the nuclear genome of CO39. Resistance to M. grisea progeny 6082 in three F₂ populations consisting of 604 F₂ progenies derived from different F₁ plants, segregated as a single dominant locus (Table 1).

TABLE 1
Segregation f resistance in F₁ and F₂ generations of crosses between
indica lines CO39(R) and 51583(S)

| | M. grisea | No. Plants | | Expected | | |
|---|---|---|---|---|---|---|
| Cross | strain | R | S | ratio | x² | P value |
| F₁ (CO39X51583) | 6082 | 5 | 0 | all R | — | — |
| F₁ (CO39X51583) | Guy 11 | 0 | 4 | all S | — | — |
| F₁ (51583XCO39 | 6082 | 4 | 0 | all R | — | — |
| F₁ (51583XCO39) | Guy 11 | 0 | 3 | all S | — | — |
| F₂ Family 1 | 6082 | 205 | 59 | 3:1 | 0.99 | 0.5-0.3 |
| F₂ Family 2 | 6082 | 150 | 47 | 3:1 | 0.14 | 0.7-0.5 |
| F₂ Family 3 | 6082 | 109 | 34 | 3:1 | 0.11 | 0.7-0.5 |
| F₂ Family 4 | Guy 11 | 180 | 55 | 3:1 | 0.32 | 0.7-0.5 |
| | (AVR | | | | | |
| | 1-CO39) | | | | | |
| Total F₂ | | 644 | 195 | 3:1 | 1.38 | 0.3-0.2 |
| Progenies | | | | | | |

Resistance to a Guy 11 (AVR1-CO39) transformant in one F₂ population consisting of 235 F₂ progenies also segregated as a single dominant locus (Table 1). Inheritance of resistance was also confirmed in F3 families derived from the F₂ populations used for mapping the resistance locus. Testing of 78 F₃ families against the Guy 11 (AVR1-CO39) transformant and 59 F₃ families to M. grisea progeny 6082 also showed that resistance is controlled by a single dominant locus. The segregation ratio of F₃ families was: all resistant: segregating for resistance: all susceptible and fit into 1:2:1 ratio (Table 2). The disease resistance locus in CO39 was designated as Pi-CO39(t).

TABLE 2
Segregation of blast resistance in F₃ families of crosses between indica
rice lines CO39 and 51583

| No. F₃ | M. grisea | | | |
|---|---|---|---|---|
| Families | strain | R:Seg:S | 1:2:1 | P value |
| 78 | Guy11 (AVR1- | 16:44:18 | 1.38 | 0.5-0.6 |
| | CO39) | | | |
| 59 | 6082 | 13:34:12 | 1.40 | 0.5-0.6 |

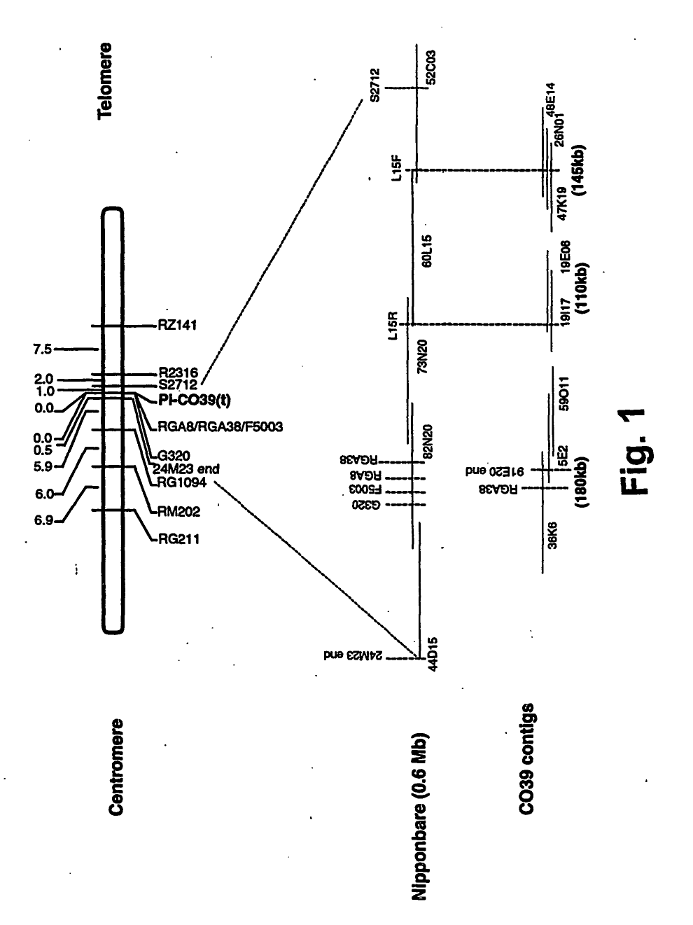

Mapping of resistance gene(s) Pi-CO39(t). Microsatellite loci were randomly selected from four rice chromosomes 4, 6, 11 and 12 that were previously shown to carry many disease resistance genes (McCouch et al., 1994). Most of the test loci were found to be polymorphic between CO39 and 51583. Microsatellite locus, RM202, co-segregated in bulked segregant analysis of resistant and susceptible F₃ progenies. The resistance locus was fine mapped on chromosome 11 in two different F₂ populations consisting of 154 and 103 progenies, respectively. The program Mapmaker Version 2.0 (Lander et al., 1987) was used to determine association between molecular markers and the resistance locus using Kosambi Centimorgan function and LOD value of 3.0. The genetic map of resistance locus with respect to co-segregating markers is presented in FIG. 1. The resistance locus, Pi-CO39(t), was mapped between RZ141 (7.5 cM) and R2316 (3.0 cM) on one side (telomeric end) and RG211 (18.8 cM), RM202 (11.9 cM) and RG1094 (5.9 cM) on the centromeric end of the short arm of chromosome 11. Three markers, RGA8, RGA38 and G320 perfectly co-segregated with Pi-CO39(t) among all the F₂ progenies tested. RGA8 and RGA38 are resistance gene analogues mapped on chromosome 11 of rice (Mago et al., 1999). These three markers have been tested on 400 individual F₂ progenies. All the resistant F₂ progenies recombinant for different mapping markers were confirmed to be of the genotype RR or Rr. The frequency of recombination for different markers is given in Table 3.

TABLE 3
Cross-over among markers:

| Marker | Number of Crossovers |
|---|---|
| RM202 | 43 |
| RG1094 | 29 |
| RZ141 | 39 |
| RG211 | 62 |
| R2316 | 15 |
| RGA8 | 0 |
| RGA38 | 0 |
| G320 | 0 |

C nstruction f a large DNA insert library f CO39. The large DNA insert library of the disease resistant genotype CO39 used in this study, was constructed from high molecular weight DNA isolated from nuclei in which more than 95% of the chloroplasts and mitochondria were removed during the preparation of nuclei. The DNA embedded in microbeads was partially digested with BamH1, size selected and ligated using a single size selection of 200-300 kb.

The library consists of 23,040 clones arrayed in 60 384-well microtitre dishes. About 65 random clones were selected, digested with Not 1 because Not1 cuts out the cloned insert DNA. The Not1 digested BAC DNAs were separated by pulse-field gel (PFG) electrophoresis (initial pulse time: 5 s; final pulse time: 15 s, 6 V/cm, 11° C., 120°, 15 h) to determine the sizes of the cloned fragments. A lambda concatemer ladder from New England Biolabs® inc. was used as a PFG marker. Insert size of recombinant clones ranged from 60-185 with an average insert size of 100 kb. All the 65 clones contained foreign DNA and one or more Not1 site within the insert DNA. This library represented about 5× rice genome equivalents with a theoretical probability of 95% coverage of each gene. Contamination of chloroplast DNA in the library was less than 1% as determined by probing with a fragment of the chloroplast gene, rbcL.

Identification of large insert clones linked to blast resistance locus, Pi-CO39(t). The library was screened with co-segregating probe RGA38 and three positive clones were identified. The identification of three clones hybridizing to RGA38 (corresponding to a single fragment on genomic DNA) is consistent with the number of clones expected from a 5× library of CO39. Restriction enzyme mapping, PCR and Southern analysis of positive clones, the genomic DNA of mapping rice parental genotypes, CO39 and 51583, was done to confirm that the clones are from the right location in the genome. Southern hybridization analysis of RGA38-positive clones digested with HindIII showed the presence of 7.5 kb fragment which was mapped in the resistant line CO39 and shown to co-segregate with Pi-CO39(t). Three RGA38 clones were extensively overlapping as shown by the HindIII restriction digestion fragment patterns. The largest clone, 36K6 (100 kb) was selected for shotgun sequencing. The CO39 library was probed with an amplicon derived from end sequence of a Nipponbare BAC clone OSJNBa91E20 which is part of contig 43 (see below) belonging to a syntenic region in the Nipponbare. Five positive clones in the CO39 library, including 36K6 and 4A14 which were positive to RGA 38, were identified. Ends of the largest clone from CO39, 5E2 (70 kb) were sequenced. Additional clones from the library are being identified to extend the contig. The CO39 library could not be screened with G320 because it is not detected by hybridization to the CO39 genome.

Screening of BAC library from Japonica rice variety, Nipponbare. Rice variety, Nipponbare is the model rice genome being sequenced by the International Rice Genome Sequencing Project Consortiun. Extensive resources are available for this genome project, including several large fragment libraries which have been end-sequenced and fingerprinted, a YAC physical map, and contig information obtained through overlapping fingerprinted clones. We screened a HindIII BAC library of Nipponbare consisting of 36,864 clones with an average insert size of 128.5 kb markers with the three markers co-segregating with Pi-CO39(t). Several positive clones were identified including five clones hybridizing to all the three co-segregating markers, RGA8, RGA38 & G320. All the BAC clones hybridizing to three markers were in one fingerprint contig no. 43. All five of the positive clones were confirmed to contain the expected restriction fragments associated with the markers by restriction digestion and probing with marker clones as well as PCR to identify the expected amplicon associated with the markers. Contig 43 consists of 77 BAC clones. The tentative physical location of RGA8, RGA38 and G320 has been assigned by restriction and Southern hybridization analysis using a subset of representative BAC clones from contig 43. A minimum tiling path of three BAC clones was developed for sequencing (FIG. 1, lower panel). Information about contigs and BAC end sequences is available on the web site of Clemson University Genome Center (www.genome.clemson.edu). End sequences from the BAC clones in contig 43 were used to walk in the CO39 library. Nipponbare is susceptible to the M. grisea strain 6082 and Guy 11 (AVR1-CO39) transformant.

### EXAMPLE 2

### Sequence Information for Selected Segments of BAC Clones Containing Co-Segregating Markers

The following sequence information has been obtained in accordance with the present invention.

CO39.RGA8seq (SEQ ID NO:1): PCR product (360 bp) amplified from rice variety CO39 using primer sequences from the published RGA8 sequence. 84% identity to the published RGA8 sequence.

CO39.RGA38seq (SEQ ID NO:2): DNA fragment of 493 bp cloned from rice variety CO39 using primer sequences from the published RGA38 sequence. 97% identity to the published sequence.

RGA38 contig.26Nippon (SEQ ID NO:3): BAC clone 82N20 from susceptible rice variety Nipponbare being sequenced and one contiguous sequence (contig) of 15.6 kb contains RGA38 sequence from 13334-13834 with 97% nucleotide identity to published RGA38 and CO39 RGA38.

RGA8 contig.30Nipp (SEQ ID NO:4): BAC clone 82N20 from susceptible rice variety Nipponbare being sequenced and one contiguous sequence (contig) of 17.87 kb contains RGA8 sequence from 13334-13834 with 88% nucleotide identity to published RGA8 sequence and 97% identity to CO39RGA8 360 bp sequence.

A preliminary sequence analysis revealed resistance like gene in contig.491 from clone 36K6 (K6P36, SEQ ID NO:5). The CO39.RGA38seq is part of a RPR1-like gene in rice. RPR1 is a defense response gene induced by an agricultural chemical probenazole for protecting rice plants against pathogens, particularly Magnaporthe grisea. The RPR1 published gene maps at 1.0 cM from our co-segregating markers RGA8/RGA38/G320 toward the telomeric end of rice chromosome 11 and belongs to nucleotide binding site and leucine rich repeats (NBS-LRR) class of resistance genes (Sakamoto et al., 1999; Plant Mol. Biol. 40:847-855). The published RPR1 gene does not provide strain specific resistance. The RPR1-like gene in BAC clone 36K6 (from resistant rice CO39) is a single exon in contig.334CO39 from 5224-2501 (bottom strand) and has 62% identity at the amino acid level to the published RPR1 gene.

Another disease resistance like gene also appears in contig.491 from clone 36K6 (SEQ ID NO:5). The gene is like the Xa-1 gene and has 42/o identity at the amino acid level. The Xa-1 gene has been cloned in rice and confers a high level of specific resistance to a Japanese race 1 of the bacterial pathogen, Xanthomonas oryzae pv. oryzae. It also belongs to NBS-LRR class of resistance genes and maps to rice chromosome 4 (Yoshimura et al., 1998 PNAS 95: 1663-1668)

### EXAMPLE 3

### Comparative DNA Sequence Analysis of Resistant and Susceptible Cultivars at the Pi-CO39(t) Locus

The relationship between the genetic map and the physical maps of the region of rice chromosome 11 associated with Pi-CO39(t) in Japonica variety Nipponbare and Indica variety CO39 is shown diagrammatically in FIG. 1. The preliminary sequence assembly of two BAC clones to give ordered contigs is shown diagrammatically in FIG. 2. Comparative sequence analysis of blast resistant (CO39 indica) and susceptible (Nipponbare japonica) rice cultivars at genomic regions co-segregating with Pi-CO39(t) showed that these two haplotypes are substantially diverged with respect to the relative number, size, orientation and location of resistance gene homologs within each cluster (FIG. 2). Gene prediction using GENSCAN, GeneMark.Arabidopsis and BLASTX indicated the presence of several disease resistance (NBS-LRR)-like genes with highest similarity to RPR1, Xa1 and Pi-ta. RPR1 (rice probenazole-responsive) is involved in induced resistance to blast in rice (Sakamoto et al., 1999); Xa1 confers a high level of resistance in rice to race 1 of bacterial blight (Xanthomonas oryzae pv. oryzae) in Japan (Yoshimura et al., 1998); Pi-ta is a rice blast resistance gene (Bryan et al., 2000. Plant Cell. 12: 2033-2045). All the three genes belong to non-TIR NBS-LRR subfamily of plant disease resistance genes suggested to be more abundant in monocot genomes (Meyers et al., 1999). Lack of significant synteny between the Nipponbare and CO39 haplotypes indicates a dynamic nature of these gene clusters with the potential to adapt rapidly to novel pathogen variants.

### EXAMPLE 4

### Detailed Sequence Analysis of BAC Inserts K6P36 and E2P5 from Indica Variety CO39 Large Insert Library

The complete sequences of BAC K6P36 (also referred to herein as 36K6) and E2P5 (also referred to herein as 5E2) are set forth herein as SEQ ID NO:5 and SEQ ID NO:6, respectively.

Genetic/Physical Mapping

Four microsatellites (di-, tri-, and hexa-nucleotide repeats) were identified in the 90 kb sequence of K6P36. Primers were designed from the flanking 100-110 bp unique sequence and amplified on genomic DNA of mapping parents (CO39/51583) as well as segregating progenies. Two microsatellites were monomorphic between CO39 and 51583, whereas other two were null for 51583 (no amplification).

Primers were designed from predicted genes in K6P36 sequence for fine mapping. About 250 homozygous susceptible F2 progenies have been tested with RFLP marker G320, microsatellites, gene specific primers, E2P5 clone end sequence to detect any recombination within KP636. The physical position of fine mapping markers in the K6P36 sequence is shown in Table 4.

TABLE 4
Physical Position of Markers in 36K6 Sequence

| | | Position |
|---|---|---|
| Marker | Size (Kb) | (kb from centromere-proximal end) |
| 36K6RPR1 | 4.1 | 8.1-12.0 |
| 36K6RPR1rg38 | 3.6 | 38.0-41.0 |
| 36K6Xa1Ex4 | 4.2 | 47.0-51.2 |
| 36K6Xa1(2.6) | 2.6 | 51.5-54.1 |
| 36K6Xa1Ex1 | 0.6 | 52.8-53.5 |
| M22/8 (Microsat) | 0.24 | 55.0-56.0 |
| M10L/R (Microsat) | 0.2 | 78.0-79.0 |
| 36K6PitaEx2 | 1.9 | 83.6-85.6 |
| 36K6PitaEx1 | 0.7 | 85.7-86.5 |
| 36K6Pita | 2.3 | 85.6-88.0 |
| E2P5 End | 0.48 | E2P5 End Seq. |

End sequence of Nippon BAC clone, 24M23 overlapping with the 44D15 end sequence has been mapped at 0.5 cM from Pi-CO39(t), as shown in FIG. 1.

Annotation of CO39 Clone K6P36

Sequence (90 kb) of K6P36 clone co-segregating with the disease resistance gene(s) Pi-CO39(t) in CO39 has been annotated using three gene prediction programs, Genescan.arabidopsis, GeneMark.arabidopsis, and GeneMark.rice. Comparative analysis of three programs with respect to gene length and similarity to published disease resistance genes indicated that GeneMark.arab followed by Genescan.arab are better than GeneMark.rice probably due to the fact that former two programs are better trained for predicting disease resistance-like genes (Table 5).

TABLE 5
Gene Predictions for K6P36 Sequence (90 Kb)

| | | RPR1-like | RPR1rg38 | Xa1-like | Pita-like |
|---|---|---|---|---|---|
| GeneScan.Arab | Length | 17409-7047 | 38580-41303 | 52897-44286 | 85741-69018 |
| | Exons | 8 | 1 | 6 | 16 |
| GeneMark.Arab | Length | 10896-7881 | 38580-41303 | 53552-47154 | 87376-83281 |
| | Exons | 2 | 1 | 5 | 7 |
| GeneMark.Rice | Length | 10896-7881 | 38580-41303 | 53552-47224 | Not identified |
| | Exons | 5 | 4 | 6 | Not identified |

A number of disease resistance genes have been cloned in Arabidopsis compared to rice. Four disease resistance-like genes were identified in K6P36 (Table 6). The annotation of RML1- is referred to as a Pi-ta-like gene (37% similarity). Pi-ta is a rice blast resistance gene in rice (Bryan et al., 2000, supra). All the four predicted resistance-like genes have signature conserved motifs present in the NBS-LRR class of disease resistance genes. A non-TIR sub-domain, so far found only in monocot NBS-LRR genes, is also present in the four predicted genes. Hydropathy analysis of all four genes showed that these are most likely cytoplasmic, soluble proteins. All have LRR domains, however the Pi-ta-like gene has a rudimentary LRR domain like the Pi-ta gene itself.

TABLE 6
Domain, Subdomain Predictions for K6P36 Genes

| | | | | | RNBS-D | LRR | Hydro- |
|---|---|---|---|---|---|---|---|
| | P-loop | Kinase-2a | Kinase-3a | GLPL | (Non-TIR) | Domain | pathy |
| Consensus | GVGKTT | LVVLDDWW | GSRIIITTRD | CGGLPLA | CFLYCALFPED | | |
| RPR1-Pub | GMGGLGKT | ENFLIVLDDV | NFQASRIIITTRQGDV | CQGLPLAIVSIGG | LRNCFLYCSL | LRR | Soluble |
| RPR1-like | GGLGKTT | SCLIVLDDVWD | PQASRIIITTR | CKGLPLALV | QKNCFLYCSL | LRR | Soluble |
| RPR1-rg38 | GGLGKTT | KCLIVLDDVWD | NFQATRVIITTR | CHGLPLAIV | RNCFLYCSL | LRR | Soluble |
| Xa1 | GNGGIGKTT | KKFLIVLDDV | GNMIILTTRIOS | LKGNPLAAKTVGS | LOOCVSYCSLFPK | LRR | Soluble |
| Xa1-like | GIAGVGKT | RTKKFLLVLDDVW | GNMILVTTR | NGNPLAAE | LQQCFLYCS | LRR | Soluble |
| Pita | GSGGVGKTT | RYPIIIEDLW | NNSCSRILTTEIEPV | KCGGLPLAITIT | CLKACLLYLS | Rudimentary | Soluble |
| | | | | | | LRR | |
| Pita-like | GAEGIGKT | RYFIVDDLWA | GSRIITITKVDE | KCGGSPLA | CLKTCLLYLS | Rudimentary | Soluble |
| | | | | | | LRR | |
| Pib | GMGGLGKTT | KSCLIVLDD | TSRIIVTTRKENI | CDGLPLAIVVIGG | LKSCFLYL | LRR | Soluble |

RNBS-D (non-TIR) domain is predominantly present in monocot genomes.
Pita, RPR1 and Pib: Rice blast resistance gene
Xa1: Rice bacterial leaf blight resistance gene

Analysis of Predicted Resistance-Like Genes in Rice Varieties

Popular rice varieties, Crocoderi (R), Cypress (R), Drew (R), M202 (S), IR64, Nipponbare (S), and Azucena (R) were characterized for resistance (R) or susceptibility (S) and tested for the presence/absence of predicted genes by PCR as well as genomic DNA hybridization to find any functional correlations. Two genes, Xa1-like and Pita-like, did not show any correlation, whereas RPL1 and RPL6 showed correlation for the presence of a common hybridizing fragment in susceptible genotypes. Pedigree lines of resistant variety CO39 are being tested for disease reaction and also for the presence/absence of predicted R genes to infer any correlation as well as trace the origin of the functional allele.

Analysis of clone E2P5 Together with Clone K6P36

Table 5 above lists genes predicted in the sequence of CO39 clone K6P36. Sequence analysis of clone E2P5 also predicts genes located in this clone. These are shown in Table 7 below, together with the predicted genes in clone K6P36. The predicted genes are shown diagrammatically in FIG. 2.

TABLE 7
Genes Predicted in the Sequence of CO39 Clones K6P36 and E2P5

| | | | % |
|---|---|---|---|
| Clone | Gene | Similarity to | Similarity |
| K6P36, | CODRL1 | Rice blast resistance gene RPR1 | 70 |
| E2P5 | (RPR1-like) | | |
| K6P36 | CODRL2 | Rice blast resistance gene RPR1 | 74 |
| | (RPR1-rg38) | | |
| K6P36 | CODRL3 | Rice bacterial blight resistance | 52 |
| | (Xa-like) | gene Xa1 | |
| K6P3 | CODRL4 | Rice blast resistance gene Pi-ta | 52 |
| | (Pita-like) | | |
| E2P5 | RSL1 (CSL1) | Barley serpin gene | 51 |
| E2P5 | RSL2 (CSL2) | Barley z-type serpin gene | 55 |
| E2P5 | RSL3 (CSL3) | Wheat serpin gene | 52 |

CODRL: CO39 disease resistance-like genes
RSL: Rice Serpin-like genes (most serpins are serine proteinase inhibitors and a few inhibit cysteine proteinases).

An alignment of the genomic region of CO39 included in BAC clones K6P36 and E2P5 with the corresponding area of the Nipponbare genome is shown in FIG. 3. Table 8 below provides a summary of the sequences of all polynucleotides disclosed herein, including sequences comprising the open reading frames identified in these segments of both the CO39 and Nipponbare varieties.

TABLE 8
Summary of sequences and SEQ ID Numbers for GenomeFragments
and Open Reading Frames of Rice Varieties CO39 and Nipponbare

| SEQ ID NO: | Genome Fragment or ORF |
|---|---|
| 1 | CO39.RGA8seq; PCR product amplified from CO39 using |
| | primer sequences from published RGA8 sequence |
| 2 | CO39.RGA38seq; DNA fragment cloned from CO39 using |
| | primer sequences from published RGA38 sequence |
| 3 | RGA38 contig.26Nippon; portion of BAC clone 82N20 |
| | from Nipponbare, containing RGA38 sequence as well as |
| | ORFs NBRS and NBR6 |
| 4 | RGA8 contig.30Nippon; portion of BAC clone 82N20 from |
| | Nipponbare, containing RGA8 sequence and ORF NBR7 |
| 5 | BAC clone K6P36 from rice variety CO39 |
| 6 | BAC clone E2P5 from rice variety CO39 |
| 7 | CODR1 ORF from CO39 |
| 8 | CODR2 ORF from CO39 |
| 9 | CODR3 ORF from CO39 |
| 10 | CODR4 ORF from CO39 |
| 11 | COSL1 ORF from CO39 |
| 12 | COSL2 ORF from CO39 |
| 13 | COSL3 ORF from CO39 |
| 14 | GTPase ORF from CO39 |

References

Amasino R 1986. Acceleration of nucleic acid hybridization rate by polyethylene glycol. Anal. Biochem. 152:304-307

Bent A E, B N Kunkel, D Dahlbeck, K L Brown, S R Schnidt, J Giraudat, J Leung and B Staskawicz 1994. RPS2 of Arabidopsis thaliana: A leucine-rich repeat class of plant disease resistance genes. Science 265: 1856-1860.

Bryan G T, K S Wu, L Farrall, Y Jia, H P Hershey, S A McAdams, K N Faulk, G K Donaldson, R Tarchini and B Valent 2000. tA single amino acid difference distinguishes resistant and susceptible alleles of the rice blast resistance gene Pi-ta. Plant Cell 12: 2033-2045.

Causse M, T M Fulton, Y G Cho, S N Ahn, K Wu, J Xio, J Chunwongse, Z Yu, P C Ronald, S B Harrington, G A Second, S R McCouch and S D Tanksley 1994. Saturated molecular map of the rice genome on an interspecific backcross population. Genetics 138:1251-1274.

Chen X, S Temnykh, Y Xu, Y G Cho and S R McCouch 1997. Development of a microsatellite framework map providing genome-wide coverage in rice (Oryza sativa L.) Theor. Appl. Genet. 95: 553-567.

Farman M L and S A Leong 1998. Chromosome walking to the AVR1-CO39 avirulence gene of Magnaporthe grisea: discrepancy between the physical and genetic maps. Genetics 150: 1049-1058.

Grant M. R Godiard L Straube E Ashfield,T Lewald, J. Sattler A Innes R W and Dangl,J. L 1995. Structure of the Arabidopsis RPM1 gene enabling dual specificity disease resistance. Science 269 (5225), 843-846.

Harushima Y, M Yano, A Shomura, M Sato, T Shimano, Y Kuboki, T Yamamoto, S Y Lin, B A Antonio, A Parco, H Kajiya, N huang, K Yamamoto, Y Nagamura, N Kurata, G S Khush and T Sasaki 1998. A high-density rice genetic linkage map with 2275 markers using a single F2 population Genetics 148:479-494.

Kiyosawa S 1981. Gene analysis for blast resistance. Oryza 18: 196-203

Lander E S, P Green, J Abrahamson, A Barlow, M J Daly, S E Lincoln and L Newburg 1987. MAPMAKER: an interactive computer package for constructing genetic linkage maps of experimental and natural populations. Genomics 1: 174-181

Mago R, S Nair and M Mohan 1999. Resistance gene analogues from rice: cloning, sequencing and mapping. Theor. Appl. Genet. 99: 50-57.

McCouch S R, G Kockert, Z H Yu, Z Y Wang, G S Khush, W R Caffman and S D Tanksley 1988. Molecular mapping of rice chromosomes. Theor. Appl. Genet 76:815-829.

McCouch S R, R J Nelson, J Tohme and R S Zeigler 1994. Mapping of blast resistance genes in rice., pp 167-186 in Rice Blast Disease edited by R S Zeigler, S A Leong and P S Teng. CAB International, Wallingford, UIK (in association with the International Rice Research Institute, Philippines).

Meyers B C Dickerman A W Michelmore R W Sivaramakrishman S Sobral B W and Young N D 1999 Plant disease resistance genes encode members of an ancient and diverse protein family within the nucleotide-binding superfamily. Plant J. 20:317-332.

Michelmore R, I Paran, and R Kesseli 1991. Identification of markers linked to disease resistance genes by bulked segregant analysis: a rapid method to detect markers in specific genomic regions by using segregating populations. Proc. Natl. Acad. Sci. USA 88:2236-2240.

Osoegawa K, P Y Woon, B Zhao, E Frengen, M Tateno, J J Catanese and P J de Jong 1998. An improved approach for construction of bacterial artificial chromosome libraries. Genomics 52: 1-8.

Sakamoto K Tada Y Yokozeki Y Akagi H Hayashi N Fujimura T and Ichikawa N 1999 Chemical induction of disease resistance in rice is correlated with the expression of a gene encoding a nucleotide binding site and leucine-rich repeats. Plant Mol. Biol. 40:847-855.

Smith J R and S A Leong 1994. Mapping of a Magnaporthe grisea locus affecting rice (Oryzae sativa) cultivar specificity. Theor. Appl, Genet. 88: 901-908.

Tao Q and H B Zhang. 1998. Cloning and stable maintenance of DNA fragments over 300 kb in Escherichia coli with conventional plasmid-based vectors. Nucleic Acids Res. 26:4901-4909.

Valent B, L Farrall & F G Chumley 1991. Magnaporthe grisea genes for pathogenicity and virulence identified through a series of backcrosses. Genetics 127: 87-101.

Wu Y, L Tulsieram, Q Tao, H B Zhang and S J Rothstein 2000. A binary vector-based large insert library of Brassica napus and identification of clones linked to a fertility restorer locus of Ogura cytoplasmic male sterility (CMS). Genome 43: 102-109.

Yoshimura S Yamanouchi,U KatayoseY TokiS WangZ X Kono I Kurata N Yano M. Iwata N. and Sasaki,T 1998 Expression of Xa1, a bacterial blight-resistance gene in rice, is induced by bacterial inoculation. Proc. Natl. Acad. Sci. U.SA. 95 (4):1663-1668.

Yu Z H, D J Mackill, J M Bonman, S R McCouch, E Guiderdoni, J L Notteghem, and S D Tanksley 1996. Molecular mapping of genes for resistance to blast (Pyricularia grisea Sacc.). Theor. Appl. Genet. 93: 859-863

Zhang H, S Choi S S Woo, Z Li and R A Wing 1996. Construction and characterization of two rice bacterial artificial chromosome libraries from the parents of a permanent recombinant inbred mapping population. Mol. Breed. 2:11-24

Zhang H, Zhao X, Ding X, Paterson A H and Wing R A 1995. Preparation of megabase-size DNA from plant nuclei. Plant J. 7:175-184.

While certain of the preferred embodiments of the present invention have been described and specifically exemplified above, it is not intended that the invention be limited to such embodiments. Various modifications may be made thereto without departing from the scope and spirit of the present invention, as set forth in the following claims.

We claim:

1. a polynucleotide isolated from chromosome 11 of Indica rice cultivar CO39, flanked by markers R2316 and RG1094, which comprises one or more genes that confer resistance to strains of Magnaporthe grisea having avirulence gene AVR1-CO39.

2. The polynucleotide of claim 1, wherein the one ore more genes co-segregates with a marker selected from the group consisting of RGA8, RGA38 and G320.

3. The polynucleotide of claim 2, part or all of which is contained on one or more BAC clones from rice cultivar CO39 selected from the group consisting of E2P5, K6P36 1L23 and 4A14.

4. The polynucleotide of claim 3, wherein the one or more genes is selected from the group consisting of: serpin-like genes, NBS-LRR genes, rice Pib-like genes, rice Pi-ta-like genes, receptor kinases, rice Xa1-like genes and GTPases.

5. The polynucleotide of claim 4, part or all of which is contained on BAC clone E2P5 or K6P36, and comprises an open reading frame selected from the group consisting of CSL1, CSL2, CSL3, CODR1, CODR2, CODR3, CODR4 and GTPase-encoding.

6. The polynucleotide of claim 4, comprising a sequence having greater than 60% identity to a sequence selected from the group consisting of SEQ ID NO:7, SEQ ID NO:8, SEQ ID NO:9, SEQ ID NO:10, SEQ ID NO:11, SEQ ID NO:12, SEQ ID NO:13 and SEQ ID NO:14.

7. The polynucleotide of claim 6, which comprises part or all of one or more sequences selected from the group consisting of SEQ ID NO:7, SEQ ID NO:8, SEQ ID NO:9, SEQ ID NO:10, SEQ ID NO:11, SEQ ID NO:12, SEQ ID NO:13 and SEQ ID NO:14.

8. A transgenic plant comprising the portion of the polynucleotide of claim 1 that confers the resistance.

9. The transgenic plant of claim 8, which is a monocotyledenous species.

10. The transgenic plant of claim 9, selected from the group consisting of rice, maize, wheat, barley and turfgrasses.

11. A method of enhancing pathogen resistance in a plant, which comprises the steps of:
a) transforming the plant with a portion of the polynucleotide of claim 1 that confers the resistance; and
b) pre-treating the transformed plant with and agent selected from the group consisting of an AVR1-CO39 gene product and a non-pathogenic organism that expresses a portion of an AVR1-CO39 gene effective to trigger expression of a CO39-specific R gene in the plants;
the pre-treating resulting in the enhancement of pathogen resistance in the plant.

12. A method of enhancing pathogen resistance in a plant, which comprises the steps of:
a) transforming the plant with a DNA construct comprising a portion of the polynucleotide of claim 1 that confers the resistance, operably linked to an inducible promoter; and
b) exposing the plant to conditions that cause the inducible promoter to induce expression of the polynucleotide, resulting in the enhancement of pathogen resistance in the plant.

13. The method of claim 12, wherein the inducible promoter is wound-inducible or pathogen-inducible.

14. The method of claim 13, wherein the inducible promoter is chemically inducible.

15. A method of enhancing pathogen resistance in a plant, which comprises transforming the plant with a DNA construct comprising a portion of the polynucleotide of claim 1 that confers the resistance, operably linked to a promoter that constitutively expresses the gene in the plant, the constitutive expression resulting in the enhancement of pathogen resistance in the plant


SEQUENCE LISTING (14 sequences)

SEQ ID NO: 1 - DNA, 358 bp, Oryza sativa
agacgacactggtcacaaatgtgtatgaacgtgaaaagatcaacttctctgctcatgcatggatggttgtgtctcaaacctacactgtggatgctctattaaggaagctgcttaggaaagttggttacacagaaccaccactgtcaagtaacattgacaaaatggatgtgtatgatttgaaagaggaaataaagcgatgctcaaagttagaaaatgcttgatcgtacttgatgatgtctgggatcaagaagcatactttcaaatacgtgatgcattccagaatgaccaaggaagtcgcgtaataatcacaacacggaagaatcatgtggcagctcttgcttcctcaacatgtccttaa

SEQ ID NO: 2 - DNA, 493 bp, Oryza sativa
  misc_feature (123)..(123): N is any nucleotide
ggtggggaagacgacattagtcacaaatatttatgagcgtgaaaaggtcaactttgctgctcatgcatggattgttgtctcccagacctacaatgtggaggctctattaagaaagctccttanaaagattgggtctactgaactgtcacttgatagctngaacaatatggatgcacatgacctgaaagaagaaattaagaaaaagattgaagatagcaaatgtttgattgtgctggatgatgtctgggacaaaaaagtgtactttcagatgcaagaagcatnccagaatcttcaagcaactcgagtcntcatcacaacttagagaatgatgttgcagcccttgctacctcagcacgccgctnaacctccagcctttgaatggcgctgatgcatttgaactctctgtagaagggctttctataacaagggccacaaatgccccaaggagctagagaaggttgctaattctatagtggataggtgcatggccttc

SEQ ID NO: 3 - DNA, 15686 bp, Oryza sativa
ttccccccccccccccccagaactagattgcacacccagtgcatgtgagaaccccttggagtataaaaggaggttcatgtccagtaagtgggagagaggacaatttggagtaggaactcctagctctggcttgtacatcagagattcagagacttcaaaagcttagaaaaatcatcacacagaagagtagggtattacgctccttagcggccagaacttgtataatcccttttgtctcccccttttgattgagggcccaaccccctcggtccttttcgatcttatcagtttcttgatttacacacgctgtcccccgccgaactaacgaaggaggggcctcaaggtcccccgcttgaggagtttatcccccgacagttatctttacatcagacttccaaccgatgtatactttaaccgtcgagcctacatttaatattttgcatcaatatcgatcgattggctcattggacatctacttcatcatattatcatcaagttgattggattgtttattcaagttcttccattatctttatctaagtgtcagaatctacattggatatatagcagattgttcaaaaccttatcgacatatgctagtatcgttacatcggctttttggcctatcggttctttatcatcaattatctatgttgtcagttatagaatcaaactgactgacacgcccgcactggagttaagcaaatttctcaggcccttgtgtgtcacgacgaattttcacatcaactgcttgatatcataacgagccagactaagccgagctgagccagcttggcatccaccttgagttacagtgtgtagcttagtatactcaagttatatttgtttcccagatcaagctagcacacaaagtaaacatgataactacatcacgatcacgattttattaataaataataaacaaatagtataaattttagttgtgacttatgaccaggttttataattttactagaaatatcgttagtgtgtcgtggatgccctgcgcattgtaatatagcacgatccgattaatctctacttttgggcttgatctggtttttgcgaacaaatttacaaagaaattgaaaaaaaaaaagcaggtattgagaatgagatcggggacctcttggcttatgcagagaccatgagttttctgaatggttctattaacttgatgctcctccgcaaccataagatttcgcatctaattttgattatctgtcttatttaattttttttaattagtatttttattattatgagatataaaacatgaatagtacatgtaacttatgtttttaattttttttatttttttaaataaaacggatgatcagagttgtgcacggaaaattatgaatgaacttacagatagaattatagggagtggttaatataatcgttttgacattaatgaaagcttcctttatactaaaaggaaaaaaaacgagtcaaccccgatgcacaaggggatcgaataggaggataagcccgtcgagtagccagaaatcgcagatggaggcagctattttgagcggtttgttgaaaatactcgcgtcgaggatgctctctctggtagatcagaagtacaatctgtacaagggattcaaaggcgacgcagaatttctgttgaaggagctccgcatgatcgccggagccattgacgagcagctcttgcggacggtgagccgtggaagcgtgctgctgctgtccatcgaagagcttcgcgacctggctcgcgacatagaggactgcgtagatcgcatcatgtatcagaaaactcgggatcagcaagcttctcttttcagtatcaattccgtcacgggaacgtcgaagcttcagttggccaaagagatgaagaagctgaggaagagagcagacgaagcgaaagagcggcgagaaagatacacggtggtcgtcggtcatcagtcctcccctgtcagctcggatgagcaacgctgctccggtgcatctgacgggagaaacctccaggctgatctcgtcggcatcgacctgccccgagaagaacttctggagcatctgaaagaagccgagccgaagaagcttaaggtgatatcgattgttgggttctgcggcttggggaagactgcacttgcaagggagttgtacaacaacagcggtctcggccggagtttcagtaaacaggcctgggtttctgcggcacatggtgatccgagcaaggtgttgagggagataatcggacaactggtatctaacccgccatcggatgcctccgttgtcgacctggaccagcttattgtaaatctcactgatcagctgaccaacttgaggtatcaacaattcttcttctaattaggctgatttaagttccacatgctctgctccattttttcatacccgtgattcgatttatgtttcccttatggagaaattttatagtccttaagaaagtaactgaagatactaagattttacactaaaatttttggtatttcaaggtaccaaattttacactagaaaaatatgatacctcctggtattttctcaaggattgtaaaattacccttcccttatgctattcaggtttcttcagattgttgatatgctttagagtggttatagagagcagagtttctttttatgttttttagaaactacagtactgtaatactgctgcctaccttggacggtatagtgttatcaccttcacaatatctccaactatgatggagatatatcatgatgtatgaagatatatatgtccagtcctcagtcctatatccgtctctgtctccctctcaagaaagcaggtggctaggcttatggtagctgtgctgaggtgaggtaatggttgtatagccgtatcacgatggtattatcgagtttcagggtgctatcatagagatagcatcatgctcgtttatagaggtattattgcgttttacattgctatcatggtacattggttttatcaccatataacctgatattgtgatactacagtgtgtcatataactccactccggtgtgtcatggtaacttggctttcagttctagatttggaactctctacttttctgtgtgtttagttgattacagtgtaatagcggtgtatagtttccatgagttatatactcatgttatcttccaaacaaacaacaaactatagactatgcagacaactaaattaacgcagtaaaataattcagtgtgaggcctacggattggacaattacagtacagtgaaaacagaggctgtgcttgcttggcccatggttgttatttgttcatacggagtggatttttactaatgtccattcccttttgttgtttcatgttattaggtatttcattgtaatagatgacatgcgaaaagatttgtggagtactattgaatctgccttcccgaaggatggtttcagcagtagaatagtggtgaccacaacagtgcaatcagtagctaaagcctgcagttcagccaatggctatgtatataaaattagaaggctagataagatacactcaaaaaaattgttcttgaagaatgcttgtccggtggaatatcaagattacattcagcctgattcagtacggattttgaagaaatgtgatggtcaagcactcgcccttcttacagtaggtcaattcttgcgaaaaatgggctggccaagagaacccaagtgtgaagatgcttgcaaccagctatgcaatcatctggaggatgacgacaccttggagagaatgcgacaggtgctgattcatgagtactctacactatcttgccatgctctcaaagcctgcttgctatattttggcatgttccccagtggtcattcaatcaggagaaaaagactactcaggcgatggtcagctgaaggatttgtagaagcactaccttcaggttcttttcctgatccagcagttgagaatttcaataagctcatggacagaaatatcattcaacccattgatctaagcagcaacgaggaggtgaagacatgtcaaacttatggcatgatgcgcgagttcattttgctcaaatctatttctcaggattttattgctgtctttggtgacaagaagctccaataccaacatgtccgtcgcctttgtctccaaaacaatagtgctgtagatagcagcaatcttgacatcgatttgactcttgtccgatctctggtagtctttgggaaagcaggtaaagctattttggatttcaagaagtatcaactgcttcgagtcttagatcttgaagaatgtactgacttggacgatgatcatctcattcaagtgtgcaacctttttcttctaagatatttgagccttgggggcaaagtcaccaaacttccagaagagataactaaactgaaacttttggagacacttgatcttaggagaagaaaggaggtgactatcaacctgtcaacagaagtcatcaagctaccatatttaattaatctgcttggaaagtttaagctcctaaataaagccaagagattgaacgaactgcagaagtttttgtccgaaaactgtagattgcagacactagcaggatttagcacagacggaagtgaaggatttccagaacttatgggccacatgaagcaattgagaaaggtgaaagtttggtgtactgaactatcagcaagtagttctgggtttactaatcttcaaaatgccattcaaaaatttatacatgacgaacagaatggattcaacgatcctcgctctttgtcgcttaactttgataattgccctgaagacttcctctacgagataaaagccccatgctaccttcgatcgttgaaattacatgctaaattactggaatcgcccaaatttgtggtactattgcgtggcctccaggagttgtgcatttcatcatcagctacaaaattgaccacaggtcttctttcagctttgagtaacctgaggaaattgaagtatctcaagctgattgcagatcaacttgaggagttcattattaaggataatgcattgccaagcctgctaagcctatgttttgtgctgaaccgtcctacattcccggtaatcaaaggaaatgctttgaggtttctcaaatcactccagcttctctgtaaaaatctagttggcctttcagagatcaacattaattgtctcaaacgccttgaggaagtcgttcttcatccatgtgtgaacaaagcaactaaagttacatgggaaagggcagctaaggaacatcccaataggccaaaagttttggtgcttgaaaaggttgatctagcagatggccatgaagaagattcagattcgaccccaactgaaattgtgacaaataaggaatctactgttgcaggaaatggagcagatattgacaaacagaactatattgacaaacagaactatcctaccagtaatatgtcacgtgcaatggtttcccctgctttgactgagccatgcagcgctgggaatggtgtggagtcctcttgtgcctgacgacacggtcagttttgtggaataattcccttctctgttcttattgcaacaaagtgaaacaaaatttgcgaaagaattctactgaattgaatatggaataaatttgcaaaaaaaattcttctttattaaatctgtacagtatttttttctgtaaagttcaagtttacaggttatcaaaagataaaagtaagaaaattttggacaacaatacaaatggatttgtattccccaaaatttcaagtgcttatatttgttagtttccatcaatcagcactttaggctgactctatgtttccaacttgacatattcatgctatcgtaattggaacctacaaatttgatacgccttctgatttcaaatataatttgtttttgatgagcacatgaatagaaaatttatcttcaaattactttgttacatctatttatactagatcggaaaatatgccatctctttgagagtgataggatttattttatttggtgtggctaaatatggaagaaaaaggatgaaaaagtgtccatgttataaaattacttagtaaagatttttttacttagtaaagagttgagagtgctaaaacaacttgtatttacattagaaatcagaaagagtaataatctagttagttgcagtttctttaaaccaatagttgcatttacgatcaactgaaattaatcttgccatgtaaattgcaagctgcttatgctgtcgctgatgattaatccgtatggcttctgttttcttttatttttaaacaaatgcttagccggattatgtgtatgtgtacctttttctttatatttctctatccaattttgtccaatactgctacagttgggccttgctttagtactcctcccaggaaaaacttagcagatcttacagctcctaagaaaaggttttcgcacccatatcccaactgaacaccaacgcgctatttgcatctaaagttgtaagcatttcatgtcttagaagtagttggacgactcagccaccaacctagcattgatgactagaaaacagctttgaccttaagtagctactagttcaaatcttttgttttttcaaaacctgttttcatgctcagtttggctcttctttctcaagagaaataaccctgtttctatatcagtgactataaaaatcagggtttgtgtgaatacaaagcggttagaaaacatattggaggagtggttttgcagtgctcttactctctaactgatcaactgtgaaactttgttcattgagatcttttacagtacttggaaggtacctccttaaccaattgatacccatcattaattcatttttttcgataacttctaactgggctcactcctggcctctgcatgaaatgcacacagctacaaaacaggatctaactagactctcaacacaaaactaagaaattagtgactatcaagccgaagactagatcgccacccatgctctagggtaaaaaactcatgtaccacctgatccaaacaacgcgataccaccacaatacctgaagacccggtttgtggaggatagacctggtgcgtagccacctggtgactaaagcaataacctgcaaaggagaagataccatcttattatcaaaaattgccccgtttcagcttagccaaatggaccaacaaatagctgttgcacccatcaaaagcagatttctcatatccttaggaacacatgtaaaccaacgcccaaacatatgattaaaattttgaggatgttgtaagttataagccatatgaatcacggatcaaaccaaacgagtaacatgacattggaagaacaaatgttggatggtttcgtccttatgacagaagcaacgtttcttattacctttccactttcgcttagctagattatccttggttagaattatgccccgacgtaagtaccaaaagaaaatctttactttgagcaatacttttaccttccaaatacatctatttatattcgggatgcccgtatggacaagagctaagtaatgagaattgaccgaaaagatcccattgggggtgagattccatcgaaattcatcttgctcctgtgccaacaccaaatcagctatgcgaggccataaattgtgccatgcggctaattttgctccgattaaatctcgtctccatgaaaagcttggaggattgttttgaaagatttatgagatagtcgattgtttatgccttgctatgttatagagacaaggatattaatctctcaacgaggcattacccaaccatttatcctcccaaaaccttatttgagagctttccctaatacgaaaggacctaaacctaagaaaatcttgtttggctttcataaggctagcccaaaaatatgagtcccccgcttttcaaaaagcctgagataaaggttgcgatcccaaatatttattacgtagcatatcttgccataattttggaacagaaaatagtattaataagggtagcttagtaattgctcaatcccagttccccattccctgcctagcggcatcatgccaaagtggtgaagtcctaagtagacttactatgttttcaaccacacacaacccacctaaggcacgttgtgtcatgttatatggaataccatttgttgattggtaagtgttgtatacttgtgtttcatggtgagattaatacaagaactactagggaaaagtttcatttgtagtcatgtgtgcaacattgctagaaaaatagtaactgtactgtaattgcgaagccaaaaggaaatcgaagcaagttatacatgctgcagggcactttagttaatatatgtaccttaattagctgaaaatctctttatccaaatatagtatcatgacggccattttgattttgcagtacattggaagagttgtttgtggattgtcattgctctgagctgatttactgtgttggtacatggtactgctagttctgttgactagaatctcagaaacaaatacggctcatttcaacgttgagaagatatattgacgacatttggttgcttccgttgcaatatctgtgctcctttgtacttcaattgcaatttgcaaggtttgtctttcttcgattcaaatcactggacatctgtctggtcaatttgcaaggtttgtctttcttcaattcaaatcactggacatctgtctggtcaaaatgaagttcaccatgttgttctttgttgttagtgcttgtaattgttgtaacatattcagaaacacattgttgtaccttgtgctaagtgagaaactatatttttgttgtaccttggtgcggtgcagcacgattgctggtctgtatctgatatgccacttatgttttttaattaataaaaaaactgtttttaataatatgggaataaacctttcaatagattgtttttctcttgcaaatcttaaccttttaacttttaggcacacctagctacctacatcctgccattgcgaataaatttgtggtttgacaccaccatgctcattactatgtgcaactcttcacaagagccgagtaggcaaccacagagcgtatgctgatcacactatgcatagtagtgagtggttcaatgagggaatgtggatcctaagtttatgcgcccaactttgatcgtccgttttatttgagaaatttttataattagtatttttgttgttatgagatgataaaatataaatagtactttactcatgacttatgtttttaaattttttcaaaaaattttcaaataagacagacgattaaagttgggcgcggaaaactatggttacacttaaaatgggacggagggagtactacccttatgtccacagttagtgacaacatcactgggaagtaaatgtcagaggaacctcctccttcgggcatcctcaatgttaaaccaaacaacaactataagacggaacccacatatatgtgtttttacttatagtcaatgctacaatgtttaagatcatctcttcaccttaagttgggactcactagaaaaaataatatctattaattcttatagtatgaaggattcaaaccaacgtacatgttgaaaagacataaagtaattttatattccttatagctagctttaaggttaagttagccatagcaatattctcttccaaacacacctccctatacataaagttgtactaagaatattatcatatggcttatggtcgcatttacatgcacattagagatgcccttcaatcagagggatagaaaaaagtgagtgatgtttagcaagactacagggcctacttgcttacatgcacaggagaaactataccaaggctctggttagtttccacacaaaaattttataccctatcacatcgaatgtttaaacacctgtatgaagtattaaatataggctaaaaaaataattaattgtgcagattgcgactaatttgcgagacgaatcttttaagcctaattgctctatgatttaacaatgtggtgctacagtaaacatttgctaatgatggattaattaggcttaataaatttatctcgttgtttaatgacggattctgtaattagttttttattagtgcccgaaacaccccatgcgacaccctatataatatccgatgtgacatgccaaaactttacagccttggatctaaacacccccaagtgatattaattgtatcatagtggattaggataatctaatttggtgcaaatagctgccattagtacaataacgatttttccgtgcggtcgaaagcggtttttgcgtgcaaatcttccgcccgtctagaacgaagtgcctgtgaaaatccgatttttttcgtgcgggtgatgcacccacacgtgtgtgcgggcacttaagtcacccgcacgagaaaataaaaaatcgaaacagaaaaaaaaaaccaaaagccgccctggaaccctaatccagccgccgccgctggcctgcctgaggccagtcaccgtcgtccgcttggtcgccggcaacggatccaaacgcccgaggctgggagaccattgccgccaccgcagtcatcgtcgctgccctgcccgaggcccgccgctatcatcccaatggtcgccggtgatggatccaccctccccacggtggccggcgacggatccgcgctccccacggtggcaggcgacggatccaccctccccgtggtggccagcggcggatccgctccgctgccaccaccctagccgatgctgccgctacactcgccaatgccgccgccgccgctctcaccgatgccgctgccaccgccgccctagccgaggctgctgccgtcgccaggagtgggtacaggggagggtggagagggagagggagatgggaggagtgagaggagagatgggaggatgagagggagagggagagggagtgggagttggagtggccgagtggtaggatgggaagagatcaagtggcgccgggagctaaaagggtgggtgggctatttttgcgtgcgggtcacttaacaggttcgcacgcgaaaataagcctattattgcgtgtggatctgttaagaggcctgcctgcaaaaattgttttttccttgcggtcattttaaagaaccgcacacgaaaatggagcgtgatttttgcagacgcgaccacttacagtccgtctgcaatataaagtgagtcctgtacaggaaaattatttctatactagtgcgcttttagattttttggcgtttggcaaactgatttttttttaatctatatgtcgcaaacagtaaaaccactaagagacatgacagttaagtaacacaattatataactgtaatttaatttgtaaaacaaactacgacggtcagtaaaataacaagcccagcgcagtgtagcacaaccaccataaagtaagcctgaagttgtgaggtcttccaacaaaaatgctgggtataggggatatagtagttcacaagtgaatagcacagtgaccagagagctagacaatatatagatggatgaccagaagcttaaatcctaaccatattatatacttgcattgatgcataatagcattgacaacattggtcctactatcttgttacatttcatttccaccgcatatatctccaaacagagagattaatatattggcaacagtacatgcaaataaggtcttataaaaaaatggatgtgctcttacatacaatgaatatcaaagtaaatgaaactacacataatgtacagaggcaccgcgaacgcaaaaaagctaggaatgataggcaactcatcggaaatcgacacctgctgcaaacagagggggaaaaaaactacaaacggcaccagccagccgcatctaaagttctaaacacgaatctctggaacatgctgcatcttctgatgcattctgtttttgtgccattgagttttgaagtccctgtgcaggtacagaagccagagcttcttcagggtccgaagggactcaatgccctcagggactatatccagctttgagagtgacacaacgtacaaaccttcaatggatggaagtgccccatccatgatctttagctggttcacattgggcatgtgctttaagacaagtgtcttcaggtggggaaaagactgtagaaagaaccaaaatgtttgcactatgcatgttgttcagtctcaaataagtgaggttcggcaaatgcgaagcgagcatccctagtggatcttcaccaagatgacaccaacttagagctagatatttaagatttgtgccgttcccgtgaaatattgggcaatcaagtgtacccttagcccatcgccctctgatgattagtctgtggagttctgttgaccttggcctaagagcctcgaagcagagttcctcattttcatcttttgcagaaagaagcaagctggaaagaaatagcgaaaatatttgcacaatcagcagaacttatgttgtcaatccacacacttcttagttgcatcaatttcttcagttgctcagccaggtctttgctagactccacagtttctagagtctgcagctcttgcaagttggaaagttctttaggggcatgcattccaacaaagtaccggaaatctgactgcctctcatcaacgtatctatcggctataaggtgtcttagcttcttgatcttaacaatacttcgtggtagcttctcaattttggtttgcttgatgtcaagcgtgtggaggttagataactttccaatagactccggaagtgatttgactttggtgcgtcgtaagccaatgtaacgtagattaaacatattcccaatagaagtcggcacttcagtaatctctgagtcttgcagctcaagaactgtaaggtagctagatccacacaaaattgaggataacatctcaaggggcaatgaggttgtcgaaagtgagatcagggtccgaaggcgcatgaatttaactgttgatacagtatcatcactccatccacatgttgacaagcgacgaacttccttatccataagcaacattgtgccaagatcattggctgaaccaaacctctcctctttagcaatagaaagagccaatacacgcacaatgtcatgcatcttacaggagtttaccctgccaatctcatcattgtccacaacttccagcatattccggtggatcagttccataaggtttccctctgcgacatcctctagcgtgttcttttctttgcctagcacaaagccttctgcaacccacaacctcagaaggctctcccgtgtcattgtgtagtcttcagggaacaagctacagtacaagaagcaatttctgaggtctcctgataggtcatggtagctcaaatttagaattgctcggacatgatcattgtttgctagctcagtccgaagctgtttgtatattttattccaaacaaattctgctgctggtcttgaagacagaaggcttcctatcgttacaattgctagtggtaggccatgacacctatccactatagaattagcaaccttctctagctccttggggcatttgtggcccttgttatagaaagcccttctacagaagagttcaaatgcatcagcgccattcaaaggctggaggttgagacggcgtgttgaggtagcaagggctgcaacatcattctctcgagttgtgatgatgactcgagttgcttgaagattctggaatgcatcttgcatctgaaagtacacttttttgtcccagacatcatccagcacaatcaaacatttgctatcttcaatctttttcttaatttcttctttcaggtcatgtgcatccatattgttcaagctatcaagtgacagttcagtagacccaatctttctaaggagctttcttaatagagcctccacattgtaggtctgggagacaacaatccatgcatgagcagcaaagttgaccttttcacgctcataaacatttgtgactaatgtggtttttcccaacccacctataccagatactgttatcactgctctatctggctcatcagagtacaaccatccagccagcaatctcttgtggtcttcaatccccacaagatcttcatctttgacaagatatgggaagttgtcgtgagaccgtggtctgccagtctcagcaagctggttgggattgagctgggaagggtgcaaccactgctctttaagctttataacttgctggatttgcttctctaacttgactacctcctcagcaatatcactaaataccacgacttaatgagaacctttaacgaagtacttcttcaagaaaccttcttcctcaagttgaatagcatgatatgagtacttgtccattacgtcctcaacatggtaggctaactttctcacctctgcaatccaattctttacaactataccagtgaggtatgaggtgcctatctgtagtataacactgttcatgattgtcagttgcttccttatttcctcgaccttctctggcatctccttcaaattagtaaccttttcagacaccttggcaatgacagcgttggcagcttcatctgctaacacgttgccaacctttttgacagaaaggagcactgcctcagccatttgttctgaaagaacacacaccattatgttacaaaagagaacaacaagtggcttgcgatttctagtctccaatttaacttatatatcaaagaaaatttttatttattgatgcctttaattatcactattttcctggagaaatgtgtgctttcattgaacagaactccaatggtttctgcatgaaatgtgtggcactgttcagacttggataataatggtccactgtgagaaaagaaataatggtactcatgcaccgtgtccatagagtaattcttttaccacaagcatctatgatctcataaaacgttttatgagaagttaatggcatctcataagatacaactcagcaaataaatgacaagctttatttgatttgcttgtgggcacagagtaggattccaaatcagaatggcaaacagtatcatatatgaaggccagtgctaagttatattttattaattgacttggaacattttttttttgacaccttactttggagttcggaaatcggctacgagattttctggacaaactatgctttcggaagagctctcctgcttggctcagtaaaagaaacattgctataatgttatacatcatgaagtacagctattctgtatgtagttgtttactttaatttgagtatgcatttattacatttcaccaacatacatgtgtttagttcctgaaattggggagaagtttagggaaagttggtagtttggaaaaaaagttgggagtttatgtgtgtaggaaagttttggatgtgatgtgatgtgatggaaagttgggagttggggggaagtttggtgtgaactaaacacaccctatacagcttcagctgaagcaacttgtgggctgaacatagttcatacatattcaaagaaaataactggttcatcaacaagacagtgagcagaaagactatacattagacaagcaagtttagcagcattacctggaacattagactaacaactatgaagaacaaaaaaatgatccgtagaacagaacacacaacaaatacactgccatactgagtagtgatttatatgcttgctcacctgctggaagaccagagaacagggctggagcgcaggtgctgtacggtcaatgtgcagaaccctgcaggatcaaacaagagaggatgagaacccagtcagcaaaacagtttgtgatcgtgcccaaccggggacaaaatttggcccaaaaaaaaaaaaaagcgccgc

SEQ ID NO: 4 - DNA, 17953 bp, Oryza sativa
caccaccctccctttcctaagccggcaccacctaggacaagatatggcgatgacgagtgccgtatcttcttccgcttgggagggcaagtgcgggggctcgagcggcggcaaaaagagaggagagaaagagaagagatggggatggcgtggtgaggatggggctcggctctcctcttattggcggcgtgagcttggcgggacgcggcgatctggcggctaggcaccgcgagtccgggcatagccggctagctgctacggcgagcggatgacgatgcggtggcgcggcgacgtgatgacgatggtgacggcgcgacgtcatgggaagcggcgatggaacgcgaggtgacgcgcggggagcgggtttggcgacgggataatggtgacgcgcgacggacggcggctacggccgggaatcgagcggtagggttcttctcagagcgaggggacgcgagcggcgtcatgcggtggtggcaacgcgacgcgagagagagagataggcgcgggctcggcggcttcccggtaggagacgggcgacaaggcgcggtgtgacaagtggagagggcgcgcgtccgcgggagcccgggataggacggcgtggcgtcacgaggaggtggcgacgggacgacgcggcgccgtgtctcccggagagaaaccggagcgagggatgggatgtgattcccactgggatatgcggcttggcagggcgcggctttggcgcgatgcggtcctggcacggcgcgcggcgcatgggagggcggctcggtgcgacgcagcactgccacgactcagtgctcgggcgagacatttcgtcgaacatgtggtgcgcgcgcgcgaccggtcggaggaggaagacaaacagcggtgggctgggccggtgcgggaaggccatgggccggcctgctcggggagagagaggagaggaaagggagttgggctgagggaagaaaaaggaaaaaaagaggcccaaatgaatagtgacctttgacctttcacattttctctaggatttggaatttgaaattttggggagtttttggaggaatctaatttgaattttgtcctagggttttgacaacaactcgaggggatttctaagagaggatttgagacctatttggcacaaaatcaaataagaaccaaatttatactcaaaatgtttaaatgacaagatttttaaataaatgaaaaattcaagggtgctacaaaattacaaagagcctctttaaggtaaataaagcatgcaacttctagacacagatttctgcaacatgcagcatcttcttgtgcatctcattaccattccattggattttgaagtctttatgcaggttcaacagggagagcttcttcagggagttaagggactcaatgccttgagggaccttgtccagcttcggcagcaacacaatgtacaaacattcaatgcatggaagggcgccatccatgatctttatctgcttgacatcaggcatctgcctcaagacaagagtctttagtttggggaatgcctttgcacgaagaactaatgttgctgcactctgcatgttgttcagttttagataagtgaggtccgacaagttcgacgcaagcatccccaatggatcttccccgagatgacaccaacttagggataaatatttgagatgtgtactgtggctacggaatatcgggtagtccaatgtactcttggcccattgccctctgacaattaacctgtggagttctgtggaacttggcttgagagcctcaaaagaaagtggctcattctcattccttgcagaaagaagcaaactggaaagtagcggcatatttgacagtgtagcaaaaatattatcacaatcagcagagcttatgttgtcaatccatacactttttagttgtatgagtttcttcaactgctcagctaagtccttgctggcttcaacagtctccagagtttgtagttctttcaggttggatagatctttaggtgcctgcattcctacaaagtatcggaactccgactgcttctcgtcaacacatctatcagcaaacaagtgtcttagcttcttgattttagtgattcctcgtggtagcttctctatctttgtttgcttcatgtccagagtgtggaggttcagcaacttttcaatggagtctgggagtgacttaaccttggtcctccgtaagccaatgtaacgtagattaaacaaattccctatagatggtggaacttgagtgatttctgaatcttgaagctcgagaacagtaaggtagcttgagtgagacaaaactgaggacaacatatcaatggaagatgaaaatgcttcaagtgatactatggttcgaagacgtagaagtttgagaattggtgcagtactgtctttccatcggtaagttgacagacgacgaacatccttatcaatctctaccattgtgccaaaatcatttgcagagccaaacttctcctctttagcagcagaaagggccaggtctcgcataatgtcatgcattccacaagtattcaccctgccgagatcatcatactctgtaacttgaagcatattcctgtatatcaattccatgagatttccctcagctactgcctctggtgtgttgttctctttcctcaggacaaagccttctgcaatccacagacgcacaaggctctcacgggagagcgggtagtcttccgggaataggctgcagtacaaaaagcagtttcttaggtctcctgacaggtcatggtagctcatatttaaaattgctcggacatgattgttctttgacaactcacttctaagttgattgtatgcttgattccaaacataatgtgaccgtgatcttgaagacaggaggcagcctattgacacaattgctagtggaaggccctgacaccgctcaactatagatttggcaactttcacgagttccgtgggacactcatggtccttaatgttataaaatgcccttctgcagaatagatcaaagccatgaatatcactcaatggctggagatcaaggtgacatgttgaggaagcaagagctgccacatgattcttccgtgttgtgattattacgcgacttccttggtcattctggaatgcatcacgtatttgaaagtatgcttcttggtcccagacatcatcaagtacgatcaagcattttctaactttgagcattcgctttatttcctctttcaaatcatacacatccattttgtcaatgttacttgacagtggtggttctgtgtaaccaactttccaaagcagcttccttaatagagcatccacagtgtaggtttgagacacaaccatccatgcatgagcagagaagttgatcttttcacgttcatacacatttgtgaccagggtggttttccccagtccgcccatacctgacactgttatcaccttactgtccagctcgtcggtgtacaaccattcagtcagcagtctcctgttgtcttcaatccccacaagatcttcatctttcacaagttctgggaagctgtcccgggacctctgtctttccatctcagtgagcgggtcagaaacaagctgggatggatgcaaccactgatccttcagttctataacttgtttgatttccttctctatcttgactacctcattagcaatttcagtgaaaacaataacataatgtgatgctttgatgaagtattttttaaggaaccactcttccgccatttgcacagagtaatacgagtatttgtccatgacatcctcaacacggtaggccaccttccttacctccccaatccaacccttgacgacttcatcagtgaggtatgtcgtgcctatctgcaatataacgttgttcatggttgtcaattgtttcctcatttgctcaatcttctcatccagatcctttagattattaaccttttcagacagtttggcaatcaattctttagcgatttcatttgctaaagcatcaccgatcttcgttagagcaaggaggaccgcctctgccatttgtgctgaacaaaataaacatattatcatgttatggacatacaaatatcacccaaagtttttggcctcagttttactttgatgttgaaaattggaaacacttttctattgtagaccatagtcagactatgataataatgacactactggatgaggctcatgatgtataacttagcaattaaatcagtaacacgggccatgaatcaaggttagatctaatctatattaatgcatttacttggaacaacctagcaacatcaaaaacaaaactgctcatgtttcagtcacgtcatacctagatcggaagataccttttcaatttttttttaaaagaactacagtaggtaagtagagagaaaccggttaattaagataaagctccaatttattgagggattaaatgataattcttggagggtatctttttaattaaaaataataagtaaaaaaactgccgacagggaaacaaacttgggtggtgaggttcaattcccaaactccagttaaaaaatttgagagaaagtgtgagttctttcattagattgtatctatatctaacatactatgctgttgggaagtgcttgggtcagtaaaagaaacattattatacacaagcgatgtaatagcaattcaaaatctgcttgtttacatcaatttgagtatcgatttactgtttagtacttaactataatataaccctttgcttgtctgagacattaattttgtggaacgaatgaatttgttgtggttcacaacaaaataataaatgaatcagcaaggtactgaatgataaaactagtattagataagcatgtaatatttcttggaacatggttgcatttataaaataaaaggaaaagtatattttacttccctaaattatttggctgaagcagttaatcctaagcaattttaagttcgctctattccctgaactactaaaactggctagcttaactctctcgttgctttgtcgtttttgtttgtccttacacaagatgtgttttaaactgaaattgtgtgagatgatatgctcgctcagggtcaaatttcagaacaaatttcccaatattttctagcaaagagactagttagtgatccctcctctcaacaaggcgaaattcacgaaagaaaaaggcagaattttccagcagacataatcagaaaagttaaagctgatgacagctacaaactagctagatgatacaaagcaagatccagtggaaatcaatctaacctgagctgcagacgatcagagcgtctagaagagcagagctggcgagacgatcagagcatgtgaggtttaagataggcaacgaagaaaatataaagcacaaagccactagcctcaaatctcaatcagatccttgtctgtttctctgaactagccgccgacgacttgacgcgaagtcgacgcggctaagcatttgcagtgccaattatcaactggccaagttgtgcgtgtaggacgggagtccgctgtggaagcttcctttttcctgatctcaaaatttatagtataccctccaaaccgcatgcagtgggggatgctggattctttttaaatggaccagaaactcgagaaattattgctcaatagattattttgcctatagtaaaaaggaaaagaaaaagtaaatcagaattgttcatatcgaaaggaatagaatatctgtagtccagtatgcaaattagaaaagatacagttttgattagtttactgtcctgcagccagctcactggtcaggctgttcgctgcatcttggacatatactcgctttgcccaaaatataagggattttggacggatgagatatttttttagtacaaggaatctggacatgtactagtaagtgttgcatctgtttaaaatcctttatatttaggtacgaatggagtaggaagaaacatattgttatatttgaggatggagggagtaggaagaaacataccgtgtcaccttaacagtccttgtacttatgactcagttgttgaggacctagttagctagtcggcaactgcaaaaatataattccagtctccttattctactcactaatgacagcttagagaaagctaacaaactgatttcttatgttttcatccaactaaaacggccggagacctccaagtcgtcttagtataaaaatctctcaagtcaagtttcctcgatttcttggtcgtcgtcttcgtccaccttttggtatgctcgattttttaaaaaaaattatgactctttttgtaaaacttatttcataaatagaccctccaaaaatctaattatggatatggactctactcggtgccggtgagcttggcatcgaggtttcatgcacccacgctggagaccttggcatcgagttagagggggagggggagagagtgtcgatgttgtagatcacgactatggtatttgcaaagtttggggatcattcgtaccagggtataagcgagactaaggtaaaatagacggagacaaggattttcatgtaggttcaggctccttatttaacaggtaatagccctactcctgctaattgaaaccagtgttgctcttattcatcagaatcacacaagtacaatatttgggataactctaatcatcgtcaacacggcggcatgaaccaccacacgttgtcgacaacagggtagtcctcctcctctaatatgaactgggcgatatcagagatagcgctagatccctcttgttagcctctgtggcaccagattggatctgtttaggtttatctcttatgttgatgtctggcggcatgtattgatgtatattctgactgttgcctgtctatttagactcggcttgtcttggtccgtctccccctcttcttttaggggtcttgtatttataaccatagatgtccccttatccaaatagaactagaaagataaatatggatacgatccgaatagtccttgtagtttccatatagaactctgcttctccttccttatctggaataccttccatatgcaagatttgatttcatataagacttggtatatggtgggtcccgttgagcttaacccaacactattgggtatgccttacccataaaactgacagggggtaggcgccatgcttcttggtgcggacaccgagttgtcccgagatggcatgggatggcatgagtgtacagcgagcaccaagagtctgagacaccatggaatggcacgtgtttggcaacttttttgacaagtagacaaacacgatcgagacacctaaacgccttgcggtgattgtagtgtcgccaaccacctcaatctagcaaaagctagaccaagatgggttttgataaactaaaccggctagcggagccgatttagatagaacaatagataactatccgtctcgtgggtaaacggaaggtttacgggacaaacctacaaaggatttcgcactctcgtagtgaagacagacgatttatggcacaaatcgacaaagatggatgaactagactagaactagattgaaatattgaaaaagcgatttaattggctaagttggattgtgtgtagattgtatgctcaattgaaccggccttgtcccttatataggggttgatcttgcctcctacaggtcctcctccacgtcgaactcgggatagaattcaaaggaaacccgaaatatagcctcctgagtaaggaatcctgagacatgacgaaaacagactcgggcttggactctgctggtctgaccggccacatgccgccggacagaccggccctcaagcagcggtctgaccgaccaacaaacgacggtcagaccggccctatggaggaaaccggcggttttcccaaatcttagcaattttccttaatttgaacagacaaacgatgatgatgagcatggcccacctcaaccctccctcttctccatttcaatccctgcctttttatcagttgattctatcttatttttcctctatgtagctcctaatgttactacttcctctatttcatactacaagactttctagcattgctcgcatataagatagaatatactacaagacttttttttcttaacggcaacatatcctatgcacacaggccctcacgtgtacacacgtgcacaccaactaaaaaatgtcaccaaaaaatttagaaaaaatcatacacatactttcaattgtattacacctagggttaaaatcttaacgtcaaattcattatattttagccgtaacaaaaaaaacaaaaaatctgacagttttaaggttgcaattttgtcagaattttatcttttttgttattctctatatagaatgaatttgaagatgcgactttgcacgttgatgtaatactattgaaagtacatgtatgaattttcctagaattttttgtgataatttttagttggtgtacacggtgtgtacacgcgaatatatatatatatatgtgtgtgtgtgtgtgttaatgaatctagacatatatatatgtgtttagatttattaacatctatataaatgtgagtaatgctaaaaagtcttataatataaaacagagtgagtaatattttttttctatggaaatcttgaatacccatcccgaaaatcatgtctctgtcactaatagggctagtggcatttggccaagcgttggtcgggttggtatgtgcaggagttactagcacgtgtggtggaggttttttttttctttttccttgataataatttcgtagggttataatcttttgtactttttcccagctttatcaatatgaaatttcacaccatcttatcttatgtgtgggccattttggaaaaaaaaaactgagctgcaattgtatcgctttcagtaacaccagaccacgcgatcatttttaccgtgactcggtggttgatgacctatagcttagctagttggcacaactgtagatccgattgctccatacaatccgtctgcttattctaatcactactggtagctaatggaacgtaaaacgaggaagtcattgccattttgattaattaaattttaattattttaaacttaaaaatggatttatttgatattttaaaacaacttgtatataacaagttttcacacgaaatataccatttaactgtttgaaaagcatgctaacgaaaaccaaggtaaaatctgtataatcaaagttagaacggggttagagaaaagccaataaaacggccgtggacctgaaaagtttcattagaatacacctctcaagtcagtctcctctgcatcctggtcgtcgccttcgtctacctctcgcctcgccgtcgccggaagaggagctaaccggctggagatcgattgatccaacatgagtgacaatcctccaggtatgtgcatgctacaagcttaaccaatcatccataggcgaactatttcacgtttgaataacatattttctgtaaattttcttactttgtgggtgttgttcatatgcaggctacttcgttggtcgcccattgaaccatgaagaacagcaagcttcacggccggctgaggaacagaacgctcaattccctggtaggtatacaggattaattagttttctgcatacatcaatttgcttagttttctgcatacatcaatttgccggatatgatgtagcagatcttgttataacttgaggttgataagtgctttgaaaagggttcttaattttcagtcttctatgcattcttctattacctcaggctgttttgtttgctatgtcctatgatagttttcaacttttcaaatttcaaatccttataaagattaagagaaatctttatcttctgattttccttttctactactaaggctactacaatggcaatccggtcagaccaaatgatgctaagggtgcacagaggaatgaaccgggcttctttaagaaactgtaagctgcaattcgtgtacagaatatcagatgcttctgtctgtagttacccattgatttgtttttctctttgcatgatgcattggctcattgaatctctgcagcttcgggtgcttcactggcggtcaaaatgtgaactagatcaacacggctgtattcgcaatgttaatcgacagttcatcaagaaatcagtggaattttgatggagaagttatcattttaggaaatacaagttttgtttcttccaaagtttttcttttcctgcagttacactgttgtgaattgtacaatcatatcccaagcaaatgagattattccgcgttatccattagtattttgcttacctgtttggatctatcaatttactatattgctactggtctcattaaaaggattgttttatccaagtcgtctttgtcaaggaaaaggtccacatgaatatgaaaactttgaactttctcttggatggttcagaaattttttttgactcaatcgcctattaattacaacgaacaatcatttgtcattttcaacgaaaaagaataataatgtttgcaaacgagagcagcgatcaatgagatgactttgacaattgaccgacttatttgcaactgcaagtcttcaagcaattgaaccaaactgtcatggattttaatttttcagcgtttgctttgagatgaaaatgactataaattaactggaaaagttctaagactcttcgctgacaaggattgataggtattctggtatttggcatgcttgtggtcttcattaattaatcctggttcagggtaaataaacttggacaataatcaattattatactcattcaaatcaaggcctggtttaattcctaactttttgttcaaacttccaacttttccatcacatcaaaacttttctacgcacacaaactttcaacttttccatcacatcgttccaatttcaatcaaacttctaattttgacgtgaacaaacacaccccaaattacacttagaatcttatattcgccaccagtccatactatactaatcaagtagacacctttactttcatctaatttccaagactaacagccaatgcatcaaccttatggactatcatattgaaacattttatatcatgttccttttgcaatacttatttacaaatacaatcctatgaaacttaattgtaggaatgaaccccaatctcagtcacagccaagcccaagcttaggagtgaactccaacatcttagcaccaacaagcaggtcacttagttactactccctccgtcctaaaatgtaagatttttagctatgaatttggacacgtatgtgtccagattcatagctaaaaattgctatattttgggacggaggtagtatattaatcttgcttctagcatggtgccattggtcaactccaaggatttttgtgctgagatgttagaagttttagggtccatttgtgcgatcagtttttaagggtttgttcttaaaataagtattgaaaaagaacacaaatgtgtaaaatcttcaggggctacttaaaagaaaaagctttttgcagacagtcaaaagtgatttacacaagtggccagccattatgtctacaatttaaggcctgcgaaaatcatgcatctttcacattgattgatattctgtcttcaaaagcatcatacatttatcattgatatgtaggtttatgattcatcagacctatatatcagtaataaagtcacatgcctttcaatgtagaagaggccgatctctttcacttgcagccaaactaacacggcccaaatatctgtaagacaatgaagttttccctgtcatgacaacttacaagatccagagtatctgtcctaactaaaacggttgccagtacggtgaaaataaaaaaaacaaacagtggatgatttgatattctcattgcatgccatgtttgcaatttcgtggaaagaaataacaaatagtggatgctttgatattctctatctcatgccatgtttgcaaattcctgatactctagttactgagctggtcttcgagtagtagtggtcgctggttggtgatgccacttgagcaacgacttgggttttaggtcgtcgcggccgaaaaaaatatcaatctctgcgcgaatcattggcgtaggccagcctgggagagaggaaggaagcatcttcctgtgcctctccttaccctcttcgtcttcttctccatctccttgtgtgctcagtagcttctcctcctacgaaaccaaccagctgccgccggtgaccgtcgatccgtcgaccgccgccgccatgagcagcgatgttcctggtacgtgtcactcactcacaagctttctcttctcttcttttttttttgtttgtatcaatcaatcatgctaaagcctaaaggaattcttgcttctttatatcagtctaagaatttttcggtgtgtgtgtacgtttgttgcagggtacttcgtggggcggccgatgaaccaagcggagcctgcgaaagaacagcagcagggcgccgacgagcagcggcctgctaccaacgcgcagatccctggtggtacgtccaacggtgcaaactcgatttgatgatcaaattttggccgcaaaagaatcggttttgcccgatgttttgctgaactttgcttcgttttcgttttatgtaatcgttttgggacacaaaaatgagtcgattttgtccggttggtttgctaaatttttgcttcgttttcattttttgggggggagaagactacttcgtcgggcgtccggcaaacccgcagcagccgccgtcgcaacctccgcgggcgcaggagagatcgagcttcttggccaaatggtactccaatttgttcttcgaaaatcgaacaacaattgtttagattgtattagatttcatatcgatttgtcactctgatgctttgctcttgctgatttctctgtttcgtttctgcagctgcccatgcctcgacagccgagctgagggatagacgaacaagtgcggcggcgggcgtgaccgtaaattcgatgggaaaattgaagcaaatttgcttggagtgataaggctcggatctgtgtttgtgttgcaagttttgctaatagatgtttgtaactgaaggttgcttctattcatttgtataccaattgttcattcgtatcatcagagtgcgaattcgatgaaattgtccattccaatccggcatgccttttctccttccttgaaatgaatattctgctccattgctggcaaatgtcaagaatgagtttgttgttttattcagtactgatgttgattgcatggttatcccgaatttgtttcccttttttttgtgaaaataaatccaaatctttaagttcatctttgataaaaaaaattctttctttgctagaaaatatatatattgttttagtcaattttgcaacttgcgtgcatatgtttttgccacagctctccacagtacatgtacgaagcaacttgatgaactaatattgtaccaattaagaaagcgaaaatcattcccagcaaaataaaagaaagagaactatgttcggattccagcggaaaatagtttaatagtgctagaattactccatggggattatttttgatgatttatttgacagctgaatgtgagtgttaacctaatgtttgattcccccaatcttgtctgcaggatgcgagaccacttttttttttgaatgaaatgatgagacgatttgtggctttaaaatatcgtatttgttttgttggacaacctagcaaccaatcattttctgctcacttggtagcgagttttttttagttgtttgatcagaatcatagttgtatcacatggaatttgtagttctacttagaaaaaatgcccgtgcgttgcaacgggaagaaaatatgagaaaattaaggagaagttgaaaaagatttacattatgtaattaacttaaatacaaatttcctgaaatagttgatattttttaagtaggtatgtgtttaaattaatttaaaagtaaattaagtgtggaaatacaatgatatggctggatttaatttttatgaagttctccatgattaattacgctctctagaattttcttgaaattttcagagcttagttgcgaattttaataatacaaacatcatttcagcatttatttaaaaaaatctaaataaaaaccctcctttctctttgggccattttcagcccgtttccttcctcagcccacagccagccgacccacttttctcccttctcctcccctccgcctcggcccgcaagtacgcatccggcccggcaaggcactacaagtctaatcatcctccccactcctttcccagtcacgtgggcacgtggtccggtgctacagggttgtctcctacctctgtctatattgtcgcatctgcccgacgacgcctgaatccacctcctccgtaaaatccggtcacatccttggagataatctgaaattaactgagaggttttgatacttatcttctcctctttccgttttcccaagaatcaaagcaaaaccacgtcgtataaatgtcggaagcgaaaccctagttttttttagtctaggcccatttctcttaaaaaaaaactgcgcaagcattcgcgagaggggggaggaattggaccatatggataaaaaaaagtgtgacgaaaggaaaaatacgaattttttaattagttaagatactaatgaattttaaggtaatacaatagttagtgcctaattgtcaagaggcaaattatatgtaaataaagataattatattatatagaagttgggtggtaaacgccctgacgcaaaaaattcatgaaaccatccgtcccaaaataaatcaatatagggttgtatgtgatattatttagtataataaatctggatatatattatattggtttattataagacgaaatgagtagcgttgtaagtatcgagtactaatccctctcttgggtaaagggacacgcagagctaccaaacgaacatactctgtttttaatagacgatgatgtttgatttttaacacaaaactttatatatttttcttattaaaaaatttgtgcaaatacattatacttaaagtatttttagtgataaaataacccacaataaaataaattataattatgtaatctttttaataagatgaatgatctaatatgtattaaaagctaacggtgccgtctattaaaagatgatgaacgtagtatattaccattcgaatttatttaagttttgcatgctccgtatgctccaactattttcttttttttaaaaaataatcaatcatcaatgtaaaagtgccatccttgtcactaggaattctctatatacccgtagcttttaactatttgccatttttagatttgacaaataactatttgtcacccctatctcaatgacatgtgggtccacatgtgtctatgacatatagatctagtggcaaaaagttaattgccacatccaaaagtggcaaatggttaaatatctccggaaatggcgccaactgaacgtgctccactggccggtgtggatggttgacttagtcagcgcactggtagcgaactatagtagtagcgattagcgatggaatggcagcgttgacttgacaaataaaaaagtgagaaaaaccaagctgcagcttgcagccagctagctcattccgtctcttctctttagctcaccctcacacctcgcccatagagtcaaaggtcgtcgcctcctctcccatcagcgagccgctccattgcgagctcatcaaccaattaatccagatacgtgatgatccatgccttcgcttgcaaatcgatttgtgctttagttaagcggacttctgcccaacatttctttctgccccgatttagacggagattaattagttttggtggatttttttgttttttagcgtttgagcatgagaagctagtggttgttgggattgtgatccggttcggatctttggtgatgaaatcgtcatgctcgcccggccacctcggttgcctgtgcgaccaacgccgtgtcgcgtcaccggtcgcccacgacgtgttcggcgtaaggcgccaaagcgaccgggagaatatgtaggccagtagcggcggcgccggcggccggattgccgattcgtgcggtgtttggagtttggcctgcagtcgatgtgatgttattattatcgccggccgcgccggcgcggccggagtgcgttgtcgcggccaccgtgccaagatgtaccactcctgctttatttctagggccaaatcttaggaaattttcgaaagttgtaatgtcctgtgcctagatggctaagctgtgttggtttacgcaaagctcgttaggattcaagtactggatttggcacaagacgtcatcaattttatgagtgctctgccacttccagctggttggagtcgtactttccgagtcttttttcctttggtcttttgtttccctcctctcttggcataagtcggtctggcttaatgcgttgtgtaacgaatattcttatcttctaatatattgacgtgcaatccttttgcgcgttcgagaaaaaaaaacaattttatgagtaggaacgcgtttttgttcaatggatatgtgtatgtggtctggggacatttgttagtgccttcaacaatttctaagcgtgtagagaacaaccgtgaccctgaccttcttgcaaacaacgctgctgctgtctttgtctttggatgtgttcttatcctatttctgtaataatgctgcaggtttcggcacaccaactgtttgggcaccactgttctgcccccccccccttccctagctcgccaacaaatcttacagcaggtgagctagcactagttcatcataattcataggaattgtcaagtgcatgagttgcatggtcagtttcttcagaatgctgctttcaggtccagtagttgttaatctgacttctttgcgaaaaagccattttttctctcttaaattactttgttgaagtgtaattttacttctcagacaactaatgttttagtttgaaattttgtggggcgatagatgatactgtaatttatattgctaaacattttcaaaaatcttcatcactatttacatgcatttccatttattagaagacaaactggtcttctgtgctcaaaatctatacattctcgtacaaattgaattgaaaacacagcttgtgcaaggagaaacaaaaaaatacaaatgcaacaagggagttaatgaaacaagctttcagttctgtttggggagcttaagattctgagaagtagctggttggtagctagcttctgataatctggaaaagttgggtttttcaacttctggcttctataactacagattcttagaacctgagtgagaatgtagactatttgaggagctggaaattctaaaagaaactgcaaatactagaagctcactcaaacagggccttagtacttgaaggagtaaagtgaactagaataaagtctagggagttaaatgcttcaacgaaatatttaggggagtgaaatatacatctttcatttatttttatgaggataaccatgcttgcttctctaattatactagttttgtgattaagtggtttattgttattttatttgacacaaagcgtgtaatttagcccatatacaaatgatatatgttggttacttaactgtagtaaatggatactcaaattgatgtaaataagatgtgaaattgttattccatcccttggtgtagactaatacgtcttttattgacccaagcacttcctaacagcatgtgtcttgagttgtttacttgtatttcccgacgctttacttacttgacagttagtaggacctgcctggtggcccattattcatgccattaacatctttagttattagaattgttctgtgccttcaaagatctgtgagctccatgtttcccacatctgattagcatgtacacaatctaatgaaagaactcgcactttctctccaacatttagaattagagttcaggaattgaacatcactacccatgtttggttcccggctggctcaaattttgtttagtttcttcttaactaatagcatatctagttcctcctaggattctaatttaataccttaataa

SEQ ID NO: 5 - DNA, 91552 bp, Oryza sativa
ggatccactaatcctcctcctcatcgtcgagctgtcgccgcacagctgcttctgcatgaggacaatgggagatggcgaggagagcgcttcttcatgctatggtgagagagaagagagggagagggctcctctttattgccaagccgctatccctccatgcattattgacccaaaggtatgctcgagtgcataattagatgggcaaatgagtaaatgactcactgtaacgccccgcaataattagagaaaaagaaaagagagaaaagagcaaactagcagcccagctaagccttgggccttggcccatgcgtgcagcccagcaagtagcaagcagcagcaggccagcccatgcatgcatgcagcccacagcagcaacatgaagcagcagtagctagcatgcagcagcgagcagagcaagagcagcagcaacagctgtagctaactttagtcccacctcgccaatttgagaagagagggagcagcttataaggaaggcagcacggtcgctaggaattctgattttggaatttttgcgttaaagcgaggtcgatagtgttaaatagtgttaagtgtgattcaactcagatggatccgataccgttttcgggctggtaaacttcgtcgagtatattgctttctctactgttagaaataacattttgagttcaacaacatagacaacttaaaagcatcagtgatacggtagtgaagtcatgagttatacccacaaatattatgcatatgtaagtgggctttcaacacgaatttaatcagtgaaatcatatacgtgctgctgattttcatctatttgagagtgtgttagggagcgtattcgtgagtgtgtgagtgtggtgttacatgtacagtgatatatgtgcatgtgagtttgttgtgtaataaaaaaaaagaattactcattataataatcttctgaacatctcgcaaaaccgaaaattttgtgatccattaacaccctaggtggctaggccagaagaagcccaaagaacacaattccacattggacctagaagaataagaccaaactagaataatcaaacatatataaacaatgctcaaattgttcatctcgagcagagttgggtcgcgtcggctcaccccagtggcaactaatcccagcccgccttatggcatagtctgggtggtggagggagcagtagccccccagcaagcgctcgccgccgacaatgatgacgacgactatagcgactgacggggcggcggcagcctgcccgcctgctttggtgccgagctcctatgcatcggccatcaagctcccatgcggcgtcggctctttcttggatttcgtggtaaggattcggactcagtttcctatggttttattgggggtttctattcaatttgttccatagagatatcccaacccaaatgtgttggtctcgattttaggggttcctctcgacgttcgtgggctagggctttcagtctttgttgctccattcttgcaggcttgcagcatgtttcatgtggctgcaaattcaccacatgtaagcaaagagggttcacagctccacagttcacacagtagtgcagcaatgtgcaagttctagcattgccatggcataatgtgggaattctctggctttctttaggttagaacaatggattagtatgtgctctcgtactccaattgtatgagcaagattgcattctccaattccagagaaaaaaaacccctaaactcaggtgatgcaatccttaaatttccctacaaattcatcaattactattatgaaagttttttccatccatgaggatgtatcaggaggtactaagtaccaccagtacgattgaaatatgtccgttcaattcgcagggacacgatagtcataaaaaaaagagtggtttgatagggctgccccatgctggtgttgactacttgactttgccttccatcctaattcatcgtcgccgttttcgtcgcccctgctagcctactctctccctactccccagcctcaaactcctcctgatctagcggcaacggcagcgcactatccggagtgtggcctcgtccccgttctcaccaccctcacctaggggggtagcggggcggggcggggccgggttggataggaacccccccgctccccgcattccctcccccccagctgttaacagtgaaatttggtaagcccggagtagtcatcggcttagagtcagcatcggctttgaagtcctgcgattagccgatgagaattgtcaagtcgtcagttgtcggattcttgctatattcggttaagaaaattgatctactaaaggaagcttatctagaagagaccgagttcaaagagaatgcggcatggcaagttatctattaattaggaatagtttgttagtttccttttatctttagaaaagtgtgtttagtatcctataaggactttatcttttccttttatctttaggaaagtttttttcttgtccgacaaggacttgtatcaacccatgggtataaatatgtacacccggggtctatgtaatctatcactacgatcaatacaattcggcgcatcgccaccctttttacttctacttttgtttttacattccggcggaacctggcacccgacgcggggctgcatcgtcttcgatctccggcgaagggataagtccaatgttccgccggtccaggcaattgtatcgtctacgtcggcgtcgttcaaggctgcatcagtacattcgacctataggattactctggtttggataatatatttgcctggctatttatcatatgtctatgttaatctagtcctagcatctcaatttagctctatcggctgtctctcactttagggtttctgccagtatcggctaaatcgctttgctagattagattagcctagacatctaccaccctgaaaatcagtcaacggcttgattgtctagatattgtgtttcttttcatacttagtgctgcatcagttaagtttgatctactaagtcgtgcttagaaccataatctctagcctgctttttgattgccaataagggtttcatcggggtttcaaccggtgagttatctggacgttgcatcggctcataaggattgcatatacatataagttggatttagccgatgacaacaaaggtttcattgtttaatctaatcttgtggatttcatgacatcggacctccagccgatgtgtgctttaaccttcggatcgatgcttatttatcatatcaatgctagccgattggcttatactggattatattgttattttattacatcatcatcagccgattgtctttatatcattatctacattggacatatagccgattgcttaaaccctatcgctatcggctggtattggcatcggctattatcggctatcggctagaaccactccatcggcttgtcagacgatcggctatttgatctactatttacatatcttgtcagttgcaggatcaaactgactagcacgctcgcatctcatcaacctttgtacctgcacaggagctaagcagatctcccagaccggtgtgttcgattttttcatcaacaccagcccccgtccccgcccccgcattggtcaccacgtggaaaacctcccccgcccctgcagggagcccgacgggtgagcggggccccgcaccatctgggtggagttggtactctattgaaacaacacaacacactgaataaacaatgacatgatcacaataacttttcaagtattattatgtgattgatgttacaagtgttgctaacatttcatatatagccatataggtacgacaactaggcaagttctatgcccacggacaagccgtgtgctgattaataaaatcttaggatagcatagcccacattgagccccatataaattaacactataaaaaaagtctagcaccataaacttttaggctaaggcctcgagccatggaccaaattcctcaagccccaaatctgcatttgtgtgtggacaaatcccataaatgtaaatagcacctaatatatgccgacgttatatatgatttaccctatcctgaagctattttaaactacactagctttacggttaccttatcttatctcattataatttaaattacaccaaagtttcgctctctattatggtgtcttatctttttcactattttagagctctttacgcgccattcacctaaaatagaataccacgtttttcactataaaaagatcaccctacctctgtcggcaaacacagaaaaacaactctcacaagagtcattttcattactaggttcgtgtccgtgcgcccgtgtaggtgaggtgtgctatggcacggcataccaaaaacgctgcccgcagagtgactcttaggaggcgtcgtgctggattgattaaaaaggtaacagagctatcaatcttgtgcagtgtccaagctagtatcgtggtgtacaacatcgatgaagcatgtgatccagtggtatggccatctattgaagaggccaaaaacatgtggagcaaactcatggacatgccagaggctgtccagaagaagtggatgcaagactccaagaccctactccagcagcaaattatgaagctccagaagaagctggacaatctgaaggctgagaactacaaacgtgagatcaccaacataatttctgagataggaggtggacaccgcaagaacttaaatgatttatctcccgagatggtcaaaaatgtcaagcgggaggcggccaagctccgggaggccattagaaaccgcatcatcgagctccacgcacaaggtgcttcttcttcggtggttgtggctccacaagtggaaatagttgcaccacatgcttctcaatttgatttgaatgaacatgcactagtttaatatggcgtgcccatagtgagggaagaggctgatctcccttgcaactaaatgtgctttgtttggtgattaccggttgcaaggaacatggctagcctattacttgttgtatgtgagaagaatattgatatagtttaattggtggtaatgtttcatctattataagcttgttatgataaataaataaattgttgttgcgtaagtaatagaaaaatattttctctggtaagaaatagttaatgacataatattgtaacgccatagttttagtgaggcattaataaaaacctaatttcaaacgtgtgaattgtgctagaatgatacgatcaataaatagtagaaaaatgattaagcaatgttttatattgatattgtttgtggtttaaagctaatattgaaaaataatgaactgcaataacaatacccaaaattaactttagaaaaatcaaatttcaagatttaaatttttgctatggctgataagcgaataagtagacacttatgatagtcttggtatcttctttaccaaaaaagaaatgacgttttggatatcgatatactagtcctcaaggtccatgctatcatttctctctcaaactatatattagcaaaaataatttaaatataactgtatgaaatatctttttttatgcaaaaactactaatatagtttctgcattttaaatgctgataattttgtagtatgtattaattaatgatttaattaagatgttaaggttacacattataacatgtcatcgaaggatagttgtaaactccgttgagtatattgcattctgtagcgttaggaagaacttgcattttgacttttttttttactacaagaacgtagatagatttgagattgtctttagtgacagctaaagtgaactatttcaaaatggtgtggcgggaacagtgcacggacacggacatccgccactgtgccgtgtcgcccgcgcaggtgaccacgccggccgtctggcaagccgtgggttccctccacgttcgctgacgcactggagcgcagggtgactccgtcatcgcctctgccggccgccgcgtggcgcgcgcctcccatcgcttcactgcgcgccgccctcaagagatgggaaaaaagagtgaagagggagaggcagatgagggagaagagatgggccgctgacacgtggggaaaatgctttatatcgtacgtccgatcaaaaccgaaatatcggacgcgtcatctactgtaaatcggtatatggataggatgaactatttcactgtaacatccatggccaattgtttccttttgttcttacctcttctctttaaatattttttcttctagattacttattcgatctacgaaccgatcacaccgttatatttgttgcaattaaatctttacaataagatatcatatgattatattatataaaaaaacatgtgtttcaaaccgttaaaactaaatatttcatacgtgatagtgatatgtttcaacatgcgtcttcagcgtatttcaaaaaattcctgatactatctgctactaccctctccctcctctctctccatctcatccatgcacacatgcatttattttagcaagcttcaaaatgatttttctcttaaattacttatccaatatatgacccgattacaccattgtattcgttttaattaaatctttacaacaagatatcgcatgactatattttgataaaggaaaaacatatgctacttgttgtttcatatgtgatagcaatatgtttcaacgtgtatcttcaccatgtttcataaaatatttaaatgttttagttgctgattttttttaccgtatataacttaatgtttcacttattggtaaactgcaacatttgaccatccgattttttgataggcatcggacgtccgatgggtagctttctccagacatatggggtccacgtgggtccccctcatcatgtcagccaaaaccgatcactgtattatcgagtgatcaaagttaacgaggtattgtgagttgtgtcagccaaaaccgatcactgtactgtggagtgatcaaagttaacaaggtattatgagttgaggatgtgctataccgtatttcggttcaggggtgaatggtagactcggcgacaaattgaggaacctaaagtgaacttataccataatgaaatgaacccgtcggcccagatcagatcggagaccaatcccctcgactctgcggcggcggagtaaacgagcccaagcaagcgctcgccggcggcggccatggacgacgaccgtcggcggcgcctcttcctggattcttcaggtaacgcccggattcagttccctgcgattttggtggggttgccccattcgattcattccatgaagatattcccaggtgtcgattttaagggtttcgtgtcaattgcgcggcagattcgtgttctagtgcgttcaattatgttttctaggtgttcatccttcttgcatgtttaatctggttaaaatactggcacatgaaatgcacagagggttcacacttgcatagtacatctgtgcagcaatgcatctgctcttgcttgcaatggcctacgatgggagttgtcagaatttcttcagctaaacaatagtagtagattacaacaaagttcaaacgctgcatcatctgattgctttagttattttttctgaaattcgagaatgcaatctaactcataaaagtgaacccaccattagataataaacaaatgctatgagatcattctgcagaacaacgatctcaaaaatgaacagaaaggaaaaacattgtctaaacgcagttcaatagtaaaagggcctcttacaacaatctccaaagcaatcaaaataacagaagaacacttcaggtccagatggataagaaagcgaatgtttcaggtagtgctagcaaagtagcaataactccataacgatgaacttattggaatggtcaatggacctgacaaatgcagaggcatcaaacaagattgctaatgtagggagaaggtaactagcactcctagtcccctacacaatcagccattttttggtccatctcactctcaatccagtgagcttcaaagtcccagtgcagaccaaccagccagagtttcttcagggtccgaagggtttcaatgccaggagggacactctccagccctgacagagcaacaatgtacaaaccttcaatgactggaagtgcaccacttataatttttagctggttgacatcaggcatgtgcttcagcacaagtgtcttcaggcatgggaaagccgccgcgccaagaaccaatctgcaacatctatatacggacctctgcaacatgcagcatcttctggtgcatcccgtcacctttccattgagtcttgaagtctttgtgcaggtccttcaaccagagcttcttcagggaggcaagggattcaatgccttgagggactttatccagcttccacaatgatacaatgtataaaccttcaatgcatggaagggcgccatccgtgatgtttatctggttgacatcaggcatgtgcattaacacaagagtcttcaggtgggggaacgcctctgcatcaagaaccaaagttttcgaactgtgcacgttgttcagtcttagataagtgaggtttgacaagtgtgatgcgagcatccccagtggatcttccccaagattacaccaacttagagctaagtacttgagatgtgtagtgtggctacgaaatatcgggtagtccaatgtgcccttggcccattgccctctgataattaacctgtggagttctttggacatgggctggagagcctcaaagcaaagaggttcattctcatctcttgcagaaagaagcaagctagaaagaagcggcatagttgataatgtagcaaaaatatttccacaatcagcagaacttatgttgtcaatccaaatacttcttatctgcattagttccttcagctgctcggccaagtccttgctggcttccacagtctcaagagtctgaagttcttccaacttagacagatctttgggtgcttgcattccaatgaaatagcgaaacactgactgcttctcgtcttcatatctatcagctagcaggtgccttagcttcttgatcttagtgattccacgtggtagcttctctattttggtttgcttgatgtccagagtttgcaggtttgagagcttctcaatagactctggtagtgagcagagtcttgtccgccttaagccaatgtaacgtaaattaaacaatttacctatgcatgctggtacttcagtgatatctgaatcttgtagctctaggacagtgaggtatttggattcagacaaaattgaggataacaatccaggagggtgtgtagttgtttcaagtagtgtgcgaagatgtggaaatttcactgttgatgcacaaccttttccattgttcaagaataatgacagacgacggacttcccaatcaaccttttccacagctccataatcatttacgcaaccgaacctctcctgtccagcaattgagagagccaggttgcgcacaatgtcatgcatcttacaagatctcaccctgccaagctcatcatactcgtcaacttcaagcatgttccggtggatcagttccatgagatttatttcggccacatcttctggtctattgtgttcggttctcaccgcaaaaccttctgcaacccagtaccgcacaaggctctcacgagatatgcgaaaatcttcagggaacaggctgcagtacaagaagcagttcttttggtcagctggtaatgcatggtagcttagtttcagaattgccttgacatcatcatttttggccagctcactccgaagctggttgtacatttgttgccaggcatgctcagtttgtagctttgtggacatcaggacacccatggtaacaagtgctaggggcagccccttacacttacttactatggaggcagccacattctcaaggtccagcgggcacctatggtcctttctgttgtaaaatgcccttctgcagaaaaggttgaatgcatcaatttcacccaaagcctgtatcttgagatggcattcagagggagcaagaactgccacatgttccatccgtgtcgtgatgataatgcgacttgcttggggattcttaagcttgccctgtatttcgaagtacacgttctggtcccagacatcatctagcacaatcaaacatgatgtactgttttcagtccttctgtttaattcttctgtcaaatcttgaacgcccattttgttgattagatcttccttagattctgatgattcttgttccatgcgtatgagctcactgactagctgcctacaaagactaaggatcgtccaagtctgtgacacagtgatccaggcatgaactgggaacttgatcttttcacgttcatatacgtctaaggccagggtggtttttcccagtccacccataccagacactgttattactttgtgacctggttcttcagagtacagtaattcaagcaaccttttcctgttgtattcaatccccacaggatcgccacattcaagcaacttccttcttccttgagattgcggtgtttcgatgtcagtgggagttcttggaatgagctgaactgtaggtaaccattccgtttgctgtcttttaacctgttcaatatcaccctttatcttcattacctcactagcaacttcactgaaaacacctgcataatgtactcttacgaacctcatcactgatccttcttgctgcagttgacaagcatagtatgagtacttgtccattatgtcttcaacacggtaagccagcttccgcagctcgtcgatccaaccctttacaacattcatgttggtatttgtggaatctaaatcttgtattacatctttcatgacacgcaattcccttctgatatactcaactttgtctggaagttccctcaagttagttaccttcccagacagtttggctatgacagctctggtggcttcatctcccaatgcaatactgatctttgagatggcaagcagcacagcttctgccattaccttagtgctgcagaacagaatcatgaatcatggagatagtcacataacgaattgataagataaaaaaggttgttgttacagttgtaaaacaattgcagctaagattgttttttgggcccttgtgggcatttgcaagccatatatatatatatttctaatgttaatcatggtgtaccaacagatataggtaaaggagctcctcattacgtaccagcctcaacagtaatcttaattgtaagggtaatgttctatggcaattgtttcataactctgcaatgtgaccaactgtgtaacttaggtcctgtttgggggagcttgtcccagctgtaacttttcccaaaagctgcttctgctagaagctaccccaaacagtccacagcttctgagaatctgtagttacagaatctgaaaaatgaactaagaagccagaagctggagaagctgggtttcagagcttttccagattctcagaatctagctaccaaacagttgcttctcagaatctaaagctcccccaaataggcccttaataggagatcaaagtaactcataatcagatagtctcatagtttagttgagtgtgttgtttcttgaagtaggtctctattcaggtttttaatggttccacagatagattgatcatttagtataactcatatgtgcccagctttttaatgcttcatacaaaggccagtatctaatatgtgtgagtatttcatgcaccatcacttcttccatgttcttaaatgcaatgaagtatatatgaaacagcccagatatatctgaacataatatcaaacatgatcatagtttgtttctaatcttacttttcttttaagcagccatttctaatgacaactctgttttggattctgctcttctagtctgttacaccatgcttagatgataggtttcttgtcagtagaaacctacttgtgaattctggcattaagctgctgaacctactgataagtacttaaagaacatgactgtgactcaatttctttttaagtgacaaggagcagaccagcaatgctcttaatatgaagtagagatgcacaccacatattccagtgttctctttctcagcatggaagtgctgaagcgcatgtggttcgattcttttgcttataacaggtagaggtcaacattttacatatccttttacaaaacacaccgtttagcaatacggaaaacacattaacgaaaaagaaaaaagttgtcgttttgagttgctgaaaagaactaccctctaactgtgaatataagagattataacccttcgatttgtccacaaatacaagggatcgtggatcctgaggtgagcaaacaacagaagggatgcagcaacattttaggccccctttgattcaaaggaaattgatagaaattttagaggatttcattcctataggaatttttcttacaaagccttttgaattaaaggaatgaatcctatggaatcctataaaattcctatggaatgcctcttcccatacaagttttggaggaattttaacaagaggtagaacctcatgaaaaaatcgttttgagtctttatctctcatcaaattcctgtgttttttctgtggtccaatcaaatggtcattcatacgttattcctgtgttttgcaatcctcttttacacttacattcctgtcagaattctatgtttttcatattcctccgtttttttattcatgtgattcaaaggggcccttagctttttcttacttttttttagtttattcaatgtgccaatcggcctaatccaattagtgcttccctttatctgtactttttcctattccttaatttctattaaaaaaaatataatccctcgtattacgaacggagggagtaccgtttagcactggtgtagatctgttgtataggcagggtcatggttgatttgttatgagatgcacttgataatgtagtgataaggggtgcgtggtttcaatcccaaggtcccatgttcaatcccaacacgcttacaatttattcttataataaaaactagcatggtggcccgcgcagactgcgcggctagcaccattatgttttttcccatataattgcatatatgttttctcattatattattcaaatatattaaaatgacaacataattttaaattttacaataactttacaaaactactaatacataatattcatattatattttatatacgtgttagttattaattatttttaatatcaaattttagttatttgtaaattatatatatttctatatggactctagactcgtgttttaatatttcttttttttaattccgaattttctgtaaattgtatttctatatagactctatgctcttcttccaatattatttatttttatttctgaatttttattatttctaattgtatttctatctggactctaaactcatctttcaatattctttaatttttaatttcgaatttcagttacttttaaattgtattcctatagtgactttaaactcttcttcccatgtttttcttaattttaaattttagttatttataaattgtatttttatactgactctaaactctacttttaattttattatgtttattccaaattttagttagttttaaattcctatatggactctatactcaacttctaatattccttatttttttatttcgaatttctattttttttcttcattgaatttctatatggactctatactctacttctaatattccttatttttaattccgaatttcagttatttcctaatcgtatttctatatggactgtatactctacttctaatattccttattttgaattccgaatttcagttatttcataattgtatttctatatggactctatactccacttttaatattccttatttttaattccgaatttcagttatttcctaattgtatttctatatggactatatactctacttttaatattccttatttttaattccgaatttcagttatttatttcctaattgtatttctatatatggactctagtctcatcttctaatattccttattttttaattctgaatttcaactatttctaaattgtatttctatatggactctagtctcctcttctaagattccatattttttaattctgaatttcagctatttctaaattgtatttctatatggactctgtcttttctttttccctaattaatgtgagaatttatagaccatgagagcaaacatagaggcttcttcttctattcctttaataatataatagatgtttgcccttcaaatctcgtttatctggttgacttgttactgtttctttatttggtcctagttgtttttgtcttccttttgtaatccatattaattctgtacagccgctatagtagcactggatcttttgtaaagcaacatttcaactatatcgcaaattgattggccattagaaaatttcgttgggctactcatctttcatgctttgttcttggtgaaatgattggtaatagtttccacgggcattcaattttagcttaagatgtccttgcagtagatggccaacaataagataataaatgataaaaatacgggaaacatgtaattaacacacagtatttaaggtgatgcactgcaaacaacagtaatcgctaaatgttgcagttgaatcagcactgaacaaaacaacaacacaaaggttctttccccatctctgaactgagaatgtgctgctggccttagatatgctcgctcagggtcaaatttcagaacaaatttcccaatattttctagcaaagagactaattagtgatccctcctctcaacaaggcgaaattcacgaaagaaaaaggcagaattttccagcagacataatcagaaaagttaaagctgataacagctacaaactagctagatgatacaaagcaagatccagtggaaatcaatctaacctgagctgcagacgatcagagcgtctagaagagcagagctggcgagacgatcagagcatgtgaggtttaagataggcaacgaagaaaatataaagcacaaagccactagcctcaaatctcaatcagatccttgtctgtttctctgaactagccgccgacgacttgacgcgaagtcgacgcggctaagcatttgcagtgccaattatcaactggccaagttgtgcgtgtaggacgggagtccgctgtggaagcttcctttttcctgatctcaaaatttatagtataccctccaaaccgcatgcagtgggggatgctggattctttttaaatggaccagaaactcgagaaattattgctcaatagattattttgcctatagtaaaaaggaaaagaaaaagtaaatcagaattgttcatatcgaaaggaatagaatatctgtagtccagtatgcaaattagaaaagatacagttttgattagtttactgtcctgcagccagctcactggtcaggctgttcgctgcatcttggacatatactccctttgcccaaaatataagagattttagacggatgagacatttttttagtacaaggaatctggacatgtactagtaagtgtcacatctgtttaaaatcctttatatttaagtacgaatggagtaggaagaaacatatcgttatatttgaggacggagggagtaggaagaaacataccgtgtcaccttaacagtccttgtacttatgactcagttgttgaggacctagttagctagtcggcaactgcaaaaatataattccagtctccttattctactcgctaatgacagcttagagaaagctaacaaactgatttcttatgttttcatccaactaaaacggccggagacctccaagtcgtcttagtataaaaatctctcaagtcaagtttcctcgatttcttggtcgtcgtcttcgtccaccttttggtatgctcgatttttttaaaaaaattatgactctttttgtaaaacttatttatgaataaatagactccaaaaatctaattatggatatggactctactcggtgccggtgagcttggcatcgaggtttcgtgcacccacgctggagaccttggcatcgagttagagggggagggggagagagtgtcgatgttgtagatcacgactacggtatttgcagagtttggggatcattcgtatcagggtataagcgagactaaggtaaaataaacggagacaaggattttcatgtaggttcaggctccttatctaccaggtaatagctctactcctgctaattgaaaccagtgttgctcttattcatcagaatcacacaagtacaatatttgggataacttatctaatcatcgtcaacacggcggcatgaaccaccacacgttgtcgacaacagggtagtcctcctcctctaatatgaattgggcgatatcagagatagcgctagatccctcttgtcagcctctgtggcaccagattggatctgtttaggtttatctcttatgttgatgtctggcggcatgtattgatgtatattttgactgttgcctgtctatttagactcggcttgtcttggtccgtctccccctcttcttttaggggtcttgtatttatacccatagatgtccccttatccaaatagaactagaaagataaatatggatacgatccgaatagtccttgtagtttccatgtagaactctgcttctccttccttatctggaataccttccgtatgcaagatttgattccgtataagacttggtatatggtaggtcccgctaagcttaacccaacactattgggtatgccttacccataaaactgatagggggtaggcgccatgcttcttggtgcggacaccgagttgtcccgagatggcatgggatggcatgagtgtacaacgagcaccaagagtctgagacaccatggaatggcacgtgtttggcaacttttttgacaagtagacaagcacgatcgagacacctaaacgccttgcggtgattgtagtatcgccaaccacctcaatctagcaaaagctagaccaagatgggttttgataaactaaaccggctagcggagccgatttagatagaacaatagataactacccgtctcgtgggtaaacggaaggtttacgggacaaacctacaaaggatttcgcactctcgtagtgaaggcagacgatttatggcacaaatcgacaaagatggatgaactagactagaactagattgaaatattgaaaaagcaatttaattggctaagttggattgtgtgtagattgtatgctcaattgaaccggccttgtcccttatataggggttgatcttgcctcctacaggtcctcctccacgtccaactcaggatagaattcaaaggaaacccgaaatatagcctcctgagtaaggaatcctgagacctgacgaaaacagactcgggcttggactctgccggtctgaccggccacatgccgctggacagaccggccctcaagtagcggtctgaccgaccaacaaacgacggtcagaccggccctatggaggaaaccggcggttttcccaaatcttagcaattttctttaatttgaacagacaaacgacgatgatgagcatggcgcacctcaaccctccctcttctccatttcaatccctgcctttttatcagttgattctaccttatttttcctctatgtagctcctaatgttactactttctctatttcatattataagactttctagcattgctcgcatatatatatatatatatatatatatatatatatatatatatatatggcaacatatcctatgcacacaggccctcacgtgtacacacggtgcacaccaactaaaaaatgtcaccaataaatctagaaaaaatcatacacatactttcaattgtattacacctagggttaaaatcttaacgtcaaattcattatattttagccgtaacaaaaaaacaaaaaatctgacagttttaaggttgcaattttgtcagaattttatcttttttgttattctctatgtagaatgaatttgaagatgcgactttgcacgtagatgtatatatatatatatatatatatatatatatatatatatatatatgtgtgtgtgtttagatttattaacatctatataaatgtgggcaatgctaaaaagtcttataatatgaaacagagtgagtaatatttttttctatggaaatcttgaatacccatcccgaaaatcatgtctctgtcactaatagggctagtggcatttggccaagcgttggtcgggttggtatgtgcaggagttactagcacgtgtggtggaggtttttttttttttccttgataataatttcgtagggttataatcttttgtactttttcccagctttatcaatatgaaatttcacaccatcttatcttatgtgtgggccattttggaaaaaaaaaactgagctgcaattgtatcgctttcagtaacaccagaccacgcgatcatttttactgtgactcggtggttgatgacctatagcttagctagttggcacaactgtagatccgattgctccatacaatccgtctgcttattctaatcactactggtagctaatggaacgtaaaacgaggaagtcattgccattttgattaattaaattttaattattttaaacttaaaaatggatttatttgatattttaaaacaacttgtatataacaagttttcacacgaaatataccatttaactgtttaaaaaatatgctaacgaaaaccaaggtaaaatctgtataatcaaagttagaacggggttagagaaaagccaataaaacggccgtggacctgaaaagtttcattagaatacacctctcaagtcagtctcctctgcatcctggtcgtcgccttcgtctacctctcgcctcgccgtcgccggaagaggagctaaccggctggagatcgattgatccaacatgagtgacaatcctccaggtatgtgcatgctacaagcttaaccaatcatccataggcgaactatttcacgtttgaataacatattttctgtaaattttcttactttgtgggtgttgttcatatgcaggctacttcgttggtcgcccattgaaccatgaagaacagcaagcttcacggccggctgaggaacagaacgctcaattccctggtaggtatacaggattaattagttttctgcatacatcaatttgcttagttttctgcatacatcaatttgccggatatgatgtagcagatcttgttataacttgaggttgataagtgctttgaaaagggttcttaattttcagtcttctatgcattcttctattacctcaggctgttttgtttgctatgtcctatgatagttttcaacttttcaaatttcaaacttataaagattaagagaaatctttatcttctgattttccttttctactactaaggctactacaatggcaatccggtcagaccaaatgatgctaagggtgcacagaggaatgaaccgggcttctttaagaaactgtaagctgcaattcgtgtacagaatatcagatgcttctgtctgtagttacccattgatttgtttttctctttgcatgatgcattggctcattgaatctctgcagcttcgggtgcttcactgccggtcaaaatgtgaactagatcaacacggctgtattcgcaatgttaatcgacagttcatcaagaaatcagtggaattttgatggagaagttatcattttaggaaatacaagttttgtttcttccaaagtttttcttttcctgcagttacactgttgtgaattgtacaatcatatcccaagcaaatgagattattccgcgttatccattagtattttgcttacctgtttggatctatcaatttactatattgctactggtctcattaaaaggattgttttatccaagtcgtctttgtcaggaaaaggtccacatgaatatgaaaactttgaactttctcttggatggttcagaaatttttttttgactcaatcgcctattaattacaacgaacaatcatttgtcattttcaacgaaaaagaataataatgtttgcaaacgagagcagcgatcaatgagatgactttgacaattgaccgacttatttgcaactgcaagtcttcaagcaattgaaccaaactgtcatggattttaatttttcagcgtttgctttgagatgaaaatgactataaattaactggaaaagttctaagactcttcgctgacatggattgataggtattctggtatttggcatgcttgtggtcttcattaattaatcctggttcagggtaaataaacttggacaataatcaattattatactcattcaaatcaaggcctggtttaattcctaactttttcttcaaacttccaacttttccatcacatcaaaacttttctacgcacacacactttcaacttttccatcacatcgttccaatttcaatcaaacttctaattttgacgtgaacaaacacaccccaaattacacttagaatcttatattcgccaccaccagtccatactatactaatcaagtagacacctttactttcatctaatttccaagactaacagccaatgcatcaaccttatggactatcatattgaaacattttatatcatgttccttttgcaatacttatttacaaatacaatcctatgaaacttaattgtaggaatgaaccccaatctcagtcacagccaagcccaagcttaggagtgaactccaacatcttagcaccaacaagcaggtcgcttagttactactccctccgtcctaaaatgtaagattttttagctatgaatctggacacgtatgtgtccagattcatagctaaaaattgctatattttgggatggaggtagtatattaatcttgcttctagcatggtgccattggtcaactccaaggatttttgtgctgagatgttagaagttttagggtccatttgtgcgatcagtttttaagggtttgttcttaaaataagtattgaaaaagaacacaaatgtgtaaaatcttcaggggctacttaaaagaaaaagctttttgcaaacagtcaaaagtgatttacacaagtggccagccattatgtctacaatttaaggcctgcgaaaatcatgcatctttcacattgattgatattctttgtcttcaaaagcatcatacatttatcattgatacgtaggtttatgattcatcggacctatatatcagtaataaagtcacatgcctttgaatgtagaagaggccgatctctttcacttgcagccaaactaacacggcccaaatatctgtaagacaatgaagttttccctgtcatgacaacttacaagatccagagtatctgtcctaactaaaacggttgccagtacggtgaaaataaaaaaaacaaacagtggatgatttgatattctcattgcatgccatgtttgcaatttcgtggaaagaaataacaaatagtggatgctttgatattctctatctcatgccatgtttgcaaattcctgatactctagttactgagctggtcttcgagtagtagtggtcgctggttggtgatgccacttgagcaacgacttgggttttaggtcgtcgcggccgaaaaaaaatatcaatctctgcgcgaatcattggcgtaggccagcctgggagagaggaaggaacatcttcctgtgcctccttaccctcttcgtcttcttttccatctcgttgtgtgcgctcagtagcttctcctcctacgaaaccaaccagctgccgccggtgaccgtcgatccgtcgaccgccgccgccatgagcagcgatgttcctggtacgtgtcactctgatcactcaaaagctttcgtttctcttcttttttttttttgtttgtatcaatcaatcatgctaaagcctaaaggaattcttgcttctttatatcagtctaagaatttttcgtttgctgcagggtacttcgtggggcggccgatgaaccacgcggagccggcgaaagaacagcagcagggcgccgacgagcagcggcctgctgccaacgcgcagatccctggtggtacgtgcaacgttgcaagctcgattcgatcaaattttgggcacaaaagaatcagttttgcccgatgttttgctgaaccttgcttcgttttcgttttatgcaatcgttttttgacacaaaaattgagtcaattttgtctggttggtttgctatttttttttttgcttcgttttcgtttttggggggagaagactacttcgtcgggcgtccggcaaacccgcagcagccgccgtcgcaacctcagcgggcgcaggagagaccgagcttcttggccaaatggtactccaatttgttctttgaaaatcgaacagcaattatttagattgtattagatttcatatcgattcgtcactctgtgctttgttttatgagttgtttgatgctttgctcttgctgatttctctgttttgtttctgcagctgcccatgcctcgccggcggcggagccgagagctagaccgacaaattcgattggaaaattgaagcaaatttacatggagtgataaggctcagatctgtgttttgtgttgcaagttttgctaatcgatgtttgtagctgaaggttgcttctattcatttatataccaattgttcattcgtatcatcagagtgcgaattcgatgaaattgtgaaattgtccattctaatccggcatgctttttctccttccttgaaatgaatattctactccattgctgggtaatgtcaagaatgagtttgttgttttattccgtaccgatgttgattaattttttcccctttttgtgaaaaaaatccaaatctttaagttcgtctttgataagaaaaaagaaattctttctttgctagaaaaaatatcttctgttttagtcaaatttgcaacttgtgtgcatacgttttggcaaaaaaaaaattgttacgtgacacctaaaaatgtgtaattagctataaggcattgcaaagacataaattagttagaggatatcataaaaacctcgtaattagctaccggacatcaacagccatattttattatatgtggtgcagattgagagagaaacaacatgaaaagtactaatttgcccctatctattatctattatctatctatctattctgttctatctattatatactaaagtccatgaaacttcctacaaacgttctcaagccaccacatggacatcctataaacgctctcaagccgccatgtggcatgtggtattttaataaattaaaaaatatcaaaagtttctaaaaataacccaccattgattttcacttaaatcagtgggcccattatttatgccatattagatctgttatagataacacaaaactaattaaatctatacctaataaaaaaaattcgtaaggtttccgtaagaatcacgttgctaaaatcgttttaatccgctcaatggtctggatggatccttttcagtctacctcaaaactaggttaatataggatatgtattaactgatataacatgtgatcacaagtagtagcttgcctacttggttattggagaaaatcttcattgtggccaccagggttctattcccaccaccctctccctttaatgcctattataacaggctttggcccatttaagtttctattgtttcctttttctttatttgtttttttcgcttttttcttgggaaaaaattcctcccgacgctttatcttttttttttccttttttatgcggtaagttaggataataacaggtagggaattcactaaaaacaaagttcaatagaaaaaacacataaataatttagttaaaacttttaaaagaatagttgaaaatttcaaatatgtagttgaaagttttcaaaattcgagttgaagtttcgaaatcttaagtgaaagtattcaattttggtataacttatttttttataacttttcaattattaaagtaatttttaatttttagtaaaaaagaaaagaagtaaagtagattgtaataccggaaactaattacgtaaaaggaaaaaaagaggtgcgaaaacagagaagaggagacatggtggcaaccaactgcgtgagttttttttcaatttttgatctgactccttgcgcaagaggtgcgaaaaaatagagaagacactgtggcaaccaattgcgtgaaaaagaagtctcgaaacaaaagataataaaaaaaagggaaaataggtcgcgcgcgactgtgcgatggatgggctcaaaacgcgcaccacttaaattcgcgacgcgtatgcactaattaggagtttcgtattattactttcactgtctatgggcctctcctaggccagattgacctaccctacctaaaacttttacacttattattactcggaataaatctactttgtctccagatctcaaatcctatttgcgcgcaatttttttctctccgccactagcacggaccatgcacatccttttttttttctctccaccattagcacagaccatacacattcgatggctgaaaagtcaaaagttttgtacctctttcttcaacagatatttagtgattctgaaatcttataatgaaccagtttttaatttaaaaattaaacataattatttttttcttttcgtcatctgcaaaacacgacatattagagtccgcaccatctatcatccacacatgagccgttaaacgatccgtctccacgcgagtccatgttggattttagaagttatatcagactcgtgccatgcaagagttgagagagagcgtgtgctatgacaatatgagtattatatcaatatgcaatggtgtttcttattagtcttgattatcattaacccaataaacaatttatctatcttgaaatattattacatacatgttaaccgtgtatttatagcatttaacctttcattatacccgtagcgaagcacgggcattttgctagtctatcaaaagaaacaaactatcctcccacccctccagccgtacgtgcgtatatagtacggagtatggacgtacgttttactccttctcccaattctttttttaactaatttttttaatggaaaaacaactaataatttaaaatccatatcacccatattttactatgtacaaaaactaaaatatattatttgatcatatcgcatcaaaacagcatccggtttttaatttatttaaacagtaattttatttgtaattaattaagtaaatcaacatctatattactattttgctctataaaaatttaaatacatcatttgaacatacgggcaacaaccaaacaacctcgaaacattaaaatgttagtgtcatcttaaatcattctacgcaataaagattcatcgaacttatcacattttaaaactagaaaaaaattcttagaaatttgagcatcattatatactaaaagtccattacatggcattcctacaaacactcctatactgccacgtggcactttcctataaaccggtggacccaacattttaaatcattagattattaattttcaccactaaatcttatcttccaaaaaaaaatatgtacgttcccccgctccgatcacaagtttacataatacgttttaaaacgaaaaaaaatgtgcacatgtatgtacgtaactccctctctccccctctaaccctccccttccatatttcctattttttttggctaacaccaaatacgtgtagtataaaaattaattagatagctaaataatctcttggtacatatgtttctatataaaaattataatacgtcacttggatgtatatacccttaattttcaggggtgaactcagaaaaaaaaaatcgcaacatcacttggatattatcagggacactacacatacaaattatttaattactgagtgatatgttaattaatatgacatgaattatctattattatatactaaaaatccattagatatcctacaaattctctcaagctatcacgtggcttcccataaacattcctaaatcaccacatggcgctctaatatatctttatataaaaaattttgagaaaaaaacatcttacccaaaccggtggaccttatttaaaatgttagatctatatcttaaaaataaaacctaccgatcgatcaggccgtcaaaatccatatgtataatgcacatatccacttccctcctcacggatgagacgaacccactggctttcgcctcttttttttcttccttacggcaattaagttaaccactaaataaaagaaaatcctaaaaagaatttaataaaaatggtgggctcattactttataccattaggcatctaacaacttctactaacttaattatatctctcaacatgtatacgaggtctatttacctatataaatatgtaaagctaaatcaatctacaaacgtacaaataaattcgtaaatccatatatgaatgaagctttttttaattactatgtctgtcctataatgaatcctaaaattttatagcaattaaggaagaatgaaaaaaatatactaagattcccctctctaaataaggctcattaaataagtggtatgtgcagagttagaagattgttagagtactgttagataaaaaaaaaatgaataatggatggttggatgggatgagtagtattaaaatcggataaaatttgaatcctttaatttatggatggaggtagtagaagcaaatagaagtatgtgattatgtctttaattagataaagaagtcatattaccatatttttaataggaaatttcttaaattaatatatcctaaaatctataaatgcatctttagtatacaagaaaaagtattacttaaaagtttaaattaagatgctaattttttaaaaaagaaacacatatagtataaatgatagtttcatatgaaccgtatccattgttgcgacattttttctcataaatttaagatgacatgattcatgtaattttagtttacaatttgtaacaatccgttctctagaacaacatatcacagcctatttaactggcactcccacttacagatatcaccccacttacactttcgttgtcgcttcatgtaaaataatcaacacgaaaatgatatggttagagggacaatgccttacaagttagttgcactcttgcttaactagcaagatggtactactaaagtgcacacaacacaaatcacatcctttaaataaaacataggccatatctaatcgaactaggatgtcacagacaccccctcaaaagacctgacgtacccattggtccaacataagtatagataaattaaatgcctagaaacatgtcccctctttgcacgtctgtacgtccagtgatcgaaactaacttttaaaacaatcccaaacaaattattgatatacacatatttagaagagaaaaaatattttttttaaaaaaatcatatacataaacattcataacaaaatttcaaacaccactaaataagatacccgcgcatcagcgggggctaccttcctagtattatatactaaaagtccattaaactccctataaacgctctcaagctgccacgtggcttcctcaaaatactctcacattgccacgtggcactctaataaaatagtgaaatccgaccatcgattttcatttaaatccgttagatctatattaaaaatcaaaaactcccaccggccccacctccgtgcagtactcacgtacgtactgctccctatgttccgttgtcttttttccctctgctcctttcttttctttttccaatcacagttaaaattgaacttatctttatagaaaagaatacccccacaaatagacattaaacgcgttaaagtccattaaatatcatataaatatttctaactgttgtgtggcactcttaaatcagttattatatactaaaaatcgattagactttctagaaatgctctcatactatcatgtggcaccctacagacactactataccgccacgtggcgctctaataaatttagaattctaaaataaatctaaccattagttttcatttaatctggtggacccattattttttactatttgatctataataaaaataaccaatccgatttttcttaaaagacccacataacccacgtagctgcatgtacgtacttacgtacttcatgcacggcaccacatccatcttttctcttttcttttctttttcgctcacgacaataaaaaccgaattaattattccaacccatatatattaactccagcctatatatatatatgcaaaaatatatgcacataccagtaatatattatatatttttatatattatagatattatttagtatgatgggccccacatccctccttttctttttttttcttttcgattcatatgacataaaaaccaaattaatattaataattatatactaaaagtctattagactttctgcaaacgctcctgaaccgtcatgtggcatctacaaacgctcttaagccgtcacgtggcactgtaataaattaaaaaaactataaatcctgagaaaagaatatctaacatccactttcacttaaattgatgggcccattattttaaccgtcggatttaatctcatataaacaaagagatggaaagaaagaccacataccttccccacctctcccctaagtcgttacgttcgccctctccgcacgtactcccctctctctcccgcccctctaagtataattcatttttcctatattagctaacgggggaaatttgattaaaaaatttcaacatttgtaacatctaaatcatatatcctgaaaaataaatatctaccctttaattttaatattaaaattagtggacataacacttcaagccattggattagatttaatttttctcatccttaaaaaaaaaagacgtccatccttccctccattttcttttaggaaatcctctatgtttttttcctcccattctctctttatgaaagcttttgcaacaataaattttaaataagcatacttctaaatacatattttttgcaacagtaaattttaaataatactaactttttaacttttaaattgtacgttcaatttttgatctatttacacattgtgttatttatgattaaaatcacaattagaatttaagacacatgttgactatattttaacttgtaaaattatattcatttaagtttttttgagaccctttacatgttattcaaacatatatgaacaattattaaaagaaagtgatattatatactatttcatcatatatcacttcaccggcatgcatagctaaaacagatttatgaacttcaaaaagttctaaaaattctgaattatgagaaaaacaaaatatagattataccatagtttgatgcatcacaaaacatgcataactaaataagatttacttaaaaagtaatgagtcatatcctagctgtttgtccacctatatatcattccgagcttaagatagaaatgacctattctgtaccaaattctacatgataaggcaatagaaattatatggaagtaaagataaagctaaatttcctttctgtatacatggtaatataacggtaatgttcagcttcagaacaagcaagcaaaagctgcatctgtatttgttctgtgtatctatatattgtatatgaaaaatccattaaacttcttagaatactctcaagccgccatgtgagactcctaccaatgctcctaagctgacacgtggtactctaataaattagagaaaaccttacgaattttgagaaaaaagaaaaaacatccaaccctcgattttcatttaattcgacggacccactatttttaaattattagattagatttattaaaaatacatctcatataataatatatatacgtatttccatactatactactcacggcaaaaaaagaaaaaaagtaattaccattgaaaaaaagatgggttaaccctaccacggtcccacctaattgctacgagagaaatgtatatacgtactgttcacaaaaaaactgaaaaataattctcacatgtacaatacgcactactatccaccggattgagaaaaaaaatctcctggacatgtacatactacgcaccgtccacccgtcccaaataaacttatcaataatgttagatattttcttgagaaacaaagtcaatgcagacgctaacaacgcacgcacactcacccctacgaacgcacacatgcacactctacctctatgagtaccttcgagagattgggacgacatattttgagattgataaagtcaccatgagtgattcgctgtcgatgggtacgtcgtctaccactaaaagaatttgtcaaatctgggacttgaaaacgagtgggcaatttccaccacaaggaacctaaccagttgagttatgtttaattcaaaataatgttggatattaaaacagccataatttttaaagtacacttttataatactcctatgccttttaaaccagtgaaaccattattttaaatcgttagatctatcttgcataaagaaatcaagcgtgtattcatggtacatcacatttcaaaaatattattatgcaattatattaacgttaggttgcgtagaacctcctacccactctacatgaaatgtttatacatggattgtctctcaaactatatcaatatgcatattaggctatataaaatttagtggagcatgataatatatatgcaataaaaaaaataaaaacacactacttttgctttacacttacttttactttcgttgaaatattttcaaaagaaaataagtctaccttcttttcaaatgatgaagaagattataaataattgcatatatatatatatatatatatatatatataaagctaataggttaaaaattataatagcaactaatgctaaaaaaacaatttacggggaaattggggtagatgttctctggttaatatatcgctttcagtgtctcaaacttagcgtatgacgcatgcccgcgcatttgcgcgggctaccttcctagttattatatactaaaagtccattaaacttcctacaaacgctctcaagctgccatgtggcaccctataaatactcttaagttgccacgtggcactttaataaaatagagaaatctaacaatcgattttcatttaaatcgtggacccattattattttagatggtaaaatccttctttcaggttgcaagtacgcacggtttccttttctacgttgagtagtatatacgtacacagggtgaaaacgtattttttcatcagcgagtagtccacgtacacgaaagattgttttcggtaattcttttctacataccgcgaaattttcttattcatttggaggttaggtgggaggcactattttttttcattattagatctaatctaaaggcctaaaattaatattgggtccatcaatttaattcaaaaattcattaaacttactgcaaacactcccaagctaccacgtggcacgctataaatacttttatggcaccacgtggcactaataaaatagagaaatctgaccatcaattttcatttaaatcgatatacctactattttagatcgttagatccatcttaaaaatcgaaaacctcctaccgtattattttagatcattacatctatctcaagatgtgcttctatgctcctcagaaaaaaaaaagcacgaatgttaaagatggtgaatggtctagattaaaaggtgattaacaggctaaacgttattaggaagggcaatttcgtccttgaggtagttacccatatcccaaacgttcaatcctgatgcagcgccgccccgcctgccgccgcattcgatcgcaagtacaagagaaaaaaagaaggcattccatgattccaaaacagcgaatcctcctctgttccggctacacatgtgcagcacctcaatccaccatttgattggcatccatgttcttgccaaatcaaaaaatgagatgggggtagagataagaagcatgacaagaaaaatcagaaatgggcgaaggcagggagcacgccgggacaggcggtgtggcgagaggaggaagaagggggtggcgcagcaaaagtatacgggcgatgcacggtggaggaagcgagcgcgatcgctgagagtcatcgatttgtaactcctcatcggctgatcgagtatgtgataacttcacaggacaaaactgcccctccctaatgacttttactctgttaagcaacttttaatctaaatcattcatgagctttaatatccgtgcaggtttttttagtgagaagcatagaagcacatcccaaattaaaattatcttttagggaaaaacccacaaatagatatcatacgcattaaaatccattaaatattctataaatgtttctaaaccattatgtggccctcttaaatttgagaaacatcccaccattggtttccacttaaattgacatacacactattttacacgttatttgatcttttatttttttttcaaaataaatatcccgaactatcttcccttcctatccatccctgtgggcattaaagtacataaacgctatgctaagctactgcttgtctcacttaaattagagaaaatcctaacaaatatgaaaaaaaattataaaagatatggccatcggttttgacttaaaaatcaacacattattttagattattttaggtcgttagataaagcatttcattcaaaaaaaaaagaaacatcccaaccctccctccatccccacatccccgggcagtgtgcggtgttacatttcttacttttctatgtttttctatctttatgataattaaaattaatattatctgtttgaactatcatattagaaaagaacacaaacgaacctccaccaatcatgtatagccagctatccatctgtcccttatgatctgtttgaactatcataattttttaactgaaatcaataatttaataccaatattggctatatttacatgctaaaaaaatacattttggacccacatccatatcaaggcctccaccaaatacaagaaaatgctaaaatttctcactaaaaaaaatcaaaatatcccaccataactttttatgaaactggtagacccatcacctatatcccattagacatctaacgactgtagcactcggggccggaagagaagaaccttgatactaaaccaagtttcgataacttcttcagctttgtcatattcatattatgtctatataagtttttctaacacaatctaaaatacatgaccgcatattaatattccatttatataagtttttcaaacacaatctaaaatatatgcccgcgtgaatacgcgggctaccttcctagttcaattaatcggcggaccctacaccgtggcgagcgagtgcttcagttcacacgcaccaccatctccaggcgtggccgccctccgcgattatgaggcgcgagtgccgcaacgactccagtggccatggcgatctttcctcgctctttgccggctccaacgacaactgcccagcgcctccgatggtggcttcgtcggcaccatccgccatgtccggccacaagagggtaggtccggcttggacaggttcattgaccaccaccccggcagctagctggagcacaaccacttgctgctattgctttttcttgcatctatttatttggagtagtatatattgcaatctgaatgcaagctagtaagtactccctccgtattttaacgtatgacgccgttgacttttcgatcaacatttgaccattcgttttattcaaaatttttgtgcaaatataaaaatatttatgttatgcttaaagaatatttgatgacgaatcaagtcataataaaataaatgataattacataaattttttgaataagacgaatggtcaaacgttgaacaaaaagtcaacaacgtcatacattaaaatacagagggagtattgagtaccaaggcgatctccacagtacatgtatgaagcaacttaattggttgttatgaaatatttgggtgatctcatctatctatctggatagagcagcaataaatctgggacacgacggttggtcgtgccccacacctcttctcgcctcacggcgattctaacaagggacgcgatggttcccaaaccatcgcgtcttctgttgccgcctctcttcctcctatatattgtaaccattgttatcaataaaggatcgctcattcattctctacattcttgtgtgcaattctctctctcactcaatctctctcgctcaatcaattctattcacaacattggtgaactaatattgtaccaattaagaaagagaaaattattcccagcaaaataaagcaaagagaaaactatgttcggatttcagcggaaagtagtttaattatgctagaattactacatgggggtaatttttgatgatttatttgacagctgaatgtgagtgttaacctaatgtttgattcccccaatcttgtctgctggatgcgagaccactttttttttttaatgaaatgattgagacgacttgtagctttaagatatttgttttgttggacaatctagcaaccaatcattttcttctcacttggtagcaagttttctgttgtttgatcagaatcatagttgtatcacatggaatttgtagttctgtaccaattaattttaagttttttttaataagataaatggtcaatcaaaaattaatggtgttgtgtattaaacgatggagatagtttattacaatttgaatttatttaagttttgcatgctcggtatatgccccatctctctttttttaaaaaagaacaacagatactttacacgatctatcacaaaactacaaaattaagaacttatttcacaaaattagagattcagtgtcttcgtttatcacaaaactatagatactttgcacaatatatcacaaaactatagatttaagaacttgttttaaaaattatagatttaatatcttcatttatcacaaaactacaagtttagggtctccattatcacaaaactacatgttttaacaatgctaaaacctgtagttttgtgataatggagacactaaacctgtagttttctaataaatgaagatattaatgtatagttctgtgataaacgatatactaaatctatagttttacgaaataagttctgtgataaacatagacactaaatctgtagttttatgaaacgagctattaaatctgtagttttgttataaattgtgcaaaatacctatagttttataaaatttactctcttttttttctaaaaaaaatgatcaaccatcaatgtaaggaaccacctatacatttgtcgacaaattttctactaaaaatttgacagatgtaattatagtacaatcgtagtgtaattacacttgtaacttgtatataattacactgtaacttgcatgtaactacagtgtaacttgtatgtaagtttcacgtaattttgaatcgttagatctattacaagatttgttctggtgaggaagaaaaaaatcacagcacatacatatgtgaaagaatttattcccaggacctctattttactgaaataacatgttaccgagagattcttaaaagttacatataagttacattatagttacagtataattacatgtaagttacagtgtaattacactacgattgtactgtaattacatctgacaaatattagggggagaaatttgatgacaaatatctagcaaaatcgaatgtgtaatgtacgtacgtacttccctcccattacttttctatatgtttttttcttcatgagaaattaattaaaaccaaaattattttcacaggcaataaagccccacaaatagactttcacctactaggtctactaactcttcctctatcactttaaacatcctacaaacgcttttaaaccgctacatagtgctattaaattagagaaaatactataaaatctaaaaaaccaaagtatccagctatcaatttctacttaaattgacagatgcattgttttagattgatagtatttttaaatagaaaaatccaaacctctctcctctccccacccctatgcgatattcaaatccattaaacatcttataaatgcttataagctactatgtgacattcttaaatcggagaaaatcttagaattttgaaaaaaaataatccgaccatcggttttgatttaaatctgtggaaactttattttacatgcccacttttttatatattttttttgtaaaacatgttaacattttcaaatcttgagtcctgatgtagaatcatctagaaacatgtatttgttataagacatttcagtttagtgatagttcacaaaaaaatactatgaccattacttcaatatttcatgagagaaaatttagaacatatgcctttaccctatgccataaatgcattcatgttttcgccccaaattgcaaagatcaaagcatttcatgcatgtttcatacaatggaaatctcccaaaaaaataggacataaaataaattgaaaactgaaaaaaaatgttggtagatttaaaacccattctaaatagaaaaaatcaaacattttatgaggattgttagaatgaaaatatataaaacttaaaataaaatcaaacaaaaaatgatattattgaacttcttttgggatgttagatatcattctggggagtcaaaataccctaaattttctcataggaaccactccattgacataatgttttctcgtaatttcaaaattgcatgatctcaagtaattttgttttagaatttatttttatatgttgcaagtgtaattgtaagaaaataactagcgttcaaagcacactaaataagcttaattttagtgtacaatatagtaaaaaatattttaataaaaagttagtcatacataaatattcataaaaacatttcactcacattatataaagtgcccgcgcatgtgcgaatgccactcttatcatggtgattttgggttatttttaattgtaatatactacatctgtcccaaaatgtaacaatagttgtctagatttatagctaaaaatacttacattttaggatagagagagtagttttcagcatttacatttggcttcatttaatttttcgcggtatgtcaaacaactgaacacttgaacaggccgcagccgtagccctggagcagaacacgaccccctgagtagcggtgtggacgcctggagacggtactccacttcaccgtgtctccttaccaataaaatccagaaattattaggagacggaaaagccagcgacgacctgacccagaaagaaataaggaaaaaaaaaagaaaggaaaagaagaagaaccaagctgcaggcagctggttcaagagctccgagcgggtcaaagtctcctcctcctctctcgtcggcgagctccttgcgattgattcatcaggtgcgtgcattccttcacttgcagaatccatggagcttgcgagcgattagtagtagttcatgctgcagagccgtttcgccgcggagaatttttgttgtctctctcgatttgctcgtgctcggcgactttagtatcacccgaagatcgcgaagaactcctccttctctcgatttcgatccgcattccgttgaccgattggtacgcgagttgactttgcaaggattgattatctccccctttgggatgaacacccagtgtccccccctttgcggcgcccgccaagtgttcgtcggaatgcccgttcccgaccgcgagtctaggcacgctgcgcgccgacgcatccggcgaagggaggccgccacgcctccgtctggtgtggttccgcggcctgcgcgcggcagcgttttttttttttttggccaaatcttgtccccggttgtgcacgatcacgatctgctttgctgactgcgttctcatcctctcttgtttgatcctgcagggttcagcacactgaccgtacagcacctgcgctccagccctgttctctggtcttccagcaggtgagcaagcatataaatcactactcagtatggctgtgtatttgttgtgtgttctgttctatggatcatttttttgttcttcatagttgttagtcgaatgttccaggtaatgctgctaaacttgcttatctaatgtatagtctttctgctcactgtcttgttgatgaaccagttattttctttgaatatgtatgaactatgttcagcccacaagttgcttcagctgaagctgtatgtatgttggtgaaatgtaataaatgcatactcaaattaaagtaaacaactacatacagaatagctgtacttcatgatgtataacagtataacaatgtttcttttactgacccaggctcccatcggatggcctatccagccaaagaaaaagccaaaatttgaattttcaaacttaattttgacattgattttgagatattttcaacgtagtttcttttacagcattggcttttaagtcaccgagaacacatatataaaagttttacctacaaattaatttttattctctaataagccgttttggcttattacgaaaaaagccaaacgataaggccgccaagcaggagagctcttccgaaagcatagtttgtccggaaaatctcgtagccgatttccgaactccaacgtaaggtgccaaaaaaaatcgttccaagtaaattaatgaaatataacttagcactggccttcatatatggtactgtttaccattctgatttggaatccttctctgtgcccatgagcaaatcaaataaagcttgtcatttatttgctgagttatatcttatgagatgccattaacttctcattaaacgttttatgagatcatagatgcttgtggtaaaagaattactctatggacacggtgcatgagtaccattatttcttttctcacagtggaccattattatccaagtctgaacagtgccacacatttcatgcagaaaccattggagttctgttcaatgaaagcacacatttctccaggaaaatagtgataattaaaggcatcaataaataaaaattttctttgatatataagttaaattggagactagaaatcgcaagccacttgttgttctcttttgtaacatgatggtgtgtgttctttcagaacaaatggctgaggcagtgctccttgctgtcaaaaaggttggcaacgtgttagcagatgaagctgccaaggctgtcattgccaaggtgtctgaaaaggttactaatctgaaggagctgccagagaaggtcgaagaaataaggaagcaactgacaatcatgaacagtgttatactacagataggcacctcttacctcactgatatagttgtaaagaattggattgcagaggtgagaaagttagcctaccatgttgaggacgtaatggacaagtactcatatcatgctattcaacttgaggaagaaggtttcttgaagaagtacttcgttaaaggttctcattacgtcatggtatttagtgatattgctgaggaggtagtcaagttagagaagcaaatccagcaagttataaagcttaaagagcagtggttgcacccttcccagctcaatcccaaccagcttgctgagagtggcagaccacggtctcacgacaacttcccatatcttgtcaaagatgaagatcttgtggggattgaagaccacaagagattgctggctggatggttgtactctgatgagtcagatagagcagtgataacagtatctggtataggtgggttgggaaaaaccacattagtcacaaatatttatgagcgtgaaaaggtcaactttgctgctcatgcatggattgttgtctcccagacctacaatgtggaggctctattaagaaagctccttagaaagattgggtctactgaactgtcacttgatagcttgaacaatatggatgcacatgacctgaaagaagaaattaagaaaaagattgaagatagcaaatgtttgattgtgctggatgatgtctgggacaaaaaagtgtactttcagatgcaagaagcattccagaatcttcaagcaactcgagtcatcatcacaactcgagagaatgatgttgcagcccttgctacctcagcacgccgtctcaacctccagcctttgaatggcgctgatgcatttgaactcttctgtagaagggctttctataacaagggccacaaatgccccaaggagctagagaaggttgctaattctatagtggataggtgtcatggcctaccactagcaattgtaacggtaggaagccttctgtcttcaagaccagcagcagaatttgtttggaataaaatatacaaagagcttcggactgagctagcaaacaatgatcatgtccgagcaattctaaatttgagctaccatgacctatcaggagacctcagaaattgtttcttgtactgtagcttgttccctgaagactacacaatgacacgggagagccttgtgaggttgtgggttgcagaaggctttgtgctaagcaaagaaaagaacacgctagaggatgtcgcagagggaaaccttatggaactgatccaccggaatatgctggaagttgtggacaatgatgagattggcagggtaaactcctgtaagatgcatgacattgtgcgtgtattggctctttctattgctaaagaggagaggtttggttcagcaaatgatcttggcacaatgttgcttatggataaggaagttcgtcgcttgtcaacatgtggatggagtgatgatactgtatcaacagttaaattcatgcgccttcggaccctgatctcactttcgacaacctcattgtcccttgagatgttatcctcaattttgtgtggatctagctaccttacagttcttgagctgcaagactcagagattactgaagtgccgacttctattgggaatatgtttaatttacgctacattggtttacgacgcaccaaagtcaaatcacttccggagtctattggaaagttatctaacctccacacgcttgacatcaagcaaaccaaaattgagaagctaccacgaagtgttgttaagataaagaagctaagacaccttttagccgatagatacgttgatgagaagcagtcagatttccggtactttgttggaatgcatgctcctaaagaactttccaacttgcaagagctgcagactctagaaactgtggagtctagcaaagacctggccgagcagctgaagaaattgatgcaactaagaagtgtgtggattgacaacataagttctgctgattgtgcaaatattttcgcttcattgtcaagcatgccatttctttccagcttgcttctttctgcaaaagatgagaatgaggaactctgcttcgaggctctcaggccaaggtcaacagaactccacagactgatcatcagagggcaatgggctaagggtacacttgattgcccaatatttcacgggaacggcacaaatcttaaatatctagctctaagttggtgtcatcttggcgaagatccactagggatgctcgcttcaaatttgccgaacctcacttatttgagactgaacaacatgcatagtgcaaacattttggttctttcaacagagtctttcccccacctgaagacacttgtcttaaagcacatgcccaatgtgaaccagcttaagatcatggatggggcgcttccatccattgaaggtttgtacgttgtgtcactctcaaagctggatatagtccctgagggcattgagtcccttcggaccctgaagaagctctggcttctgtacctgcacagggacttcaaaactcaatggcacaagaacggaatgcatcacaagatgcagcatgttccagagattcgtgtttagatgcggctgacaggtgccgtttgtagtagttttttttttcctcgtctgtttgcagctcaggtgttgatttccaatgagttagcttttttgcattcgcggtgcgtctgtacattttgtatagtttcatttactttgatatttatctatctatctatctatatctattatatactaaaagtccattaaacttcctataaacactcctaagccgctatgtggcatcctataaacgctctcgagacgccacatgtcactctaacaaaatagaaaaatttgaccatcgattttcatttaaattggtggacctattaattttgaccgttagatttatttttacattaaaataaaatccccctacgacgcctccacaatgtacgtactatccatcaatctatcagcaacgtatgtacgtacgatccctccgtttccattttttttcatttttctatttccataataattaaattagaaaaatgcatgcctaaaaaatccatcaatctactagatctattcttcctctagcacatcctaataagtccattaaacatcatataaatattaccaaacatctatttgacacctaaattagagaaaatctaaaaagatataaaacattcgttcatcgatttacattattttaggttattatatatatctttttcttacgtaaaacatcccaaactcccttcgcgctcctatatggcgttaaaacccattaaccatccttataaatgtttgtaaaccactctgtaacacttcgtatatagagtaaataataaaaatttaagaataaaacatataatcattggtttcgacttaaatcgttgggcataccatttaggttgttatgagatttgtatttaaaaaaagaaacaaacctcctttcctctctcccccacccaacaatgtcgaagccttatacattaaacatccaacacattattttagtcattatatcaatcttttatttttttgaaaaaaaatatatcaataatttatttaagaaaaacatctcgcccaccttatccctatgtggtgtgcatcgttcatccttttctcttatttttattcttataataattgaaattaaaattatctttatgggaaaaaaccacaaatggacctccacataacaggtctagctagctctcccatgatcccttttcttctctttctcatcttactaacatttatgggaacaatataaataaaataatataatcttttaaatcatatattcaacttatgatttgtttgaactattgtattttcttagtgaaatcaactttaagatccatattgagtatatttaaatgctaacataaaaattacattccggacccacatccatcttgaggcatccacttagtaaaaaaatgaaaattcatcattataaatcaaaatatcacatcttaatttttaataaagcctgtagacctattacctgtatccaattagacacgtaacaactttctatcaacttaattaatttctctcaacatgtatatgggggtaatttgcctatacaatttgactttggtgctttttatcaataaatgatatttttaggagataaaattatatataatctatgaagtcactaataaattagaggaaaaaaatctttaaatgataacaattttatgtcataatgtgtactccatctagcgttaattttttatagacaacattttaaaagtaaaaaacatgctcagttgaacatccaactgatagtaccaaacatgtggattgcaccgagaaatcttttttttttgaggaaatgcaccaagaaatcttagcagtgaaacaactctgaggtggcattaccaatatagtgaatatagtgagttaattccatatggaattaaagaccatgttcatttatttctttttggtaaaacttgctatgctacatcgtaaattcataatatttactgtagaataatatagaaatgtgcatttattatatgacctctattttcatattcttactcaattccagagagataattagaatagatgattttaccccatgtcaggttgtaggcttaacccacatatatttataacccaaattgcagatatcaaatcatttcaaagatgcttggtagaatggaattctccaatttattaaaaatggacatgaaaagaattcaaaatttatgtaaatgttttaaaaaattgttgggtttaaaacccgttctaaatggaaatattcaaatattttagcaggactaatagaacgaaaatatctaaaatttcgaatagaatctgataaaaatttggactttttgagcaaatgtttggggaaagatatccccatgggtagttgaaatacccaaaattttttcataggaactacatctattgtctcgacataatattatctcataaattaaaaactgtacgattttaagtaattttagtttagaatttatgtgctacaagggtaattagaagaaaatcataaccaacgttgaaagcacattaaataagttaaataataatttatatatagtaaagtaatattttaatacaaagtcacatatatagatattcgtaataaaatttcacacatattaaattaggtgcacgcgcatgtgcgcgggctacctttctagttattattattattatatactaaaagtccattaaactctctataaacactctcaagctgccatgtggctacctcaaaacgctctcatgttgccacgtggcactctaataagaaacagaaatctgaccattaattttcatttaaatcggtgggcccattattttacatcgttagatctatattagaaaaccaaaacgtccacaactatggtcccacatctccttccgtgtaggtaaggtacgtacatgagcgtacgtacgtacaagaccgaagaaagacatccgttttttctttttcccctctctttttctctttctcagaaatactcgtacaaatgcgtacatttgtcttatttctggctagctactttgtaaaggtgagcaaggttaatgcaaatataaatacactgtgatcatgtgataggtaattatatatagattcacgtagattatgtgagactaaattagctatgtaatatttaatatttaaaaataaagtttacaccattatatatttacccaataaataaataatgtaagatagtttcctaaaagtccattaaactccctataaacattctcaagctgccatgtggctccctcaaaacgctctcatattgccacgtggcactctaataaaatagataaatcattattttacatcgttagacctatcttaaaaaccaaaacctctcaccggccccacatccgtgcagtacgcacgtactgctcgcctgttccgttctcctttttttttccctttgttcctttcttttgttttttcaatcacaattaaaattgaacttatctttatagaaagaatacccccacaaatgttaaagtccattaaatatcatataaatgtttctaatcgttgcgtggcacttttaaattagagaaacatcgaatcattggtttccactaaaatcgatggacacattattttacatcattatttgatatgtattaactaaaagtccatgaaactttctacaaatagtcctaaatcgccacgtggcatcatagaagtgttcctaagccaccacatgccactctaacaaataaccgttgattttcattatatttgatggacctattgtttacaacattaaatttttcttaaataaaaagttaaccacccaaacctaatccacccatcctgtaaatagtaagtacgtacttatatctcatatacaactacctattatatatgaaaataataataatatacctcatatattattattattattattattattattattattattattattattattattattattattattatgttgaccgtaacatatatgtcacgtgtctgccattctctctactttctattttatagctagcaattcaaggtaaacatgatgagctaatttgtcatgatttaatttataattaaacaaattaatttaagatcaataaaatctaaaagttctaagtaaaatgcaaaacatcatattatcgatttacacttaaaccgagaaaaatattattttgatgattagattagatttattaaactaattaatccctcactgctgatctcctccgtaatttatgattaaaaaattcaaaaagttgtgagtaacaggcaaaatatggcaccatcgatttacacttatcctattatttttaaccattatagatctattaaactacttaatccctctgtcctaatctcctaggtaattacataattataataattgattcatattacaccaccctcttcctagattaacacttcattcttatgtttttatactttattaggtacaaagagatatcttcgattatatattaaaagtccattaaacttcgtacaaacgctcctaaattaccatgtggcattctacaaaacgctactaaaccgttacgtggcgttctaataaaccggtggacccactatttgtttaataaatcgataggcccactatttgtttaataaatcgatagacccactattttcaactattatatttatctttataaaaaaacattcatcataggaactcataatttcctatgaggaattatccatagctatgaccaatcgatgtttgccatcaaataataaattgattatcaatttttctacaaaagtgggtagagccattaattatatatggttgtatatgtaatgtcttttataacttaagttttccctcttttcctatggaatctcaagagcatatgtcttaggtaccattttttataccgtaagttaccaaccataatctccaataggtaattttataaataataattaaattcgtcatgcaattctaacaaagaaaattcattaaaaaacacaattttaaaactttgcagatattatttcgttggtataccgcctttataagtttctcaaatttatcctacgtatgagacatgcccgcgcgaatgcgcgggctaccttcctagttgtatgaaagagcaaatccattttttttataagacctcatttgcatgtattgttgccaatatattaatctctgtgcttggagatatatgcagtggaaatgagatgaaacaagatagtaggacaaatgctatcaatgttattatgcatcaacgcaagcatataataggatttaagcttctggtcattcatgtatacagtctagatttttgcctgctcaggaggcagcctgagcgagtgcttgtttggttgcagttgtatgcctgaggataacagtacatatcattctttgataattacctcaataaacaatatcaagagacttcattaacgtgaaaaacaaatagaaatatactcctgcgttttgtctatatggttctacatcgaaacttgaaaataaacaagagtaaataaatgatgtagggcagagtccggcgttggattttgtaggcctgagccaggccaggccggccacccatgaaacgatccaggacatcaaccaaacaagtactacgagacaggcgccagggaaacgacaggtcaggcgaaaacacttcagttcaggactccaaccaaacagtcccccacatgcctagttgatgtcacatgccaacgtcagtgcctatgataacatgacgaccaactgcctagtttagtactaactaataactctgagcatgtgttgattgtgaagataacttggtggaatgcagaattgtacctttggaaggaataaatcgataccagttgggtaaataaaagaagttcctgtcaagccacccaataattcaattatgaagacacaagactcccatcgattttgaccttcagcttgcttcctagcgttctgcactggtcatctagctcttttctgcaaccatagatatccagttcttccagcgaaggtgggagacccttttctggcagcttcctgatgccatagcaacacaagatgtccaacctcttgagggagtgaaggctgtgcagacctgcaggaagatcttgaagagagtagcaatgcgtaaagcggagctcttgcagggtcgtgaggagctggagcgctctctcttgctcatccgttagtctccactcttcgcttctgaaaccgtaaatctttaggtattcaagggaagtgaggtgcttgcagaatgacgtggtaaggatagaggtatcatcgatgcacaacttttctagtcgcagatggccttgccatgacaaattatccaaacatggaggcaatctggggcattcgtgtacttgcaaactcctgaggccatagaggaattgcaagccctccagtacggcgagtgttttacaatatttaatcgtcaactcttcgagtgccgtgcatgattgcagctctagagatatcaaaaattcagagtggtgcacctctaatttttttaggcaggtgagattcatttgaaagcgggggcacagggtttcaataaaacagttgttgatgcaaagttcctcaagtgattgtggaaggaggtaacccatgtgagctctaaggccctttacctgcagcagccttaggttgccgagcgattgcaagccttccagagaattaagcgattgcaagccttccagagaattactagtaatttgtaagcgcgcacaacgaattgtcagctcttggagtgctgtgcaggaatgtaactgcagagatgtcaaacttacctctctcattagaaacagttttttcaggcgggtgaggttccctggaaagcagagttgaagcatttcaaagggaccatcatattgtacaataagttcttcaagggatatagggaggagccatcttctattcgcctgctcaacatttccatcattatgcaataaggaatcgatgagcttggggcataccgaaatctctagctgctcaagggaggtaaatccagccaaaccatccttgctcccatgaaatgttaaatcagagcaccatttgatatatatcttcttcagagagcatatgatatttaacggaaggcgtggaagtttgttttcagcggaaaatcccaatgatggagtctccgtagctgacataagatttggttgaccacattcttcctctcctatagatagacctgttatctgctcgcagtcttgtaacctcaactcttgtagggtcttcatgtgttgtagcaacagagatagccacttccctgttattccacaatttctaatatcgagaagtttaagacataggagggtgttgtgatctgcagctgccatgtcttcacgggtatctgatgggacatttggagcgaaaagttctaggcagttggatatttccaaactcttcaaagacctgagttgtcttaaactgtccaatgatatagtcgtaagattttgacaaccacatattaccaatccacttaagaacctcaagttacggaacgccataactttgtcatccaacgttatcatctgatcagaagattcatcccattcatccatccaatcatatgaaaatccgattcttaatgttccactagatgacccctcgattgatggaagtgttgaaactcgggtgatggataatttctcgacattaggtgaaggcggaagaggtttgtgcacacgcaaatgagggcaaccatagatggtaatctcccttagacaggaaaaccatgacgactgctcaatctcaaattgcgggtagttctcaaacaatggaaagacctccagtgcagggcaactcttaatcttcaaaacctttaaattgtcattcaagttcctgatggaagtgcaggagcatgccttcaagcttatcaattcagttaatacaagctcctccactgatggaattgagacttctgttgcattcctcattttgatcaacacaagctttctaagtaacgttagcctttctaaaggaagtctttgccatttttcacatttttctaggtgaagtgtttgcagacaggtaagtgaagacggaaaccaagttggggaggtagctccattatacccagatatccgtagatgcttgagactgtgatgtggttcaagaccatcaagcacctcacgtgctatgcctggcaaattttttaaaactggggatatatttgtatcagccatcgtcagtccctccatcagaggttctatgtcactgtcagtttcattttcagaactcatgtctgtgtcatactcattttcagagctcatgccactgtcatatccatcctttgcatccttcgaggacaaatgtagcttttctaaattatgtttgtctcttagtcttgcaccacaagcttcgattctagtcgtaatagtttcaagtccagacacaccgagctgtacaagttggttcaaggactgtagttggttcaagcccaaagaattatgaacactaaagtcatttaattcctgaagagaggtcattttgccaatgctagtgatggacgagaacacttgctttgctgctacaagatgcctcaggctaacgaggttatccatatcattaggtataataagatcagattctgaaccagcatcaaatacttggagatgataaaacttgccgacagatagaggcaaagctccgtcatcaaactttatatagcgaatatgggtaggattcaccagattcaataacgaggggtcaatatcagtaaatggtgcagagatttgcaagacacgtagatgatgttccttctggactatatctttgaaggatttgaggaatatatggttatgctgcccaattagcaccaaagttctcaaatgtttcactggtttaactgcatttctaattctttcttcaaccttgccacaatcaggatcttcttggtgtgtagaatcggttaatattgacaagtgacgtatagttggcaacattttattgcactgtggattatctatagttgcgtactctgttctcgaaaccatccttgcaaaatcatgaataagcccacacataacataggatgtcttgtcaacttgagtgaactgatttgtctgtgcacctggctgaaagaagccggagtttaccaaattagaaaggtactccctcccgatctcttccagtctcttacttgaagagttacgatgcacaaaaccttgagaaatccaaatatggatcaactcctgtccaaggaagcaataaccactagggaatattgaacaatacaagaaacattgttgtaaatagtaaggcagctgatcatagctaagcttcaaagaaggcatgattcctctacttatattcagggatttccaatcttcattcctcagagtgttgctccagtgatcaattgtaagatgctttcttaatatttgccctgctgtttctgctgccaatgggttaccatttaacttgtcagctattttctgcccaatgatgtttagacttggatgtgctttataattttcgtcatcaaatgcgcatgctttaaaaaataaccaaaagtcctcatttgacaaagaatctaacttaatcggttcgactgtccccacccgttgtgcaagagacaaaattctagttgtcacaagtatcatattgccttttgcacgatttgatttcaatggagctaatagaatgttccatgtgttatcatccatgctattccatacgtcatccaaaacaagcaggaattttttcgtgcggatgtccatatgccttttcaagatctcctgaagtttggcaaaactacttattgcattgtgtctttcttttctttgatgagatacttcatgtctttcttgagagacaaaatctagaatctccatcgtgagcatcactccatcgtagtctttagatacccaaacccatacctgatcaaagttatgtttcaccattggatcattgtatacaagttgagcaagagctgtcttcccaactcctgcaatgcctacaatgggcactacagttagtccatcgtaactgtcatctgtaataatcttcaggatggcattcttctccgcgtctcttccataaattttgtgtgggctaagacttgacgttcggatcagggtcgttgttgtacttgtacgatgatttgagtttccaacaaggtccgctccatgtagcgtgagaacctcactaacagccctgatggcaacttgtaacccaccagttatttgctgtatcctgctagaaaattcagccttattccaagggcgtgaatcgactgcattgttgttccaagggtgtgaatcgactgcattgttgtgtgttgactcatcaatattcctcatcctttttctgccggaattaatcttcatcctttttctgctgccggaaccaccgagttgatcagtaatggtgatatttgctgtggtgtcagaagtactgcaaaattaaaaaggaaaaaaaaaagaaggatcacacaatcgctgctgacgtgatgaaccatgcaagtatgttacattagttaaaaatagtgccgggtaaaaacaagaagaagaaaagaacataaatcctacctcgataggtttgggtagcttgctcgacgcttgttcttacaactgatgctatggaggtgattacgcaaaaccgacgtccctgtgtgagaaccacactcgagcacagtgttacgaacacatcttgccttcaaaggctttccattttccaattcagtgatacgtaagttctcccatgccccggaccggcctgttttgccaccactgctactcgaaatatcagtggtacttttcggtgtcactgcctctgcttgcagtggtgcatgtttatccgcgctttcaggagcgccttgccatgcatctgaaccaagagaaagcggaatgaatgcacctccctgggcagaaactaaaacggatacatcttacatatcctatcttactataaagttataacccactaactcctaaagcttaacatgcaaagatgccacatcatcattcactaacaagttgacacatcatcatctattaacaatcaatacatttaatatcatacaaacatgctatattatctataatttaaaatttattacattttagagcttttaaaataaaggcatgttaaattattatcacacataattcacataatatttcaatatatatcttcattatttattataatatcttatgcacctatgtaagttcatcctcacttggttaatattatttttctcttctctaattaaactattattttggctgtgtacaccctcgcaaggtgtagaggccgggtttaatataatccattatctaaaaaaaattaagctattatcacatcaattgattttagtacttatgtaagtttatgttgttaagctatcttacacattatttttacttattattattatattgaagtcccgcagcaacacgcggggtttcatctagttacgtttatattacctccttggacctggtgctggagcctgtagtagtcgaggtcgtcgactactgcattgttgtgcgttgactcaccaataatcctcgtcctttttctgctgccggaaccacagagttcaacagtaagagcgatattagttcagcacttacttttttaactatttttaaagaaaaagaaaagaagagaacataaatcctacctcgattggtctttacgcttatccgtacaacggttgctacggaggtgaatatgcaaagccgacgccccattttgacacttgagctcagtgttacagaaactacatttgaccttcacagcctttccattttcatattcagtggcaacaaagtacttccataccttggaccatcttttgccaccaggcccaccactgcttctcgaaatatcagtggtaaattttggtctcactacctgatctgcttgcactggtgcatgtttatgcgcgctttcaggagcgccttgttgccatgcatttgaaccaacagacgaaagaggtgatgtacatcttacatgcgtacggacgcttatattacctccttggacctggtgctggagcctgtagtagtcgagctcatcgacggcattgtcggcgtcgtagagcagctccctgagacgaccgagcgatcgggtcagcttgttcccgattgctctctgcctaacggcagcgaccaccactttcaccctctccatctctgacttaagcttctcggtggcatcagagagcccaacctgacgaatccactcgtccagcttgtcgctttccaggttctccaggatggtctgcgccagccactcgatcccgccctccagcaaagtgacttccacctctgccatcaggttcggcggaaaactgctagcagttgctgggcaattggcaatggcatgaaatgatggttgtaatgattaaggtgctgaaataatggttgaagtgatatataattgggatgattaagggactattattttttgattaagggaaccggattatcacagacaagcttcctaaactcaagttttttttttaaaaaacaaatcttcaataaatttttttaattagaatattttttaagaacaatttttcaaatttgtaacaattaattcattcatttctgtcctcgaataaattgtaatgtcatggtgaaggttatgttgtgatgaacaatttgcaaatatgttcagacaagttgaatttcatgtatatgacttcttcgtagtttttgtacattacatgttacatataatactctattgttgcactaatttaaatcaagttggcaagaacttgtgctcggaatatggtttagatattattctcatgtgcatgcattggttgaggccgcagaagcgtggttggtgactggatcgggactaaacacagagggttaaggtccatatagtcaaggatgtcatatgggatgctcaggctagagaactacatgggaggtgtgcgtatatggataaagaagttaaacttggaggaagtccaaatttggagggatctgaatcctaatttggaaagatagaaaagagttggaatatgacggagtcctagtcctagttagattaggaactgtacttggttatgaaggatatgggtatataaacactagagggagtgaccaaatatgaagagaaaaaaaaagggtttcacatgtttgctcttgcatctagagttcagtttgtgagatcatagaggagtgcttttgtatgcactttgtaaacactttgatgcaaaggaatacgtggtaatctcactatcttttacattgttctagttaatttggttttttttgcttgattttattttatcttaataaaattgtgctagaacaatagttaagagttctccttttggtcttttcttttgggtgaatatttattgtctctagaggcggatccagcatggggacggcagggtctcgagtccccactactggcgcgagtactatgggagcccccacaaagcccccgctaaatctttttgctatatgtatgcgggaatggggctgtagctttatctagtttattaagaccccccctactatctcttcctagatcttccactgattgtcccagatttgttttgtttttggttccgctaccaatcaccccacctctagtaggttcggttagctgtgtttcaatcctaaaagttatatcagagccacgtgtactcttgttatggcctataggttttcaaccaagctcatagtttttcttggagttgacttatagttaattcttgaaatctcggaggaaatcttacatcatttctcaaggttttgatatttagcaaaaagtttcaactgcttatacgtttcctttgggtattattgatgcaaacaaagttgcttcttaatacaacaattgcatagctcaaaatttgattttgagtggtgtttctagttctggttttgattggatttctcatcttgaaaccgtgaatgagttgtggaaagttctgagtatcacaaaggctcttttactatcaaggaagtgtgtaaagtatcttcaatattcttcaatatgtatacatatacatatacatatacatatacatatacatatacatatacatatgcatatatatatatatatatatatttgcactcgagctcacatgcacccacaatctataccattgatgtgttccaagactcgtgctgcaaaatgtacctaactttgatcaaatgaattcttactatagaaagaaaagattaggttggtacacaacacatataataagtatgttgcatttttttaccaattttttttttcagtaacaatcatatatgaaatttggcaaaatctaaatcaagaggcctctcgttttagtgctaacgagagaccctatctagatgccttaactgtagtactagtccccatcaagtaagtaggaaatatattcctatcacctatgtttcaaataaaattttttcttaaaccactcatccgatctatgattcgattacacggttgtgttcgtaagaactaaatctttataacaagatctcatatgattatattttgatgataaattataaattacttttatagtatatctaaattacttttagatttcactaaattacttcttagacatataaaagtaatttcagtaaagcttaaaagtaatttacatatattatagaagtaacttaagataaaaaggaagtaattttgtcatgatattaaaattagatccgttatggaggtaaaaacgtgagtctatctaaaaattaaatttaatcaacacaaattatgaaattgactaaaaaagataataaaataaacaactcacaacattatgattgtataataagtaatttaatcaaatcgaaaagtaacttatatatatatatatgataaaagtaacttacatatataataaaagtaatttacaacataaatatttcttctatcaaaatatagtcatgtaagatcttgttgtaaagatttaattgcaacaagcacaatggtttaatcggattataaatcggataaataatttaaaagaaaaatctataaaacatatatcatttggtgtattagtatgcaaccaagtctaagacaagtctctggttaagacaatatagagtgcggtctctatgtagtcatgtccatgaaatttgtgcatggtatttctctaacccttatttttttataacatgatatttagtcaatatgagaaaatgttacatctattatatcaccaaattatcatcttgcaaaaatgatatatcctaccatccaaaactacataaacgaggtaattatataaacaagataatttatataaacaaggtaaatttgatctgataatttgataacctcaatgttttacatataaaaaaattttacatcaacaaggtattttgcacttgtaaaaaatattgtataaagtaaacatcatgtatgttacattttgttagtacctaatgtacctgccctaagattcgtgcaacctatatacagggtgtagattgtgggtgcatgtgtgctcgagtgcaaaaaatccaaaatttacatatatgtatatctatatatatgcatatgtatatacatatatgtatatctatatatgtatatgtatatctatatatgtatatatatagatatgtatttacataaacttatatatatatatatatatatatatatatatatatatatatatatgcatatgtatacatatacatatacatatacatatacatacataaatctagtttaaagcaagtttgaactgaatttgaacttgtttttgttagattttgattctaggtatatagcatccaaacatataattagttaggtatgtaatcatggattgtgaactaaagttaaatttgagttgttttgttgataggtataggtatgaatcgaattcttataactaggtatatactcacaatttgaaaaaattgagtgaatttatatatttgataccatggtgtccgggaaccatggagccccatatgagacaaatatttgaagaggatcacttgcgtttgattattaacaagttttatcaaaatttgaagtgtcaagaacaagaaaagaggtggaccaaatacattgattaattaaacattgtaaataataaagctaataaatagcttggaaaagattatctaggcaattcatgtaacacccctggcccatttgaagccaatagtagttgcgatgttggccgacgtgggccaataaaatgtgatatagaggtggtggtttaacaccaactttgatgttgctgatggcctgctaaggctttgtgctctaaccatgacacacctgagttaactcttttacatgaagcaatcacaaaaatacaaggaaggtactatgcgtatgttcgcacgctcaacatgtagagccgactaataagaattttgaatgcgggcgttttgtgagaataaacctatgtaatctcgtggatgttttgccggctgcttgcagtaaggcaatgttgtatctgaactaccgcttcttataatacaattgtatgtaagccttttgcatattccccaaaaaaaaggtggggtagtccaatgccgatgatttttttttagaaatcgtagtagctttgaaagcatggtatatgaataatccgatgatgtattatcccccttgatctgaaaagagtggggactaataatcaggttcttctaacactcagttatatccagattgaaacattattagttcttaaaaatttccagattgaaacttgaaagtgcacactgcacatatacagggcctgaaaccattatttccgccctgagactaagcatttgattctatctatctattatacactaaaagtcttttaaactttctacaaatgctcctagatcgtcaaatggcaatctataaacactcctaagctactacatggtaaataaaaaaaatctaaccttcaatttgcatttaaattggtggactcattcttttggaccattagattagatgtaggaacgttcttttcttttccttctattatatctatcatttttctttctattagttagaaagcaaaatttaaaacatttaaacaactatagcatttaaatcatacgtgcatttttgatatgcttatgttgtttaaataaaacaaatctaaccttcaatttgcagttaaattggtggactcattttttttaaccattagattagatgtaggaatgttcttctcttttccttctattatatctctcatttttctttctattagttagaaagcaaaatttaaaacagttaaacaactaaagcatttaaatcatacatgtattattgatatgcttatgttgttagattatttatgattaataaatttatagttaaaaactcaaatttactgcaagtatgtagtattatggatgaggtatatatcttatacttgaaaacgtaacacccaataattatcacctcccaaaattggacccacctaagccttacaaccattagatttatcttaaagaaaaagtttacctcccaaaatcgggctcacctaagcatgtatgtacatacgtaccttattatttttttaaaaaagtacatcctccttttcacctattccttctcctatttttatgatttattagttagaaaagcaaaagttttgctttataacatctaaatcatataactaatttttgatatgttttaaattgtttatgactaaatttatagttgaagaataaatttcactataattcaactttgaaaagaaatgatattttgagttatattttttgacttttgtgtgccattcaagctaaaaatatagattttatcatcgtatgtaacttcataatttacattagatctacaatcacaaaagtaaaatgaatataactatgagtaatattcctaaaatatattagttagaaaacaaaagtaatgctataaagtaagcaactataacatctaaattatacgactactattgatctgtttatgattattttacatttaaagagccatattcactataattaacttttcatatgtcactttgaggttatattttatgacttttgcgtgacattcaaacaattatgaaattatgaaaaagaattatgtagattttattgttgtatataattgcacaataattaaattagatctataatgatataagtaaaatgataaaaggactaacttgtactatttgaagaaaatctaataagtttgtaaaagaattagttcttactctttcctaaaattggaccaaccattgcatgttccgttttcataacaattttgagaaaattccttctataccccagaaagtttagccaatcccttctacgcccccgagttttgtctactcccttgtatgcccctgaattttggttttgatccctttcatacccattccgttagtcaacttgtaatacaacattatttctgtaaagattattgctattgaggtggaagaatatttgttagaacaagatgcacgcatgctttgttggattctgatcaagcagctgaaaatagctagcactttatcgccatgggtgaaaggattaataccagttttattctgaagattaatcatgtacatgtacttgaatttaacactcctctctctacagcttgttatgtgctatatgtagcagttcatgaacagaatctatgtgattggagaaagaaaaataaatcagcccgacaggtgtgctttggttgttctcagtaaatttttgaatttattttaactttcgttatgtatggaagtaaactgaggcattgatttcttttctttcaatgatttatttcttaccaacacagattccatgttgtctgttttatgtgtaatcttggtatgaattttgtttttctctatttttccacccagggaatgccagacatgatttaatgcaaattattttctcagtttcaatgctttctgagcatcgtctggaactatgggacatcatcttgctaccttgaaggaattagatggcaatggtgatgtatgaaccaatctcttccctttttttaccagctattgcaataatcaggtgccactgatccccatgaatgatgcatcgtggttatttcatttgaaaagcttggtgcagtgcttcattgaaattgcagcaagtagcgttggaggtagcaatctaaatgttaatctactttggggcttttgacttcaggaatcaggaggaaagtggagcaaaaaaggaggatcatgtccatgatacatattacaaggtccttttccgactatcaattaaagatctgacatgtaaccttttctcattttgtattgatgctagcatgacttgtggtctaagattattgttcaagtaacatttggaatttgtacaaaggattagacactcaagttgtcactttttacaactctctggttggagcatcgacagaatgtgatttactcctcttttattatgttacgatgatgataaggctaatgggaatcagcaagatgcactccttttactaaatatataatatgaagaggtgtaatacttctaataaccagatggtttcactaatgtttgcttagcaatacttagaattctgtattaaatgattttatcattcttttgatattcctatatgaatcgaattggtgttgtataaaaactatccaatgaagtgcactaatatattatggttaagtacagttttcagattatgaagttattatctaaatcaagaacatgctatttccgttctggatcgtattttgacgaatattattggagtgatgatgtgctgcttattccttttttataggtgtatgaggatgttctatacaaattttattcatcaaacatattatgtttcagaagctgcagttctaaattttttaatgtaatcatgtattttgtataatataatatgaagaaaaacatgccttgcacgtgcatggttactagttagttagaaaagcaaaagttttgttttataacatctaaatcatataactaattttcgatatgttttaaattgtttatgactaaatttatagttgaagaataaatttcactataattcaactttgaaaagaaatgatattttgagttatatttttcaagttttgcgtgccattcaagctaaaaatatagattttatcatcatatataacttcataatttacattagatatacaattgtcggagatatgggcccgggggtatgtggagtaaaggagagctaattccctttccagccacgtggctctgtgagctggtcccacccacatacctcgtgagtcactaaggcgatggggagagcctcgggggtgacgtcacccccctcgagctctcccgtcgttttagcccgagtccctccctcggggggaagtgtgagaggggagtccaggctgggcggccataaatgtgcagcgccccaaccgtccctcgttgcattcaatgcggcgagggcagacgtgcggcgtgcccgaacgacccctgtcagtcggatgcgaccggtctgtgaccagtctattgccggtcacggccgattgaacgggtggtcgtgcccccacgtcgcctctgtctccgcggagtggcggtaggtacgccccgtcacatccggacgtcgttcgatgcagggctgccgcaagtcctcactcatttatgagggaatgacagggctgccccccgtgtcaggcgggagacggcgctgggccccactcaaggaccaaccgatgcttagcttccagcaaaaggcacgggttgaaacccggtggcaggagcgagcagggccccctcccctccctccgatggaggaggagggtagagacggcgcatgtggtaaccccttaagctataaaaggaggaccttgcccacaaaaaggggggggggcttttggaggggaaagcaaggggaaccttgtaagagttcactgataatcccaaacgcaggagtagggtattacgcttcagagcggcctgaacctgggtaatcgaattgtgtgctatctaaccggggatcggagggacgaacacgcgacttcggagagacgagtctctgccctcggccgaactcacgaaaggggggtcacgcgactccccgcgatcgggggtcttccctcgacagctggcgcgccaggtagggggcagttgcgcgtttgagaagaagtcgcttccttccgccccaagtcggaccaggccctagaccgtcatggtcgaggtcctggaagcggaggaagcggtggcgtttacccccaccccgtctgggagtggggattcgcgcgaaaaccgtgaccctcgtcaccatcgccatggctctcgaagtcctccccgccgcgagccttcgcggagggaggatccaacccgttcaagtggtacggccctctccccgctcccaggcggagggaggtcgcaacgcgtcggagcacgtcgccgtctcgactacggagacggtggttcgccccaaggcgcgcttcaggaggtgggcgcccttctgcggcatcccccggtgaacccggagccggagacccccgtacggcgctggctggacgacgtggccaacttggtcaccgctgcccagcggcagttggccacgggtggccaatccaccgccacaggtacatcaagtgccccggctaccctttcctcatcggcgaggagaagggcccgtcgatcggccacaacctcgaggcgatccacggctccaacgacatcggaggtctcgggaaggcggaggtgtcacgatgacctctacggggcgcaggacgctcgggtcaacatcgagcgacgccgggacgagcgccgggctacccgcatgggggaaggcgcctcctcatctggagtgccacgctcttcttcatgaggcagccctccccccacgctcacccctggggggcaggttgtagggctttcgttgcgagtctccggaatgtccggtggcccccgaagtttcgccccaacctcacggagaagtatgatggcagcatcagcccctccgagttcctccaaatctacaccacgatcatcgtggcggcagggggtgacgaccgggttatggcgaactactttcccatggcccttaagggtcaggcgcgtggctggttgatgacccagccccccgactccattcactcctgggaggatctgtgccagcagttcgtcacgaacttccaggggacatatccccgccagagggaagaagcggacctgcatgctgtgcggcggaaggacgacgagtccctccgttcgtatatacagcgcttctgccaggtccgccacaccataccgtgcattccagcccacgcagtagtgtatgcattccggaacggcgtgcggcataaccgcatgctggagaagatcgcctccaaggagcccaagaccaccaccgagctcttcgagctggcggacaaggtggcccggaaggaggaggcgtgggcctggaactctcctggcaccggtgcggcggctgcggctgcccccgagtctgccccccgctctaagcggcgagataggagaggcaagaggaaaccggcccgttccgacgacgagggccatgtccttgcggcagacaggcccacgcgggccccgtgcaaaggaaagattaccggcgataagccgagctccaccgccccctctgacgaaggccggtcggcggacaagtggtgttcggtgcacaacacttaccgccacagtctcgccgacttgccgctcggtcaagaacttggccgagcggttccgaaaggccgatgaggaaaagcggcagggtcgacgggagggcaaggctcccgtgacctcaaccagtgaccagcgagaggaggccaagaacaaggcccccgctgacgatggcggaggtagtgaggatctggatttccagataccccaagggaccgtcgccacattcgatgggggggcttgcgctcgcacttctcgtcgagggttcaaggccatgaggcgggaacttctggccgctgttcccacccacgaggcgatccggaaggagcgctggtcggaggtaaagctcaccttcgaccaaagcgaccatccgacggtactcgctcggggagggaagttggccctggtggtctccccgactatccacaacgtcaagatgaagcgtgtgctggtggacggcggggccagtctgaacatcatctccccggctgcctttgacgcgctcaaggccccggggatgaagctccaaccgtcgctaccaatcattggcgttactccgggacacacgtggccgcttggtcacgtcgagctcccagtaaccttcggcgactccaccaatttccgcaccgagcggatcgacttcgatgtggcggatctcaatctgccctacaacgcggtcctgggcagacccgcgttggtgaagttcatggccgccacccattacgcctaccttcagatgaagatgccaggtcctgccggtcccatcaccatctttggtgatgtcaaagtcgccctcgcctgtgcagaacagcgcgcggacaacctggcggtggccacggggccgcaggccccggaggcccccgcgtcccgcgcccccaagaagcgcctcaccttggccgacgaggttcccgtcaaggagattccccttggcgatgatccgtccaaaaccgctaagattggcggaaccttggacgccaaataggaaggcgcgctcgtctccttcctgcgggcgaattctgacgttttcgcatggaagccgtcggatatgcccggggtccccagggaggtgattgagcaccgccttgccgtgcgactggatgcgcgaccagtccggcaaaaagtgcggcgtcaagccccggagcgacaagccttcatcagggaggaggtggcgcggctcctggaggctgatttcatccacgaggtgattcatccggagtggctggcgaacccggtggtcgtcccgaaggccaacggcaagctgcggatgtgcatcgactacacagacctcaataaggcatgccctaaagatcccttccctctaccacgtatagatcagatagtcgactccactgcggggtgcgtccttttgtgttttctagatgcatactctgggtaccattagattcgcatggctagggaagatgaagaaaaaactgccttcattactcctgtaggcaccttttgttatacaaccatgccttttgggttaaaaaatgccggtcctacctttcagcgcatgactcgaattactttaagtaatcagatagggcgcaatgtagaggcgtatgtcgatgacctagtggtaaaaacgcgccgccaggacacattgctgcaggacctggccgagactttcgatagtcttaggtccacgcgcgtaaagctgaaccccgataagtgtgtgttcggcgtgccggcgggcaagcttcttggttttctagtctcttcccgaggcatagaagcaaatcccgagaaaatacgcgcgatagagaggatgcgccccccagcaagctcagggatgtgcaatgtgtcgctggatgcatggccgccctgagtcgatttatatcaagactgggcgagagagtgctgcccctattcaagctcctcaagcgccccgggccgtttgtatggacggaggaagctgagcaagccctcaatcagttgaaagcttacctcacttctccccctatcctggtggccccggggccggaggagccattgctacatctacttggctgcgaccccccacgtggtgagtgcccgccctagtagttgaacgcgaggagaccgagcgggaggtccctccgactcgtgatggcccctcgtcccccaaagcccccagcccccgggaggaccccgaggcccccggaggagaaggcgaggctttggcaggaggccccgagccctgtgatcccaagataatccggaatcccacgagggcccccgagcaggcgcgccccggatcatccgcccctgacaacacgaaccggccccgccgaacggtgcagcggcccgtctactttgtcagcgaggcgcttcgggatgcgaaaacccggtacccgcaggcccagaaaatgctttacgccatattgatggcctcgaggaagttgcgccattacttccaagcacatcgggtttccgtggtaacatcgtaccctctcggccaaatcttgcacaaccgagagggcaccggacgggtggtaaaatgggccatcgagctggctgagttcgacctgcacttcgaaccgcggcacgcgatcaaaagtcaagtcttggctgacttcatcgtggaatgggccccagtggacgaccctgttccgtccaaacccccttcctctcccgaagagaaagaggacccaaacgccgacattcgtggcggacactggattatgcatttcgacggctccctcacccttcaaggcgcgggagctggagtcacactaacctcgccaagcggagacgtcctcaaatacgtcgttcggctcgacttccgagccacgaacaacatggcagaatacgaagggctcctcgtaggattgagggctgcagccggaatgggcatccaccgtctcctggtccaaggtgacttctagctggtcgtaaatcaagtctccaaagagtatcaatgcaccgaccctcaaatggatgcatatgtccgcgaagtacggcgcatggaatgccacttcgacggaatcgagctccggcatgtgccccggcgcgacaacacgctcgctgatgagctgtcgcgtgttgcgtcggtgcgagccccacttcccccggggacctttgaagaaaggcttgtccaaccatcagcacgaccgaacccctcgaggggctccaacgacatgccctccgccccgactcctgccgacccgcgcccctcggagcctgagggggtcgaccccgaccctcctccgtgccccccggggcccgagggggtcgaccctgacccccctcgccaagtcgcatggatgactgatatccgggcgtatctcgacggcaatactcttcctgaggaccacgcagaagctgaaaagcttgcgcgcatctctaagcggtacgtcctcgtagaagggaccctctaccggcgtgccgccaacgggatactcttgaagtgtgtttctcgagagcagggcatcgagctcatagccgacacccatcagggcgagtgcggggcccattctgcctcacgaactttagtcgggaaagccttccggcaaggcttttattggccgactgcattacaagatgcccaagaatgggtccagcggtgtaaagcatgccagttccacgccaagcagaaccaccagccggcccaagccttgcaggtcatccccctctcttggccttttgcggtctggggctagacatccttggacctttcaaagcggctcgaggcgggtatcagcacctgtacgtcgccatcgacaagttcaccaagtggcccgaggcttaccctgtcgtcaagatcgacaagcattctgccctcaaattcatcaggggcatcacctctcggttcggagtgcccaaccgcattatcacagacaacggcacccagttcaccagtgagctgttcggcgattactgtgacgacatgggcatcaagttatgctttgcctcgcccgcccaccccaagagcaacggccaagtcgagcgagccaatgccgaaatcctcaaaggcctaaaaaccaagacatacaacgtcttgaagaggcacggggattcatggctcgaggagttgcccgccgtgttgtgggcaaatcggaccaccccaagccgcgccaccggtgaaacgccgttttttctggtgtacggcgccgaagcggtcctaccctccgagctctccctgggatcgcctcgcgtcacactgtacagcgagaccaaccaggatgaccttcgccgcgatgatctcgattatcttgaggaacggagaaggcgagcggccttgcgtgccgcccgctaccagcagagcctgcgacgctaccatcagcgtaacgtccgggcccgatcactgcaagttggcgacctcgtcctacgccgcgttcagtcgcgcctagggctgagcaagctctcgccaatgtgggaaggcccatacaaagtgatcggcgtgccctggccaggctccgttcggttaaccacggaggacggcacagaattgcctaacccctggaatatcgaacacctccgtcgcttctatccataatatcgagacttttttgctttcttggagctcgggccagcccccgcacaacccctcggtttgtgtgcgcctggctgggggctaccactgtgtatccttttttcctcttacaagcaatgaattcttatgtacttttacccaaagaccgtttttttcccttcctctggcttgtcctggtttaaggggtaatctgcgtttgctctgaatcctaagtgctgtgaggcggcctggaaaccccgaaagggagcacgaacgcgggccgtgcccagggttgcgccgaaccccggaggatcggaagggcctacacacggccgtccccgacccccggtcgacgcggcctcggtctggccagtcccgctaggcggctccctcggagcagtgggtttgcccatcactacttaggtgccctggaccaaacgcggattcaccatctcgctagaacaagcccgaggggggggatggggtcaaagcggcatctcgatcacggactgattatccccttttcccctgttttttttgttgttctctctgtttcaaagacattcagtggaaggagcaatgaatagaagaaacgaaaggggaaaaggagctaaggaagtttcaattgcaaaacaaggcaggatggcacgacggcctaaatacaagaggtgcccgctacgggcgcacaacaagagggaattgaaggcgcggatagtcacggggtgcccggccctccgaggccggcgccgctgacaatgtcgtcccagtcttcgtcgctatcgtcgcccccgctcccgctttcttcgtccgaggccagcccggaggtgaagcggggggccgatccctcgaagcccgagacaatcgcgtcggctgcctcccgaacttgctcccgggccctcgcttcagttcccagggaaaaatcttcaagtgcgcgccacggcatgaagtcggggtcgcgcgcttggtggcttgcgagaaccagctccaccgcggcccgtgccaaagaagctgacgaggactttatggtctcgccgacctcctcttccagtctctccaggcccgccgctagcccgtccaaccggaacgcgagcgctggttgtgtgggcggaagttggctatgctggcgcacgggaacgccgactcggcgcgccgcgcgctccagccgagcgacggcgtcggagagctgcccgggcccgagttcgttggtgaggcggagggccgcaatctccctagcctgatcttgcaccaggcgccccaggtcggcaagggtactctgagccttggcgagcgagctcgccaaccccgcgccgccgggcggcccgccggctgccaggtgcttctcccgggcctccagttcagaagccctcgccgttatcgccgcgcgctcggcgcgagcgctctccagatgccgggcctcgcgcctagcagcagcctcctcgcgcttcgcaagctgcttgccaagccggcgcaaggctacctcgcggcggtttacctcggcttcgcgctcggcgagtgcggcgtcccgttcctggcacacctcctcccgcagccgtagctcttccgcgcggcggcccgcggaagcttccgcggcaagagtgatccgatcatgctcagccgcggcctcttcacgaaggtgaagggaggcctccgtttccgccgccgttctctcgtgggtggccaaggtggactcccgctgatccagcaggcgtgcttgctcccccagctcctgggcccgctgtgcctgttcctggccgcgcgcgttttgttcgcgctggctccgatcaaggacctcaatccgccgacggatcgtggcttcaaactcctgcacggagcgggcacgatcgtccaaggccttgcgttcagcctcgagtgccgcagtccgggcctgcaacgaggcgtcggcctccgctgctcgccgctcgtgggcagcggctgcgtcgagggtgccacgcgcattcctgaccctctccgctaggtcctcggcatgggcctcgtactgaaggcggatatcgcccagcgcttcgtccagggcgctcgaggcgatcagcgccgcctgccgctcctcctccgtctcccgcatgacggagtccagcacctcctcgcgcgcctagattttggctaggtgggcttagtgttttttgcgccccaccctcaccatgtcctccacggcacggcgcccctcgtcgactcgcgcgcggtccgccacaagctgagcccattcggcatccagggtcgtccgctcttccgccagggcttgaacttgggtattgagcccctcccggacagtggagtcggcggcggcgagcacctgcagaagtggctccatgctcgccggggatggggaagaaaagcccacgggtcacccccgggacgacggagtgctgctggaccccccacgagacgggggctgctctcggccccccttctgcgaggccagccccgagggcgccccaaacgcaagggggagcggctgttcttttcgtcccccgcttccagtcctcgggtggattcggcctcggcctcgggctcagggccgccctgtcacatcccaaaaatcctaaatttataaattgttgtttaattggaatttttagaaattaaattaaaagcctacaagctaaaccttaattttctaggaaaattttcaacataaaaatgagctaaataaaattttattaaatactatgcttgctcctctattttctagatttttctgggaattatttgagcaagggaagtatttttaataattgaaacaacattttacaaattgttttattcaaaaaagttcaaaaagtccccttcttggtccttgggccgaatccggcccatctctcctctcttctctctctttccccgcgctcggcccagctcggcccaagccgcgcccgcgcccgcgcgcccctctcacccggaggctgacaggtggggctcacctgtcaggtcgtcttcaacctcccgccgccgccgcgcccgcgccgaaccctagccgtttttcccgcatcgattccggccaaatccgatgcgatctcttcaaattgattgaggggttttgttcctctcggtctcctcttccttttcccccaagaatcaaagcaaatggttgagttttggatgagatttggattcggtttcgagtttatctccaaaaataagtccgaattcttcccgattcgattccatccgtttgcggttcgatctcttcgatttgagctcatataaatgttccccgtgatctcctctctcgtttgccccctttcctgagtcctcccgtgcctcctagcgccgtcgccgcgtgcttttagccgtcgccgtcgccggccgtccttcggcattgccgcgccgccgtctccgtcgtcgccgccggtcgccgccaccgccattagcttcgccgtgaggaggagaaggccgtccgcccctccgtttgcgtcgccgaccaccggagcacccccgacgccgtcgacccgagccgccgccgccttcttcttcgtcgccggccgccgtcgttcatcgtctcgtgtcggggtgggtaccgttgagttcctctcgtcgccctctacgcgttggtgccctccaatctcgtcgccgaggtccgtagcgccggcgagctcgtcgtcccgagccggccgccgtcgctgacgtcaggctgacgtcaaccctagggcagacgtcacctctgccagggctgacgtcatccttttcctttttccctttttctcgatttttaaatagattaaaactttgaaaaatcataactaaataacctgtagatccgttttaggtggttcaagtttctaaattcttctaaaatcaagatctacatgttaaaaatatccacatgtactgtttatgcttgtttttgtactgttttgttgattttgtttatttgctttagtttccgacgttccggaggagagcgtttccgttgaggaaggttccgaagcgtttgtggaagcccaaggcaagtcacacagatcccaaacaaccctttgagcatgttgatcctgtttaaagctattattttatttcaacttatgcattattttcgaatgtcatcgggtggtgaacctttcccagttaattatggccgaagttgactttattttcctatgggttataatttgattagcatgaaccttatatattggattggttcagctaaatgctatatatctaggtttgcttagccatgcttagaaacattagctaactaaaagggttaatggtaaactacttcattattttatgttaataattgtggttattttaatggtagctcacgatggtcaattgtgtgataataattaattgataactaaaacctggctaaggtgggttgtgagcatatggttttgatggttgtgctcatgacaattaaggaccggttcgcgagctactgttgtgatacatttatcgtgccaaccacaagccagcgtgggcaacggctttatcttttgtatagcatagttcattaaagtgcgccagactgagaagtggcgagaagtccatgggggtcgctggggagtccatgcctctggttgtagagggggtgattatgatccaggtacggtgcactgtggtgaattgtgttatgtgagggataatgtcacaattcctttccgagataccgtggtggtattgaggcacatggtaacatgatgtggggttgtgtcttgtgggtacagtggtacacctctgatcagagtttaatctattcgaatagccgtacccgcggttatgggtgagttgagcaatgtttttcgtgattagtctcacactactcattaatggtaataatgtgataattaatttaattcctggtttggaatggttaattcctggtttggagggttaatttgttcagccggggttggttgatcaaatgtggttgggcctatgcaacacgggtgtgttgtatggtgttgatttaatattgattaattatataactgttttattattctcttaaatatttattaaatgctgttttctgcaaatgagctatattatgccatcctttgttatcctgtgcacttgcatatttgctgtgtggcttgttgagtatgtcatatgctcattcttgcaatattcattcatcagaggaggagtacttcagtgaaggtgatgatcttgagaattgagcttattctggttaagttgcctgtggagtggagttgccgtcgctgttccgtcgtttgttttagttttatctttccgctgcgtagaatgttttattttctttgagaggaactatctacctctgtaatattttaatttgcgagtatttaatagttaattatttgtactctattatcaatattgtcattgtgtgcctcggttgattcctggacgagggcttaacacacatgtaagcgtttggaattttggatagaaattccgggcgtgacacgcccccagcctcgacctcaacctcgggctcacgaccgtccgcaccaaccgatcctgggtggcgctccgcatggaccccagtctcggagccgcccccgggaggggcgtcgacgccaccgccgcccacggtaggtcgagtcccggctaagggtcgctcgggatcgggcccagtacgtgtcctgtggaccccaccgcgaggttgcgaagaccctgtggtcttggattggccaggcccggaccgctccggttgtgcggatttcgtcctccgtgaggggtccgaccccgaggtcccctgagaaggaatatcgctacaagggaacccagatcgcgttaacaaaagaaaaataaaggggagggaacaagaggccgagctaggtaagagcaccgaagtgagcgccagcgtttgtacctgcggggaggacagttgaaggtccactttgggggctcgatgaagctccctcggcacggctccgtttgccctatcttccggagccgtttcttcttccgaccggcctcgggcccggcaggtcgcttgtgacccgccggtggaaggtctgtcgcgcgctcggcgcctcctccacgcgggggagatggcggccaagattcggtcgccttccgcttccccttcgggtcacttgccgggtcgtctctgggtttgcggcccgagccgggcgcccgtgagtcgctaccaccgcctggaccgcttggccgcctcccgcccacggacgcgccgccaccaccggcgctggcggcgctaccgctggcgcccccacggccaggccggcccctgccggcgccgacgaccgacatcacggccaagatgctctcccggtcgcggtcgcagcagagggggaggatcccatccgggatcagtgtttgctcggcggagtccaggcccagcacccgccgaatcaccatcttggcgtcctcctcgccccagtcccagcgctggcccgcatgcgtgcgcatggggtcgttggggccggtatactcccacgcgccacgggatcgttcctggagaggggcgatacggcggtgaaagtagtcgccgaagaccattgcccctgtgaggcccaagcttcgcaggcccctgagacgatcccacaccgcgtcatattcctcgcccagctccgaagccgccagccatgcggcggatctctcgggaggaccggcgggaactcgaagtcgtgggtgatccggcagcgcagtgtagaaccagtctttcttccagtcctcccttttcttgcggaggtggctctcgatgtactgccccgccgtgcctggtcggggctggaagtaacatccgcccaccaccgcaccctatagcagctgcgggacgaagaaggcctggaacaaccgcatcgtcggtcgcaccctgatgaacatctcgcacaggtgcgcaaagatgctcagtgtcatcacggcgttgggcgcgagatgcaaggcatggatttcgtagaaatcaagaacctcgtggaaaaatcgggagaaaggcggaatcaatcctgccatggcgaaggacaagaggtgcacggaccgctccggatatcgcggccgtggccgtgactcccccgccctcacgacggcgccgtccggcataattttgcgggggaggctaagatgtctgtctgccgagatgcgggaagaagggagcactgtgtcgccggtgtcttgagccatgatggccggcggggcagtgacagaaggcgtggcgcgcaaagctgcgtggagtggtgagagtgttagggttcgagaggcgagaggacgagtcggcggttagcgaaaggaaggaggggaggtccctgcctccactcccttttaattctcgcctcatccgcgctcgcctcctcgtttcccgccctcataattgcccccatgtccgcagttgtctttccatttcccgcgcccgttcaccacgtcgcccgtcgtatccgcgtcccacctttagtgcgcctatcaccggcgcactcgaggtggaatgcgtaacggctataagcgcaagctgagtgaaccgtcaccacaaccgccgtaggaatagcgggcgaggtaatcgaggcgtggtctaaccgatcccctgattatcgcgtcccaagcatcctcgtctttagcgcgatcagaggcgaccgaaccgccgctatggctggcccgctaatctggccgctcggcaatactcctttaaactccccgccgggcccataaccccagctagggaggtcgtgaggcaccatgtcagaccgatcccaggaagcccgcgtctgacatggcgccgggggctactgtcggagatatgggcccgggggtatgtggagtaaaggagagctaattccctttccagccacgtggctctgcgagctggtcccacccacatacctcgtgagtcactaaggcgatggggagagcctcgggggtcgacgacatcccctcgggggtgacgtcacccccctcgggctctcccgtcgttttagcccgaggccctccctcggggggaagtgtgagaggggagtccaggctgggcggccataaatgtgcagcgccccaaccgtcccttgctgcattcaatgcggcgagggcagacgtgcggcgtgcccgaccgacccctgtcagtcggatgcgaccggtctgtgaccagtctattgccggtcacggccgattgaacgggtggtcgtgcccccacgtcgcctctgtctccgcggagtggcggtaggtacgccccgtcacatccggacgtcgttcgatgcagggctgccgcaagtcctcactcatttatgagggaatgacagggctaccccccgtgtcagccgggagacggtgctgggccccactcaaggaccaaccgatgcttagcttccagcaaaaggcacgggttgaaacccggtggcaggagcgagcagggccccctcccctccctccgatggagggggagggtagagacggcgcatgtggtatccccttaagctataaaaggaggaccttgcccacaaaaagggggggcttttggaggggaaagcaaggggaaccttgtaagagttcactgataatcccaaacacaggagtagggtattacgctccagagtggcctgaacctgggtaatcgaattgtgtgctatctaaccggggatcggagggacgaacacgcgacttcggagagacgagtctctgccctcggccgaactcacgaaaggggggtcacgcgactccccgcgatcgggggtcttccctcgacaacaatcataaaagtaaaatgtccagtcaaaaaaatcataaaagtaaaatgaatataactatgagtaatattcctaaatatattagttagaaaacaaaagtaatgctataaagtaagcaactataacatctaaattatatgactactattgttctgtttgtgactattttatatttaaagagccatattcactatatttaacttttgatatgtcactttgaggttatattttctgacttttgcgtgacattcaaacaattatgaaattatgaaaaagaattatatagattttatcgttgtatataattgcacaataattaaattagatctatcatggtataagtaaaatgataaaaggactaacttggactatttgaataaaatctaataagcttgtaaaagaattagttcttactctttccttaaattggaccgaccattgcatgttacgttttacaattttgagaaaattccttctatacccctgaaagtttagccaatcccttctacgcccccgagttttgtccactcccttgtatgcccctgaattttggttttgatccctttcgtacccattccgttagtttaccgttggttgaatgtttaattccaatgaaaaggacctttttgcccttagacaggaagggaatctgaaagttgtagactagttgtattgtcggaagtgtataattttgttatataagactattatattcatggctttattgtacaaggtattattcatggcaaagtttatttttacataatgacataaaaataatatttatttacttattaaaagagcatatgtagcaattttttggtagttcaacatattttctcgagtaaatttcacaaaactacaggtattttgactaaattatcacaaaactacacatttaaggagttgtatcacaaaactacacatttagcaccaaatttatcacaaaactgcagattttaggttaagtatcacaaaaatgcatatttaatattaaacttatcacaaaactacaacttttggctataaacattattaatttatgattaaattggttctaaacctgtagttttatgataatttagttactaaacatgtagttccgtgacacttcatcttaaatgtgtagttttgtgataaatttggtgttaaatgtgtagttttgtgatacactgagttaaatatgtagttttgtgataatttagtcatagtatctgtagttttgtgaaatttactctattttctcctgttgtatatattatattggaatgctttccaaaactagatttatctttaaaaaatgctacacatactttattaatatatattaatttttttgtacgacaaagttatgaatatgataatcacacaacaaattttatacttccaacaatatagtttacagatttctacatattcctcacatattgaagggtaaaactgacttttttatacctattaaacggtcaattaatggcaagggtacggaagggatccaaatcaaaattcagaggtatacaagggagtgcagaaaatttaggggcacataagggattggctaaaattcaaggatatggaaggaattttctcagtttttaaatgcttagatattaatggccttatatctattctgtatcatagaattatgagtttcttcgcatattattgcatatataaggtagttaaaatgttatatttgttactattaaatttaaatttttggacttatgttaattaggtattttcttttaaatatatatatatatatatatatatcacgcatatcacgaggaagcgaggtctacgacatatctttgttatgtaagtatgcaacagcttgcgtcaatctgaaccaattaaaaggatgactaacttgatgctacaaataaaacatatttgttaatgataacagttctttagaatgtgttaaaaaatccactaaacattcaattaaataagacaaaacaccttacttgcttttcagttagacaagttttataaacagactagatctaactctatactgaagaaatcatatataactatttatagggatgtcttaaatattacatgtttcaaaagcaaacaaatagtttaaaaaaaaattacctaagcgaagaaaatttatggagaaatactgcttttgaaacatgacaggtgtcatccggttgatattccatctatataagtttttaaacgcaatctacaatacatgcccgcgcgaatacgtgggctaccttcctagttttgtttttctttttttgataatctaccttcctagttattattaaaacatgataaagaaataatcctccaagttcgtttcgtgacctagctagaaagctatcatattaataagaaagcaaataaatatttggtttcattttgtaacagtactcagactgatcccacacaaattagagtactacaggaggtacgattatatttggaataaaacaaatatatccatgtgtaaattgcaatgaaattgtagatagttctacgtaaaacaagtagaagttctagcattcgatataatgtggtcggagttggagatgcaatagacatacctaacttgcggtcttgggaagactttggaggatcggttgcttggaatgtcctgtgtacaaaccaacgtagaccataaagaaatgaatgtacgagacgagaaagagaggtagatacatataagagtgattttacggttggaccttgagtacacctgaagatatatatgagattttgcattataaaatttagatacctcctggtacatgaagtactaagagggagcaaaaattttacgttataaaatggtactttgaggttcatctttgaaaatcataaaatttctccataagtttcagtttgcacatgaaacacaattaaaatttaatttattattataggtatagtcggagaataagaaaactacaaggcttataaggtccaggaagtgtgtcagataacatgtttgttgtaattgagaaatgcttttgttgtgtggtggactgatgggtgtaaacttttttccctacctcggtgtttaaatcgaataaaggtattggggcttccaaaacacaaaaaccagttattgtgatttgcttccaggaacctcatgttttagggcaaaatgtttcaaaaagccccatatttatgtaaaatcctcgtatttcatttttacatatgattatttgtacacaaattcatgttaactcaaagtacacattccgcttgtctcatttatcttctcccccccccccaaatcttttattatatactaaaattccattaaacttcctacaaatgctcttaagccgccacgtggcaatcctacaaactctcctagatcgccacatggcactatataatctcaccgttgattttcacttaaattggtggtcccgatattttagaccattagattgcatctgttattaaaaaataataccttcctttcacaggagaaatatataactgttgcctaaaagataaataccttcctcacgtacgtacgactaaaagaaaaaaaaaagctaaaccattaattgtttgctgcaagaaaaagaaaaagaagaccacgtgcatgtacttataagatagagaaaaataaagaaaaaaaagaacgtagtaccgttccaaagaaaaatcgacgttatttcatacaatttacaaatttacgcaaatatacgtagctctttaaacattccatcttttaaattattttaaatttatatccaatttgtgatactattgtattcctttacttaaaaagatgtctatgatccaaaatacatcatagcaccagcacatgggacccacatgtatataattgatgttatgttatctatctattatccatctattatctatctatctattatataataaaagtccattaaacttactacaaacgctactaagccgccacatagcaaccctacaaatgctcctaagtctctacgtggcgctataattatttctaaaaaaatcaaaagaaggcaaaacatctgatttttttaatctattcccgatggacccattgttgtcaaccattagatctatttttaaaaacgatcaattaaatttatctccatcaatcccacggacacgtgcatatatacaagcacagcacgtacgtatctaagtaaccagctggcctcatgtccctctttttcttttctttttctttctaaaataaaaataaataaatacaccgttagaaaccattagatctatcatgaaaacctaacagttaattttatcgctaaaaatcactaaatacacgtgcacataagcaccgtatgtaagtacgtagacacgttggcctcacaaccctctatgtctgttttttttttcattctgaaatccaaaaaaaataaaataaaacaaagctacatttagggtttaaaagtaaaaataaatcaagctataagcttccgattataggatagaaaaaacagtatgcaatttgaattccttattgattcatcattgtattatcctttggtttccacaaatatgtcgtagcaattctgaaggaaaaaaaatcattttctacctataccatgcaagaaacacacaaaaaaatttaagtccattaaacatcatgcagaatgctcctaaaccgctacatggaactttaataaattagaaaaaatcatataaaaattataagaaaaaagaaaaatacttagctatccgttctaacttaaatcggtggacccattatttctaaccattaaatcaatccttcaaaacacaaatcccttccacctcccactccctcccgtacataatcactcctccctctccaacatccatatgtacaaagcatatatcttatatacaactcaattagattaaaagtaaaaaaaatgacacataagttgtttcgtactcccccgtcccattttaagtgcaaccataaaatttcatgcccaatgttgatcgttcatcttatttggattttttttataattagtatttttattattatgagacaataagacatgaatagttactttatatgtaacttaagtttttaattttttttaagttttttaaaagacggacggttaaagttgatcacggaaaatcatggctatacttagaaatgggagtagtaagtagatttttaaggaataagtttacattaatcactgtctttcttataaataatggaggtacagtttcaaccttttaatataaatgaatggttacgatgctcaaatgtgctttattgcgcactcatatcaaaatagacaaattatcagtttgaagtatatattttttatatctttataactaagaaaatacttagaacatgtgacgagttataatgcacgcataatgttatgatgccggactcagtcatcatatagaatataattattttaatattttactgcctaattaaatatgtcaatgattgttagaataactaacgctcaacatttaattttgaaaatatcaaacactcatattcaaaagaaaaagatatagtagtataattttgaacgatattaacaaagtgcccatagagtgagctataaattataatcttggtggtttataaatctatacaatctagaaagtatcataactacttaaaaacatttttaggacaaatatcatatatttaaactattatcaaataacatcttaagtaaaagtcgtcgtatatccctacggcaataaaataaaaccaagatgtccatgcatgttttattccttcacatagcatgtaggcaatcaagctgctaatggaaagtttgtttggttatgatttatgcattgatagatgatattttgatctaggtgggttaataaattttcaattcccatgattttctacactccattaaaacatccgttcttttcaatactctattttacacatgtattcctatcatatttttgtacattttctattctttttttttcaatcatgtgttacaaaggaacctttaattttttttcaccgagaggcaaaatcataacatttcacaatcctaataacacaaattatgatggtttaaataagatgcccgcgcagatgcgtgggctaccttcctagtttttcactaaaaaaaataaataatcctaactaatattgatataataatttgttaaatatacgtataaatgatcaaactactacgatcgcaaaatgttgaaaatcatctatgtacttaaaaacgtctcatatatattttttacaattgttatagataaatatattagttaaattgatgatataaatttttaggtgaggtttaatgaaatattttgcccccaaacttgaggttttatgaaacaaattataaaatctagggatttgcatcatggattcctcaaaacctgaagttatgtgaaatttgctccaacataaatttcgtgaggtttgaggatgtttatcctttgactagtattttaaggataaactctcatgtgtttgtgggtgacataaacgcttgttatatacttattatactatcggcaagtataaacaacccatacctgctatcgtcaaggttacgctcatcctctggttccatctttagagttcggtgttgtttggttaaccaatcatacggagaacacctttctcccggacacgctttcacagagtgcatctcccttttaaagcattcatatggtaaacaattattattgtgaaaaatccctaaataagatggtaaagaaatatcataaaaaaactaaataggatggcttcctatttatgagaacaatccacacctgctatcggaaaggttccttgtatcttctagttctctctttgttgtttggcgtatgtctatctctattcggtatcgtttgcttgaagaaccatcttcgtcagagctctgtttttgtctgtccagatgattagcttcctgaacctgactgacttttccaagaactttgaaggtaggaccgtacggatgcttgccaatggcattcttgcaaacagattcggcagctccccggtcagattcgatagcaccagtggcaattccaattcttgcactgatatccgtaaggtttaataggtgctccacgcccaaaagcgcaataccatatccctcacctctatgggcatcgaaaactagatacagcctctgaagattgggcattgccccttcctgaaactctattcgcagtttatgacacctgaattcaaagaatatcagagcagaaaatgtaccttttttgaacacgatgccttttgcagtgggttcctggacatacagtgagagagctgcaagggtacccaattctgtcaggatatccatgtcgctatccaggaattccctgatcacaattttcaaaatacggagttctcgaaaatgagcaatccactcgggaatttctgaaaagatgcactcagatggcaacagctccagtgtttgaacgggaggagaggatgcgctcgttgaatcatggaggctagcagtcgtgtgtgaggtactaggatccatactgtgattcttgatgccatcagtaactcctggttgatttggcataccttggaactggagtggtaagaaactgagatgaagaacattcgatggaacaactgcgtcgtttgcatttatttccagtgtctgcaagtgtttaaaactctgcatctgatctggcagtttcactgtaacgctacaattgacctgcatatatctcaatagaaccagttgataaatttctgtgaggtctatcatgtttccatcttcacacgaaaaatcaagacatgcaacacgaagaagcttaaactctttaattgaaggcatgcagttaaatagcccaataaatataaaagaccgaatttctgatagcccaatattaaccggtgtagtagcatatgttgcactgccgaagtgtagggacaatcgatgaaccttgtaagtaagtcttactgtcgtttgagaataatcaattgcagtgacaaaattctcttcctgggccttgcgtgtgataaattcatgtacaatatggtgaactgcataggtcaatatgtcagatttatacttgatatccctacattggatgagtcccagattgacaagcttattgaaataactcatggcaacttccatggcattttcccgtgttgacgagcagacaaaatcttgggctatccattgattcaccaaatcttccttcaaaagtatgtagttatccggatatacactaagatatagcaaacatgtcttcaaacaacaaggaagactgttgtagcaaaggttcagtacttgcttcagtatctcatcagaagtagcatttaccctcaaattattgcacaagaaattttctacatattgccagtacttcaatctatcttccaagttctgtactgtctcatgttggattgctagaagactgcccatgatgataattgctagtggtgagccaccacattttcttgcaatctcttgtgtgactttgtggaattgaccgggatgctctccagagccaaaagttctactgattagcaacttcttcgactcgtcgacactaagagctttcatcttgaatatgtactgagggttataaacgcaacaagccagagcaacttcatcaacttttgtggttgttattattctgctcccacaattattcacaggaaaagcatgtctaacaacatcccatacggatggagcccataaatcatcaattacaataaagtacctgacaaataatttggtctgtatcagacgtgttttgtcaagcaaaagagaaaatatccaagaaaaatatcatgacaatggatttttgttctaaaaaacaaaaagaagatttgcaaacctcctgttttttagatgattcttgacatcgtcgatgaggttggtcacagcacaagggacgactggttggagcggccgaacttgagagagtatattcctcagaatcatccttatatcaggctttttggccgtctgcacaaaagcccggcactcgaattttccttcaagttcacgccatagtagttcggcaagcttggtcttaccgattccttcagcaccgagaatgcataacaccttgagattttcatcttcgtcttcagccatccaaccacgcagcttgtttctcgggccatccatgccaacagggtcaacaaccttgctgaacacagttggaagatgatgatgtacaaccatgtttgtgggacttgatccagcatcagcatcaagattatagatgctccatcgctcattcgcctccttgacaagagtcaagaatcctgagatggtgtcatcccaacccatcttggcaccagcatcagcgttgaccaacaagtcgatgcagtcctccatgtcgtagcaaagctcgcgcacatccttcatccagtaaatcaccgtgagggatggatctcgcacatttgaaagcttctccaaacgatcttgtatgacgctaagctcagatatgagttgatggatagtcgggctatcgagctccttcaacttctgtagaagggagcccatggcacccagcgaagcgctaatcggagcttccattcttgtttctgcctgcagttgatcaaaagggaacacatcatgagctgctatcagccagaacctcatatggacgaaaatcatgaaataggaggtaattcattggaaagaaaaaggaacatcattaatcgaaaagaaaaccgcagctaaccatgagattatgatatgttaggggagacacggcagtactgcaaaggtacgacaggagttatttggagagtgatttctctcttcttcgttttaagggcaacgaagaacaacaatatatcctgttcctcgctccgacctttacatgtccaatcccaacagctccgatacccccacgcatgcccaccaggcacccatacaacagattttgtttatactcaagtggcacaaccctgtctcagcatttttttatttaaattttaaaattaaatctcctatcatactttttaaaaaggctactactatgttttagtatattatagggctgtgttcgttccagcccattcccaacacgaacagtatgcacggataatggagtggtccattagtgcgtgcttaattaagtatttgttagggttacgggttagatataccctctacccttcgatacacgtcacatatcgatatggcatgcccgcgcatatacggacgttctccgtatacactaaggaaatccttcacggaggttgcagagaataagagttcaactcggacaggactgggtcgtcaatgtatcgtgtagggtccgatgtctccgagttcgacttggaaaccgcctgacctgcgatataaaagggacccctgggaggacccgagggatcgaatctcagatccaacacaaccaccacagcctacgaagtcggagcctgcaggagccaaatcgtcgggatagctagtcgaacctactcgattacgttctcgatggtttcatcgagttccatcgttccccttttgtaacctgtgatttccatcatataatcccatatcaactggattagggctattacctgttaaggggcctgaaccagtataaatcttcgttttctgtttgttcgatgtcgtattacgtagatccttgtaccaacgtaccccaataccctctatatctggtctacgggtatcacccgtcgacagtggcgcgccaggtaggggcacttggtactcaaggttctactattacgtcatccagctccgttgacaacagcatcagcgtcaacctcaaccagggaatccctgctcaacaatgctggtgtggactcaagtcggcgaaatcgtcttcccggttttcaccacgatgccgatctcagacaataatcagaaacgagaacgcagtggctacagcccaagacgatcccatgtcgaaggatcctcccgccgaagcggagaacggaacatcgactacatccaaactcgagaagaattctactgcggccaaaccttgtcgttacgacgagaaccacgagccgacgagaataacttccgaagcgacaaggtcctggtgtcctatccacaaaaccaggaaacacactttgcaagcttgccgggttttcctcgacgtccacgccgaaattctcgcttgcaaagagcgtggaattcagcgtatctctccaactcgtgatgtctactgtcctattcacaagacaaagaatcacgacctctcaagctgcagggtctttctcagcgccacaaagacgtcatctcctaaggtccatcagtcaggcataccccttagagaggaagacaaggaacaagaaatgctgatctcagatcgattcgttagcgaaatcaacattggttcccacgaaccatcaggcctgcatttactcgaggactatggctcatcgccaacgagtacgccacgcgaggtattggctatcgacgggaccagcacatcagcacatgctaatgcggaagcgcaaaatcaagtgactacactggcccagcacatcaggaccatcaacgcaatattgagggagaccccttacgatcccgtcctaaatgatgacctcgcacagtggatggaacgtctacgggagtcggtaaccaacctcagcaatgcgttcgaagaggccgccgccgcagcacaccaggaacagccaccaagaggcgatgtcgatggcgaaaatcccgaacgtcaggagtcaccccttcaagctacccctccacctcgcagcaccgttgatctccgcgaccatctcaatggccgccgagaggcacgacgcacgcaagacgacgggaatcgctcccgacatcgcgtctcttcacgacatcgcgaggaacgagagggccgctcgaacgaagaccaagaccgtgctaatcgccacaaccgccacgatcatgatgatcgcgaaaggcggatgcaaggtgatactggtcgaggacaccgccataacgacgagaacgacagagatcggcgccgggacaacaacggaggacgacgacaggattctcgagaaccgagccgacgacctcgtaatcccacaccggatccaagcgatccatcgtcatcatcttcttcatcttcgtcgtcagacagacatccacgaaagacacacgatcgccgacaagccaccgcccccagcgctgggtgtagggcttccggccgttccctccgtgatgtccgatggcctgagagattccgacccggagcaatagagaagtacgatgggagtactgacccagaagaattccttcaagtctactccacggtactctacgctgctggagcagacgacaacgcgttggcaaattatctgccaaccgcattaaaaggctctgcacgttcatggctaatgcatctcccgccctactcgatttcttcgtgggcagacctgtggcagtagttcgtcgccaactttcaaggaacttacaagcgtcacgcgatagaagacgacctacatgcgttgacacagaactcgggtgaatccttgagggaatatgttcggcgcttcaacgagtgcagaaatacaatccccgacatcaccgatgcttctgtaattcgcgccttcaagtccggtgtccgagatcgctatactacccaagagttggcgacaagacgcatcacaactactcggagactattcgagatcgttgagcgatgcgcccacgtagacgatgcgttgagacgcaagaacgacaaaccgaagacggggggagaaaagaaactggccatgaacgcacccgagtcaagcaagaagaaaaatcgcaagagtgggaaaaggaaagctcaggcggaagttctcgcagcagaatacgcgaacccacccaagcgcccagacccacaagatagcaacgcaaagaaagcatggtgccccatacacaagatgaacagacattctatggaagattgccttgttttcaaaaagtcgctcgagaagcacatggcgcttgaaaaaggcaagcgagtacgagtgatcgagaaggatgcggaggcggctgctcaggaatcggattcagcatatccagactctaacctccacgtctctcacattttcggaggctccacagcgtactcctcaaaacgagaatacaagaaggtcgaacgcgaagtctgttcgacatcacagggggctacacccaagatgaagtggtctgaacagaagattaaattttcagaagaagaccatcccaatactgccgtcatcccaggacgctatccgatcgtggtcgaacccactattcggaatatcaaggtcgcgcgggttctcatcgacggtggcagctcaatcaaccttctcttcgccagcactttggacgcgatgggaatcccacgaagcgagttgacacccaccgatcaacccttccatggaattactccccaatcctcgtccaaaccattgggcaaaattacgttgcctgtgacttttggccaagcgaacaacttccgaacggagtagatcacctttgatgtcgctgaattcgatatagcatataacgccatcatcgggagaactgcacttgcgaagttcatggccgcatctcactacgcatatcaagtgctcaagataccgggaccaaaagaaacaatcagtattcaagggaacgccaagctagcggtacagtgcgacaagcagaacctcgacatggtcgagcacacgcctagcccacccgctacaactgagcaacccaagaaagtggccaagacaaacaagacgccgaagccagacggcgcgatcaagattgttccactctctagtgccaaccccgacaagaccgtcaagatcgaggcatcactaagtgagaaataggaattcgcgctcatcaccttcctccgcgacaatgctgacgtgttcgcttggcagccgtccgacatgccgggggtccccagggaggtgatcgagcacaaactcatggtgcgacccgatgccaagccagtaaagcaaagactgcggcgatttgcaccagaccgaaaacaggccatacgagaagaactcgataaacttctcaaagctggctttatcagagaggtacttcatccagagaggtactttatctagtttattttctggattctacaactttaacttctcagaatctgtactaaaagcgagcttttgattttgggaaaagttgcatgtgctataagctccacaaacaagtcctaaaatagctcacacgttaaatataagatgagttagaccttgtttggatcgtttagtctatggactaaaattagtctctggactaaaaactttagtgccctactgtttggatccg

SEQ ID NO: 6 - DNA, 69300 bp, Oryza sativa
  misc_feature (26297)..(26395): N is any nucleotide
ggatccatccagaccattgagcggattaaaacgattttagcaacgtgattcttacggaaaccttacgaattttttttattaggtatagatttaattagttttgtgttatctataacagatctaatatggcataaataatgggcccactgatttaagtgaaaatcaatggtgggttatttttagaaacttttgatattttttaatttattaaaataccacatgccacatggcggcttgagagcgtttataggatgtccatgtggtggcttgagaacgtttgtaggaagtttcatggactttagtatataatagatagaacagaatagatagatagataatagataatagataggggcaaattagtacttttcatgttgtttctctctcaatctgcaccacatataataaaatatggctgttgatgtccggtagctaattacgaggtttttatgatatcctctaactaatttatgtctttgcaatgccttatagctaattacacatttttaggtgtcacgtaacaattttttttttgccaaaacgtatgcacacaagttgcaaatttgactaaaacagaagatattttttctagcaaagaaagaatttcttttttcttatcaaagacgaacttaaagatttggatttttttcacaaaaaggggaaaaaattaatcaacatcggtacggaataaaacaacaaactcattcttgacattacccagcaatggagtagaatattcatttcaaggaaggagaaaaagcatgccggattagaatggacaatttcacaatttcatcgaattcgcactctgatgatacgaatgaacaattggtatataaatgaatagaagcaaccttcagctacaaacatcgattagcaaaacttgcaacacaaaacacagatctgagccttatcactccatgtaaatttgcttcaattttccaatcgaatttgtcggtctagctctcggctccgccgccggcgaggcatgggcagctgcagaaacaaaacagagaaatcagcaagagcaaagcatcaaacaactcataaaacaaagcacagagtgacgaatcgatatgaaatctaatacaatctaaataattgctgttcgattttcaaagaacaaattggagtaccatttggccaagaagctcggtctctcctgcgcccgctgaggttgcgacggcggctgctgcgggtttgccggacgcccgacgaagtagtcttctccccccaaaaacgaaaacgaagcaaaaaaaaaaatagcaaaccaaccagacaaaattgactcaatttttgtgtcaaaaaacgattgcataaaacgaaaacgaagcaaggttcagcaaaacatcgggcaaaactgattcttttgtgcccaaaatttgatcgaatcgagcttgcaacgttgcacgtaccaccagggatctgcgcgttggcagcaggccgctgctcgtcggcgccctgctgctgttctttcgccggctccgcgtggttcatcggccgccccacgaagtaccctgcagcaaacgaaaaattcttagactgatataaagaagcaagaattcctttaggctttagcatgattgattgatacaaacaaaaaaaaaaaagaagagaaacgaaagcttttgagtgatcagagtgacacgtaccaggaacatcgctgctcatggcggcggcggtcgacggatcgacggtcaccggcggcagctggttggtttcgtaggaggagaagctactgagcgcacacaacgagatggaaaagaagacgaagagggtaaggaggcacaggaagatgttccttcctctctcccaggctggcctacgccaatgattcgcgcagagattgatattttttttcggccgcgacgacctaaaacccaagtcgttgctcaagtggcatcaccaaccagcgaccactactactcgaagaccagctcagtaactagagtatcaggaatttgcaaacatggcatgagatagagaatatcaaagcatccactatttgttatttctttccacgaaattgcaaacatggcatgcaatgagaatatcaaatcatccactgtttgttttttttattttcaccgtactggcaaccgttttagttaggacagatactctggatcttgtaagttgtcatgacagggaaaacttcattgtcttacagatatttgggccgtgttagtttggctgcaagtgaaagagatcggcctcttctacattcaaaggcatgtgactttattactgatatataggtccgatgaatcataaacctacgtatcaatgataaatgtatgatgcttttgaagacaaagaatatcaatcaatgtgaaagatgcatgattttcgcaggccttaaattgtagacataatggctggccacttgtgtaaatcacttttgactgtttgcaaaaagctttttcttttaagtagcccctgaagattttacacatttgtgttctttttcaatacttattttaagaacaaacccttaaaaactgatcgcacaaatggaccctaaaacttctaacatctcagcacaaaaatccttggagttgaccaatggcaccatgctagaagcaagattaatatactacctccatcccaaaatatagcaatttttagctatgaatctggacacatacgtgtccagattcatagctaaaaaatcttacattttaggacggagggagtagtaactaagcgacctgcttgttggtgctaagatgttggagttcactcctaagcttgggcttggctgtgactgagattggggttcattcctacaattaagtttcataggattgtatttgtaaataagtattgcaaaaggaacatgatataaaatgtttcaatatgatagtccataaggttgatgcattggctgttagtcttggaaattagatgaaagtaaaggtgtctacttgattagtatagtatggactggtggtggcgaatataagattctaagtgtaatttggggtgtgtttgttcacgtcaaaattagaagtttgattgaaattggaacgatgtgatggaaaagttgaaagtgtgtgtgcgtagaaaagttttgatgtgatggaaaagttggaagtttgaagaaaaagttaggaattaaaccaggccttgatttgaatgagtataataattgattattgtccaagtttatttaccctgaaccaggattaattaatgaagaccacaagcatgccaaataccagaatacctatcaatccatgtcagcgaagagtcttagaacttttccagttaatttatagtcattttcatctcaaagcaaacgctgaaaaattaaaatccatgacagtttggttcaattgcttgaagacttgcagttgcaaataagtcggtcaattgtcaaagtcatctcattgatcgctgctctcgtttgcaaacattattattctttttcgttgaaaatgacaaatgattgttcgttgtaattaataggcgattgagtcaaaaaaaaatttctgaaccatccaagagaaagttcaaagttttcatattcatgtggaccttttcctgacaaagacgacttggataaaacaatccttttaatgagaccagtagcaatatagtaaattgatagatccaaacaggtaagcaaaatactaatggataacgcggaataatctcatttgcttgggatatgattgtacaattcacaacagtgtaactgcaggaaaagaaaaactttggaagaaacaaaacttgtatttcctaaaatgataacttctccatcaaaattccactgatttcttgatgaactgtcgattaacattgcgaatacagccgtgttgatctagttcacattttgaccggcagtgaagcacccgaagctgcagagattcaatgagccaatgcatcatgcaaagagaaaaacaaatcaatgggtaactacagacagaagcatctgatattctgtacacgaattgcagcttacagtttcttaaagaagcccggttcattcctctgtgcacccttagcatcatttggtctgaccggattgccattgtagtagccttagtagtagaaaaggaaaatcagaagataaagatttctcttaatctttataagtttgaaatttgaaaagttgaaaactatcataggacatagcaaacaaaacagcctgaggtaatagaagaatgcatagaagactgaaaattaagaacccttttcaaagcacttatcaacctcaagttataacaagatctgctacatcatatccggcaaattgatgtatgcagaaaactaagcaaattgatgtatgcagaaaactaattaatcctgtatacctaccagggaattgagcgttctgttcctcagccggccgtgaagcttgctgttcttcatggttcaatgggcgaccaacgaagtagcctgcatatgaacaacacccacaaagtaagaaaatttacagaaaatatgttattcaaacgtgaaatagttcgcctatggatgattggttaagcttgtagcatgcacatacctggaggattgtcactcatgttggatcaatcgatctccagccggttagctcctcttccggcgacggcgaggcgagaggtagacgaaggcgacgaccaggatgcagaggagactgacttgagaggtgtattctaatgaaacttttcaggtccacggccgttttattggcttttctctaaccccgttctaactttgattatacagattttaccttggttttcgttagcatattttttaaacagttaaatggtatatttcgtgtgaaaacttgttatatacaagttgttttaaaatatcaaataaatccatttttaagtttaaaataattaaaatttaattaatcaaaatggcaatgacttcctcgttttacgttccattagctaccagtagtgattagaataagcagacggattgtatggagcaatcggatctacagttgtgccaactagctaagctataggtcatcaaccaccgagtcacagtaaaaatgatcgcgtggtctggtgttactgaaagcgatacaattgcagctcagttttttttttccaaaatggcccacacataagataagatggtgtgaaatttcatattgataaagctgggaaaaagtacaaaagattataaccctacgaaattattatcaaggaaaaaaaaaaaaacctccaccacacgtgctagtaactcctgcacataccaacccgaccaacgcttggccaaatgccactagccctattagtgacagagacatgattttcgggatgggtattcaagatttccatagaaaaaaatattactcactctgtttcatattataagactttttagcattgcccacatttatatagatgttaataaatctaaacacacacacatatatatatatatatatatatatatatatatatatatatatatacatctacgtgcaaagtcgcatcttcaaattcattctacatagagaataacaaaaaagataaaattctgacaaaattgcaaccttaaaactgtcagattttttgtttttttgttacggctaaaatataatgaatttgacgttaagattttaaccctaggtgtaatacaattgaaagtatgtgtatgattttttctagatttattggtgacattttttagttggtgtgcacgtgtgtacacgtgagggcctgtgtgcataggatatgttgccatatatatatatatatatatatatatatatatatatatatatatatgcgagcaatgctagaaagtcttataatatgaaatagagaaagtagtaacattaggagctacatagaggaaaaataaggtagaatcaactgataaaaaggcagggattgaaatggagaagagggagggttgaggtgcgccatgctcatcatcgtcgtttgtctgttcaaattaaagaaaattgctaagatttgggaaaaccgccggtttcctccatagggccggtctgaccgtcgtttgttggtcggtcagaccgctacttgagggccggtctgtccagcggcatgtggccggtcagaccggcagagtccaagcccgagtctgttttcgtcaggtctcaggattccttactcaggaggctatatttcgggtttcctttgaattctatcctgagttggacgtggaggaggacctgtaggaggcaagatcaacccctatataagggacaaggccggttcaattgagcatacaatctacacacaatccaacttagccaattaaattgctttttcaatatttcaatctagttctagtctagttcatccatctttgtcgatttgtgccataaatcgtctgccttcactacgagagtgcgaaatcctttgtaggtttgtcccgtaaaccttccgtttacccacgagacgggtagttatctattgttctatctaaatcggctccgctagccggtttagtttatcaaaacccatcttggtctagcttttgctagattgaggtggttggcgatactacaatcaccgcaaggcgtttaggtgtctcgatcgtgcttgtctacttgtcaaaaaagttgccaaacacgtgccattccatggtgtctcagactcttggtgctcgttgtacactcatgccatcccatgccatctcgggacaactcggtgtccgcaccaagaagcatggcgcctaccccctatcagttttatgggtaaggcatacccaatagtgttgggttaagcttagcgggacctaccatataccaagtcttatacggaatcaaatcttgcatacggaaggtattccagataaggaaggagaagcagagttctacatggaaactacaaggactattcggatcgtatccatatttatctttctagttctatttggataaggggacatctatgggtataaatacaagacccctaaaagaagagggggagacggaccaagacaagccgagtctaaatagacaggcaacagtcaaaatatacatcaatacatgccgccagacatcaacataagagataaacctaaacagatccaatctggtgccacagaggctgacaagagggatctagcgctatctctgatatcgcccaattcatattagaggaggaggactaccctgttgtcgacaacgtgtggtggttcatgccgccgtgttgacgatgattagataagttatcccaaatattgtacttgtgtgattctgatgaataagagcaacactggtttcaattagcaggagtagagctattacctggtagataaggagcctgaacctacatgaaaatccttgtctccgtttattttaccttagtctcgcttataccctgatacgaatgatccccaaactctgcaaataccgtagtcgtgatctacaacatcgacactctctccccctccccctctaactcgatgccaaggtctccagcgtgggtgcacgaaacctcgatgccaagctcaccggcaccgagtagagtccatatccataattagatttttggagtctatttattcataaataagttttacaaaaagagtcataatttttttaaaaaaatcgagcataccaaaaggtggacgaagacgacgaccaagaaatcgaggaaacttgacttgagagatttttatactaagacgacttggaggtctccggccgttttagttggatgaaaacataagaaatcagtttgttagctttctctaagctgtcattagcgagtagaataaggagactggaattatatttttgcagttgccgactagctaactaggtcctcaacaactgagtcataagtacaaggactgttaaggtgacacggtatgtttcttcctactccctccgtcctcaaatataacgatatgtttcttcctactccattcgtacttaaatataaaggattttaaacagatgtgacacttactagtacatgtccagattccttgtactaaaaaaatgtctcatccgtctaaaatctcttatattttgggcaaagggagtatatgtccaagatgcagcgaacagcctgaccagtgagctggctgcaggacagtaaactaatcaaaactgtatcttttctaatttgcatactggactacagatattctattcctttcgatatgaacaattctgatttactttttcttttcctttttactataggcaaaataatctattgagcaataatttctcgagtttctggtccatttaaaaagaatccagcatcccccactgcatgcggtttggagggtatactataaattttgagatcaggaaaaaggaagcttccacagcggactcccgtcctacacgcacaacttggccagttgataattggcactgcaaatgcttagccgcgtcgacttcgcgtcaagtcgtcggcggctagttcagagaaacagacaaggatctgattgagatttgaggctagtggctttgtgctttatattttcttcgttgcctatcttaaacctcacatgctctgatcgtctcgccagctctgctcttctagacgctctgatcgtctgcagctcaggttagattgatttccactggatcttgctttgtatcatctagctagtttgtagctgttatcagctttaacttttctgattatgtctgctggaaaattctgcctttttctttcgtgaatttcgccttgttgagaggagggatcactaattagtctctttgctagaaaatattgggaaatttgttctgaaatttgaccctgagcgagcatatctaaggccagcagcacattctcagttcagagatggggaaagaacctttgtgttgttgttttgttcagtgctgattcaactgcaacatttagcgattactgttgtttgcagtgcatcaccttaaatactgtgtgttaattacatgtttcccgtatttttatcatttattatcttattgttggccatctactgcaaggacatcttaagctaaaattgaatgcccgtggaaactattaccaatcatttcaccaagaacaaagcatgaaagatgagtagcccaacgaaattttctaatggccaatcaatttgcgatatagttgaaatgttgctttacaaaagatccagtgctactatagcggctgtacagaattaatatggattacaaaaggaagacaaaaacaactaggaccaaataaagaaacagtaacaagtcaaccagataaacgagatttgaagggcaaacatctattatattattaaaggaatagaagaagaagcctctatgtttgctctcatggtctataaattctcacattaattagggaaaaagaaaagacagagtccatatagaaatacaatttagaaatagctgaaattcagaattaaaaaatatggaatcttagaagaggagactagagtccatatagaaatacaatttagaaatagttgaaattcagaattaaaaaataaggaatattagaagatgagactagagtccatatatagaaatacaattaggaaataaataactgaaattcggaattaaaaataaggaatattaaaagtagagtatatagtccatatagaaatacaattaggaaataactgaaattcggaattaaaaataaggaatattaaaagtggagtatagagtccatatagaaatacaattatgaaataactgaaattcggaattcaaaataaggaatattagaagtagagtatacagtccatatagaaatacgattaggaaataactgaaattcggaattaaaaataaggaatattagaagtagagtatagagtccatatagaaattcaatgaagaaaaaaaatagaaattcgaaataaaaaaataaggaatattagaagttgagtatagagtccatataggaatttaaaactaactaaaatttggaataaacataataaaattaaaagtagagtttagagtcagtataaaaatacaatttataaataactaaaatttaaaattaagaaaaacatgggaagaagagtttaaagtcactataggaatacaatttaaaagtaactgaaattcgaaattaaaaattaaagaatattgaaagatgagtttagagtccagatagaaatacaattagaaataataaaaattcagaaataaaaataaataatattggaagaagagcatagagtctatatagaaatacaatttacagaaaattcggaattaaaaaaaagaaatattaaaacacgagtctagagtccatatagaaatatatataatttacaaataactaaaatttgatattaaaaataattaataactaacacgtatataaaatataatatgaatattatgtattagtagttttgtaaagttattgtaaaatttaaaattatgttgtcattttaatatatttgaataatataatgagaaaacatatatgcaattatatgggaaaaaacataatggtgctagccgcgcagtctgcgcgggccaccatgctagtttttattataagaataaattgtaagcgtgttgggattgaacatgggaccttgggattgaaaccacgcaccccttatcactacattatcaagtgcatctcataacaaatcaaccatgaccctgcctatacaacagatctacaccagtgctaaacggtactccctccgttcgtaatacgagggattatattttttttaatagaaattaaggaataggaaaaagtacagataaagggaagcactaattggattaggccgattggcacattgaataaactaaaaaaaagtaagaaaaagctaagggcccctttgaatcacatgaataaaaaaacggaggaatatgaaaaacatagaattctgacaggaatgtaagtgtaaaagaggattgcaaaacacaggaataacgtatgaatgaccatttgattggaccacagaaaaaacacaggaatttgatgagagataaagactcaaaacgattttttcatgaggttctacctcttgttaaaattcctccaaaacttgtatgggaagaggcattccataggaattttataggattccataggattcattcctttaattcaaaaggctttgtaagaaaaattcctataggaatgaaatcctctaaaatttctatcaatttcctttgaatcaaagggggcctaaaatgttgctgcatcccttctgttgtttgctcacctcaggatccacgatcccttgtatttgtggacaaatcgaagggttataatctcttatattcacagttagagggtagttcttttcagcaactcaaaacgacaacttttttctttttcgttaatgtgttttccgtattgctaaacggtgtgttttgtaaaaggatatgtaaaatgttgacctctacctgttataagcaaaagaatcgaaccacatgcgcttcagcacttccatgctgagaaagagaacactggaatatgtggtgtgcatctctacttcatattaagagcattgctggtctgctccttgtcacttaaaaagaaattgagtcacagtcatgttctttaagtacttatcagtaggttcagcagcttaatgccagaattcacaagtaggtttctactgacaagaaacctatcatctaagcatggtgtaacagactagaagagcagaatccaaaacagagttgtcattagaaatggctgcttaaaagaaaagtaagattagaaacaaactatgatcatgtttgatattatgttcagatatatctgggctgtttcatatatacttcattgcatttaagaacatggaagaagtgatggtgcatgaaatactcacacatattagatactggcctttgtatgaagcattaaaaagctgggcacatatgagttatactaaatgatcaatctatctgtggaaccattaaaaacctgaatagagacctacttcaagaaacaacacactcaactaaactatgagactatctgattatgagttactttgatctcctattaagggcctatttgggggagctttagattctgagaagcaactgtttggtagctagattctgagaatctggaaaagctctgaaacccagcttctccagcttctggcttcttagttcatttttcagattctgtaactacagattctcagaagctgtggactgtttggggtagcttctagcagaagcagcttttgggaaaagttacagctgggacaagctcccccaaacaggacctaagttacacagttggtcacattgcagagttatgaaacaattgccatagaacattacccttacaattaagattactgttgaggctggtacgtaatgaggagctcctttacctatatctgttggtacaccatgattaacattagaaatatatatatatatggcttgcaaatgcccacaagggcccaaaaaacaatcttagctgcaattgttttacaactgtaacaacaaccttttttatcttatcaattcgttatgtgactatctccatgattcatgattctgttctgcagcactaaggtaatggcagaagctgtgctgcttgccatctcaaagatcagtattgcattgggagatgaagccaccagagctgtcatagccaaactgtctgggaaggtaactaacttgagggaacttccagacaaagttgagtatatcagaagggaattgcgtgtcatgaaagatgtaatacaagatttagattccacaaataccaacatgaatgttgtaaagggttggatcgacgagctgcggaagctggcttaccgtgttgaagacataatggacaagtactcatactatgcttgtcaactgcagcaagaaggatcagtgatgaggttcgtaagagtacattatgcaggtgttttcagtgaagttgctagtgaggtaatgaagataaagggtgatattgaacaggttaaaagacagcaaacggaatggttacctacagttcagctcattccaagaactcccactgacatcgaaacaccgcaatctcaaggaagaaggaagttgcttgaatgtggcgatcctgtggggattgaatacaacaggaaaaggttgcttgaattactgtactctgaagaaccaggtcacaaagtaataacagtgtctggtatgggtggactgggaaaaaccaccctggccttagacgtatatgaacgtgaaaagatcaagttcccagttcatgcctggatcactgtgtcacagacttggacgatccttagtctttgtaggcagctagtcagtgagctcatacgcatggaacaagaatcatcagaatctaaggaagatctaatcaacaaaatgggcgttcaagatttgacagaagaattaaacagaaggactgaaaacagtacatcatgtttgattgtgctagatgatgtctgggaccagaacgtgtacttcgaaatacagggcaagcttaagaatccccaagcaagtcgcattatcatcacgacacggatggaacatgtggcagttcttgctccctctgaatgccatctcaagatacaggctttgggtgaaattgatgcattcaaccttttctgcagaagggcattttacaacagaaaggaccataggtgcccgctggaccttgagaatgtggctgcctccatagtaagtaagtgtaaggggctgcccctagcacttgttaccatgggtgtcctgatgtccacaaagctacaaactgagcatgcctggcaacaaatgtacaaccagcttcggagtgagctggccaaaaatgatgatgtcaaggcaattctgaaactaagctaccatgcattaccagctgaccaaaagaactgcttcttgtactgcagcctgttccctgaagattttcgcatatctcgtgagagccttgtgcggtactgggttgcagaaggttttgcggtgagaaccgaacacaatagaccagaagatgtggccgaaataaatctcatggaactgatccaccggaacatgcttgaagttgacgagtatgatgagcttggcagggtgagatcttgtaagatgcatgacattgtgcgcaacctggctctctcaattgctggacaggagaggttcggttgcgtaaatgattatggagctgtggaaaaggttgattgggaagtccgtcgtctgtcattattcttgaacaatggaaaaggttgtgcatcaacagtgaaatttccacatcttcgcacactacttgaaacaactacacaccctcctggattgttatcctcaattttgtctgaatccaaatacctcactgtcctagagctacaagattcagatatcactgaagtaccagcatgcataggtaaattgtttaatttacgttacattggcttaaggcggacaagactctgctcactaccagagtctattgagaagctctcaaacctgcaaactctggacatcaagcaaaccaaaatagagaagctaccacgtggaatcactaagatcaagaagctaaggcacctgctagctgatagatatgaagacgagaagcagtcagtgtttcgctatttcattggaatgcaagcacccaaagatctgtctaagttggaagaacttcagactcttgagactgtggaagccagcaaggacttggccgagcagctgaaggaactaatgcagataagaagtatttggattgacaacataagttctgctgattgtggaaatatttttgctacattatcaactatgccgcttctttctagcttgcttctttctgcaagagatgagaatgaacctctttgctttgaggctctccagcccatgtccaaagaactccacaggttaattatcagagggcaatgggccaagggcacattggactacccgatatttcgtagccacactacacatctcaagtacttagctctaagttggtgtaatcttggggaagatccactggggatgctcgcatcacacttgtcaaacctcacttatctaagactgaacaacgtgcacagttcgaaaactttggttcttgatgcagaggcgttcccccacctgaagactcttgtgttaatgcacatgcctgatgtcaaccagataaacatcacggatggcgcccttccatgcattgaaggtttatacattgtatcattgtggaagctggataaagtccctcaaggcattgaatcccttgcctccctgaagaagctctggttgaaggacctgcacaaagacttcaagactcaatggaaaggtgacgggatgcaccagaagatgctgcatgttgcagaggtccgtatatagatgttgcagattggttcttggcgcggcggctttcccatgcctgaagacacttgtgctgaagcacatgcctgatgtcaaccagctaaaaattataagtggtgcacttccagtcattgaaggtttgtacattgttgctctgtcagggctggagagtgtccctcctggcattgaaacccttcggaccctgaagaaactctggctggttggtctgcactgggactttgaagctcactggattgagagtgagatggaccaaaaaatggctgattgtgtaggggactaggagtgctagttaccttctccctacattagcaatcttgtttgatgcctctgcatttgtcaggtccattgaccattccaataagttcatcgttatggagttattgctactttgctagcactacctgaaacattcgctttcttatccatctggacctgaagtgttcttctgttattttgattgctttggagattgttgtaagaggcccttttactattgaactgcgtttagacaatgtttttcctttctgttcatttttgagatcgttgttctgcagaatgatctcatagcatttgtttattatctaatggtgggttcacttttatgagttagattgcattctcgaatttcagaaaaaataactaaagcaatcagatgatgcagcgtttgaactttgttgtaatctactactattgtttagctgaagaaattctgacaactcccatcgtaggccattgcaagcaagagcagatgcattgctgcacagatgtactatgcaagtgtgaaccctctgtgcatttcatgtgccagtattttaaccagattaaacatgcaagaaggatgaacacctagaaaacataattgaacgcactagaacacgaatctgccgcgcaattgacacgaaacccttaaaatcgacacctgggaatatcttcatggaatgaatcgaatggggcaaccccaccaaaatcgcagggaactgaatccgggcgttacctgaagaatccaggaagaggcgccgccgacggtcgtcgtccatggccgccgccggcgagcgcttgcttgggctcgtttactccgccgccgcagagtcgaggggattggtctccgatctgatctgggccgacgggttcatttcattatggtataagttcactttaggttcctcaatttgtcgccgagtctaccattcacccctgaaccgaaatacggtatagcacatcctcaactcataataccttgttaactttgatcactccacagtacagtgatcggttttggctgacacaactcacaatacctcgttaactttgatcactcgataatacagtgatcggttttggctgacatgatgagggggacccacgtggaccccatatgtctggagaaagctacccatcggacgtccgatgcctatcaaaaaatcggatggtcaaatgttgcagtttaccaataagtgaaacattaagttatatacggtaaaaaaaatcagcaactaaaacatttaaatattttatgaaacatggtgaagatacacgttgaaacatattgctatcacatatgaaacaacaagtagcatatgtttttcctttatcaaaatatagtcatgcgatatcttgttgtaaagatttaattaaaacgaatacaatggtgtaatcgggtcatatattggataagtaatttaagagaaaaatcattttgaagcttgctaaaataaatgcatgtgtgcatggatgagatggagagagaggagggagagggtagtagcagatagtatcaggaattttttgaaatacgctgaagacgcatgttgaaacatatcactatcacgtatgaaatatttagttttaacggtttgaaacacatgtttttttatataatataatcatatgatatcttattgtaaagatttaattgcaacaaatataacggtgtgatcggttcgtagatcgaataagtaatctagaagaaaaaatatttaaagagaagaggtaagaacaaaaggaaacaattggccatggatgttacagtgaaatagttcatcctatccatataccgatttacagtagatgacgcgtccgatatttcggttttgatcggacgtacgatataaagcattttccccacgtgtcagcggcccatctcttctccctcatctgcctctccctcttcactcttttttcccatctcttgagggcggcgcgcagtgaagcgatgggaggcgcgcgccacgcggcggccggcagaggcgatgacggagtcaccctgcgctccagtgcgtcagcgaacgtggagggaacccacggcttgccagacggccggcgtggtcacctgcgcgggcgacacggcacagtggcggatgtccgtgtccgtgcactgttcccgccacaccattttgaaatagttcactttagctgtcactaaagacaatctcaaatctatctacgttcttgtagtaaaaaaaaaagtcaaaatgcaagttcttcctaacgctacagaatgcaatatactcaacggagtttacaactatccttcgatgacatgttataatgtgtaaccttaacatcttaattaaatcattaattaatacatactacaaaattatcagcatttaaaatgcagaaactatattagtagtttttgcataaaaaaagatatttcatacagttatatttaaattatttttgctaatatatagtttgagagagaaatgatagcatggaccttgaggactagtatatcgatatccaaaacgtcatttcttttttggtaaagaagataccaagactatcataagtgtctacttattcgcttatcagccatagcaaaaatttaaatcttgaaatttgatttttctaaagttaattttgggtattgttattgcagttcattatttttcaatattagctttaaaccacaaacaatatcaatataaaacattgcttaatcatttttctactatttattgatcgtatcattctagcacaattcacacgtttgaaattaggtttttattaatgcctcactaaaactatggcgttacaatattatgtcattaactatttcttaccagagaaaatatttttctattacttacgcaacaacaatttatttatttatcataacaagcttataatagatgaaacattaccaccaattaaactatatcaatattcttctcacatacaacaagtaataggctagccatgttccttgcaaccggtaatcaccaaacaaagcacatttagttgcaagggagatcagcctcttccctcactatgggcacgccatattaaactagtgcatgttcattcaaatcaaattgagaagcatgtggtgcaactatttccacttgtggagccacaaccaccgaagaagaagcaccttgtgcgtggagctcgatgatgcggtttctaatggcctcccggagcttggccgcctcccgcttgacatttttgaccatctcgggagataaatcatttaagttcttgcggtgtccacctcctatctcagaaattatgttggtgatctcacgtttgtagttctcagccttcagattgtccagcttcttctggagcttcataatttgctgctggagtagggtcttggagtcttgcatccacttcttctggacagcctctggcatgtccatgagtttgctccacatgtttttggcctcttcaatagatggccataccactggatcacatgcttcatcgatgttgtacaccacgatactagcttggacactgcacaagattgatagctctgttacctttttaatcaatccagcacgacgcctcctaagagtcactctgcgggcagcgtttttggtatgccgtgccatagcacacctcacctacacgggcgcacggacacgaacctagtaatgaaaatgactcttgtgagagttgtttttctgtgtttgccgacagaggtagggtgatctttttatagtgaaaaacgtggtattctattttaggtgaatggcgcgtaaagagctctaaaatagtgaaaaagataagacaccataatagagagcgaaactttggtgtaatttaaattataatgagataagataaggtaaccgtaaagctagtgtagtttaaaatagcttcaggatagggtaaatcatatataacgtcggcatatattaggtgctatttacatttatgggatttgtccacacacaaatgcagatttggggcttgaggaatttggtccatggctcgaggccttagcctaaaagtttatggtgctagactttttttatagtgttaatttatatggggctcaatgtgggctatgctatcctaagattttattaatcagcacacggcttgtccgtgggcatagaacttgcctagttgtcgtacctatatggctatatatgaaatgttagcaacacttgtaacatcaatcacataataatacttgaaaagttattgtgatcatgtcattgtttattcagtgtgttgtgttgtttcaatagagtaccaactccacccagatggtgcggggccccgctcacccgtcgggctccctgcaggggcgggggaggttttccacgtggtgaccaatgcgggggcggggacgggggctggtgttgatgaaaaaatcgaacacaccggtctgggagatctgcttagctcctgtgcaggtacaaaggttgatgagatgcgagcgtgctagtcagtttgatcctgcaactgacaagatatgtaaatagtagatcaaatagccgatcgtctgacaagccgatggagtggttctagccgatagccgataatagccgatgccaataccagccgatagcgatagggtttaagcaatcggctatatgtccaatgtagataatgatataaagacaatcggctgatgatgatgtaataaaataacaatataatccagtataagccaatcggctagcattgatatgataaataagcatcgatccgaaggttaaagcacacatcggctggaggtccgatgtcatgaaatccacaagattagattaaacaatgaaacctttgttgtcatcggctaaatccaacttatatgtatatgcaatccttatgagccgatgcaacgtccagataactcaccggttgaaaccccgatgaaacccttattggcaatcaaaaagcaggctagagattatggttctaagcacgacttagtagatcaaacttaactgatgcagcactaagtatgaaaagaaacacaatatctagacaatcaagccgttgactgattttcagggtggtagatgtctaggctaatctaatctagcaaagcgatttagccgatactggcagaaaccctaaagtgagagacagccgatagagctaaattgagatgctaggactagattaacatagacatatgataaatagccaggcaaatatattatccaaaccagagtaatcctataggtcgaatgtactgatgcagccttgaacgacgccgacgtagacgatacaattgcctggaccggcggaacattggacttatcccttcgccggagatcgaagacgatgcagccccgcgtcgggtgccaggttccgccggaatgtaaaaacaaaagtagaagtaaaaagggtggcgatgcgccgaattgtattgatcgtagtgatagattacatagaccccgggtgtacatatttatacccatgggttgatacaagtccttgtcggacaagaaaaaaactttcctaaagataaaaggaaaagataaagtccttataggatactaaacacacttttctaaagataaaaggaaactaacaaactattcctaattaatagataacttgccatgccgcattctctttgaactcggtctcttctagataagcttcctttagtagatcaattttcttaaccgaatatagcaagaatccgacaactgacgacttgacaattctcatcggctaatcgcaggacttcaaagccgatgctgactctaagccgatgactactccgggcttaccaaatttcactgttaacagctgggggggagggaatgcggggagcgggggggttcctatccaacccggccccgccccgccccgctacccccctaggtgagggtggtgagaacggggacgaggccacactccggatagtgcgctgccgttgccgctagatcaggaggagtttgaggctggggagtagggagagagtaggctagcaggggcgacgaaaacggcgacgatgaattaggatggaaggcaaagtcaagtagtcaacaccagcatggggcagccctatcaaaccactcttttttttatgactatcgtgtccctgcgaattgaacggacatatttcaatcgtactggtggtacttagtacctcctgatacatcctcatggatggaaaaaactttcataatagtaattgatgaatttgtagggaaatttaaggattgcatcacctgagtttaggggttttttttctctggaattggagaatgcaatcttgctcatacaattggagtacgagagcacatactaatccattgttctaacctaaagaaagccagagaattcccacattatgccatggcaatgctagaacttgcacattgctgcactactgtgtgaactgtggagctgtgaaccctctttgcttacatgtggtgaatttgcagccacatgaaacatgctgcaagcctgcaagaatggagcaacaaagactgaaagccctagcccacgaacgtcgagaggaacccctaaaatcgagaccaacacatttgggttgggatatctctatggaacaaattgaatagaaacccccaataaaaccataggaaactgagtccgaatccttaccacgaaatccaagaaagagccgacgccgcatgggagcttgatggccgatgcataggagctcggcaccaaagcaggcgggcaggctgccgccgccccgtcagtcgctatagtcgtcgtcatcattgtcggcggcgagcgcttgctggggggctactgctccctccaccacccagactatgccataaggcgggctgggattagttgccactggggtgagccgacgcgacccaactctgctcgagatgaacaatttgagcattgtttatatatgtttgattattctagtttggtcttattcttctaggtccaatgtggaattgtgttctttgggcttcttctggcctagccacctagggtgttaatggatcacaaaattttcggttttgcgagatgttcagaagattattataatgagtaattctttttttttattacacaacaaactcacatgcacatatatcactgtacatgtaacaccacactcacacactcacgaatacgctccctaacacactctcaaatagatgaaaatcagcagcacgtatatgatttcactgattaaattcgtgttgaaagcccacttacatatgcataatatttgtgggtataactcatgacttcactaccgtatcactgatgcttttaagttgtctatgttgttgaactcaaaatgttatttctaacagtagagaaagcaatatactcgacgaagtttaccagcccgaaaacggtatcggatccatctgagttgaatcacacttaacactatttaacactatcgacctcgctttaacgcaaaaattccaaaatcagaattcctagcgaccgtgctgccttccttataagctgctccctctcttctcaaattggcgaggtgggactaaagttagctacagctgttgctgctgctcttgctctgctcgctgctgcatgctagctactgctgcttcatgttgctgctgtgggctgcatgcatgcatgggctggcctgctgctgcttgctacttgctgggctgcacgcatgggccaaggcccaaggcttagctgggctgctagtttgctcttttctctcttttctttttctctaattattgcggggcgttacagtgagtcatttactcatttgcccatctaattatgcactcgagcatacctttgggtcaataatgcatggagggatagcggcttggcaataaagaggagccctctccctctcttctctctcaccatagcatgaagaagcgctctcctcgccatctcccattgtcctcatgcagaagcagctgtgcggcgacagctcgacgatgaggaggaggattagtggatccgatggtggagcttgggctgcggcaagtcccgggcgatgaatccggcggcggtaagtcttccgtggccatgttggacttcaactgctgcgggtcaatgacagcaacgccacggcgaggatggtggctaggctacatcgacatggcctccttgctctctcccatccacaagccgcatgcatcagtggcgcggtagcatccgacggcatcaagaaggggaaggccacatccaacggtgtagggcattaccgtgtcttgagattagtcaaatttccatgaggtagtagtgtgacctcatgaaataaaaaatattactgcatcatgaaattagttaaaaattccatcacatattttctatacactgatgtacaaactggattgaaaaaacaagaaccactgaaaactaggatcaaatggacacaaaggggttttttcccttgaaatgagcaaacttgaaaaggcatggacaaattggtagttaggcttcaaaacaggattaaaagcgcaaatgccccattgtttattgattataactataaaagacggaatattgccaccaactaaactatcgaaatattctcctttcatgcaacaagtgatagactagctatgtgaggaccatgttccttgaaaccagtactcatgtaaaagtcttgccaaacaaagcacctttagttgcaggggagatcgacctcttccctcactatgggaggatcatcatcaaccgctgcaggttcattcaaatcaaattgaaaagcatgtggtgcaactatttcctcttgtggagccacaaccaccgaagaagaagcaccttgggcgcgaagctcgacaatgcggtttctaatagcctccctatgcttcgccacctccagcttgacacctttgaccatctcgggagatagatcatctaggttcttgcagagaccaactgatagctcagaaattatattggtgatctcacgtttgtagttctcagccttcagattgtccagcttcttctggagcttcgcaatttgctgctgaagtagggtcttggagtcttgcatccacttcttctggacagcctctggcatgtccatgagtttgctccacatgtctttggcctcctcaatggatggccatgccactggatcacctgtctcatttatgttgtacaccatgatactagccttgacactgcacaagattgatagctctgttacctttttaatcaatctagcactacgcttcctaagagtcactttgcgggcaatattgttggcctggtaagctttgccatggcacacctcacctatgcgcgcgcacagacacggacttggtaatggaaatgacttgtaatttgtttttgtgtgtgtttgcaaatagaggtaggtgacctttttatagtgaaaatcatggtattctattttaggtgaatagcgtataaagggctccaaaagagtgaaaaatataatacgccatatagggagaaaaactttgatgtaatttaaattctaatgagagaagataaggtaaacacaaagctagtatagtttaaatatcttcaggatagggtaaatcataacgtcggcatagattaggtgctatttatgtttgtgggatttgttgttcacacatatgcagatctgggccttgaggaacctggtccatgtctcgaggccttggactaaaagttcatggagctagactttttcataatgttagtttatatgggagctcaatgtgggctaggccatccctggacttattaatcaacacacggttgatccaccgacatagaacaagcctagttgtcaacttgtcatatatatatatggaatggttaggaacacttgtaacatcagtcatataatacatgaaaaattattttcatgtcatcgtcctatatctctaactctatgttatctgttttgtgctatgtaaattgctagagtgcgcggtaagtaaaggttgccacaccttgtaaatatttggtactttgatcttttcctgtccgaccacgcatatacaaacgtgacacacgcaagtaaatatcacgggtttaacttggtaagatacatttcataccccgcaagaacgcgcatctacataaatccccttgaggtgatcaaggatacatccgaactcgaattattttgcatctaccttatagccgtgcacataccttgaacagatgtacgcttcacgtggaattttgtaaatagtttcaattttgttacatatttatcaacaaaatttcttttaaaaagttgacaaatataattatagtacaatcgtacatagtgtaatgtacttttttagtcaaagttctttaggtttgagcaaatttatagaaaaattagtaacatctataacaccaaattagtttcattaaatccaacattgaatatattttaaaaaaatatttgttttgtatttgaaatatcactatatgtttctacaaacttatcaaactttaaaaaatttgactaagaaaaaaaatcaaatgacttatcatataaagcggagggagtaatttttatataattttgaactgttagatttgtttcaagatttgtgcaaacaaggaagggaaaaaattatgtgatttcagtgtcaacaaaaatttcacgaatttcggttttttcaaaacttttttacacgaaaatatttgaaatttaactatttttactaaatttgaattcataaaaattactaaaaaacctgaaaattgcgagggagattgttgcttgccagggggtccaaaattagtagcgcgaaaacatcgtctatatataggatatggattttcatccctctacaattatgcaagaccaaatttaacttctacaagttgcaacaaaaagaacaaaataagttgaaaatagttattcaacttctacgaaaactatcaatccaatcaatagctatcaccatgactaccatggaagacgccggcggccgcnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnngcggccgcgacggagaacctcatcgacacgatcctcccgccggggtcggtgagcacggacacgggtctcgtggtcaccagcgccatctacttcaacggcacatggcagacgccattccgaaagcaggacaccaagaaggacaagttccacctcctcgacggccacggcaccgtcgacgcggacttcatgcgcaccggcgaggaccagtacatcgcggcgcacgacgggttcaaggtgctcaagatgccgtacgcccacgaccacgcggcgccacaaccctcgccgcggtactactccatgtacatcctcctccccgacgagagcgacggcctgtccagcctcgaggacaggatggcggcggccggtggcggcggcggcggcgagggcttcctcagcgagcacatgccggtgcgacgcgtcgaggtcggcgagttcaggatccccaggttcaagctctccttcagccgcagcgtcgtgcgcgcgctccggggtgtcggggtgaacgccgtgttcgatcgggccgagctgccggacatgatcgagggcgagccgctgcgcgtgtcggacgtcctgcacaaggccgtgatcgaggtgaacgaggagggcaccgaggcggcggcggccacggcagtgttaatggaaggtgcggcgcggtatgcgccgccgccgccgccaagggaggacttcgtcgccgaccatccgttcgccttctttgtggtggaggaatcgtcgggcgcggtactcttcgcggggcatgtcgttgacccgacgaaatcttagtcaacagaagtatagtatcattgtttttggaaattgctcgagtcttaacgctaatgtctatttctatccgtcccaaaaaaaaaaaaactgttttctcttttttattctcaaagataatttccaaaaaaaaaaaagaaaaactgttcttctaatgagaaactgcgtgggtatttaaatggactgcacgctaaattctttttttttctcaaacattagataattagaaaattcccaaaaaaagcaaagtgagaatttttctcttttttttcagttaattagagttttcgattgatttgttaagaaaaactgcgtgggtagttaaatggactgcataaggcggacaaagtaccgtaccttggcttgcgaagttgcgatgggaaatagatatttaagttctttcagaaaacatattgttcttcacgtgggcctgtatgataacttattcaaaataggttattcggttattcctcgtccatcagatccaacattagctattgatttgtagataaaaacttgatctgtactggtcacaacctaattaaacatgtctagccgcacatcatatctactgcttcctctatttcacaatgtaagtcattttagcatttcccacattcatattaatgttaataaagattcattaacatcaatataaatgtggaaaatgatagaatgacttacattgtgaaacggatggagtactacgtatacctgttgcataatatatgggattttggtgaagtactaggatatgtcacatccactcaaaatcattcatattaatatgtcgcatccacctaaaatcatttatagtactacgatatgtcacattcacccagaagctcttagggtgtgttcgattgggtggatgagcctgaatgggagatggccgcccgatttttcgtgttgtttgattggagggcatgatggatggggcgacctgcaatatgaaatattccctcgatatgctggatgagtgcattcgatcgatttggctggatgaagctactcagttttggtctgggtgatgtgtcatattctgattggttgtggttggtttaatttcttgttaagcaagctctaaatgattttttttctcctaaaccgtttattcaatttatgatttgattacaccattatatttgttataattaaatctttaaaacaagatctcgaatgattatattttgataaaaaaatatatatgacaaattggtctcactaacttttggcttttcccatccatgttatatccctccaaccaagaaattggatcgccatatctatacaaaccaaacaaaaaattagattggcatatccaacaaagtatagatggccatatctcatccatgtatgactgtgacccaaacacacccttatattatgggacatagggagtatcttttacgttggctccctattctggcgtgttagatctaatcatggtggcatctccgatgatcgaataggaaaaaaatgcattaaagttaaaataaaccgccaacatctctccggtttaggagaatttagagtatttaccatccaattcgcatgccccaacttttttactacccaattcatatgccctctataatatatccttaggactgctaattagtctatttggtaccactttatttgtttttcatgtatttctcttttcttaatcccatttgtattttttttaagttttgtggactgttttgcccctggacccacctgttagaacaaagggtgagaacaaagtctggatcgatcgatctctctgcccgagcatcgacatccactccaccgtagcgggccacgagcgccaccaccctgtcaccgtgcgccgccaaccccgccgacgtgtgccgctgtcggagagtgaactcctagagcaggggactaaaggggccccctttcagttcggccaggggcagcgggtgagaatcgagggttggtagaaagaggggtcgggcctctcgagaacaatgagagaaaatagatttatacaggttccggccgctgagaagcataacaccctactcctgtgtgtgtttgatttgtgaggaggcgcatgtacttgttggatctatccgagagtcatcctccacttcttcactaggcctggacctccttttatacctcaaggggttaccacagtggcccaaacgatctacctagggaagaaggaagacatcatttacttttattacacgtcttatttctctgacaaaggaaatcatgccttcggcgctccagcatggctgtatcaaatcacaacaggaaacacgggggacgagtgcgtcatttcccattaaccatctaaagactttgggtacccgttcccaccaaggaatggataccctaatttaaattagagtgggatattgtccccgacttgcacacggggcgcgatttctctcatcatgatgatgtgaccagtcggcatcggccacacgccgcctgacaaagggccagtgcgcaggcgcactcaccctatcacaccggtcactcaagcgtcacttagcctgtcaaccactcctgcacttcgcattaaatgcggcgtggcggggctgcagctcctgccttaagtgttgttagttgcacgctaggcgtgcccacctaacccgcgcatgttggcaggtgcatgcagggggtcatgacggcctgaccaccgactgggagggggcagctgtcactccaccccggtccgactcccctcgggggcaatgccacgtggtcctgacgacggcttcctccctctagtcatgatacccatgggcccatatcaccgagagccaccaaccccgcccccaacgccgaggagtgccaccgccaagcaccaccacttccgccgccgccgagcgccaccaccgatgcccccaccgccacagttctcagaatcaacaatgtccaactccagtgcttgtaattgttcctcctggcatgtaacccttgtcttctggaatgccttaagagaccacttgctggaactcggcaccgaaatctgctttgataggagatgcaaagttgattcgagctaggccggtgttggcgatcggtggcggtgctcggcggcggggtggcggtgctctcctagtcctggagctagggcgcgctcggcagtggaggcggtaggggcattgagctaggccgacggcggtgctcggcggcggggtggtggcgctcggcggtggggcatcgagctggggcagcggcggtgcttggtggcggcgctcagcagcggggttgtggcgctccggcagcggtgctcggtggcactcggcggtagggtggtggcactcagcgttggaggcggagtatgccagtcactggcggaggtgctctggagagatcgatcgatccagagaagagaaaacctctccttttgttctgacatgtggcccaggggcaaaacggtccacaaagcttgaaaaaaaatacaaatgggattaagaaatgagaaatatataaaacagagaaaatgttaccaaatagactaattagtagtcctaatgacatattatagagggcatgcgaattgggtagtaaatactctaaattctcccgatttaggtcatgtttagttcgtgaacaaaaaattttggcatgacacatcggatatacggacacacatttgaagtattaagtgtagaccaataacaaaacaaattacagattccgcctggaaactacgagacaaatttactaagcctaattaattcgtcattagcaaatatttactgtagcaccacattatcaaatcatggcgcgattagacttaaaagatttgtctctcaatttccgttagaactatgtaatttttttttgtccacatttaatactttatgtatttatctaaatattcgatgtgatgggtgaaaagtttttttttaactaaacaaggcctgatgcaggcaggcggcagccgttaaaaaaaaaatgcaagaggggtttggatggggctaaaacgttatagcccctgtcgactagggttgcagttaggtgaacctcaccactgcagcaaattggcatgtcatgttagcacacgaagtgggttgcaggggacatggtgcggagagcttgaagcgaggttcagcagcgaagcaatggcaggctcgagtgggagcacagcacggtaggcgtctcaaatatccaagtaagttaaaacctttgcatctcttattcaatgcagggaaaaaaactccacttaattactcgcctgctgttatttgtgattctggttcagaggccggtggatttcatggagcgagaacaatccatctcggaggttcctagctatcttgtgcaagggctcgggtgagtagtttgtgaatgtcatttggtcgattactttaatgtttgaaccattttgacttgggtcgagtttagttccaaccttttttcaaacttctaacttttccatcacatcaaaacttttctacacacacaaacttccaatttttccatcatatcgttccaattttaacaaaacttttaattttaacgtgaactaaacacacccttggttgtttcatttttgaatgagcaaaatgatggatgcaatatctggtagtgatatgaaccggtgacaacgccgtatttgtctacactgttcatgattttttttttgaggaaccgacgacaggagtttctcctgccggaatttatagaagaagagaatcggcccagttaataagagaaaccgggcccaaaaaccaaacaagcacggttaattaaatgggaaaccggcgaaaacctatacagaaaaggaaaagaaaacaactattgagacctcacaaaagtcatcgttgccgagcttgcccaaggcaagcagcttcgacaacataatgcacgaggtctccagaaccaccaaacatcctctagcggagcagaaaccttctgagcgaagccttcgacaagggaacaacatcaataatgctgtcaacgcccgttccaaaaggaacccagttttcacctgaagggagagagagccatcatgatgatacctccaaggaggaaacggcacccacaagcgttgatatcatcaacctagaaaaacactaggcaaggttttcacccgagactctctcaaattgctccaccaccaaaccctcatcgtgttgcctggacgtctgttgagggtcatcgttgccgagcttgcaagacaagcagctccgactctgcctcaacagtcaatcacattgcagctcgaccagagatcacctcacacacaggaacccaactgcagcaaacctacggcgcggccgacgaccagtgagacaggacgccggaaaccaactccacccgccacccctaccacacaaaggagctccccggcaatgactggaaacagcttagcctaagaagggaggaggacctaagaagagggcagcggacgccacaccgccggtgagcactggaagacgaacaactaaagaagaggggaaccggatctggagaaataggagtgagggagatggaggcgccgtccccgtcgccggcaaaatcgcagcaggtcggcagcccccagctaggtcagcggcagccggcgccggagcgcgccaccgagagccggactggagccgccgccgacgaatgcgagccgccgccgacccgatgccgtggtcgccgacgagcccacgccggagaagcccgtcaccggtccatccgcctacgcccgagcggcgtcggagtcgaccaagcccgtcaccggtcaccccggccagaggggcaaccggatctggcgactccgtcgcctgtcccgtcgccggagccgaccgccacggggagccccaccgccgcgccaccgtcgcggtggagagccccaccgccgcgcagtcccatcgccacgacctcgccgtcccgctgccatcgtcgccacgcccgcgccgggacgagacggagcggaggatgatggccccgccgccgccatcccagcgcgtcgcccggctttgccgtcaacgagctccggcggcggcgaagcacggaggagggaggaggaggggtggcggcggctagggtttcgcccccgagcagccgcgcgagggcgacgcgagggctgttttggatttcccttccactgttcatgatttgaagatagagaactctaaaataagagcatcccttgtcaatgctagagttgagattgatcaacttatcgttgcgcgcagtgtacacgaagcacagtgcagaaggggcgcgtctagtatacgaatgttcgcgtagcatgaacacaatagagggtaaattccgacaagtccagtccgtgtgcgtgggcaccttttactttcgaccgacgtgctagctggctcgttcgcttgcagcccaatagatacaacccacaatattttagcctaattcatttgtgtgtataaactttgtaggcatatactatatacctatagactttgcacatataaaatttataccatagaaaaaatagtgtagggattttacgcacataaacacatttagtatgcaaactttacaccatggaaaaatttgctgtagcaattttacacatagaaacaattagtatacaaatttatacacataattttctttgagagaaatacacggaaatatttggtatagaaattttatatttgcatggtccatgttttcttgctacagcgtagatgcccttcatttgaattaggtccaagcccatcataagagtaaaaaagaagaaagtctggcctcaatgagacaggcttggtccgaaaaacatatgggtccacatacttaaaaaatattaaagaactaaacacggcctgtgtagtgatgttatttgctctgttgcttagagcaggtataataagtcctaatcagcaggctgaagcagtggcgaagccaccgtaggggccagggagggccaacacccccctaacctctatgtgacctcccctcccctctcttctcccaaggatgattagcacttataatctagctattaattatatcattagttgcagatgagactaactatataggacaggacaactgcctagctatatatatatatgactcatgaaatgacttagctaaatgatttatcattttagaagtgattgcaagactccaattaatgaaacaaactgttcaatcatccatatattaaaaatattactgaattttaaattggcaaatcatgatatatgggttcttgttctctcttgtagctaatcgctgtaatatgtatgatatcgagttaccgatcatacatcaacgctataaaatcactgttcactaaaccttaagtgagttgccatgtctcgtagatggcaccctccatctctaaatcttggcttcgccactgggctgaagggtttgccatgtcatcaaaatcctaggtggaggagtgagaagagaagagagagaatgaagcgggcggctcctctatcgctcggttgaagcgcatcgtccgagaaaaatctctcgctttccagcccggtgcgagcgctaagggcacccacaatggttatctataggctctctacaagagatccatgtcagcatattttcctacatggaagatattaaatgaagagagagagcaaaactatctactaaattagagatggtctatagagaaaaacgaggcaatgcatgagagagatatagatacccatgtagacatactattgagatggtttactattaatctagtctattgctgagatgtacatgttttatagatagcaccttactttaccattgcgggtgctctaagggcacccacaatggttatctatgggctctctacaagagatccatgtcagcatattttcttacttggaagagattcaatgaagagagagagcaaagctatctactaatctggagatagtctatagagaaaaacgaggcaatgcattagagagctatagatacccatatagacataccattaaagtggtttactattaatctagtctattgctgagatgtacatattttatagatagcaccttactttactattgcgggtgctctaagccgctgctttgttcctccctccgatcccatgcatctctgccgccgccctcttccctagctcgcctcctctacgcggaaaattgccccagcctccctccaaatcgagtcccccctgctacaattcatagtataaaacactcaaatccatccccatgccaccaaattcgtcctcaaattgcctcctccaaaccatagacgccatttgtccccaaaccatagcaaccacagcaaggttgaacggcaaagcaagaggacgtttgtaccagcgtctctggaggtctcgccgccggcgactcagcccgtcacgccgccgccacctcagatcgtcctactgccatccttcctttcacatacagatctagagactggagccaggtttcccccagcaacaccacagtccatgagcatgtgtatcttgtttgatgaaatagccacaatagatcatatctcatgcatgcattaaatgcctaaatatttatcaaaggtagattaaccaccaacctatcagccggcctattatataagtgaaggctttaggtgatgtgtcactagtataacgcccactgctggcgttcttattatccttgctcttatctggttcaggctagatcgatatggatattctgtttgcctcgccttttgtgttatctgaaatttatatggcacaggaatgatcttttgttgtgtatgtatgtttgttcaaaatcttaaatgaatggaatgaattatcccttcagcattttgtggttagtattttaatttcgatgagatgaatgaccaactgtaatatttgtcatattgaatattttgtgagactgtcagttatgtcgtgaaaattgccatccataatatatgtcagattgacaactgtcactgtcaactcatagtaaaaaaaaaaatcagatcggaccgaccgttcaactggaaaaaaatcgaaaccaaagatcttcaaggttcggttcgaatgtaagaccggacatacatgtaaaactaccttgaaccggctattaaaccagcaccgattaaacctgatgaggccacaacagtcgtttaatggccttaatgtggcccaattagtctgccatgcacactcagttggcaaaaaatgaaaaaaatatatcctcacatgaatccggctgtcctagcagttgcgttatagctttaacatatattatatataaaaattgaaccgcggttcaaccacctggtgctcgattcatagtttgaccagcggaccccgtaaacccaggtgcccgattcatggtttttcgtactataccggcaagcaaacaattggctgtcttcggctgcccctcttcctaatccctcctctcgttttctatgcgcgagcttttcaaactgttaaacggtgtatttttttaaaatgtttatatacgaaagttgcttaaaaaatcaaattaatctatttttttaaaaatattagctaatacttaattaatcacgtgctaataaaccgttccgttttccgtacgtacaccggtagggaagcaatggtgaacacagccctaaacaggtcaacctcaggtgttgactagagtactagacaggtcaaaagggtggccccaccgcaccacccaacggaatgcccaattgctcaccgaaacagcagcagtcacaacctgtccacgcgtttatcccccgtggacacggtccacgctctaaatgacaataccttagccttggagtctctcattgcacgtgagaggagtgcatacaccgcgtccacgcccacattcacacgccccccggtaattcctcgtccctactacagtactaccagtagtaaaactttcctgcgtctcctcccgtccaatggcgactccaccggcggccggggcgccgcgccggcggtggtgcgcgatccagggtctcgtcgtgttgttcctcgtctacgtgcttgcggtgcttgtgctcgccggcggcgagctcttccacgacgaccagctccagccccgcttcccatgtacgtgctcccggccggattcctggtttatcttttttttttcccccacgtttaattttttccccctttttttctctcttccgttctgcaagattggttcgtgatttccacgcgtttctgatctgtgtagcctctcctggaatcggatcctcttcctcctcctcctccgctcgaattctcctctcgcctcgctcgatgatccgccgtctcggggagatcgctcgccgtggtggaagcaggcggtggtggactggtggtgttcgaccggaatcgggctctccaagaagcgagggaggaaacagttcagccacggatcaggcgtgttcgcggcggtgcgccgcctccggcctggcggggatggcgctgcgcctcgccgagcgtctgtccttggaggaggacagcgtcggcggcggcaacctcgtgttctcgccgctgtccatctactccgcgctcaccgtggtcaccgccggcgcgcgggggaccaccctggccgagctgctcgccgccctcggcgcgccgtcctcccgcgacgcgctcgccgaggacgccggcgagatcgtgcgcgccctccccggcggctccggcacggcgacgggcgggccccgcgtcgcgcacgcgtgcggcctctggcacgatcggaggaggaacgtcaagccggccttccgcgacgccaccgccgcgtccttccaaggcacgacgcgcgccgtcgacttcctcgccaacgtaagtgatcgaatcatcgatctatatcaatctaatgtcacgccattaacgaatgacgatctccattgaaacggacatgcgcgacgcgtgtagccggaggaagcgaggaacgcgatcaacagctgggtcgcggcggcgacggagaacctcatcgacacgatcctcccgccggggtcggtgagcacggacacgcggctcgtggtcgccagcgccatctacttcaacgccacttggcagacgccattccgcaagcaggacaccaagaaggacaagttccacctcctcggcggcggcggcgacgtcgacgcggacttcatgcgcagcggcgacgaccagtacgtcgccgcgtacgacgggttcaaggtgctcaggatgccgtacaacacgcgtgcctcccgtacgcacacgcaaccgcagtactcgttgtgtgtcttcctccccgacgagcgcgacgggctgtggaccctcgccgacaggatggaggccggcggcggcgaggtcttcctccgggagcacatgccggagaagcgcgtcaaggtcggcgagttcaggatccccaggttcaagctctccttcgacggcagcatcaagaccgcgctccagggtgtcggggtcagggccgtgttcgatccggcagcggccgacctgtccgacgtgctggaggagggcaactccggcgatccgccgctgttcgtgtcggacgtcctgcatggggcagcgatcgaggtgaacgaggagggcacggaggtggcggcggccacggtggttattatgaaagggagggcgaggcggccatctccggcgccggcgccggtggacttcgtcgccgaccatccgttcgccttctttgtagtggaggagtcgtcgggtgcggtgctcttcgccggccacgtcgttgaccccactaatcctagtcaactgtaatgaaatttgcatggatcttaaatggtcgtggcaactagagagcaaattgagccttttattcgttttaaattggatatctaccctatagtatgaatttaaactggctagttctacgtttgctatattttagggtgaatgaaatagaaaattgctttgggtgtgttcggacctctagtttcccaacccaaatcccttgtttttcgcgcgcacgcttttcaaactgctaaacggtgcatttgttataaaaagtttctatacgaaagttgcttaaaaaaacaaattaatctattttttttaaaaaaaattagctaatacttaattaatcacgtgctaatagatcgttctgttttccgtgtggaggcactatgttcccatcatccacatgctaggcattcatatagacatgagaagacggctcactcagatctactggatcagcaagaaggcgctggcttcagttgagagtactggagctgtgtgcttttagtgagggaaaatctattcaggtttcagcgaaacgtttaagcagttttacattaacgccgtgtttagttgcaaattattcttcaaactttcaattttttcatcacatcaaaatttttctacacacacaaactttcaacttttccgttacatcgttccaatttcaaccaaaattccaattttggtgtgaactaaacacagtctaagatgctgctctttctagtgatatttgtactggtcatcgaagtcgttcacataggattggttgaggtgattgtaattgtcaaatgtgctgtcgtgcaacaaaaataaaggattacattcgagatggaaatttttgcttgtgtacataatagagcaagagtacagtagcatgctccgccgctggatctgggagggagggaggggccgggagccgccgccaccctcctccgccgccgctcgccgccaccctccgcctcccgccggtagccgccgccctccactgccgccgtacgcgtggccaccgccttccccccgcccccctcaggccagatctgggagagagtgagggaggggagtgccgccggccaccgccctccgccgccgcctccaccggtagccgccgccctccgctgccatcagcgccggggagaggaggagaggtgcggggaggggagaggtgaggcaaagaggacttagaaaaaaatctagggtatacagcctctatttatatttcctatgtattttcgcaggcggaggtccgcctgcaaaaatgaagggtatttttgcaggaagaccgtttaagtggtccacatgcaaaaatggattttcgcaggcgggcagcgggtttcaccccgatttacattttcgtagacggctatttgtatccgaaggttatgtccacctgcgaaaataagtatccaacatcggcaaaaatgccttttcttgtagtgtaatagagatgcgacatatcctaagactataaatttggagagatagttatctagatttatagttatataatatattacatttgtattaggttgcatttatattccagagggagtaatagctgccctatgtggcaagtggatcagtagatcaattggcatcaaacaatattggtgtgcaatctttcgatcattggttgtttttttagaaaacaaaaatattttcggactggcttgaatgctttcaaaacatgtaattgaacactcgccatcgagtcagaaaatcgttttcaatctactaattgatcggttaactaggtcctgtcatcgtgaagcaacataatgcaatctaataatctgtacacaagtcgaaggaagcatgtaactaagcaacaagaagctgcatatacatcatggagtagtacgcacgaatccaggtcaattggcaaactacacatttggcgcggcgaccctgaatctgaattgaacccatcataggcctgcctcatcaaactggttcgggcgggagagtctcagtagcacctgaggtcctgggtttgactccccgtgggagcgaatttttcaggatttaacggcattgtgctttcagtggtaggcgacgtacccgtcgacagcgaggcgcccgtggtgacttcgtcaatctctccaagatttgccagtccagtcttcgaagatgctcatagatatagggtttgcgtgcgcgtgttcataggggtcagtgcgcgtgcgttgtgagtatctgcgttgtactgtgtaatttttttttaaaaaaaaaactggttcgggcagggccccgtacgtggacctcgacgcctcgagcgccctctgcatctttccaaaaccctacaagtttgaggcggcgatcgatcggacaggcagcggggagacagatcgagcgagcggtaggcatatgggttttaggtcgttgtcgctgctgggctctctcgttcgcgggagtatccgctgttcctcctcctcgccgggatcgggaatcgctaatcgtctactatccctggtaaatcaaactcagaaacttttaactaataattatattaatcataatacatgatatatcgctagattcatattttcactctaatttcaaattcgttaataacgcaacatgaataaaccaatagtcaaaatatgccattgaaaatcgtgtaaaaaatatagatattataatttgagacggagggagtagtaatctgcttcttttgatcttttctcatctcagatctatacatcgtatgctttctccgctagcggctcataattattctcgcagaggaacgcgctgttcttctactccgatgcccgtcgtaagagtcttgtggcggccaacacctctgctttcgccgccagatcatcccgttgtctgcacagcatggtaaatttctttcaaatcttgctgtgtgtgtttccctcctttttcaatcccacagacacacacattcacgcgcgtgtttccatcttgctcccgttatttcgttcgttaattcatcgttgtgcaggggtctctggggtcgccggcggggaggagagcacgccggttccacgacttggtaaatttccttcatcgtttatctatctaagccttgttttacttccagataagctcacgttgaatcctcgtggtgcagagggatgtggtcttggaacatgccgatgctgctaccgctacgttgtctgttgttgttttctctgcggttgctggaattttgcatttgatgcaagatacaggtgagtttttaggtactccatgtgaaactagctgcctttgactgcatgtttcatatatgagctttgttctggtccagcagttggtacctccaggtactggtaccacaggtaccaaacgaaatccggccgttcaataaactggggtacgattggaaatgaagtaaagcagccagctagcgtgcgacgacgaaatctcctcatcttcacaggcatatccgagtcgccgctgccgtcgcattctgctctccttgacgcatgacatcgttcctcagctcgctccgtcgcgcacgtctctgtcaccgccgtcgctgctgcctgtcctccccccagccccagcgtgtccttgctctgcggcaacgacagtgggaccgatggccgacgctgccgcaacatgtatcctgacgcgccggactttgtcgccaccgccgctgtagtctgcaccctataattgttccggaatggaggttgtgttggagcttttcgcatgagtattgattttgtgtttgtctgacaaatcaacctgaagccatttaggcaaatcaacttcatgcttctctttttttttgcggggaacttcctgcttctcaatttattcctgtatgtatctttcttgctcaaaaactgttcaaccaaatcaaccatcgtcgtcgctagcaatgacccgtggagtgatgccgtcgtcgccgaagacaattatcggagcgctgccaccgcagttgcagatgatccctggagcaccgccgtcactgccgataactcctggaatgctgccgttgtcgccgctgacaacaactcctggagcaccgttgtggcctcagaagactggtggagcgcaccgtaattcctgaagcgtcgccggctcgccgccactgccgtagattttcctctgcaaagccttatccaaaacacgaatcaaatcgacgaaattcctttccaatgtgcaaatacaattttacccctccacctcattcccctttggcaggtaccacgtagggagtaaatggtacttgaaggtaccaattgatgtcagccatccgatcatacctatccagcggtccacatttggtaccgggcttcactgctggacagtaaaaaaactcttcatatataaccatcaatggttatgggaaattatttcgatcgcataagaatgtaccctcgtttgcttaaaagtcatccaaattgttatgaaaaattctggaaaattttgacaacactcatacaaaatatctatacaactttataaaatcttggctccaaactcaagtcacacgtcgacgtataagaaagacaaattcagtgtatgaatagcaatgtactgttcacgtctaattttgtcttttcgatgtgtagattgaatttgaacttgatttttaatggaccgatacatatcactgtactctacattgtcgatatttttttcagattttttcacaactatttgtatcgaattttgaaaaaaaaagcacacacgaagggataacccctcaagagattagaacccactcctcatactacatgttaaacacccttcagatttctcgtctttagctttccttttatgtttttcgccgggaagctttccttttatgttacattgccctcaaccctcacgagttcatactgtctgtacgtagacgaagctcggaaggaggacacacccactaagacaaatccttacttccgttccaaccagttgatggaagacttgatacgagagagggcgtcttacaacgaaacaacgcctgacgagaagaccgtggttcgagagtacatggaggatgatcaagacataagggcaaggttcaaggattggatgaaggagcatggcaggacatacaagcaggacgaggtggaggaggctaggcgattcaaaatattcaagtcagttgcaagattttctgatgcagccaacgatgattctgccaacgcagggcactcgacccgttttggcctgaatgagttttcagactggaaccaggaagaacttgctcggatgtgttgttgtatgccagctaggagtgatggtgggtacttcgaaatggtcctgtcttatcttgttccgcaggggatctggcatcaggtatagctgacatggagtacaaggtatgtattcccaatttcatctatacaatgtatattttaatttgtttttctgatagacatggatcgagtgcattaactggagaagcccttagcttgatctgtcccaaaatgtaacgtgttttggaacggagggagtagtagtaacctatcttgtacttcctgtgactcttttttggaatcggcaaattaagtgttaattatttctcagttggcttctcactcgtgaagcagttagtgtactggcttgaccttgaattccatgttgtgataggatcagggttgttttaaatctccgactatagctgccgctatgtccggttatatctgtttgagaagagttttagctcttttctcttccgtataatttagccgctatagatgcccctataactgttgagagggccaaccgctaaattagcctgctataaaacaatggttaggatgaataagaaatgcatttttgaactacctaattattatctgcgctagttttacttaaagagaaatatttgtgtactgattagtaaataccccgatcatatatatcttggatactggcagtttagttttgttatttctatcgtgtggcggcttcacaatcatgagttctcaattgttgattgcttgctgtattgatcgcttgctaagcacttgtaaagtagtttgattttgacacagaaatggcctttattcaggctagctaacgtcaatcatgtttgttgacaaatcaaatactccctataaatgcaatttcataaatatggtatcaactttgcactttgatgcttacggttctgtgtatttttttcttgaagcaaagtagttggagaattgttttgttttccttttcagccaaaaacggttttctattcaagtctctgtttttctgtaatcaatggatcttattaagctcttcattgagaaactctagggtggtggaccttaagaagtgaattttctctcacaggctaacagctacagcgcataaaccagctatagtttctgtaagttgtcatgatttatgttcatgtaatggtacagtagcagtgtgaatgttgatttacagcagcctgaccgtatcgaaaagtaacataaattgggtgcaatgtagagttttaaccattttcctgggacaaaaaaaaatttatacaaaattgccttgctcaaatctgatactttatttttgctttaatttgtatgctcgctggttgcacctttgattcttcgtctgtgtacttctctttcattatggggcttcttcagagctatagcctgcattctaattaatttcctttctcaaaagcaggagttttaagattgagaagggtcgagacgctatatatatatatatatatcatacgaagaccaacaagctgtcatgtgaaaaccagccaaaatgaatctgtttcagatgtcaaatgtccatatcttccgtttttgtaccctctaatgctacctgtctgaatagcataatgctagagttgttatggttatatgagctatcttgtaagggtggtagatgttgaagttaatataaatgggaatttctgaagctaattaagagtttgttttcccattggacaattgcattcttgtcctaactttgatgatcaaactctatatcacacaatactgaaagttacagtcgcgtgtactgtcacaccgagtcgttccctcgctggtggattaatatccccaacaccaacgatctaggctccagaatacgttcccatacgttatgccaagttctttgatcggtagttggaatatatctcttgttcaactaattctaaactctatcaaacatttgttgtcctagtatatttcgggtattctcatgtgattcccacaagctcaaatttattccattgtcatgtcatgcctagttttccatttgttcttcaaaataatgaaagaaaataaccgacctacaaaactcatgctcaatccaatcaagttactcgtcgtgtcgtcatgctttttttggccaaactcccttcgaagaagaatctgtcaccagagagactaaaacaataaaataagttgatccccgtgatgggcctatctgaggcatcattccttcgtcttggggccctcacattctaggcctcatcaaattaactagtttggtgtaccgcgcaaatgtccaacctttttaaggccgctcgtaatatagacgctatccttgccctcgaacccccgcatccttccaaaccctagcggcatgtttgccaagagaagaggattgggggtttgaaaggaggaagttgatggagggattggtcttgggagttccatttttactccccgtacattatttgggtagtagagatgacaattatggcgagtttgggtataaattaaattttacgtgatgatgtatggccgagatctatctcttacagttagaaggaaataggtgtcggttttaaataaactgacacctataggatattaggtgttttctagtagtgcacccttcttagcggtgccactcctggttgtagctttctttgcacgtcttccgacatcagcaaaatggaccacaattcaatgaaaaacatactttatgtattagctcagtagggtcatgagatttcttgtttttaaaacatgctgcatttttggctttccatatggtctagatcacagctgcaatctccacttccatagagggaatatgagctagatgattacggcctgttttggaagtgatttcgtcatcgtcgatcgtctgcattttttttccttcgatggtgacaacattgagtttagatcagggggctagagcgtcgagcttcctacaacaatcatttgtgtctatttcactctcttcgtcgtcgctggcaatagcgttctaagcttgggctacttcgttgttgtcacgccccaccctatcgcacagccaccaccaaggagggtgtgccgcaggagacagtatgccattctcacgcataaccatgataatgttacccactataacaacataaatttttatagtaccaataaaaaattataaattaattattggactgctaagagcaggtacaatagatgaattttgcccgcgatactacatgcatgcagcctaatatggtgagttggcagagaaatttggggagaaagagggtgagccggcgactaattagtcaccgcctccacgcgtgtgtcgaggcaagaaaagcaacatcatacgttttcctactggaccatacagttgtggggtccacgtgattagtagtatgtggatgagaagatgacatgtgtagacatgacactataaaaaaaagaaaagaaatgttttgtcattgagtggaggcagcttgtagactattgttgtacaaattagctatttggtgaaagagtattttattatacttgtcatgatttggagctgacagcttactcctctattaaacttgatctaataggatacatttatacttttaggttgcaattggttgcaaattcaggtagatcgggaagtatagcttttattctttggattgcaggatttgaggcctgccccttttttctactccatcagtttcatattataagtcattttgatttttttcctaataaaacttttttaaagtttaaccaaatttatagaaaaatatagtagcattttcaacacaaaatatatatattatcaaaatctattaaatctttcatctgatgaaactaatttggttttatacatgttactaaaattttctataaacttgattaaatctaaaaaaatttgattaaaaaaagtaaaaacaacatataattaaataatgggggtactaataatagataggttttaaaccatggcgatttcatgctatatatacgaaattgctaaaaaaactgtcgcaagtttaactataatgtaattacagtataatgtaattacagtatagttacattgtaaattacgctgtaactatatagttacattgtatctgcactgtaagtatattgtaatatctatataagttagatataaaactgtatatattattttaatacttatataaaagttatataatagttatagtggaatcatattacaattatactataattaaagtttttttcttaaagaacttagggcagttttttaatagtccctaacaatatacgatatactatgggcctgttcactttgatgccatttttaatcttaccaaattttggtaaagttgtcaaaaaagtggctacatttagtttactgccaaattttgaagatctatctatataacacccgacagatttttgttataaatcacacagatttttctaaccaccaaacggccaacttgctagccaaattaaaaagcaacgcctatagtgtagattaaggcgtgttaatccaagccacaaaactaactacttgtcagcacatatactaaaggctttttttaaaaaaaattacaccctaattacactatacttacatttgtaactatagtgtaattatagtataactaatatgtaagtactgtgtaactaatatataattatagtgtaactacactaaaaagtttttttaaaaaattacaccctagttacactatacttacacttgtaactatagtgtaattatagtgtaactacaccaaaatctttcttttaaaaaaattacaccctaattatactgtacttacgaatgtaactatagtgtaattatagtgtaactacaccagaatctttcttttaaaaaaattacaccctagttatactgtacttacgaatgtaactacagtgtaattgtagtgtaactaatatgaaattaatacactagtggagaaaccctttgtagtcccggttcgtaaccccctttagtcccggtttccaaaccgggagtaccaatccgggactaaagatcgctatctttagtcccgggtgaaataaccgggactaaagatcgagctgaccttttttttttgcatggccctgttgctgcatggtttatatatatatatatatatatatatatatatatatatatatatatatatatatatatatataaaaaccatgcatatatatgtttcaatacatatatatgcatacatatatatatgtatatatatatatatattatattatatgtatatacatgcataatatatatgtatttatacatatatatgttatgcaaatgcatatgtatatagatatatatatgaccaaaatatatttacattatagaaaatgtgtacacatgcatatagaaacatatgtagtcatatattaattacaatccattttatgtcctagctagctacgatttcgacgtagtagtagtcgctgctagctcagaagctaaggaccggtgaattgtgtttccgtcgtagtagaattcacccttgggatcaaggatatcttcgttgatgaatcccatgagttgttcttgaaccgctgcgataaattccttgtgtgtggtcaggttatccctcatgcgaataaactatatgaatgtatgaaattgattagtaattaaacaagaaatcgttacgtaatgaaattttgaatttatttgtacgtacatcgagctctcttgtggtgatgatttggtctgtaaggcagtggcaatactcacacacgtagtagccgcacaagttagttccctgcttttgctttgcgcactacaaataaatgacaaattaacggcgtgagatctaattaatatacagttgtatgtattaatttgaatcggagaaatataaatgtagagcatgtgtactcacaggaaatttgaacttccgcctaagtctttctctccatttgctgcggaccaaatgacggaaccgataccaagccctacatggagtttaaagctatgattcgtagtagcaattaacataacaagattttcttaattaacaaaggaacttatgacggtacctgtctataagttcgaaaaccttgtcaaacgtagactcttttttatccattgagtcatatacgttgacggtgcaggcctccaggtcgaagagtaaaagcacccagtggaatctgcaatgtgcgtgggatgcattacatatgaaacacttagcaagttcgtacgggaatgaaatttggtatgtaggaagacagtaaaattaaactaactctgtgttgtacggtaacagtatgaacgtcttgtaatgctgcgccttcaggagatggacgagattgtcctctgtttcctgcggatattggtcgagcattgcaacgtttactctccgagggtcgatgaatccagtatcgaaaaccctccgccgtcgggccctttgaatctccattctacaacgataaaagggagtattttattttatatagtctacttatatacaaggacctaattaattaaaggagacgtagtaattaagaattataattgaacgatatacttacaaaatccagcaactcataatagagacgtcgagggcgtccagctggtatagttcgtagattcccctgaaattgatccagagaatgtcatctccttgcaagaagtccgtgtccctgatcgtcgctccgatcatctctctaccggtggcgctcatctccatgtacagctgatggaacttgtacatttgtgtgggtagggactgcagcagctcaggcttgacaagcggtttaccgagttcgtacatgtatttcactttcgccttttcgattggtgcgactcctagcaattgatccgtagttagaccggtgtcagtaataaattgttctatcgtcatttctttactggtcaccaacggctcgatctcctggtttggttgctctccaagctgagggactggtttggactttccagaagatgctttcttcagcgttcgctcatagtccgatagcttgatggcctccttgacagatgcagacatacccctgaagaagttcctgacactggggtctataggaatcttcttttctggacttcgaggcttgaattgtctcttgacttctgatgcaacgtaagcgtcaagctcctcttgcgtgcgatcgtaccccgggtccttgttcttggcggcgtcaactttcgccttcttcactgcccttgtgtgggcaggcggtggagctgggggggcccttgactttgaaggtgccggaagaggagcttgcgggggagtaggtgtaggagcacgaggaggagtcggtgcaggatcctgaggaggagtcgatgctggagcctgaggtggagacggtgctatcggagcatgaggaggagacggtgcaggatcctgaggaggagatggtgcacgagacgccgcttgtcgcccagggaggatgatgtaccgcctgcgtcatagaataatggcatggcttgtttctcgtagatgcgtctcaccgtctcctccagggtaatccagctcgaggtcctcgtacgcgccttcgaccaactcaacttcgaccttcgagtatcctgctggaatcggcctgcagtggtaagtacctgaagggtccgttgggatggccatgcccgacgccaccttcatgttgtgagagattatttattagtaatcaacatatataagcagcaactagcggaatggctaaatcatacctttattgataagttcttgaaaggaatatgcagctcacatggtgtccgttgagtgatgtcatcaacgggacaggtcgattcgtcctgtgtttgcatggcgtccatgctctgtgatcctacctgccccgttgaggcgcagctgctacggtttcctgacaggcttaccattgcaggaggaatggtcagctggggatcatgggaccgatgtgcggccatgcgttcatcaaccttccttgccacctcctcttgcatgttgagttcgtagctcgatacctggaactctaggtctgcaatcttcgcctcggtatctctcttgctcctcatccaactcctgtacgtgtggatgtcctccttgaaaccaatcttccaaggaatcacccctttccctcgtgttcgtcctggatgctctggagtctgcagggcgagtgtcagctcgtccctatctctgtctggtcggaacgtgccctgagaggaggcttccactgcatctgttagtcgtcgcgcagcctctcgtatctgatcgccgaagaccagtgagccatcagctgggttgagcgttccaccgtgagcatagtaccagaacttcgatcgttccggccaattggctgttgccggttcgatacccctctcaatcaagctagcctccatctgctcccacttcggcatcgcgacgctatagcctcctgaccccaagtggtgatggtacttcttcttggcggcattttctttgtttctttccatcatcgcctgcccttgttcacctgtcttatatgcaacaaactcgtcccagtgatcccttagctttgggaatgtgtcgaagttcggtgtctgccccttcagtatatacttttggtacagatctcccttgaagctctgaaactgttctgccattttcttcagagtccaccttttcactttgtcctctgtacccgcaggaagggtgaatgtctcgagcattgtggtccacagcatctctttctctgaatctgggacaaagctctcatgatctccgcgtgcccttgttcttcgccagtacaccgtactgacaggcacgttatcccgcacaacccaaccgctgtgacgtacatagttcttggcggcttcggccggggcactaggacgtccatcttcttgcacttccgttatgatgtgccgaccctcaagcttcttcgctgcacctcgttgcccgcgtgccctcttctgtccaacggagggttgactaccactagcctcctcctcctggttcccctccacatccctttccacgtgcccctgctggttcccctccgcatccctctccacgttcccttcctcgttcaagtactggtttggatcctcgttcccctcttcttcattccagtactggctgcttccctccgcgattgtatcgtacaatatctgttcctcatcgcggtcagccatctgttgatacaagaaacatctgctaagtcacgagacatttatgttttaacaatctaagtcatgtatgctaagtgtaaatgtcgtacattagaaccctacacaaccttacgtgctaagtctaattacaaatcgtgatataagctaagtctaggtgacgtatattatataaccctagctagtaaataattatatactaagtctaattacaaataaaataatcttatattaaaactcaaataatattacatgctaagactacaatagtcatgtatatgctaagtgtttcgtgcgtatatgctaagtctaattaagactacaatagtcaattgctacttgtcgcaccaattccgtagtcaataattacatactaagtctaattacaaataaagtaatcttatattaaaactcaaataatattacatgctaagactaaaatagtcatgtatatgctaagtgtttcgtgcgtatatgctaagtctaattaagactacaatagtcaattgctacttgtcgcaccaattccgtagtcaataattacatactaagtataattacaaataaagtaatcttatattaaaactcaaataatattacatgctaagactaaaatagtcatctatgctaagtgtttcgtgcgtatatgctaagtctaattaagactacaatagtcaattgctacttgtcgcaccaattccgtagtcaataattatacatactaagtctaattacaaataaagtaatcttatattaaaactcaaataatattacatgctaagactaaaatagtcatctatgctaagtgtttcgtgcgtatatgctaagtctaattaagactacaatagtcaattgctacttgtcgcaccaattccgtagtcaataattatacatactaagtctaattacaaataaagtaatcttatattaaaactcaaataatattacatgctaagactaaaatagtcatctatgctaagtgtttcgtgcgtatatgctaagtctaattaagactacaatagtcaattgctacttgtcgcaccaattccgtagtcaataattatacatactaagtctaattacaaataaagtaatcttatattaaaactcaaataatattacatgctaagactaaaatagtcatctatgctaagtgtttcgtgcgtatatgctaagtctaattaagactacaatagtcaattgctacttgtcgcaccaattccgtagtcaataattatacatactaagtctaattacaaataaagtaatcttatattaaaactcaaataatattagaacactacacaatcttatatgataagtccaattacaggtctaataatcttaattaattgcattacaaatataacaatcatttatattattacgtcacttgcctgctccaattccttctagggctagctcttccactcaggcttgacggacgactccgacaacacgtcttttctgcaagatacaaaaagatgtattaggataaatgcgaagtaacatatatatatatatatatatatatatatatattgcatttcacgttaacgttcgtcctcgttcgttcgctcgttcacgttccctagctcgttcacgttcgttcgctcattctcattcacgttcgttcgcttgttctcattcacgttcgttccctcgttctcgcttgtcttcgatcgatcgttctcgttctcgctctcattcgttcgatcgttctcgttctcgttcacattctcgcggcaacgtacgttgacgtgttcgttctcgctctcgttcgttcgatcgttctcgttctcgttcgttaacggcggtgtggctggcatcggggcggcggttggcgcggcggcdgtggcggcggcggcggcggcgtggcgacggcggcggcggcgtggcgttgcggggccgtggcggcggcggcgtggcgtggcggcggcggcattgcgcgcgcggcggcgaaggcggcggcggctgtggcgacggcggcggcggcgtggctgttggcatacggcggcggtgatggcagcggcgtggcgcgtcgtcgtcgtggtgcgttgtcgtcgtggcgcggcgtctcgtggcgggcggcgcgacggagagagatcgatatcggagagatcgagatgcatgaagggagatctggcggcggcggctctcaccccagagatgcggcggcagcggaggaggcggaggcggcggcgaggacgacggcgagatacggcggcgtcggcgcggggatttgccctaggcagtggcggcgaaggagagaggccgagagagatcgatcggccgaaaacgtctaagtgttggtgaagaagacgagggcgcaccgggaatatatatacccccggatctttactcccggttgttgagaacaaccgggactaaagatatctttagtcccggttgttgagaacaaccgggagtaaagaaaagatatttactcccggttgttgagaacaaccgggactaaagatgatctttagttccggttggtattaccaaccgggagtaaatatcttttcccgctatttcgaaattctttttaaacccggttagggttaagaaccgggactaaagattatagcactacatatgattttcgcttacatatgtgtttataaaatagaagttaatgcattaacattatttaaagtacaaagattaatgacaattacttcgaaaaattatttttgtctgtaataaattaagtggataaatcgtaataagcaaatatatgacttataattaataaaaattccaatatatatatatatatacttagcatatatatgaatgatattattatattggtaaaaactctaattaagatatgtatgtacatactgcatgatttaaaattaataaaaattgtaatcatcatatatacttagcgtaggaattatattaaattaaatgctaaaaactctaattaacatatgtaaatgccacatgcatgatttaaaccttttaaaaattcgaatagtcatatataagtagcatatgaaagatagtaaattaaattgtgaaaaactctaatatatgaatgatagttttaaaaatatattaaatttaatacttttaaaaattctccagtcaattataattagcatatgaatgatagtaaattaaatgttaaaaactctaattaacatatgaaattgccacttgcgtgatttaaaacttttaaaaatcctctagtcaattataattagcatatgaatgctagtaaattaaatgttaaaaactctaattaacatacatatgaaattgccacctgtgtgatttaaaacttttaaaaattgtaagtatcacatataactagcatatacatgatttaaacttgttgaaacctctaataatcgtgcgtaattaacatatgccatacatcaattgatttatatggataatcaatacatccaaaatagtttacatcatgtacacaataattacggcattacatcggcggtgacggttgaccgcacgtactttctcctcactattgttccctccttgtgatcgctgcgtgagtaaggggtgtcttcatttgataggaggatgctagggtcaatcgtcaccgtgaaagggggttgcccatccaactgatcgtaatcctcgtcagtcttgtcctcaactccgacgatttttcttttgcctgggagaaccacttgacgcttaggctcatcaggccctctgcccttctttcctttgctagacatgtccttcacgaaaaagacttgcgttacatcattggcaaggacaaaaggttcgtcggagtatccaaccttgttaaggtcaacagttgtcatcccactgtcatcaatcattacgcctccaccagtcaacctaacccattggcaccggaacagaggaaccttgagaggaccatagtcaagttcccatatgtcctcgatggtaccataatacgtggcagttgttccatcgtgtcccatggcatcgacacgaacagcgctgttttggttcgtgctcttcatgtcttgggctctcgtgtagaatgtgtacccattgatctcatatccctggaatgtcgcgatcgacccagacggtcccctcgccaggaaggcaagttgttggttgatcgactcgttacccatgagatgttgtcgtagccacgcggggaaagtatcaatgtgatgccgtgtaatccatgcatcggacttaccgatgttcctggcgcgaactagagccaagtgctcctcgatgtaaggagctaccaatgaagagtgttgcagaacagtgaaatgggctttacggaataaattgttgtctaccgtcattattgctttccttccgagagttccctttccccgtagtctcccttcatggcgtgattcaggtaccccgattgggcgaaggtcttcgataaattctacgcaaaattcgatgacctcctctgttccataacccttggcgatgcttgcctctggacgagcacggttacgaacatacttcttcagaacgcccatgtacctctcgaaaggaaacatgttgtgtaggtacataggcccgagaatacggatctctttcacaaggtgacaaagcagatgcgtcattatattgaaaaatgaaggtggaaatatcaactcaaaactgacgagacattgcaccacttcattctgaagggcttctaatctatccggatcgatgaccttctgcgaaattgcgttcatgaatgcacatagctttgttattgttgcccggacattgtctggaaggataccccttattacaactggtagcagttatgtcatcaacacgtgacagtcatgagactttaggtttgtgaacttcttctccttcgtgcttattattcgcttgatattcgtggagtatccagacggtacctttatgctctccaagcattcaaacatactttccttctctgccttgctaagagtgtagctggctggactcaagtaatggcttcctttctcctttggttccgggtgaaggtcgccgcgttgttccatatgcttcagatcattacgtgcttccagtgtatctttcgactttccgtatacacctaggaagccaagaaggtttacgcaaaggttcttagtgaggtgcatcacgtcgattgcgtggcgtacgtccaagaattcccaatatggtaactcccaaaatatagagttctttttccacatcgccgcgtgaccatcttcgctctctatatgctggcttccaggcccctttccgaacactactttaagatctttaaccatagcaaacactgttttcccgctgcgatgtttaggcttcgtacggtagtcggccttatgtttgaagtgcttgcctttcttccgtaccgggtggtttgctgcaaggaatcgacgatgacccatgtatacaaccttcctacagtgcttaagatacgtactttctgtttcatccatacagtgagtgcaagccttgtaccccttgttggactgtccggataggttgctaagtgcaggccaatcgttgatggttacgaacagcagcgctcgtaggttaaactcctcctgtttgtcctcgtcccacacggggacaccttccttcttccacaactgtttaagatcttctaccagtggtcttaggtacacgtcgatgtcgttaccaggttgcttggggccttgaataataatcggcatcattatgtacttcctcttcatgcatagccagggggggaggttgtagatacacatcgtaacgggccaagtgctatggccgctgctcatctctccaaaaggattcatgccatccgtactcaaaccaaaccgtatgtttcgtgcgtcctttccaaattctttaaattttctgtcgatgtttcgccattgcgaaccatcggcggggtgtctcagcatcccgtcctgttgacgctcttcagcgtgccatcgcaacattctagcattccccttgttcctgaacaaacgccttagccgtggtattatagggaaataccacatcaccttagcaggaattctcttctttgttagctgcccgtcaacttctcctggatcgtcccgtctaatcttgtatcgtagtgctttgcaaacagggcatgcttctaggttctcatactcctcaccgcgatataggatacaatcgttcggacatgcgtgaatcttatgaacttccagtcctaacgggcagactatcttcttagcctcgtacgttgtctcgggcaatttgtttccccccggaagaatgttcttgacgagtttcaataaatcgccaaatgccttgtcactaaccctattttttgccttccattgcaacaactccagagtggtatccaactttttgtgcccctgctcgcaacctgggtacaacgaagttctgtggtcctccaacatcttgtccaatttatgggcctccttttcactttcgcagtcctccctggcgtcctgcaacatctgaccaagatcatccgcaacgtcgttaccatcagcagctatttcctcctcgcccgtttgatttccttcaaatccaacatactgagcaaagtccggaatattgtcgtcttccacttcatcttcttccatttcaacaccttgctctccgtgggatgtccaacaattatagcttggcatgaaccccgactcaaacaagtggaaatgaatagtcctggatgcagaatactccttctgattcttacacttattgcatggacaacaaataaaacccctttgcctgttagcttcggccactctcaaaaaatagtgcacgccgtcaataaactctttggaccaccggtcagcgtacatccattgccgatccatctacatgagtcaaaaaaactgtacacaaataattattcttacaataatgacagtcatacaataattataagatatctttattcatacaaaaatcataaaatattacaccaaataattcttaaaaactatttgtaaagttttaaactaattttaatgtgttttacttcttttaattttcattttagtttgttttctattatttaagattaactttcactagagttttccttctcaattattttataaaaccttgtttagcaaatgaactaaatttctatatattctttcttccccacacacattttctctctcatcaacttacaactaaattttggagacaaaaaaagtgtgcaaaacataggtgcaaatgtagtatgacaaaaaaaacattggaggatgaagttgcaaaccttttaggcacctccgatttgtatgtaatcaccaaaataaattcaatagaaattttggcatgacctcccctcttttttgaagaaattttgaagcttgctcgggcagctggaggaggaagaagacatatatatagggatgggactttagtcccggttggtagcaccaaccggggctaaagatcaccgggatctttagtcccggttggtattaccaaccgggactaaagttacgatctgttaccaaccgggactaaagatcccgggggggcctgacaggccctgacagcattcaaaccgggactaaagatgatctttagtcccagttggtaacacaaaccgggactaaagatcaaatatgcccgttacccttttgaaccgggactaaagatcatctttagccccggtttttattgcatccgggactattgtggaaatcggccgaccgacgaaagatggtttctccaccagtgatatgtaagtacaatgtaactaatatatataattatagtgcaattataatataattaatatgtacatacagtataactaatacagtataagtaagtattgttttggtgaaacaattagttgctccttcttgctcagctagttatttcacgttcaaagcatcagatcttgggatcgactcccacggaggttacagtagtccttttttttggttgtaacggcagctagcacaaatcataatgcaatttagacggtggaaaatctgggagatttctaaaaaaaaaaatttgtcgacaaatttttagcaaaaccaaatttggataactatataagaaatcctgctaaaattttagtaagttgtcaaaattttgacaactatatcaaagttttggtaatgccaaattttggtaaggtcttattttgcatcaaagtgaacatgccctatgttattattacttcagcattcatggattataggttctataaaagccttgggccgtacgtcttgtgatcgagttgtgcaaatagatctctgagacaggcgtcgaacacctttttccaccatggaatcgtgtgcacggcggtgcgccgtctccggcctgatggcgctgtccatgcgcctcaccaagcagctctccgccgccgccgccgccagcaaggctggcgctgccggcaacctcgtcttctcgccgctgtccatctactccgcgctctccgtggtcaccgccggcgcgcgcgggcgcaccctgacagagctcctcggcgccctcggcgcagagtcccgcgagaagctcgccgcgaacgccggcgagatggcgcgtgctctccccgcccccggcggcggcgcggcacagccgggcggcggcccgcgcgtcgcgcacgcgtgcggagtctggcacgagcggacgcggacggttaggccggcgttccgcgacgccgccgccgcgtcgttcaacgccgcggccctcgccgtcgacttcctcaacaacgtaagcgaactatatatatatacacatgacaagatctaccaatctaatctgtcatgtatgcgtgtagccggaggaagcgaggaaggagatcaacagctgggttgcggcggcgacggagaacctcatcgacacgatcctcccgccggggtcggtgagcacggacacgggcctcgtggtcaccagcgccatctacttcaacggccaatggcggactcctttctgtaaggagataaccgagaagcgggcgttccaccgcctcgacggcggcgacgtcgaggcggacttcatgcgcagcggcgaggaccagtacatcgccgtgcacgacgggttcaaggtgctcaagatgccgtacgcggcttgtgtgagcgcgaggacgacgacgacgccgaggtactcgatgtacgtcttcctccccgacgagcgcgacggcctgtggagcctggaggacaggatggcggccggcggcgagggcttcctccgcgagcacacgccggagcggcgcgtcgaggtcggcgagttcaggatccccaggttcaagctctccttcgacgacagcgtcgtgggcgccctccagcgtctcggggtcagggacgtgttcaagccgttcgtggcggacctggccgacgtgctggaagcggagaactctggcgatgatccgccgctgtttgtgtcggacgtcaagcacaaggccgtcatcgaggtgaacgaggagggcaccgaggcggccgccgccaccgcggtttgtcttaccttcgcatcggcggcgccatcgtcacggcggccggcgagggtggatttcgtcgccgaccatccgttcgcgttcttggttttggaggagtcgtcgggtgcggtgctcttcgccggccacgttgttgacccgacagatgagtaattccatcttcacgtcatgaaatttgttttagctgcatttttgattcggttatgaaatttgtggacttccaattaagaacgatttgagttcctcgactccgaggagtggaattcaggttgtaagggtaggactaagccatgtttcgtcttgttcattggcatagagagacgcttctttcgtctcattatattgacacaatgtcacaatacacagtgaagagcggctccgataggactaagctatgtagtagatttttcttcgcaatccatttggtcagaaaaaaaaaaagaacagggcgttttactcatggaaacacaatgaacatatgccacaaactataattgcacagaacaattctcctttcttgagatgccacaaactaggatgagacgaaatttaggatgtaggggtttgtcttccgttttgctgtcatgacgctacttgtaaggacagccttttggtttctttctgcttattttgttgcttttagagctggtagcaagtttgggagtctttcatgttttccttttggagggctttgaacctccctatgctataaactggtgtctgggtgttcccagcacaatcctctggctttcaatagaagccgggccgtacggcctttctcttaaaaaaaataatggtaacatggagaaggcgtgcataagaacaattaagaagaaagctagttgttgtgggaaccctaatcaattagaataattccatcttctcctaataaaattcacacaaatggtcgaaccctaattgttgtgggaattacagagagcaaaacgaagtttttactaagaaattgcatggaggtgccttcccattgatttagacttgatgcaaattaaatttgttgaattccttttgaatgatcgattcaagttctattttcctttctcaaacacgcaagagagcgtcattatattattaattgagattttctcccattcttggaggggtatatacctaattacaatttgagagccataacccaacaaccaaacaaactttatgaagaaaaactaatagagaaactgcacccaacaaccaaacaaaatttatgaagaaaaactaatagagaaactgcttgtttagtacaaactttatgggaaaaaaaactaatagagcaaatttctgagaactacaagttcaatgactaaactatgaacttgctgcaactttagcaacttatttcagtttgcacggtaacacctttaaaaaaaaagtcctagcattatagttgcaaaccaactatagataggactgaaaatcaaattcccctttaagatatgcacgtagcgaggaggtgttgcacattttcttcctcttgatcagaaagctgccattaccccacgtgatcacgtctatggcgagccaaccgaaaggcaggccaacatctatcttttttttttaggccaatcaaacaaagagaacgcactagtacagaaaacgctattggtgccggttggtaactggcaataggtgccggtttcccaatcggcaccaattggtcggcaccaatacatccttccggcacctataggtaaaacagaaccggcacctataggtccacacgagccaaaaaaaaaacctcgagccgatccagaaccgaaatatccccacatcccgaatccagaagaacccctccacattctgtaatcccctccatccacaaatccagaataatatccccacatccaaaatcaaaatccaaagaacacatcaataacaataacaattcctacattcatttaattataattttcatccaatccaaatcttcatgttcttggtacacacatgaacaagagaagagaaattaaacaagagaagaaattaaaaactcgaaccacggcggcggtgacggggcccttggccgcctcctccttccccttaagcgtcgtcgggatgctgggaaggggagcaccgccgcccacgggaggggagggcgcggcggtgctgccgcctccgtgggagcccgccgcctcccctctcgccggatctggcgaaggggagggcacggccgccgccggcgtagcgggagctcgccgcctccctactggctctcatgtcgccggatctggaggagggagcccaccacctcccctcctgccggatctagcggaggggaggatgcggccggcgccgctgccacctctgccaagcagtcaccgatcgcgcgagcccgccgccacccacaagtggtcgccacctccgccaggcaaccgccggccgcgggagcccgccgcctccaaatcgagcgagagagagaggggaggtagacgcgagtgagagagagaggggagggagagagagggatgcgagagtagataaggacggtgggctcggttagtcggctaggctcggtttttttcaaaggtgccggttctattaaaaaaccggcacctctactatatgtgtcggtttttttaagaactgacacctatacttatattataggtgtcggttctaaacaaaaaaccgacacctatagtatatgtgttggttttttaatagaaccgctatagtgtttaaaggtgtcgattctacttttttttcatcgtgtggaggtgggaaaagggtcataggtgtcggttttaaatgaaccgacacctataaggccggtccctatgtgggtttttgtagtagtgatgacaacgtggtggaacctatgtacaacaaattacctaccaagggttatactccctctgtcccaaaatatttgacgtcgttgactttttaaaatatacttaaccgttcgttttatttaaaaacttttttgaaatatgtaaaactatatatatatatacataaaagtatattgataaatcaaatgataggaaaagaattaataattacttaatttttttgaataagacgaacgatcaaacatatttaaaaaagttaacggcgtcaaatatttagggacggaggagtactcaacagccctctacgaagtaacgaagctatgacgattttgttgagaaaactttcaatggggacgacgtctaaaaatgctgatcctctgttctaggggtgatattgcctccaagcgtgttgtataattagagatattccagtaatgcctttgccctcgagtcgtcccttatatgataaagcctcctatggtgatttttggggcagttcagaagttgtttcattgttccctttcttgaattgtttcattgccgacaacgctgaatttctcgggcggctgcagcgtcaagcttactacaatggtgatatgtgcctatttcatcactttggtggctcaacgtcttcccatagttgttcaactcctctttgtggatgttcaatgttcaatttgtaaataatgtttgactaggatgtcagttgttcattgaatcctcttgtatcttatcctaaggactcgttagtttgcttttatttttatttctagttcgattttggcgcacatcttctcttcttccgcatgtcgcatgcgcaaccagggtttaccttaccgccccggtaactacacggttaccgcggttaccaagcttactgcgatgtacggtaatataaataccatggtaacctccttaaattcaaataaatttaaaaaataatttgaatttttaataaattttgcacagtttttcacggttaccacgtttaccgtgtggtaatcgtgcttacctctaggacgcggtaaccctggccccggcggtttgaggaaccctgtcacaatgtgcgttggcgcgtggatacgatgaccgtacagcgcagcgtctggcggccactttgcctgggagcacgagccatcggatcgctcgtcgtcgtctcacgacctcgcctggattcctgttcgtgaggggggcttcttgcaaatataccaagatgcagacggttccaaaaaataattaaacagtaacggctggtaccaaggtaactgccatagtgaaattagatgtttggaaatgataataacttattaataatctattgtgcaaaaaaaagcatctatcagaggcacacaacgataataatatattaatttattacattacataaaaacatctatcagagacacacaacgataatataataacttattacagtgcagaaaaaacatatatcagagacaacgataatataactagatagatataaaagcagtcgggaaaccaggcggcagtaacccatcatggtgcagtgacctatttatgcctgggataatacgagtagaaaattatgcataagtcagtgattgtaccaactgaaatataagagataatctgagataacttgttttctctttgctagttacaccaggaacaaaaatataatgcaaggtaattaaagtcatttatgactgaaatagaggcgtcactacaataatgaaacgacactaaacttcaactgggtctaattgtggttgccatgctcggcgttctgaatgcattgtgctttagggtggattggtggctgacccgattgtgttgtgctctcgtatggcaaacacaacacaaacggcaaacttttatttagtgaaatatggcatccccagcagtaaaacctaaatatgcataaataaaatacagcataatcattgcactataggtgagactcaccccgaaacgaaattttttcttcaccctccatgtgggcaactccttacatgatatcttccctttgttcctacagcagagaggaaagggggagggagggagagtgagctttgcaatagcaacataaataaaatgttcaagtgaaagcagggttttaaatctcccgctaaactgccgctatctcccgctatagctgtttgagaaggattctagttatttgttctcatgtacaatttagccgctatatatagctccatttagcccgctatagctgctgctatagctgtttgagaagctaaccgctaaatgccttagctagctatttaaaacattgagtgaaaggaacaatcgcagtttaccagaacaaacattgaaaactgggcacctaccagtatgcaggagagcaacaggttatgatatggcacatgatgctataaggacctggaatatctttttcttctgcaattactccatctgttgcttgtgataatgtgccattactgaggtggtattgactaagatagattttggtgtctggcatgacaatggtgtcaggaacaatggtagggaacgccttagagctgacacagagtgacctcctaccaatgaagagtgcattgccatctatggatgtcacatggacagtccttcccagcataagatcagctagcctgtaaactgagtagtaaaccgcatcccattttgtgccaatcaccaaaatctcagagttgcattctaccagacggtaggaatacaatcgaaagctttcatcgcttgttggccatctaaattttgcaatcaactttggtgatggctccgataaacagtgctggggtgggtcgatctgtatgacctcatgctcacctctcaccgaatgaggtcgcagcagcacataaagcttcccatggaatggcaatgtagaagaacggtggttgatgcgatcccaggttgtagccaccctccattgctgatcaccggaggtagcaaaacatatgttccacacgccgatcggccgcatcattaccctgacgagtccatctcctacacccacgctgatgcaggcgccacagatgtttcgaagggagcaccaccgagacgcctcggagcggcagcgcacccgcggcaggagggtatccagcggcgggagctccgcggtgtcgccggtgaaggggtggaggaggcggatggctgtgtcgtggtccctctgcagcaggaggacgccgtcggcgtcgtagaggacgcagtggtccctgaaatggggcagcctgacgcggacgacggcgccggtcgacaggtggaaggagcggacgtggccgcggagcttgccgtggcccgggtggaggccgcggccctcgggcagcagcatccacccgcgcgggtggaagcgcgggtcgaggatgccgcggcggcgcgggcaggtggtggcggagcgccactggggacacacggcccggaagcggatgtagtcgcggaagtcgccggctgccactcgccatccgattaggctcaccaagtcctcgtgcagagacgcccacgacgaggatggatcgccggcgttaaccttccgcctactgctgcacgcaactaaggccatatgacatgccgctgcaacaaatattagcagcacatctagttcaccaaactatattcgcccaaggaacaaaaatagtacaatttttattagggaatcacctgcaggcaaaggaccagcggcagatgcaaaccttggcgttcgcagagggcggcgtatcagatgggggaggaagccctagggctaagccgctgcgccctacaaggccgacgagcctaaggctcagaagcccattaacgggacagagagcccaaaacatacggtctatagatagactcttgggactctaatctaaaccaccgccgggccagcacgcacgtccgcacggcctcctgcgccgggaggagggagaggatgcggtgcagtattgcgtccgggagtagggatggcaacggggtgggttgggctggaacggccccgtccccgacccctgacctctccacccggcccggcctcggcccctgccccgagtgaagatgcggggccaaaccatcccctgcccccggcccctgcagggatccg

SEQ ID NO: 7 - DNA, 11460 bp, Oryza sativa
tttattttagcaagcttcaaaatgatttttctcttaaattacttatccaatatatgacccgattacaccattgtattcgttttaattaaatctttacaacaagatatcgcatgactatattttgataaaggaaaaacatatgctacttgttgtttcatatgtgatagcaatatgtttcaacgtgtatcttcaccatgtttcataaaatatttaaatgttttagttgctgattttttttaccgtatataacttaatgtttcacttattggtaaactgcaacatttgaccatccgattttttgataggcatcggacgtccgatgggtagctttctccagacatatggggtccacgtgggtccccctcatcatgtcagccaaaaccgatcactgtattatcgagtgatcaaagttaacgaggtattgtgagttgtgtcagccaaaaccgatcactgtactgtggagtgatcaaagttaacaaggtattatgagttgaggatgtgctataccgtatttcggttcaggggtgaatggtagactcggcgacaaattgaggaacctaaagtgaacttataccataatgaaatgaacccgtcggcccagatcagatcggagaccaatcccctcgactctgcggcggcggagtaaacgagcccaagcaagcgctcgccggcggcggccatggacgacgaccgtcggcggcgcctcttcctggattcttcaggtaacgcccggattcagttccctgcgattttggtggggttgccccattcgattcattccatgaagatattcccaggtgtcgattttaagggtttcgtgtcaattgcgcggcagattcgtgttctagtgcgttcaattatgttttctaggtgttcatccttcttgcatgtttaatctggttaaaatactggcacatgaaatgcacagagggttcacacttgcatagtacatctgtgcagcaatgcatctgctcttgcttgcaatggcctacgatgggagttgtcagaatttcttcagctaaacaatagtagtagattacaacaaagttcaaacgctgcatcatctgattgctttagttattttttctgaaattcgagaatgcaatctaactcataaaagtgaacccaccattagataataaacaaatgctatgagatcattctgcagaacaacgatctcaaaaatgaacagaaaggaaaaacattgtctaaacgcagttcaatagtaaaagggcctcttacaacaatctccaaagcaatcaaaataacagaagaacacttcaggtccagatggataagaaagcgaatgtttcaggtagtgctagcaaagtagcaataactccataacgatgaacttattggaatggtcaatggacctgacaaatgcagaggcatcaaacaagattgctaatgtagggagaaggtaactagcactcctagtcccctacacaatcagccattttttggtccatctcactctcaatccagtgagcttcaaagtcccagtgcagaccaaccagccagagtttcttcagggtccgaagggtttcaatgccaggagggacactctccagccctgacagagcaacaatgtacaaaccttcaatgactggaagtgcaccacttataatttttagctggttgacatcaggcatgtgcttcagcacaagtgtcttcaggcatgggaaagccgccgcgccaagaaccaatctgcaacatctatatacggacctctgcaacatgcagcatcttctggtgcatcccgtcacctttccattgagtcttgaagtctttgtgcaggtccttcaaccagagcttcttcagggaggcaagggattcaatgccttgagggactttatccagcttccacaatgatacaatgtataaaccttcaatgcatggaagggcgccatccgtgatgtttatctggttgacatcaggcatgtgcattaacacaagagtcttcaggtgggggaacgcctctgcatcaagaaccaaagttttcgaactgtgcacgttgttcagtcttagataagtgaggtttgacaagtgtgatgcgagcatccccagtggatcttccccaagattacaccaacttagagctaagtacttgagatgtgtagtgtggctacgaaatatcgggtagtccaatgtgcccttggcccattgccctctgataattaacctgtggagttctttggacatgggctggagagcctcaaagcaaagaggttcattctcatctcttgcagaaagaagcaagctagaaagaagcggcatagttgataatgtagcaaaaatatttccacaatcagcagaacttatgttgtcaatccaaatacttcttatctgcattagttccttcagctgctcggccaagtccttgctggcttccacagtctcaagagtctgaagttcttccaacttagacagatctttgggtgcttgcattccaatgaaatagcgaaacactgactgcttctcgtcttcatatctatcagctagcaggtgccttagcttcttgatcttagtgattccacgtggtagcttctctattttggtttgcttgatgtccagagtttgcaggtttgagagcttctcaatagactctggtagtgagcagagtcttgtccgccttaagccaatgtaacgtaaattaaacaatttacctatgcatgctggtacttcagtgatatctgaatcttgtagctctaggacagtgaggtatttggattcagacaaaattgaggataacaatccaggagggtgtgtagttgtttcaagtagtgtgcgaagatgtggaaatttcactgttgatgcacaaccttttccattgttcaagaataatgacagacgacggacttcccaatcaaccttttccacagctccataatcatttacgcaaccgaacctctcctgtccagcaattgagagagccaggttgcgcacaatgtcatgcatcttacaagatctcaccctgccaagctcatcatactcgtcaacttcaagcatgttccggtggatcagttccatgagatttatttcggccacatcttctggtctattgtgttcggttctcaccgcaaaaccttctgcaacccagtaccgcacaaggctctcacgagatatgcgaaaatcttcagggaacaggctgcagtacaagaagcagttcttttggtcagctggtaatgcatggtagcttagtttcagaattgccttgacatcatcatttttggccagctcactccgaagctggttgtacatttgttgccaggcatgctcagtttgtagctttgtggacatcaggacacccatggtaacaagtgctaggggcagccccttacacttacttactatggaggcagccacattctcaaggtccagcgggcacctatggtcctttctgttgtaaaatgcccttctgcagaaaaggttgaatgcatcaatttcacccaaagcctgtatcttgagatggcattcagagggagcaagaactgccacatgttccatccgtgtcgtgatgataatgcgacttgcttggggattcttaagcttgccctgtatttcgaagtacacgttctggtcccagacatcatctagcacaatcaaacatgatgtactgttttcagtccttctgtttaattcttctgtcaaatcttgaacgcccattttgttgattagatcttccttagattctgatgattcttgttccatgcgtatgagctcactgactagctgcctacaaagactaaggatcgtccaagtctgtgacacagtgatccaggcatgaactgggaacttgatcttttcacgttcatatacgtctaaggccagggtggtttttcccagtccacccataccagacactgttattactttgtgacctggttcttcagagtacagtaattcaagcaaccttttcctgttgtattcaatccccacaggatcgccacattcaagcaacttccttcttccttgagattgcggtgtttcgatgtcagtgggagttcttggaatgagctgaactgtaggtaaccattccgtttgctgtcttttaacctgttcaatatcaccctttatcttcattacctcactagcaacttcactgaaaacacctgcataatgtactcttacgaacctcatcactgatccttcttgctgcagttgacaagcatagtatgagtacttgtccattatgtcttcaacacggtaagccagcttccgcagctcgtcgatccaaccctttacaacattcatgttggtatttgtggaatctaaatcttgtattacatctttcatgacacgcaattcccttctgatatactcaactttgtctggaagttccctcaagttagttaccttcccagacagtttggctatgacagctctggtggcttcatctcccaatgcaatactgatctttgagatggcaagcagcacagcttctgccattaccttagtgctgcagaacagaatcatgaatcatggagatagtcacataacgaattgataagataaaaaaggttgttgttacagttgtaaaacaattgcagctaagattgttttttgggcccttgtgggcatttgcaagccatatatatatatatttctaatgttaatcatggtgtaccaacagatataggtaaaggagctcctcattacgtaccagcctcaacagtaatcttaattgtaagggtaatgttctatggcaattgtttcataactctgcaatgtgaccaactgtgtaacttaggtcctgtttgggggagcttgtcccagctgtaacttttcccaaaagctgcttctgctagaagctaccccaaacagtccacagcttctgagaatctgtagttacagaatctgaaaaatgaactaagaagccagaagctggagaagctgggtttcagagcttttccagattctcagaatctagctaccaaacagttgcttctcagaatctaaagctcccccaaataggcccttaataggagatcaaagtaactcataatcagatagtctcatagtttagttgagtgtgttgtttcttgaagtaggtctctattcaggtttttaatggttccacagatagattgatcatttagtataactcatatgtgcccagctttttaatgcttcatacaaaggccagtatctaatatgtgtgagtatttcatgcaccatcacttcttccatgttcttaaatgcaatgaagtatatatgaaacagcccagatatatctgaacataatatcaaacatgatcatagtttgtttctaatcttacttttcttttaagcagccatttctaatgacaactctgttttggattctgctcttctagtctgttacaccatgcttagatgataggtttcttgtcagtagaaacctacttgtgaattctggcattaagctgctgaacctactgataagtacttaaagaacatgactgtgactcaatttctttttaagtgacaaggagcagaccagcaatgctcttaatatgaagtagagatgcacaccacatattccagtgttctctttctcagcatggaagtgctgaagcgcatgtggttcgattcttttgcttataacaggtagaggtcaacattttacatatccttttacaaaacacaccgtttagcaatacggaaaacacattaacgaaaaagaaaaaagttgtcgttttgagttgctgaaaagaactaccctctaactgtgaatataagagattataacccttcgatttgtccacaaatacaagggatcgtggatcctgaggtgagcaaacaacagaagggatgcagcaacattttaggccccctttgattcaaaggaaattgatagaaattttagaggatttcattcctataggaatttttcttacaaagccttttgaattaaaggaatgaatcctatggaatcctataaaattcctatggaatgcctcttcccatacaagttttggaggaattttaacaagaggtagaacctcatgaaaaaatcgttttgagtctttatctctcatcaaattcctgtgttttttctgtggtccaatcaaatggtcattcatacgttattcctgtgttttgcaatcctcttttacacttacattcctgtcagaattctatgtttttcatattcctccgtttttttattcatgtgattcaaaggggcccttagctttttcttacttttttttagtttattcaatgtgccaatcggcctaatccaattagtgcttccctttatctgtactttttcctattccttaatttctattaaaaaaaatataatccctcgtattacgaacggagggagtaccgtttagcactggtgtagatctgttgtataggcagggtcatggttgatttgttatgagatgcacttgataatgtagtgataaggggtgcgtggtttcaatcccaaggtcccatgttcaatcccaacacgcttacaatttattcttataataaaaactagcatggtggcccgcgcagactgcgcggctagcactctacttttaattttattatgtttattccaaattttagttagttttaaattcctatatggactctatactcaacttctaatattccttatttttttatttcgaatttctattttttttcttcattgaatttctatatggactctatactctacttctaatattccttatttttaattccgaatttcagttatttcctaatcgtatttctatatggactgtatactctacttctaatattccttattttgaattccgaatttcagttatttcataattgtatttctatatggactctatactccacttttaatattccttatttttaattccgaatttcagttatttcctaattgtatttctatatggactatatactctacttttaatattccttatttttaattccgaatttcagttatttatttcctaattgtatttctatatatggactctagtctcatcttctaatattccttattttttaattctgaatttcaactatttctaaatctctacttttaattttattatgtttattccaaattttagttagttttaaattcctatatggactctatactcaacttctaatattccttatttttttatttcgaatttctattttttttcttcattgaatttctatatggactctatactctacttctaatattccttatttttaattccgaatttcagttatttcctaatcgtatttctatatggactgtatactctacttctaatattccttattttgaattccgaatttcagttatttcataattgtatttctatatggactctatactccacttttaatattccttatttttaattccgaatttcagttatttcctaattgtatttctatatggactatatactctacttttaatattccttatttttaattccgaatttcagttatttatttcctaattgtatttctatatatggactctagtctcatcttctaatattccttattttttaattctgaatttcaactatttctaaattgtatttctatatggactctagtctcctcttctaagattccatattttttaattctgaatttcagctatttctaaattgtatttctatatggactctgtcttttctttttccctaattaatgtgagaatttatagaccatgagagcaaacatagaggcttcttcttctattcctttaataatataatagatgtttgcccttcaaatctcgtttatctggttgacttgttactgtttctttatttggtcctagttgtttttgtcttccttttgtaatccatattaattctgtacagccgctatagtagcactggatcttttgtaaagcaacatttcaactatatcgcaaattgattggccattagaaaatttcgttgggctactcatctttcatgctttgttcttggtgaaatgattggtaatagtttccacgggcattcaattttagcttaagatgtccttgcagtagatggccaacaataagataataaatgataaaaatacgggaaacatgtaattaacacacagtatttaaggtgatgcactgcaaacaacagtaatcgctaaatgttgcagttgaatcagcactgaacaaaacaacaacacaaaggttctttccccatctctgaactgagaatgtgctgctggccttagatatgctcgctcagggtcaaatttcagaacaaatttcccaatattttctagcaaagagactaattagtgatccctcctctcaacaaggcgaaattcacgaaagaaaaaggcagaattttccagcagacataatcagaaaagttaaagctgataacagctacaaactagctagatgatacaaagcaagatccagtggaaatcaatctaacctgagctgcagacgatcagagcgtctagaagagcagagctggcgagacgatcagagcatgtgaggtttaagataggcaacgaagaaaatataaagcacaaagccactagcctcaaatctcaatcagatccttgtctgtttctctgaactagccgccgacgacttgacgcgaagtcgacgcggctaagcatttgcagtgccaattatcaactggccaagttgtgcgtgtaggacgggagtccgctgtggaagcttcctttttcctgatctcaaaatttatagtataccctccaaaccgcatgcagtgggggatgctggattctttttaaatggaccagaaactcgagaaattattgctcaatagattattttgcctatagtaaaaaggaaaagaaaaagtaaatcagaattgttcatatcgaaaggaatagaatatctgtagtccagtatgcaaattagaaaagatacagttttgattagtttactgtcctgcagccagctcactggtcaggctgttcgctgcatcttggacatatactccctttgcccaaaatataagagattttagacggatgagacatttttttagtacaaggaatctggacatgtactagtaagtgtcacatctgtttaaaatcctttatatttaagtacgaatggagtaggaagaaacatatcgttatatttgaggacggagggagtaggaagaaacataccgtgtcaccttaacagtccttgtacttatgactcagttgttgaggacctagttagctagtcggcaactgcaaaaatataattccagtctccttattctactcgctaatgacagcttagagaaagctaacaaactgatttcttatgttttcatccaactaaaacggccggagacctccaagtcgtcttagtataaaaatctctcaagtcaagtttcctcgatttcttggtcgtcgtcttcgtccaccttttggtatgctcgatttttttaaaaaaattatgactctttttgtaaaacttatttatgaataaatagactccaaaaatctaattatggatatggactctactcggtgccggtgagcttggcatcgaggtttcgtgcacccacgctggagaccttggcatcgagttagagggggagggggagagagtgtcgatgttgtagatcacgactacggtatttgcagagtttggggatcattcgtatcagggtataagcgagactaaggtaaaataaacggagacaaggattttcatgtaggttcaggctccttatctaccaggtaatagctctactcctgctaattgaaaccagtgttgctcttattcatcagaatcacacaagtacaatatttgggataacttatctaatcatcgtcaacacggcggcatgaaccaccacacgttgtcgacaacagggtagtcctcctcctctaatatgaattgggcgatatcagagatagcgctagatccctcttgtcagcctctgtggcaccagattggatctgtttaggtttatctcttatgttgatgtctggcggcatgtattgatgtatattttgactgttgcctgtctatttagactcggcttgtcttggtccgtctccccctcttcttttaggggtcttgtatttatacccatagatgtccccttatccaaatagaactagaaagataaatatggatacgatccgaatagtccttgtagtttccatgtagaactctgcttctccttccttatctggaataccttccgtatgcaagatttgattccgtataagacttggtatatggtaggtcccgctaagcttaacccaacactattgggtatgccttacccataaaactgatagggggtaggcgccatgcttcttggtgcggacaccgagttgtcccgagatggcatgggatggcatgagtgtacaacgagcaccaagagtctgagacaccatggaatggcacgtgtttggcaacttttttgacaagtagacaagcacgatcgagacacctaaacgccttgcggtgattgtagtatcgccaaccacctcaatctagcaaaagctagaccaagatgggttttgataaactaaaccggctagcggagccgatttagatagaacaatagataactacccgtctcgtgggtaaacggaaggtttacgggacaaacctacaaaggatttcgcactctcgtagtgaaggcagacgatttatggcacaaatcgacaaagatggatgaactagactagaactagattgaaatattgaaaaagcaatttaattggctaagttggattgtgtgtagattgtatgctcaattgaaccggccttgtcccttatataggggttgatcttgcctcctacaggtcctcctccacgtccaactcaggatagaattcaaaggaaacccgaaatatagcctcctgagtaaggaatcctgagacctgacgaaaacagactcgggcttggactctgccggtctgaccggccacatgccgctggacagaccggccctcaagtagcggtctgaccgaccaacaaacgacggtcagaccggccctatggaggaaaccggcggttttcccaaatcttagcaattttctttaatttgaacagacaaacgacgatgatgagcatggcgcacctcaaccctccctcttctccatttcaatccctgcctttttatcagttgattctaccttatttttcctctatgtagctcctaatgttactactttctctatttcatattataagactttctagcattgctcgcatatatatatatatatatatatatatatatatatatatatatatatggcaacatatcctatgcacacaggccctcacgtgtacacacggtgcacaccaactaaaaaatgtcaccaataaatctagaaaaaatcatacacatactttcaattgtattacacctagggttaaaatcttaacgtcaaattcattatattttagccgtaacaaaaaaacaaaaaatctgacagttttaaggttgcaattttgtcagaattttatcttttttgttattctctatgtagaatgaattt

SEQ ID NO: 8 - DNA, 3159 bp, Oryza sativa
tgatatataagttaaattggagactagaaatcgcaagccacttgttgttctcttttgtaacatgatggtgtgtgttctttcagaacaaatggctgaggcagtgctccttgctgtcaaaaaggttggcaacgtgttagcagatgaagctgccaaggctgtcattgccaaggtgtctgaaaaggttactaatctgaaggagctgccagagaaggtcgaagaaataaggaagcaactgacaatcatgaacagtgttatactacagataggcacctcttacctcactgatatagttgtaaagaattggattgcagaggtgagaaagttagcctaccatgttgaggacgtaatggacaagtactcatatcatgctattcaacttgaggaagaaggtttcttgaagaagtacttcgttaaaggttctcattacgtcatggtatttagtgatattgctgaggaggtagtcaagttagagaagcaaatccagcaagttataaagcttaaagagcagtggttgcacccttcccagctcaatcccaaccagcttgctgagagtggcagaccacggtctcacgacaacttcccatatcttgtcaaagatgaagatcttgtggggattgaagaccacaagagattgctggctggatggttgtactctgatgagtcagatagagcagtgataacagtatctggtataggtgggttgggaaaaaccacattagtcacaaatatttatgagcgtgaaaaggtcaactttgctgctcatgcatggattgttgtctcccagacctacaatgtggaggctctattaagaaagctccttagaaagattgggtctactgaactgtcacttgatagcttgaacaatatggatgcacatgacctgaaagaagaaattaagaaaaagattgaagatagcaaatgtttgattgtgctggatgatgtctgggacaaaaaagtgtactttcagatgcaagaagcattccagaatcttcaagcaactcgagtcatcatcacaactcgagagaatgatgttgcagcccttgctacctcagcacgccgtctcaacctccagcctttgaatggcgctgatgcatttgaactcttctgtagaagggctttctataacaagggccacaaatgccccaaggagctagagaaggttgctaattctatagtggataggtgtcatggcctaccactagcaattgtaacggtaggaagccttctgtcttcaagaccagcagcagaatttgtttggaataaaatatacaaagagcttcggactgagctagcaaacaatgatcatgtccgagcaattctaaatttgagctaccatgacctatcaggagacctcagaaattgtttcttgtactgtagcttgttccctgaagactacacaatgacacgggagagccttgtgaggttgtgggttgcagaaggctttgtgctaagcaaagaaaagaacacgctagaggatgtcgcagagggaaaccttatggaactgatccaccggaatatgctggaagttgtggacaatgatgagattggcagggtaaactcctgtaagatgcatgacattgtgcgtgtattggctctttctattgctaaagaggagaggtttggttcagcaaatgatcttggcacaatgttgcttatggataaggaagttcgtcgcttgtcaacatgtggatggagtgatgatactgtatcaacagttaaattcatgcgccttcggaccctgatctcactttcgacaacctcattgtcccttgagatgttatcctcaattttgtgtggatctagctaccttacagttcttgagctgcaagactcagagattactgaagtgccgacttctattgggaatatgtttaatttacgctacattggtttacgacgcaccaaagtcaaatcacttccggagtctattggaaagttatctaacctccacacgcttgacatcaagcaaaccaaaattgagaagctaccacgaagtgttgttaagataaagaagctaagacaccttttagccgatagatacgttgatgagaagcagtcagatttccggtactttgttggaatgcatgctcctaaagaactttccaacttgcaagagctgcagactctagaaactgtggagtctagcaaagacctggccgagcagctgaagaaattgatgcaactaagaagtgtgtggattgacaacataagttctgctgattgtgcaaatattttcgcttcattgtcaagcatgccatttctttccagcttgcttctttctgcaaaagatgagaatgaggaactctgcttcgaggctctcaggccaaggtcaacagaactccacagactgatcatcagagggcaatgggctaagggtacacttgattgcccaatatttcacgggaacggcacaaatcttaaatatctagctctaagttggtgtcatcttggcgaagatccactagggatgctcgcttcaaatttgccgaacctcacttatttgagactgaacaacatgcatagtgcaaacattttggttctttcaacagagtctttcccccacctgaagacacttgtcttaaagcacatgcccaatgtgaaccagcttaagatcatggatggggcgcttccatccattgaaggtttgtacgttgtgtcactctcaaagctggatatagtccctgagggcattgagtcccttcggaccctgaagaagctctggcttctgtacctgcacagggacttcaaaactcaatggcacaagaacggaatgcatcacaagatgcagcatgttccagagattcgtgtttagatgcggctgacaggtgccgtttgtagtagttttttttttcctcgtctgtttgcagctcaggtgttgatttccaatgagttagcttttttgcattcgcggtgcgtctgtacattttgtatagtttcatttactttgatatttatctatctatctatctatatctattatatactaaaagtccattaaacttcctataaacactcctaagccgctatgtggcatcctataaacgctctcgagacgccacatgtcactctaacaaaatagaaaaatttgaccatcgattttcatttaaattggtggacctattaattttgaccgttagatttatttttacattaaaataa

SEQ ID NO: 9 - DNA, 12024 bp, Oryza sativa
tttatttttttgaaaaaaaatatatcaataatttatttaagaaaaacatctcgcccaccttatccctatgtggtgtgcatcgttcatccttttctcttatttttattcttataataattgaaattaaaattatctttatgggaaaaaaccacaaatggacctccacataacaggtctagctagctctcccatgatcccttttcttctctttctcatcttactaacatttatgggaacaatataaataaaataatataatcttttaaatcatatattcaacttatgatttgtttgaactattgtattttcttagtgaaatcaactttaagatccatattgagtatatttaaatgctaacataaaaattacattccggacccacatccatcttgaggcatccacttagtaaaaaaatgaaaattcatcattataaatcaaaatatcacatcttaatttttaataaagcctgtagacctattacctgtatccaattagacacgtaacaactttctatcaacttaattaatttctctcaacatgtatatgggggtaatttgcctatacaatttgactttggtgctttttatcaataaatgatatttttaggagataaaattatatataatctatgaagtcactaataaattagaggaaaaaaatctttaaatgataacaattttatgtcataatgtgtactccatctagcgttaattttttatagacaacattttaaaagtaaaaaacatgctcagttgaacatccaactgatagtaccaaacatgtggattgcaccgagaaatcttttttttttgaggaaatgcaccaagaaatcttagcagtgaaacaactctgaggtggcattaccaatatagtgaatatagtgagttaattccatatggaattaaagaccatgttcatttatttctttttggtaaaacttgctatgctacatcgtaaattcataatatttactgtagaataatatagaaatgtgcatttattatatgacctctattttcatattcttactcaattccagagagataattagaatagatgattttaccccatgtcaggttgtaggcttaacccacatatatttataacccaaattgcagatatcaaatcatttcaaagatgcttggtagaatggaattctccaatttattaaaaatggacatgaaaagaattcaaaatttatgtaaatgttttaaaaaattgttgggtttaaaacccgttctaaatggaaatattcaaatattttagcaggactaatagaacgaaaatatctaaaatttcgaatagaatctgataaaaatttggactttttgagcaaatgtttggggaaagatatccccatgggtagttgaaatacccaaaattttttcataggaactacatctattgtctcgacataatattatctcataaattaaaaactgtacgattttaagtaattttagtttagaatttatgtgctacaagggtaattagaagaaaatcataaccaacgttgaaagcacattaaataagttaaataataatttatatatagtaaagtaatattttaatacaaagtcacatatatagatattcgtaataaaatttcacacatattaaattaggtgcacgcgcatgtgcgcgggctacctttctagttattattattattatatactaaaagtccattaaactctctataaacactctcaagctgccatgtggctacctcaaaacgctctcatgttgccacgtggcactctaataagaaacagaaatctgaccattaattttcatttaaatcggtgggcccattattttacatcgttagatctatattagaaaaccaaaacgtccacaactatggtcccacatctccttccgtgtaggtaaggtacgtacatgagcgtacgtacgtacaagaccgaagaaagacatccgttttttctttttcccctctctttttctctttctcagaaatactcgtacaaatgcgtacatttgtcttatttctggctagctactttgtaaaggtgagcaaggttaatgcaaatataaatacactgtgatcatgtgataggtaattatatatagattcacgtagattatgtgagactaaattagctatgtaatatttaatatttaaaaataaagtttacaccattatatatttacccaataaataaataatgtaagatagtttcctaaaagtccattaaactccctataaacattctcaagctgccatgtggctccctcaaaacgctctcatattgccacgtggcactctaataaaatagataaatcattattttacatcgttagacctatcttaaaaaccaaaacctctcaccggccccacatccgtgcagtacgcacgtactgctcgcctgttccgttctcctttttttttccctttgttcctttcttttgttttttcaatcacaattaaaattgaacttatctttatagaaagaatacccccacaaatgttaaagtccattaaatatcatataaatgtttctaatcgttgcgtggcacttttaaattagagaaacatcgaatcattggtttccactaaaatcgatggacacattattttacatcattatttgatatgtattaactaaaagtccatgaaactttctacaaatagtcctaaatcgccacgtggcatcatagaagtgttcctaagccaccacatgccactctaacaaataaccgttgattttcattatatttgatggacctattgtttacaacattaaatttttcttaaataaaaagttaaccacccaaacctaatccacccatcctgtaaatagtaagtacgtacttatatctcatatacaactacctattatatatgaaaataataataatatacctcatatattattattattattattattattattattattattattattattattattattattattattatgtygaccgtaacatatatgtcacrgtgtctgccattctctctactttctattttatagctagcaattcaaggtaaacatgatgagctaatttgtcatgatttaatttataattaaacaaattaatttaagatcaataaaatctaaaagttctaagtaaaatgcaaaacatcatattatcgatttacacttaaaccgagaaaaatattattttgatgattagattagatttattaaactaattaatccctcactgctgatctcctccgtaatttatgattaaaaaattcaaaaagttgtgagtaacaggcaaaatatggcaccatcgatttacacttatcctattatttttaaccattatagatctattaaactacttaatccctctgtcctaatctcctaggtaattacataattataataattgattcatattacaccaccctcttcctagattaacacttcattcttatgtttttatactttattaggtacaaagagatatcttcgattatatattaaaagtccattaaacttcgtacaaacgctcctaaattaccatgtggcattctacaaaacgctactaaaccgttacgtggcgttctaataaaccggtggacccactatttgtttaataaatcgataggcccactatttgtttaataaatcgatagacccactattttcaactattatatttatctttataaaaaaacattcatcataggaactcataatttcctatgaggaattatccatagctatgaccaatcgatgtttgccatcaaataataaattgattatcaatttttctacaaaagtgggtagagccattaattatatatggttgtatatgtaatgtcttttataacttaagttttccctcttttcctatggaatctcaagagcatatgtcttaggtaccattttttataccgtaagttaccaaccataatctccaataggtaattttataaataataattaaattcgtcatgcaattctaacaaagaaaattcattaaaaaacacaattttaaaactttgcagatattatttcgttggtataccgcctttataagtttctcaaatttatcctacgtatgagacatgcccgcgcgaatgcgcgggctaccttcctagttgtatgaaagagcaaatccattttttttataagacctcatttgcatgtattgttgccaatatattaatctctgtgcttggagatatatgcagtggaaatgagatgaaacaagatagtaggacaaatgctatcaatgttattatgcatcaacgcaagcatataataggatttaagcttctggtcattcatgtatacagtctagatttttgcctgctcaggaggcagcctgagcgagtgcttgtttggttgcagttgtatgcctgaggataacagtacatatcattctttgataattacctcaataaacaatatcaagagacttcattaacgtgaaaaacaaatagaaatatactcctgcgttttgtctatatggttctacatcgaaacttgaaaataaacaagagtaaataaatgatgtagggcagagtccggcgttggattttgtaggcctgagccaggccaggccggccacccatgaaacgatccaggacatcaaccaaacaagtactacgagacaggcgccagggaaacgacaggtcaggcgaaaacacttcagttcaggactccaaccaaacagtcccccacatgcctagttgatgtcacatgccaacgtcagtgcctatgataacatgacgaccaactgcctagtttagtactaactaataactctgagcatgtgttgattgtgaagataacttggtggaatgcagaattgtacctttggaaggaataaatcgataccagttgggtaaataaaagaagttcctgtcaagccacccaataattcaattatgaagacacaagactcccatcgattttgaccttcagcttgcttcctagcgttctgcactggtcatctagctcttttctgcaaccatagatatccagttcttccagcgaaggtgggagacccttttctggcagcttcctgatgccatagcaacacaagatgtccaacctcttgagggagtgaaggctgtgcagacctgcaggaagatcttgaagagagtagcaatgcgtaaagcggagctcttgcagggtcgtgaggagctggagcgctctctcttgctcatccgttagtctccactcttcgcttctgaaaccgtaaatctttaggtattcaagggaagtgaggtgcttgcagaatgacgtggtaaggatagaggtatcatcgatgcacaacttttctagtcgcagatggccttgccatgacaaattatccaaacatggaggcaatctggggcattcgtgtacttgcaaactcctgaggccatagaggaattgcaagccctccagtacggcgagtgttttacaatatttaatcgtcaactcttcgagtgccgtgcatgattgcagctctagagatatcaaaaattcagagtggtgcacctctaatttttttaggcaggtgagattcatttgaaagcgggggcacagggtttcaataaaacagttgttgatgcaaagttcctcaagtgattgtggaaggaggtaacccatgtgagctctaaggccctttacctgcagcagccttaggttgccgagcgattgcaagccttccagagaattaagcgattgcaagccttccagagaattactagtaatttgtaagcgcgcacaacgaattgtcagctcttggagtgctgtgcaggaatgtaactgcagagatgtcaaacttacctctctcattagaaacagttttttcaggcgggtgaggttccctggaaagcagagttgaagcatttcaaagggaccatcatattgtacaataagttcttcaagggatatagggaggagccatcttctattcgcctgctcaacatttccatcattatgcaataaggaatcgatgagcttggggcataccgaaatctctagctgctcaagggaggtaaatccagccaaaccatccttgctcccatgaaatgttaaatcagagcaccatttgatatatatcttcttcagagagcatatgatatttaacggaaggcgtggaagtttgttttcagcggaaaatcccaatgatggagtctccgtagctgacataagatttggttgaccacattcttcctctcctatagatagacctgttatctgctcgcagtcttgtaacctcaactcttgtagggtcttcatgtgttgtagcaacagagatagccacttccctgttattccacaatttctaatatcgagaagtttaagacataggagggtgttgtgatctgcagctgccatgtcttcacgggtatctgatgggacatttggagcgaaaagttctaggcagttggatatttccaaactcttcaaagacctgagttgtcttaaactgtccaatgatatagtcgtaagattttgacaaccacatattaccaatccacttaagaacctcaagttacggaacgccataactttgtcatccaacgttatcatctgatcagaagattcatcccattcatccatccaatcatatgaaaatccgattcttaatgttccactagatgacccctcgattgatggaagtgttgaaactcgggtgatggataatttctcgacattaggtgaaggcggaagaggtttgtgcacacgcaaatgagggcaaccatagatggtaatctcccttagacaggaaaaccatgacgactgctcaatctcaaattgcgggtagttctcaaacaatggaaagacctccagtgcagggcaactcttaatcttcaaaacctttaaattgtcattcaagttcctgatggaagtgcaggagcatgccttcaagcttatcaattcagttaatacaagctcctccactgatggaattgagacttctgttgcattcctcattttgatcaacacaagctttctaagtaacgttagcctttctaaaggaagtctttgccatttttcacatttttctaggtgaagtgtttgcagacaggtaagtgaagacggaaaccaagttggggaggtagctccattatacccagatatccgtagatgcttgagactgtgatgtggttcaagaccatcaagcacctcacgtgctatgcctggcaaattttttaaaactggggatatatttgtatcagccatcgtcagtccctccatcagaggttctatgtcactgtcagtttcattttcagaactcatgtctgtgtcatactcattttcagagctcatgccactgtcatatccatcctttgcatccttcgaggacaaatgtagcttttctaaattatgtttgtctcttagtcttgcaccacaagcttcgattctagtcgtaatagtttcaagtccagacacaccgagctgtacaagttggttcaaggactgtagttggttcaagcccaaagaattatgaacactaaagtcatttaattcctgaagagaggtcattttgccaatgctagtgatggacgagaacacttgctttgctgctacaagatgcctcaggctaacgaggttatccatatcattaggtataataagatcagattctgaaccagcatcaaatacttggagatgataaaacttgccgacagatagaggcaaagctccgtcatcaaactttatatagcgaatatgggtaggattcaccagattcaataacgaggggtcaatatcagtaaatggtgcagagatttgcaagacacgtagatgatgttccttctggactatatctttgaaggatttgaggaatatatggttatgctgcccaattagcaccaaagttctcaaatgtttcactggtttaactgcatttctaattctttcttcaaccttgccacaatcaggatcttcttggtgtgtagaatcggttaatattgacaagtgacgtatagttggcaacattttattgcactgtggattatctatagttgcgtactctgttctcgaaaccatccttgcaaaatcatgaataagcccacacataacataggatgtcttgtcaacttgagtgaactgatttgtctgtgcacctggctgaaagaagccggagtttaccaaattagaaaggtactccctcccgatctcttccagtctcttacttgaagagttacgatgcacaaaaccttgagaaatccaaatatggatcaactcctgtccaaggaagcaataaccactagggaatattgaacaatacaagaaacattgttgtaaatagtaaggcagctgatcatagctaagcttcaaagaaggcatgattcctctacttatattcagggatttccaatcttcattcctcagagtgttgctccagtgatcaattgtaagatgctttcttaatatttgccctgctgtttctgctgccaatgggttaccatttaacttgtcagctattttctgcccaatgatgtttagacttggatgtgctttataattttcgtcatcaaatgcgcatgctttaaaaaataaccaaaagtcctcatttgacaaagaatctaacttaatcggttcgactgtccccacccgttgtgcaagagacaaaattctagttgtcacaagtatcatattgccttttgcacgatttgatttcaatggagctaatagaatgttccatgtgttatcatccatgctattccatacgtcatccaaaacaagcaggaattttttcgtgcggatgtccatatgccttttcaagatctcctgaagtttggcaaaactacttattgcattgtgtctttcttttctttgatgagatacttcatgtctttcttgagagacaaaatctagaatctccatcgtgagcatcactccatcgtagtctttagatacccaaacccatacctgatcaaagttatgtttcaccattggatcattgtatacaagttgagcaagagctgtcttcccaactcctgcaatgcctacaatgggcactacagttagtccatcgtaactgtcatctgtaataatcttcaggatggcattcttctccgcgtctcttccataaattttgtgtgggctaagacttgacgttcggatcagggtcgttgttgtacttgtacgatgatttgagtttccaacaaggtccgctccatgtagcgtgagaacctcactaacagccctgatggcaacttgtaacccaccagttatttgctgtatcctgctagaaaattcagccttattccaagggcgtgaatcgactgcattgttgttccaagggtgtgaatcgactgcattgttgtgtgttgactcatcaatattcctcatcctttttctgccggaattaatcttcatcctttttctgctgccggaaccaccgagttgatcagtaatggtgatatttgctgtggtgtcagaagtactgcaaaattaaaaaggaaaaaaaaaagaaggatcacacaatcgctgctgacgtgatgaaccatgcaagtatgttacattagttaaaaatagtgccgggtaaaaacaagaagaagaaaagaacataaatcctacctcgataggtttgggtagcttgctcgacgcttgttcttacaactgatgctatggaggtgattacgcaaaaccgacgtccctgtgtgagaaccacactcgagcacagtgttacgaacacatcttgccttcaaaggctttccattttccaattcagtgatacgtaagttctcccatgccccggaccggcctgttttgccaccactgctactcgaaatatcagtggtacttttcggtgtcactgcctctgcttgcagtggtgcatgtttatccgcgctttcaggagcgccttgccatgcatctgaaccaagagaaagcggaatgaatgcacctccctgggcagaaactaaaacggatacatcttacatatcctatcttactataaagttataacccactaactcctaaagcttaacatgcaaagatgccacatcatcattcactaacaagttgacacatcatcatctattaacaatcaatacatttaatatcatacaaacatgctatattatctataatttaaaatttattacattttagagcttttaaaataaaggcatgttaaattattatcacacataattcacataatatttcaatatatatcttcattatttattataatatcttatgcacctatgtaagttcatcctcacttggttaatattatttttctcttctctaattaaactattattttggctgtgtacaccctcgcaaggtgtagaggccgggtttaatataatccattatctaaaaaaaattaagctattatcacatcaattgattttagtacttatgtaagtttatgttgttaagctatcttacacattatttttacttattattattatattgaagtcccgcagcaacacgcggggtttcatctagttacgtttatattacctccttggacctggtgctggagcctgtagtagtcgaggtcgtcgactactgcattgttgtgcgttgactcaccaataatcctcgtcctttttctgctgccggaaccacagagttcaacagtaagagcgatattagttcagcacttacttttttaactatttttaaagaaaaagaaaagaagagaacataaatcctacctcgattggtctttacgcttatccgtacaacggttgctacggaggtgaatatgcaaagccgacgccccattttgacacttgagctcagtgttacagaaactacatttgaccttcacagcctttccattttcatattcagtggcaacaaagtacttccataccttggaccatcttttgccaccaggcccaccactgcttctcgaaatatcagtggtaaattttggtctcactacctgatctgcttgcactggtgcatgtttatgcgcgctttcaggagcgccttgttgccatgcatttgaaccaacagacgaaagaggtgatgtacatcttacatgcgtacggacgcttatattacctccttggacctggtgctggagcctgtagtagtcgagctcatcgacggcattgtcggcgtcgtagagcagctccctgagacgaccgagcgatcgggtcagcttgttcccgattgctctctgcctaacggcagcgaccaccactttcaccctctccatctctgacttaagcttctcggtggcatcagagagcccaacctgacgaatccactcgtccagcttgtcgctttccaggttctccaggatggtctgcgccagccactcgatcccgccctccagcaaagtgacttccacctctgccatcaggttcggcggaaaactgctagcagttgctgggcaattggcaatggcatgaaatgatggttgtaatgattaaggtgctgaaataatggttgaagtgatatataattgggatgattaagggactattattttttgattaagggaaccggattatcacagacaagcttcctaaactcaagttttttttttaaaaaacaaatcttcaataaatttttttaattagaatattttttaagaacaatttttcaaatttgtaacaattaattcattcatttctgtcctcgaataaattgtaatgtcatggtgaaggttatgttgtgatgaacaatttgcaaatatgttcagacaagttgaatttcatgtatatgacttcttcgtagtttttgtacattacatgttacatataatactctattgttgcactaatttaaatcaagttggcaagaacttgtgctcggaatatggtttagatattattctcatgtgcatgcattggttgaggccgcagaagcgtggttggtgactggatcgggactaaacacagagggttaaggtccatatagtcaaggatgtcatatgggatgctcaggctagagaactacatgggaggtgtgcgtatatggataaagaagttaaacttggaggaagtccaaatttggagggatctgaatcctaatttggaaagatagaaaagagttggaatatgacggagtcctagtcctagttagattaggaact

SEQ ID NO: 10 - DNA, 18155 bp, Oryza sativa
  misc_feature (3312)..(3312): N is any nucleotide
cggtctggccagtcccgctaggcggctccctcggagcagtgggtttgcccatcactacttaggtgccctggaccaaacgcggattcaccatctcgctagaacaagcccgaggggggggatggggtcaaagcggcatctcgatcacggactgattatccccttttcccctgttttttttgttgttctctctgtttcaaagacattcagtggaaggagcaatgaatagaagaaacgaaaggggaaaaggagctaaggaagtttcaattgcaaaacaaggcaggatggcacgacggcctaaatacaagaggtgcccgctacgggcgcacaacaagagggaattgaaggcgcggatagtcacggggtgcccggccctccgaggccggcgccgctgacaatgtcgtcccagtcttcgtcgctatcgtcgcccccgctcccgctttcttcgtccgaggccagcccggaggtgaagcggggggccgatccctcgaagcccgagacaatcgcgtcggctgcctcccgaacttgctcccgggccctcgcttcagttcccagggaaaaatcttcaagtgcgcgccacggcatgaagtcggggtcgcgcgcttggtggcttgcgagaaccagctccaccgcggcccgtgccaaagaagctgacgaggactttatggtctcgccgacctcctcttccagtctctccaggcccgccgctagcccgtccaaccggaacgcgagcgctggttgtgtgggcggaagttggctatgctggcgcacgggaacgccgactcggcgcgccgcgcgctccagccgagcgacggcgtcggagagctgcccgggcccgagttcgttggtgaggcggagggccgcaatctccctagcctgatcttgcaccaggcgccccaggtcggcaagggtactctgagccttggcgagcgagctcgccaaccccgcgccgccgggcggcccgccggctgccaggtgcttctcccgggcctccagttcagaagccctcgccgttatcgccgcgcgctcggcgcgagcgctctccagatgccgggcctcgcgcctagcagcagcctcctcgcgcttcgcaagctgcttgccaagccggcgcaaggctacctcgcggcggtttacctcggcttcgcgctcggcgagtgcggcgtcccgttcctggcacacctcctcccgcagccgtagctcttccgcgcggcggcccgcggaagcttccgcggcaagagtgatccgatcatgctcagccgcggcctcttcacgaaggtgaagggaggcctccgtttccgccgccgttctctcgtgggtggccaaggtggactcccgctgatccagcaggcgtgcttgctcccccagctcctgggcccgctgtgcctgttcctggccgcgcgcgttttgttcgcgctggctccgatcaaggacctcaatccgccgacggatcgtggcttcaaactcctgcacggagcgggcacgatcgtccaaggccttgcgttcagcctcgagtgccgcagtccgggcctgcaacgaggcgtcggcctccgctgctcgccgctcgtgggcagcggctgcgtcgagggtgccacagcgcattcctgaccctctccgctaggtcctcggcatgggcctcgtactgaaggcggatatcgcccagcgcttcgtccagggcgctcgaggcgatcagcgccgcctgccgctcctcctccgtctcccgcatgacggagtccagcacctcctcgcgcgcctagattttggctaggtgggcttagtgttttttgcgccccaccctcaccatgtcctccacggcacggcgcccctcgtcgactcgcgcgcggtccgccacaagctgagcccattcggcatccagggtcgtccgctcttccgccagggcttgaacttgggtattgagcccctcccggacagtggagtcggcggcggcgagcacctgcagaagtggctccatgctcgccggggatggggaagaaaagcccacgggtcacccccgggacgacggagtgctgctggaccccccacgagacgggggctgctctcggccccccttctgcgaggccagccccgagggcgccccaaacgcaagggggagcggctgttcttttcgtcccccgcttccagtcctcgggtggattcggcctcggcctcgggctcagggccgccctgtcacatcccaaaaatcctaaatttataaattgttgtttaattggaatttttagaaattaaattaaaagcctacaagctaaaccttaattttctaggaaaattttcaacataaaaatgagctaaataaaattttattaaatactatgcttgctcctctattttctagatttttctgggaattatttgagcaagggaagtatttttaataattgaaacaacattttacaaattgttttattcaaaaaagttcaaaaagtccccttcttggtccttgggccgaatccggcccatctctcctctcttctctctctttccccgcgctcggcccagctcggcccaagccgcgcccgcgcccgcgcgcccctctcacccggaggctgacaggtggggctcacctgtcaggtcgtcttcaacctcccgccgccgccgcgcccgcgccgaaccctagccgtttttcccgcatcgattccggccaaatccgatgcgatctcttcaaattgattgaggggttttgttcctctcggtctcctcttccttttcccccaagaatcaaagcaaatggttgagttttggatgagatttggattcggtttcgagtttatctccaaaaataagtccgaattcttcccgattcgattccatccgtttgcggttcgatctcttcgatttgagctcatataaatgttccccgtgatctcctctctcgtttgccccctttcctgagtcctcccgtgcctcctagcgccgtcgccgcgtgcttttagccgtcgccgtcgccggccgtccttcggcattgccgcgccgccgtctccgtcgtcgccgccggtcgccgccaccgccattagcttcgccgtgaggaggagaaggccgtccgcccctccgtttgcgtcgccgaccaccggagcacccccgacgccgtcgacccgagccgccgccgccttcttcttcvtcgccggccgccgtcgttcatcgtctcgtgtcggggtgggtaccgttgagttcctctcgtcgccctctacgcgttggtgccctccaatctcgtcgccgaggtccgtagcgccggcgagctcgtcgtyccgagccggccgycgtcgctgacgtcaggctgacgtcaaccctagggcagacgtnacctmtgccagggctgacgtcatccttttcctttttccctttttctcgatttttaaatagattaaaactttgaaaaatcataactaaataaccgtagatccgttttaggtggttcaagtttctaaattcttctaaaatcaagatctacatgttaaaaatatcccatgtactgtttatgcttgtttttgtactgttttgttgattttgtttatttgctttagtttccgacgttccggaggagagcgtttccgttgaggaaggttccgaagcgtttgtggaagcccaaggcaagtcacacagatcccaaacaaccctttgagcatgttgatcctgtttaaagctattattttatttcaacttatgcattattttcgaatgtcatcgggtggtgaacctttcccagttaattatggccgaagttgactttattttcctatgggttataatttgattagcatgaaccttatatattggattggttcagctaaatgctatatatctaggtttgcttagccatgcttagaaacattagctaactaaaagggttaatggtaaactacttcattattttatgttaataattgtggttattttaatggtagctcacgatggtcaattgtgtgataataattaattgataactaaaacctggctaaggtgggttgtgagcatatggttttgatggttgtgctcatgacaattaaggaccggttcgcgagctactgttgtgatacatttatcgtgccaaccacaagccagcgtgggcaacggctttatcttttgtatagcatagttcattaaagtgcgccagactgagaagtggcgagaagtccatgggggtcgctggggagtccatgcctctggttgtagagggggtgattatgatccaggtacggtgcactgtggtgaattgtgttatgtgagggataatgtcacaattcctttccgagataccgtggtggtattgaggcacatggtaacatgatgtggggttgtgtcttgtgggtacagtggtacacctctgatcagagtttaatctattcgaatagccgtacccgcggttatgggtgagttgagcaatgtttttcgtgattagtctcacactactcattaatggtaataatgtgataattaatttaattcctggtttggaatggttaattcctggtttggagggttaatttgttcagccggggttggttgatcaaatgtggttgggcctatgcaacacgggtgtgttgtatggtgttgatttaatattgattaattatataactgttttattattctcttaaatatttattaaatgctgttttctgcaaatgagctatattatgccatcctttgttatcctgtgcacttgcatatttgctgtgtggcttgttgagtatgtcatatgctcattcttgcaatattcattcatcagaggaggagtacttcagtgaaggtgatgatcttgagaattgagcttattctggttaagttgcctgtggagtggagttgccgtcgctgttccgtcgtttgttttagttttatctttccgctgcgtagaatgttttattttctttgagaggaactatctacctctgtaatattttaatttgcgagtatttaatagttaattatttgtactctattatcaatattgtcattgtgtgcctcggttgattcctggacgagggcttaacacacatgtaagcgtttggaattttggatagaaattccgggcgtgacacgcccccagcctcgacctcaacctcgggctcacgaccgtccgcaccaaccgatcctgggtggcgctccgcatggaccccagtctcggagccgcccccgggaggggcgtcgacgccaccgccgcccacggtaggtcgagtcccggctaagggtcgctcgggatcgggcccagtacgtgtcctgtggaccccaccgcgaggttgcgaagaccctgtggtcttggattggccaggcccggaccgctccggttgtgcggatttcgtcctccgtgaggggtccgaccccgaggtcccctgagaaggaatatcgctacaagggaacccagatcgcgttaacaaaagaaaaataaaggggagggaacaagaggccgagctaggtaagagcaccgaagtgagcgccagcgtttgtacctgcggggaggacagttgaaggtccactttgggggctcgatgaagctccctcggcacggctccgtttgccctatcttccggagccgtttcttcttccgaccggcctcgggcccggcaggtcgcttgtgacccgccggtggaaggtctgtcgcgcgctcggcgcctcctccacgcgggggagatggcggccaaagattcggtcgccttccgcttccccttcgggtcacttgccgggtcgtctctgggtttgcggcccgagccgggcgcccgtgagtcgctaccaccgcctggaccgcttggccgcctcccgcccacggacgcgccgccaccaccggcgctggcggcgctaccgctggcgcccccacggccaggccggcccctgccggcgccgacgaccgacatcacggccaagatgctctcccggtcgcggtcgcagcagagggggaggatcccwtccgggatcagtgtttgctcggcggagtccagcccagcacccgccgaatcaccatcttggcgtcctcctcgccccagtcccagcgctggcccgcatgcgtgcgcatggggtcgttggggccggtatactcccacgcgccacgggatcgttcctggagaggggcgatacggcggtgaaagtagtcgccgaagaccattgcccctgtgaggcccaagcttcgcaggcccctgagacgatcccacaccgcgtcatattcctcgcccagctccgaagccgccagccatgcggcggatctctcgggaggaccggcgggaactcgaagtcgtgggtgatccggcagctgcagtgtagaaccagtctttcttccagtcctcccttttcttgcggaggtggctctcgatgtactgccccgccgtgcctggtcggggctggaagtaacatccgcccaccaccgcaccctatagcagctgcgggacgaagaaggcctggaacaaccgcatcgtcggtcgcaccctgatgaacatctcgcacaggtgcgcaaagatgctcagtgtcatcacggcgttgggcgcgagatgcaaggcatggatttcgtagaaatcaagaacctcgtggaaaaatcgggagaaaggcggaatcaatcctgccatggcgaaggacaagaggtgcacggaccgctccggatatcgcggccgtggccgtgactcccccgccctcacgacggcgccgtccggcataattttgcgggggaggctaagatgtctgtctgccgagatgcgggaagaagggagcactgtgtcgccggtgtcttgagccatgatggccggcggggcagtgacagaaggcgtggcgcgcaaagctgcgtggagtggtgagagtgttagggttcgagaggcgagaggacgagtcggcggttagcgaaaggaaggaggggaggtccctgcctccactcccttttaattctcgcctcatccgcgctcgcctcctcgtttcccgccctcataattgcccccatgtccgcagttgtctttccatttcccgcgcccgttcaccacgtcgcccgtcgtatccgcgtcccacctttagtgcgcctatcaccggcgcactcgaggtggaatgcgtaacggctataagcgcaagctgagtgaaccgtcaccacaaccgccgtaggaatagcgggcgaggtaatcgaggcgtggtctaaccgatcccctgattatcgcgtcccaagcatcctcgtctttagcgcgatcagaggcgaccgaaccgccgctatggctggcccgctaatctggccgctcggcaatactcctttaaactccccgccgggcccataaccccagctagggaggtcgtgaggcaccatgtcagaccgatcccaggaagcccgcgtctgacatggcgccgggggctactgtcggagatatgggcccgggggtatgtggagtaaaggagagctaattccctttccagccacgtggctctgcgagctggtcccacccacatacctcgtgagtcactaaggcgatggggagagcctcgggggtcgacgacatcccctcgggggtgacgtcacccccctcgggctctcccgtcgttttagcccgaggccctccctcggggggaagtgtgagaggggagtccaggctgggcggccataaatgtgcagcgccccaaccgtcccttgctgcattcaatgcggcgagggcagacgtgcggcgtgcccgaccgacccctgtcagtcggatgcgaccggtctgtgaccagtctattgccggtcacggccgattgaacgggtggtcgtgcccccacgtcgcctctgtctccgcggagtggcggtaggtacgccccgtcacatccggacgtcgttcgatgcagggctgccgcaagtcctcactcatttatgagggaatgacagggctaccccccgtgtcagccgggagacggtgctgggccccactcaaggaccaaccgatgcttagcttccagcaaaaggcacgggttgaaacccggtggcaggagcgagcagggccccctcccctccctccgatggagggggagggtagagacggcgcatgtggtatccccttaagctataaaaggaggaccttgcccacaaaaagggggggcttttggaggggaaagcaaggggaaccttgtaagagttcactgataatcccaaacacaggagtagggtattacgctccagagtggcctgaacctgggtaatcgaattgtgtgctatctaaccggggatcggagggacgaacacgcgacttcggagagacgagtctctgccctcggccgaactcacgaaaggggggtcacgcgactccccgcgatcgggggtcttccctcgacaacaatcataaaagtaaaatgtccagtcaaaaaaatcataaaagtaaaatgaatataactatgagtaatattcctaaatatattagttagaaaacaaaagtaatgctataaagtaagcaactataacatctaaattatatgactactattgttctgtttgtgactattttatatttaaagagccatattcactatatttaacttttgatatgtcactttgaggttatattttctgacttttgcgtgacattcaaacaattatgaaattatgaaaaagaattatatagattttatcgttgtatataattgcacaataattaaattagatctatcatggtataagtaaaatgataaaaggactaacttggactatttgaataaaatctaataagcttgtaaaagaattagttcttactctttccttaaattggaccgaccattgcatgttacgttttacaattttgagaaaattccttctatacccctgaaagtttagccaatcccttctacgcccccgagttttgtccactcccttgtatgcccctgaattttggttttgatccctttcgtacccattccgttagtttaccgttggttgaatgtttaattccaatgaaaaggacctttttgcccttagacaggaagggaatctgaaagttgtagactagttgtattgtcggaagtgtataattttgttatataagactattatattcatggctttattgtacaaggtattattcatggcaaagtttatttttacataatgacataaaaataatatttatttacttattaaaagagcatatgtagcaattttttggtagttcaacatattttctcgagtaaatttcacaaaactacaggtattttgactaaattatcacaaaactacacatttaaggagttgtatcacaaaactacacatttagcaccaaatttatcacaaaactgcagattttaggttaagtatcacaaaaatgcatatttaatattaaacttatcacaaaactacaacttttggctataaacattattaatttatgattaaattggttctaaacctgtagttttatgataatttagttactaaacatgtagttccgtgacacttcatcttaaatgtgtagttttgtgataaatttggtgttaaatgtgtagttttgtgatacactgagttaaatatgtagttttgtgataatttagtcatagtatctgtagttttgtgaaatttactctattttctcctgttgtatatattatattggaatgctttccaaaactagatttatctttaaaaaatgctacacatactttattaatatatattaatttttttgtacgacaaagttatgaatatgataatcacacaacaaattttatacttccaacaatatagtttacagatttctacatattcctcacatattgaagggtaaaactgacttttttatacctattaaacggtcaattaatggcaagggtacggaagggatccaaatcaaaattcagaggtatacaagggagtgcagaaaatttaggggcacataagggattggctaaaattcaaggatatggaaggaattttctcagtttttaaatgcttagatattaatggccttatatctattctgtatcatagaattatgagtttcttcgcatattattgcatatataaggtagttaaaatgttatatttgttactattaaatttaaatttttggacttatgttaattaggtattttcttttaaatatatatatatatatatatatatcacgcatatcacgaggaagcgaggtctacgacatatctttgttatgtaagtatgcaacagcttgcgtcaatctgaaccaattaaaaggatgactaacttgatgctacaaataaaacatatttgttaatgataacagttctttagaatgtgttaaaaaatccactaaacattcaattaaataagacaaaacaccttacttgcttttcagttagacaagttttataaacagactagatctaactctatactgaagaaatcatatataactatttatagggatgtcttaaatattacatgtttcaaaagcaaacaaatagtttaaaaaaaaattacctaagcgaagaaaatttatggagaaatactgcttttgaaacatgacaggtgtcatccggttgatattccatctatataagtttttaaacgcaatctacaatacatgcccgcgcgaatacgtgggctaccttcctagttttgtttttctttttttgataatctaccttcctagttattattaaaacatgataaagaaataatcctccaagttcgtttcgtgacctagctagaaagctatcatattaataagaaagcaaataaatatttggtttcattttgtaacagtactcagactgatcccacacaaattagagtactacaggaggtacgattatatttggaataaaacaaatatatccatgtgtaaattgcaatgaaattgtagatagttctacgtaaaacaagtagaagttctagcattcgatataatgtggtcggagttggagatgcaatagacatacctaacttgcggtcttgggaagactttggaggatcggttgcttggaatgtcctgtgtacaaaccaacgtagaccataaagaaatgaatgtacgagacgagaaagagaggtagatacatataagagtgattttacggttggaccttgagtacacctgaagatatatatgagattttgcattataaaatttagatacctcctggtacatgaagtactaagagggagcaaaaattttacgttataaaatggtactttgaggttcatctttgaaaatcataaaatttctccataagtttcagtttgcacatgaaacacaattaaaatttaatttattattataggtatagtcggagaataagaaaactacaaggcttataaggtccaggaagtgtgtcagataacatgtttgttgtaattgagaaatgcttttgttgtgtggtggactgatgggtgtaaacttttttccctacctcggtgtttaaatcgaataaaggtattggggcttccaaaacacaaaaaccagttattgtgatttgcttccaggaacctcatgttttagggcaaaatgtttcaaaaagccccatatttatgtaaaatcctcgtatttcatttttacatatgattatttgtacacaaattcatgttaactcaaagtacacattccgcttgtctcatttatcttctcccccccccccaaatcttttattatatactaaaattccattaaacttcctacaaatgctcttaagccgccacgtggcaatcctacaaactctcctagatcgccacatggcactatataatctcaccgttgattttcacttaaattggtggtcccgatattttagaccattagattgcatctgttattaaaaaataataccttcctttcacaggagaaatatataactgttgcctaaaagataaataccttcctcacgtacgtacgactaaaagaaaaaaaaaagctaaaccattaattgtttgctgcaagaaaaagaaaaagaagaccacgtgcatgtacttataagatagagaaaaataaagaaaaaaaagaacgtagtaccgttccaaagaaaaatcgacgttatttcatacaatttacaaatttacgcaaatatacgtagctctttaaacattccatcttttaaattattttaaatttatatccaatttgtgatactattgtattcctttacttaaaaagatgtctatgatccaaaatacatcatagcaccagcacatgggacccacatgtatataattgatgttatgttatctatctattatccatctattatctatctatctattatataataaaagtccattaaacttactacaaacgctactaagccgccacatagcaaccctacaaatgctcctaagtctctacgtggcgctataattatttctaaaaaaatcaaaagaaggcaaaacatctgatttttttaatctattcccgatggacccattgttgtcaaccattagatctatttttaaaaacgatcaattaaatttatctccatcaatcccacggacacgtgcatatatacaagcacagcacgtacgtatctaagtaaccagctggcctcatgtccctctttttcttttctttttctttctaaaataaaaataaataaatacaccgttagaaaccattagatctatcatgaaaacctaacagttaattttatcgctaaaaatcactaaatacacgtgcacataagcaccgtatgtaagtacgtagacacgttggcctcacaaccctctatgtctgttttttttttcattctgaaatccaaaaaaaataaaataaaacaaagctacatttagggtttaaaagtaaaaataaatcaagctataagcttccgattataggatagaaaaaacagtatgcaatttgaattccttattgattcatcattgtattatcctttggtttccacaaatatgtcgtagcaattctgaaggaaaaaaaatcattttctacctataccatgcaagaaacacacaaaaaaatttaagtccattaaacatcatgcagaatgctcctaaaccgctacatggaactttaataaattagaaaaaatcatataaaaattataagaaaaaagaaaaatacttagctatccgttctaacttaaatcggtggacccattatttctaaccattaaatcaatccttcaaaacacaaatcccttccacctcccactccctcccgtacataatcactcctccctctccaacatccatatgtacaaagcatatatcttatatacaactcaattagattaaaagtaaaaaaaatgacacataagttgtttcgtactcccccgtcccattttaagtgcaaccataaaatttcatgcccaatgttgatcgttcatcttatttggattttttttataattagtatttttattattatgagacaataagacatgaatagttactttatatgtaacttaagtttttaattttttttaagttttttaaaagacggacggttaaagttgatcacggaaaatcatggctatacttagaaatgggagtagtaagtagatttttaaggaataagtttacattaatcactgtctttcttataaataatggaggtacagtttcaaccttttaatataaatgaatggttacgatgctcaaatgtgctttattgcgcactcatatcaaaatagacaaattatcagtttgaagtatatattttttatatctttataactaagaaaatacttagaacatgtgacgagttataatgcacgcataatgttatgatgccggactcagtcatcatatagaatataattattttaatattttactgcctaattaaatatgtcaatgattgttagaataactaacgctcaacatttaattttgaaaatatcaaacactcatattcaaaagaaaaagatatagtagtataattttgaacgatattaacaaagtgcccatagagtgagctataaattataatcttggtggtttataaatctatacaatctagaaagtatcataactacttaaaaacatttttaggacaaatatcatatatttaaactattatcaaataacatcttaagtaaaagtcgtcgtatatccctacggcaataaaataaaaccaagatgtccatgcatgttttattccttcacatagcatgtaggcaatcaagctgctaatggaaagtttgtttggttatgatttatgcattgatagatgatattttgatctaggtgggttaataaattttcaattcccatgattttctacactccattaaaacatccgttcttttcaatactctattttacacatgtattcctatcatatttttgtacattttctattctttttttttcaatcatgtgttacaaaggaacctttaattttttttcaccgagaggcaaaatcataacatttcacaatcctaataacacaaattatgatggtttaaataagatgcccgcgcagatgcgtgggctaccttcctagtttttcactaaaaaaaataaataatcctaactaatattgatataataatttgttaaatatacgtataaatgatcaaactactacgatcgcaaaatgttgaaaatcatctatgtacttaaaaacgtctcatatatattttttacaattgttatagataaatatattagttaaattgatgatataaatttttaggtgaggtttaatgaaatattttgcccccaaacttgaggttttatgaaacaaattataaaatctagggatttgcatcatggattcctcaaaacctgaagttatgtgaaatttgctccaacataaatttcgtgaggtttgaggatgtttatcctttgactagtattttaaggataaactctcatgtgtttgtgggtgacataaacgcttgttatatacttattatactatcggcaagtataaacaacccatacctgctatcgtcaaggttacgctcatcctctggttccatctttagagttcggtgttgtttggttaaccaatcatacggagaacacctttctcccggacacgctttcacagagtgcatctcccttttaaagcattcatatggtaaacaattattattgtgaaaaatccctaaataagatggtaaagaaatatcataaaaaaactaaataggatggcttcctatttatgagaacaatccacacctgctatcggaaaggttccttgtatcttctagttctctctttgttgtttggcgtatgtctatctctattcggtatcgtttgcttgaagaaccatcttcgtcagagctctgtttttgtctgtccagatgattagcttcctgaacctgactgacttttccaagaactttgaaggtaggaccgtacggatgcttgccaatggcattcttgcaaacagattcggcagctccccggtcagattcgatagcaccagtggcaattccaattcttgcactgatatccgtaaggtttaataggtgctccacgcccaaaagcgcaataccatatccctcacctctatgggcatcgaaaactagatacagcctctgaagattgggcattgccccttcctgaaactctattcgcagtttatgacacctgaattcaaagaatatcagagcagaaaatgtaccttttttgaacacgatgccttttgcagtgggttcctggacatacagtgagagagctgcaagggtacccaattctgtcaggatatccatgtcgctatccaggaattccctgatcacaattttcaaaatacggagttctcgaaaatgagcaatccactcgggaatttctgaaaagatgcactcagatggcaacagctccagtgtttgaacgggaggagaggatgcgctcgttgaatcatggaggctagcagtcgtgtgtgaggtactaggatccatactgtgattcttgatgccatcagtaactcctggttgatttggcataccttggaactggagtggtaagaaactgagatgaagaacattcgatggaacaactgcgtcgtttgcatttatttccagtgtctgcaagtgtttaaaactctgcatctgatctggcagtttcactgtaacgctacaattgacctgcatatatctcaatagaaccagttgataaatttctgtgaggtctatcatgtttccatcttcacacgaaaaatcaagacatgcaacacgaagaagcttaaactctttaattgaaggcatgcagttaaatagcccaataaatataaaagaccgaatttctgatagcccaatattaaccggtgtagtagcatatgttgcactgccgaagtgtagggacaatcgatgaaccttgtaagtaagtcttactgtcgtttgagaataatcaattgcagtgacaaaattctcttcctgggccttgcgtgtgataaattcatgtacaatatggtgaactgcataggtcaatatgtcagatttatacttgatatccctacattggatgagtcccagattgacaagcttattgaaataactcatggcaacttccatggcattttcccgtgttgacgagcagacaaaatcttgggctatccattgattcaccaaatcttccttcaaaagtatgtagttatccggatatacactaagatatagcaaacatgtcttcaaacaacaaggaagactgttgtagcaaaggttcagtacttgcttcagtatctcatcagaagtagcatttaccctcaaattattgcacaagaaattttctacatattgccagtacttcaatctatcttccaagttctgtactgtctcatgttggattgctagaagactgcccatgatgataattgctagtggtgagccaccacattttcttgcaatctcttgtgtgactttgtggaattgaccgggatgctctccagagccaaaagttctactgattagcaacttcttcgactcgtcgacactaagagctttcatcttgaatatgtactgagggttataaacgcaacaagccagagcaacttcatcaacttttgtggttgttattattctgctcccacaattattcacaggaaaagcatgtctaacaacatcccatacggatggagcccataaatcatcaattacaataaagtacctgacaaataatttggtctgtatcagacgtgttttgtcaagcaaaagagaaaatatccaagaaaaatatcatgacaatggatttttgttctaaaaaacaaaaagaagatttgcaaacctcctgttttttagatgattcttgacatcgtcgatgaggttggtcacagcacaagggacgactggttggagcggccgaacttgagagagtatattcctcagaatcatccttatatcaggctttttggccgtctgcacaaaagcccggcactcgaattttccttcaagttcacgccatagtagttcggcaagcttggtcttaccgattccttcagcaccgagaatgcataacaccttgagattttcatcttcgtcttcagccatccaaccacgcagcttgtttctcgggccatccatgccaacagggtcaacaaccttgctgaacacagttggaagatgatgatgtacaaccatgtttgtgggacttgatccagcatcagcatcaagattatagatgctccatcgctcattcgcctccttgacaagagtcaagaatcctgagatggtgtcatcccaacccatcttggcaccagcatcagcgttgaccaacaagtcgatgcagtcctccatgtcgtagcaaagctcgcgcacatccttcatccagtaaatcaccgtgagggatggatctcgcacatttgaaagcttctccaaacgatcttgtatgacgctaagctcagatatgagttgatggatagtcgggctatcgagctccttcaacttctgtagaagggagcccatggcacccagcgaagcgctaatcggagcttccattcttgtttctgcctgcagttgatcaaaagggaacacatcatgagctgctatcagccagaacctcatatggacgaaaatcatgaaataggaggtaattcattggaaagaaaaaggaacatcattaatcgaaaagaaaaccgcagctaaccatgagattatgatatgttaggggagacacggcagtactgcaaaggtacgacaggagttatttggagagtgatttctctcttcttcgttttaagggcaacgaagaacaacaatatatcctgttcctcgctccgacctttacatgtccaatcccaacagctccgatacccccacgcatgcccaccaggcacccatacaacagattttgtttatactcaagt

SEQ ID NO: 11 - DNA, 2921 bp, Oryza sativa
taggagaagatggaattattctaattgattagggttcccacaacaactagctttcttcttaattgttcttatgcacgccttctccatgttaccattattttttttaagagaaaggccgtacggcccggcttctattgaaagccagaggattgtgctgggaacacccagacaccagtttatagcatagggaggttcaaagccctccaaaaggaaaacatgaaagactcccaaacttgctaccagctctaaaagcaacaaaataagcagaaagaaaccaaaaggctgtccttacaagtagcgtcatgacagcaaaacggaagacaaacccctacatcctaaatttcgtctcatcctagtttgtggcatctcaagaaaggagaattgttctgtgcaattatagtttgtggcatatgttcattgtgtttccatgagtaaaacgccctgttctttttttttttctgaccaaatggattgcgaagaaaaatctactacatagcttagtcctatcggagccgctcttcactgtgtattgtgacattgtgtcaatataatgagacgaaagaagcgtctctctatgccaatgaacaagacgaaacatggcttagtcctacccttacaacctgaattccactcctcggagtcgaggaactcaaatcgttcttaattggaagtccacaaatttcataaccgaatcaaaaatgcagctaaaacaaatttcatgacgtgaagatggaattactcatctgtcgggtcaacaacgtggccggcgaagagcaccgcacccgacgactcctccaaaaccaagaacgcgaacggatggtcggcgacgaaatccaccctcgccggccgccgtgacgatggcgccgccgatgcgaaggtaagacaaaccgcggtggcggcggccgctcggtgccctcctcgttcacctcgatgacggccttgtgcttgacgtccgacacaaacagcggcggatcatcgccagagttctccgcttccagcacgtcggccaggtccgccacgaacggcttgaacacgtccctgaccccgagacgctggagggcgcccacgacgctgtcgtcgaaggagagcttgaacctggggatcctgaactcgccgacctcgacgcgccgctccggcgtgtgctcgcggaggaagccctcgccgccggccgccatcctgtcctccaggctccacaggccgtcgcgctcgtcggggaggaagacgtacatcgagtacctcggcgtcgtcgtcgtcctcgcgctcacacaagccgcgtacggcatcttgagcaccttgaacccgtcgtgcacggcgatgtactggtcctcgccgctgcgcatgaagtccgcctcgacgtcgccgccgtcgaggcggtggaacgcccgcttctcggttatctccttacagaaaggagtccgccattggccgttgaagtagatggcgctggtgaccacgaggcccgtgtccgtgctcaccgaccccggcgggaggatcgtgtcgatgaggttctccgtcgccgccgcaacccagctgttgatctccttcctcgcttcctccggctacacgcatacatgacagattagattggtagatcttgtcatgtgtatatatatatagttcgcttacgttgttgaggaagtcgacggcgagggccgcggcgttgaacgacgcggcggcggcgtcgcggaacgccggcctaaccgtccgcgtccgctcgtgccagactccgcacgcgtgcgcgacgcgcgggccgccgcccggctgtgccgcgccgccgccgggggcggggagagcacgcgccatctcgccggcgttcgcggcgagcttctcgcgggactctgcgccgagggcgccgaggagctctgtcagggtgcgcccgcgcgcgccggcggtgaccacggagagcgcggagtagatggacagcggcgagaagacgaggttgccggcagcgccagccttgctggcggcggcggcggcggagagctgcttggtgaggcgcatggacagcgccatcaggccggagacggcgcaccgccgtgcacacgattccatggtggaaaaaggtgttcgacgcctgtctcagagatctatttgcacaactcgatcacaagacgtacggcccaaggcttttatagaacctataatccatgaatgctgaagtaataataacatagggcatgttcactttgatgcaaaataagaccttaccaaaatttggcattaccaaaactttgatatagttgtcaaaattttgacaacttactaaaattttagcaggatttcttatatagttatccaaatttggttttgctaaaaatttgtcgacaaatttttttttttagaaatctcccagattttccaccgtctaaattgcattatgatttgtgctagctgccgttacaaccaaaaaaaaggactactgtaacctccgtgggagtcgatcccaagatctgatgctttgaacgtgaaataactagctgagcaagaaggagcaactaattgtttcaccaaaacaatacttacttatactgtattagttatactgtatgtacatattaattatattataattgcactataattatatatattagttacattgtacttacatatcactggtggagaaaccatctttcgtcggtcggccgatttccacaatagtcccggatgcaataaaaaccggggctaaagatgatctttagtcccggttcaaaagggtaacgggcatatttgatctttagtcccggtttgtgttaccaactgggactaaagatcatctttagtcccggtttgaatgctgtcagggcctgtcaggcccccccgggatctttagtcccggttggtaacagatcgtaactttagtcccggttggtaataccaaccgggactaaagatcccggtgatctttagccccggttggt

SEQ ID NO: 12 - DNA, 10883 bp, Oryza sativa
tggtactataaaaatttatgttgttatagtgggtaacattatcatggttatgcgtgagaatggcatactgtctcctgcggcacaccctccttggtggtggctgtgcgatagggtggggcgtgacaacaacgaagtagcccaagcttagaactgctattgccagcgacgacgaagagagtgaaatagacacaaatgattgttgtaggaagctcgacgctctagccccctgatctaaactcaatgttgtcaccatcgaaggaaaaaaaatgcagacgatcgacgatgacgaaatcacttccaaaacaggccgtaatcatctagctcatattccctctatggaagtggagattgcagctgtgatctagaccatatggaaagccaaaaatgcagcatgttttaaaaacaagaaatctcatgaccctactgagctaatacataaagtatgtttttcattgaattgtggtccattttgctgatgtcggaagacgtgcaaagaaagctacaaccaggagtggcaccgctaagaagggtgcactactagaaaacacctaatatcctataggtgtcagtttatttaaaaccgacacctatttccttctaactgtaagagatagatctcggccatacatcatcacgtaaaatttaatttatacccaaactcgccataattgtcatctctactacccaaataatgtacggggagtaaaaatggaactcccaagaccaatccctccatcaacttcctcctttcaaacccccaatcctcttctcttggcaaacatgccgctagggtttggaaggatgcgggggttcgagggcaaggatagcgtctatattacgagcggccttaaaaaggttggacatttgcgcggtacaccaaactagttaatttgatgaggcctagaatgtgagggccccaagacgaaggaatgatgcctcagataggcccatcacggggatcaacttattttattgttttagtctctctggtgacagattcttcttcgaagggagtttggccaaaaaaagcatgacgacacgacgagtaacttgattggattgagcatgagttttgtaggtcggttattttctttcattattttgaagaacaaatggaaaactaggcatgacatgacaatggaataaatttgagcttgtgggaatcacatgagaatacccgaaatatactaggacaacaaatgtttgatagagtttagaattagttgaacaagagatatattccaactaccgatcaaagaacttggcataacgtatgggaacgtattctggagcctagatcgttggtgttggggatattaatccaccagcgagggaacgactcggtgtgacagtacacgcgactgtaactttcagtattgtgtgatatagagtttgatcatcaaagttaggacaagaatgcaattgtccaatgggaaaacaaactcttaattagcttcagaaattcccatttatattaacttcaacatctaccacccttacaagatagctcatataaccataacaactctagcattatgctattcagacaggtagcattagagggtacaaaaacggaagatatggacatttgacatctgaaacagattcattttggctggttttcacatgacagcttgttggtcttcgtatgatatatatatatatatatagcgtctcgacccttctcaatcttaaaactcctgcttttgagaaaggaaattaattagaatgcaggctatagctctgaagaagccccataatgaaagagaagtacacagacgaagaatcaaaggtgcaaccagcgagcatacaaattaaagcaaaaataaagtatcagatttgagcaaggcaattttgtataaatttttttttgtcccaggaaaatggttaaaactctacattgcacccaatttatgttacttttcgatacggtcaggctgctgtaaatcaacattcacactgctactgtaccattacatgaacataaatcatgacaacttacagaaactatagctggtttatgcgctgtagctgttagcctgtgagagaaaattcacttcttaaggtccaccaccctagagtttctcaatgaagagcttaataagatccattgattacagaaaaacagagacttgaatagaaaaccgtttttggctgaaaaggaaaacaaaacaattctccaactactttgcttcaagaaaaaaatacacagaaccgtaagcatcaaagtgcaaagttgataccatatttatgaaattgcatttatagggagtatttgatttgtcaacaaacatgattgacgttagctagcctgaataaaggccatttctgtgtcaaaatcaaactactttacaagtgcttagcaagcgatcaatacagcaagcaatcaacaattgagaactcatgattgtgaagccgccacacgatagaaataacaaaactaaactgccagtatccaagatatatatgatcggggtatttactaatcagtacacaaatatttctctttaagtaaaactagcgcagataataattaggtagttcaaaaatgcatttcttattcatcctaaccattgttttatagcaggctaatttagcggttggccctctcaacagttataggggcatctatagcggctaaattatacggaagagaaaagagctaaaactcttctcaaacagatataaccggacatagcggcagctatagtcggagatttaaaacaaccctgatcctatcacaacatggaattcaaggtcaagccagtacactaactgcttcacgagtgagaagccaactgagaaataattaacacttaatttgccgattccaaaaaagagtcacaggaagtacaagataggttactactactccctccgttccaaaacacgttacattttgggacagatcaagctaagggcttctccagttaatgcactcgatccatgtctatcagaaaaacaaattaaaatatacattgtatagatgaaattgggaatacataccttgtactccatgtcagctatacctgatgccagatcccctgcggaacaagataagacaggaccatttcgaagtacccaccatcactcctagctggcatacaacaacacatccgagcaagttcttcctggttccagtctgaaaactcattcaggccaaaacgggtcgagtgccctgcgttggcagaatcatcgttggctgcatcagaaaatcttgcaactgacttgaatattttgaatcgcctagcctcctccacctcgtcctgcttgtatgtcctgccatgctccttcatccaatccttgaaccttgcccttatgtcttgatcatcctccatgtactctcgaaccacggtcttctcgtcaggcgttgtttcgttgtaagacgccctctctcgtatcaagtcttccatcaactggttggaacggaagtaaggatttgtcttagtgggtgtgtcctccttccgagcttcgtctacgtacagacagtatgaactcgtgagggttgagggcaatgtaacataaaaggaaagcttcccggcgaaaaacataaaaggaaagctaaagacgagaaatctgaagggtgtttaacatgtagtatgaggagtgggttctaatctcttgaggggttatcccttcgtgtgtgctttttttttcaaaattcgatacaaatagttgtgaaaaaatctgaaaaaaatatcgacaatgtagagtacagtgatatgtatcggtccattaaaaatcaagttcaaattcaatctacacatcgaaaagacaaaattagacgtgaacagtacattgctattcatacactgaatttgtctttcttatacgtcgacgtgtgacttgagtttggagccaagattttataaagttgtatagatattttgtatgagtgttgtcaaaattttccagaatttttcataacaatttggatgacttttaagcaaacgagggtacattcttatgcgatcgaaataatttcccataaccattgatggttatatatgaagagtttttttactgtccagcagtgaagcccggtaccaaatgtggaccgctggataggtatgatcggatggctgacatcaattggtaccttcaagtaccatttactccctacgtggtacctgccaaaggggaatgaggtggaggggtaaaattgtatttgcacattggaaaggaatttcgtcgatttgattcgtgttttggataaggctttgcagaggaaaatctacggcagtggcggcgagccggcgacgcttcaggaattacggtgcgctccaccagtcttctgaggccacaacggtgctccaggagttgttgtcagcggcgacaacggcagcattccaggagttatcggcagtgacggcggtgctccagggatcatctgcaactgcggtggcagcgctccgataattgtcttcggcgacgacggcatcactccacgggtcattgctagcgacgacgatggttgatttggttgaacagtttttgagcaagaaagatacatacaggaataaattgagaagcaggaagttccccgcaaaaaaaaagagaagcatgaagttgatttgcctaaatggcttcaggttgatttgtcagacaaacacaaaatcaatactcatgcgaaaagctccaacacaacctccattccggaacaattatagggtgcagactacagcggcggtggcgacaaagtccggcgcgtcaggatacatgttgcggcagcgtcggccatcggtcccactgtcgttgccgcagagcaaggacacgctggggctggggggaggacaggcagcagcgacggcggtgacagagacgtgcgcgacggagcgagctgaggaacgatgtcatgcgtcaaggagagcagaatgcgacggcagcggcgactcggatatgcctgtgaagatgaggagatttcgtcgtcgcacgctagctggctgctttacttcatttccaatcgtaccccagtttattgaacggccggatttcgtttggtacctgtggtaccagtacctggaggtaccaactgctggaccagaacaaagctcatatatgaaacatgcagtcaaaggcagctagtttcacatggagtacctaaaaactcacctgtatcttgcatcaaatgcaaaattccagcaaccgcagagaaaacaacaacagacaacgtagcggtagcagcatcggcatgttccaagaccacatccctctgcaccacgaggattcaacgtgagcttatctggaagtaaaacaaggcttagatagataaacgatgaaggaaatttaccaagtcgtggaaccggcgtgctctcctccccgccggcgaccccagagacccctgcacaacgatgaattaacgaacgaaataacgggagcaagatggaaacacgcgcgtgaatgtgtgtgtctgtgggattgaaaaaggagggaaacacacacagcaagatttgaaagaaatttaccatgctgtgcagacaacgggatgatctggcggcgaaagcagaggtgttggccgccacaagactcttacgacgggcatcggagtagaagaacagcgcgttcctctgcgagaataattatgagccgctagcggagaaagcatacgatgtatagatctgagatgagaaaagatcaaaagaagcagattactactccctccgtctcaaattataatatctatattttttacacgattttcaatggcatattttgactattggtttattcatgttgcgttattaacgaatttgaaattagagtgaaaatatgaatctagcgatatatcatgtattatgattaatataattattagttaaaagtttctgagtttgatttaccagggatagtagacgattagcgattcccgatcccggcgaggaggaggaacagcggatactcccgcgaacgagagagcccagcagcgacaacgacctaaaacccatatgcctaccgctcgctcgatctgtctccccgctgcctgtccgatcgatcgccgcctcaaacttgtagggttttggaaagatgcagagggcgctcgaggcgtcgaggtccacgtacggggccctgcccgaaccagttttttttttaaaaaaaaattacacagtacaacgcagatactcacaacgcacgcgcactgacccctatgaacacgcgcacgcaaaccctatatctatgagcatcttcgaagactggactggcaaatcttggagagattgacgaagtcaccacgggcgcctcgctgtcgacgggtacgtcgcctaccactgaaagcacaatgccgttaaatcctgaaaaattcgctcccacggggagtcaaacccaggacctcaggtgctactgagactctcccgcccgaaccagtttgatgaggcaggcctatgatgggttcaattcagattcagggtcgccgcgccaaatgtgtagtttgccaattgacctggattcgtgcgtactactccatgatgtatatgcagcttcttgttgcttagttacatgcttccttcgacttgtgtacagattattagattgcattatgttgcttcacgatgacaggacctagttaaccgatcaattagtagattgaaaacgattttctgactcgatggcgagtgttcaattacatgttttgaaagcattcaagccagtccgaaaatatttttgttttctaaaaaaacaaccaatgatcgaaagattgcacaccaatattgtttgatgccaattgatctactgatccacttgccacatagggcagctattactccctctggaatataaatgcaacctaatacaaatgtaatatattatataactataaatctagataactatctctccaaatttatagtcttaggatatgtcgcatctctattacactacaagaaaaggcatttttgccgatgttggatacttattttcgcaggtggacataaccttcggatacaaatagccgtctacgaaaatgtaaatcggggtgaaacccgctgcccgcctgcgaaaatccatttttgcatgtggaccacttaaacggtcttcctgcaaaaatacccttcatttttgcaggcggacctccgcctgcgaaaatacataggaaatataaatagaggctgtataccctagatttttttctaagtcctctttgcctcacctctcccctccccgcacctctcctcctctccccggcgctgatggcagcggagggcggcggctaccggtggaggcggcggcggagggcggtggccggcggcactcccctccctcactctctcccagatctggcctgaggggggcggggggaaggcggtggccacgcgtacggcggcagtggagggcggcggctaccggcgggaggcggagggtggcggcgagcggcggcggaggagggtggcggcggctcccggcccctccctccctcccagatccagcggcggagcatgctactgtactcttgctctattatgtacacaagcaaaaatttccatctcgaatgtaatcctttatttttgttgcacgacagcacatttgacaattacaatcacctcaaccaatcctatgtgaacgacttcgatgaccagtacaaatatcactagaaagagcagcatcttagactgtgtttagttcacaccaaaattggaattttggttgaaattggaacgatgtaacggaaaagttgaaagtttgtgtgtgtagaaaaattttgatgtgatgaaaaaattgaaagtttgaagaataatttgcaactaaacacggcgttaatgtaaaactgcttaaacgtttcgctgaaacctgaatagattttccctcactaaaagcacacagctccagtactctcaactgaagccagcgccttcttgctgatccagtagatctgagtgagccgtcttctcatgtctatatgaatgcctagcatgtggatgatgggaacatagtgcctccacacggaaaacagaacgatctattagcacgtgattaattaagtattagctaattttttttaaaaaaaatagattaatttgtttttttaagcaactttcgtatagaaactttttataacaaatgcaccgtttagcagtttgaaaagcgtgcgcgcgaaaaacaagggatttgggttgggaaactagaggtccgaacacacccaaagcaattttctatttcattcaccctaaaatatagcaaacgtagaactagccagtttaaattcatactatagggtagatatccaatttaaaacgaataaaaggctcaatttgctctctagttgccacgaccatttaagatccatgcaaatttcattacagttgactaggattagtggggtcaacgacgtggccggcgaagagcaccgcacccgacgactcctccactacaaagaaggcgaacggatggtcggcgacgaagtccaccggcgccggcgccggagatggccgcctcgccctccctttcataataaccaccgtggccgccgccacctccgtgccctcctcgttcacctcgatcgctgccccatgcaggacgtccgacacgaacagcggcggatcgccggagttgccctcctccagcacgtcggacaggtcggccgctgccggatcgaacacggccctgaccccgacaccctggagcgcggtcttgatgctgccgtcgaaggagagcttgaacctggggatcctgaactcgccgaccttgacgcgcttctccggcatgtgctcccggaggaagacctcgccgccgccggcctccatcctgtcggcgagggtccacagcccgtcgcgctcgtcggggaggaagacacacaacgagtactgcggttgcgtgtgcgtacgggaggcacgcgtgttgtacggcatcctgagcaccttgaacccgtcgtacgcggcgacgtactggtcgtcgccgctgcgcatgaagtccgcgtcgacgtcgccgccgccgccgaggaggtggaacttgtccttcttggtgtcctgcttgcggaatggcgtctgccaagtggcgttgaagtagatggcgctggcgaccacgagccgcgtgtccgtgctcaccgaccccggcgggaggatcgtgtcgatgaggttctccgtcgccgccgcgacccagctgttgatcgcgttcctcgcttcctccggctacacgcgtcgcgcatgtccgtttcaatggagatcgtcattcgttaatggcgtgacattagattgatatagatcgatgattcgatcacttacgttggcgaggaagtcgacggcgcgcgtcgtgccttggaaggacgcggcggtggcgtcgcggaaggccggcttgacgttcctcctccgatcgtgccagaggccgcacgcgtgcgcgacgcggggcccgcccgtcgccgtgccggagccgccggggagggcgcgcacgatctcgccggcgtcctcggcgagcgcgtcgcgggaggacggcgcgccgagggcggcgagcagctcggccagggtggtcccccgcgcgccggcggtgaccacggtgagcgcggagtagatggacagcggcgagaacacgaggttgccgccgccgacgctgtcctcctccaaggacagacgctcggcgaggcgcagcgccatccccgccaggccggaggcggcgcaccgccgcgaacacgcctgatccgtggctgaactgtttcctccctcgcttcttggagagcccgattccggtcgaacaccaccagtccaccaccgcctgcttccaccacggcgagcgatctccccgagacggcggatcatcgagcgaggcgagaggagaattcgagcggaggaggaggaggaagaggatccgattccaggagaggctacacagatcagaaacgcgtggaaatcacgaaccaatcttgcagaacggaagagagaaaaaaagggggaaaaaattaaacgtgggggaaaaaaaaaagataaaccaggaatccggccgggagcacgtacatgggaagcggggctggagctggtcgtcgtggaagagctcgccgccggcgagcacaagcaccgcaagcacgtagacgaggaacaacacgacgagaccctggatcgcgcaccaccgccggcgcggcgccccggccgccggtggagtcgccattggacgggaggagacgcaggaaagttttactactggtagtactgtagtagggacgaggaattaccggggggcgtgtgaatgtgggcgtggacgcggtgtatgcactcctctcacgtgcaatgagagactccaaggctaaggtattgtcatttagagcgtggaccgtgtccacgggggataaacgcgtggacaggttgtgactgctgctgtttcggtgagcaattgggcattccgttgggtggtgcggtggggccacccttttgacctgtctagtactctagtcaacacctgaggttgacctgtttagggctgtgttcaccattgcttccctaccggtgtacgtacggaaaacggaacggtttattagcacgtgattaattaagtattagctaatatttttaaaaaaatagattaatttgattttttaagcaactttcgtatataaacattttaaaaaaatacaccgtttaacagtttgaaaagctcgcgcatagaaaacgagaggagggattaggaagaggggcagccgaagacagccaattgtttgcttgccggtatagtacgaaaaaccatgaatcgggcacctgggtttacggggtccgctggtcaaactatgaatcgagcaccaggtggttgaaccgcggttcaatttttatatataatatatgttaaagctataacgcaactgctaggacagccggattcatgtgaggatatatttttttcattttttgccaactgagtgtgcatggcagactaattgggccacattaaggccattaaacgactgttgt

SEQ ID NO: 13 - DNA, 1957 bp, Oryza sativa
ttttttttgggacggatagaaatagacattagcgttaagactcgagcaatttccaaaaacaatgatactatacttctgttgactaagatttcgtcgggtcaacgacatgccccgcgaagagtaccgcgcccgacgattcctccaccacaaagaaggcgaacggatggtcggcgacgaagtcctcccttggcggcggcggcggcgcataccgcgccgcaccttccattaacactgccgtggccgccgccgcctcggtgccctcctcgttcacctcgatcacggccttgtgcaggacgtccgacacgcgcagcggctcgccctcgatcatgtccggcagctcggcccgatcgaacacggcgttcaccccgacaccccggagcgcgcgcacgacgctgcggctgaaggagagcttgaacctggggatcctgaactcgccgacctcgacgcgtcgcaccggcatgtgctcgctgaggaagccctcgccgccgccgccgccaccggccgccgccatcctgtcctcgaggctggacaggccgtcgctctcgtcggggaggaggatgtacatggagtagtaccgcggcgagggttgtggcgccgcgtggtcgtgggcgtacggcatcttgagcaccttgaacccgtcgtgcgccgcgatgtactggtcctcgccggtgcgcatgaagtccgcgtcgacggtgccgtggccgtcgaggaggtggaacttgtccttcttggtgtcctgctttcggaatggcgtctgccatgtgccgttgaagtagatggcgctggtgaccacgagacccgtgtccgtgctcaccgaccccggcgggaggatcgtgtcgatgaggttctccgtcgcggccgccggcgtcttccatggtagtcatggtgatagctattgattggattgatagttttcgtagaagttgaataactattttcaacttattttgttctttttgttgcaacttgtagaagttaaatttggtcttgcataattgtagagggatgaaaatccatatcctatatatagacgatgttttcgcgctactaattttggaccccctggcaagcaacaatctccctcgcaattttcaggttttttagtaatttttatgaattcaaatttagtaaaaatagttaaatttcaaatattttcgtgtaaaaaagttttgaaaaaaccgaaattcgtgaaatttttgttgacactgaaatcacataattttttcccttccttgtttgcacaaatcttgaaacaaatctaacagttcaaaattatataaaaattactccctccgctttatatgataagtcatttgattttttttcttagtcaaattttttaaagtttgataagtttgtagaaacatatagtgatatttcaaatacaaaacaaatatttttttaaaatatattcaatgttggatttaatgaaactaatttggtgttatagatgttactaatttttctataaatttgctcaaacctaaagaactttgactaaaaaagtacattacactatgtacgattgtactataattatatttgtcaactttttaaaagaaattttgttgataaatatgtaacaaaattgaaactatttacaaaattccaccgtgaagcgtacatctgttcaaggtatgtgcacggctataaggtagatgcaaaataattcgagttcggatgtatccttgatcacctcaaggggatttatgtagatgcgcgttcttgcggggtatgaaatgtatcttaccaagttaaacccgtgatatttacttgcgtgtgtcacgtttgtatatgcgtggtcggacaggaaaagatcaaagtaccaaatatttacaaggtgtggcaacctttacttaccgcgcactctagcaatttacatagcacaaaacagataacatagagttagagatataggacgatgacatgaaaataatttttcatgtattatatgact

SEQ ID NO: 14 - DNA, 6823 bp, Oryza sativa
tatatatatatatatgttacttcgcatttatcctaatacatctttttgtatcttgcagaaaagacgtgttgtcggagtcgtccgtcaagcctgagtggaagagctagccctagaaggaattggagcaggcaagtgacgtaataatataaatgattgttatatttgtaatgcaattaattaagattattagacctgtaattggacttatcatataagattgtgtagtgttctaatattatttgagttttaatataagattactttatttgtaattagacttagtatgtataattattgactacggaattggtgcgacaagtagcaattgactattgtagtcttaattagacttagcatatacgcacgaaacacttagcatagatgactattttagtcttagcatgtaatattatttgagttttaatataagattactttatttgtaattagacttagtatgtataattattgactacggaattggtgcgacaagtagcaattgactattgtagtcttaattagacttagcatatacgcacgaaacacttagcatagatgactattttagtcttagcatgtaatattatttgagttttaatataagattactttatttgtaattagacttagtatgtataattattgactacggaattggtgcgacaagtagcaattgactattgtagtcttaattagacttagcatatacgcacgaaacacttagcatagatgactattttagtcttagcatgtaatattatttgagttttaatataagattactttatttgtaattatacttagtatgtaattattgactacggaattggtgcgacaagtagcaattgactattgtagtcttaattagacttagcatatacgcacgaaacacttagcatatacatgactattttagtcttagcatgtaatattatttgagttttaatataagattactttatttgtaattagacttagtatgtaattattgactacggaattggtgcgacaagtagcaattgactattgtagtcttaattagacttagcatatacgcacgaaacacttagcatatacatgactattgtagtcttagcatgtaatattatttgagttttaatataagattattttatttgtaattagacttagtatataattatttactagctagggttatataatatacgtcacctagacttagcttatatcacgatttgtaattagacttagcacgtaaggttgtgtagggttctaatgtacgacatttacacttagcatacatgacttagattgttaaaacataaatgtctcgtgacttagcagatgtttcttgtatcaacagatggctgaccgcgatgaggaacagatattgtacgatacaatcgcggagggaagcagccagtactggaatgaagaagaggggaacgaggatccaaaccagtacttgaacgaggaagggaacgtggagagggatgcggaggggaaccagcaggggcacgtggaaagggatgtggaggggaaccaggaggaggaggctagtggtagtcaaccctccgttggacagaagagggcacgcgggcaacgaggtgcagcgaagaagcttgagggtcggcacatcataacggaagtgcaagaagatggacgtcctagtgccccggccgaagccgccaagaactatgtacgtcacagcggttgggttgtgcgggataacgtgcctgtcagtacggtgtactggcgaagaacaagggcacgcggagatcatgagagctttgtcccagattcagagaaagagatgctgtggaccacaatgctcgagacattcacccttcctgcgggtacagaggacaaagtgaaaaggtggactctgaagaaaatggcagaacagtttcagagcttcaagggagatctgtaccaaaagtatatactgaaggggcagacaccgaacttcgacacattcccaaagctaagggatcactgggacgagtttgttgcatataagacaggtgaacaagggcaggcgatgatggaaagaaacaaagaaaatgccgccaagaagaagtaccatcaccacttggggtcaggaggctatagcgtcgcgatgccgaagtgggagcagatggaggctagcttgattgagaggggtatcgaaccggcaacagccaattggccggaacgatcgaagttctggtactatgctcacggtggaacgctcaacccagctgatggctcactggtcttcggcgatcagatacgagaggctgcgcgacgactaacagatgcagtggaagcctcctctcagggcacgttccgaccagacagagatagggacgagctgacactcgccctgcagactccagagcatccaggacgaacacgagggaaaggggtgattccttggaagattggtttcaaggaggacatccacacgtacaggagttggatgaggagcaagagagataccgaggcgaagattgcagacctagagttccaggtatcgagctacgaactcaacatgcaagaggaggtggcaaggaaggttgatgaacgcatggccgcacatcggtcccatgatccccagctgaccattcctcctgcaatggtaagcctgtcaggaaaccgtagcagctgcgcctcaacggggcaggtaggatcacagagcatggacgccatgcaaacacaggacgaatcgacctgtcccgttgatgacatcactcaacggacaccatgtgagctgcatattcctttcaagaacttatcaataaaggtatgatttagccattccgctagttgctgcttatatatgttgattactaataaataatctctcacaacatgaaggtggcgtcgggcatggccatcccaacggacccttcaggtacttaccactgcaggccgattccagcaggatactcgaaggtcgaagttgagttggtcgaaggcgcgtacgaggacctcgagctggattaccctggaggagacggtgagacgcatctacgagaaacaagccatgccattattctatgacgcaggcggtacatcatcctccctgggcgacaagcggcgtctcgtgcaccatctcctcctcaggatcctgcaccgtctcctcctcatgctccgatagcaccgtctccacctcaggctccagcatcgactcctcctcaggatcctgcaccgactcctcctcgtgctcctacacctactcccccgcaagctcctcttccggcaccttcaaagtcaagggcccccccagctccaccgcctgcccacacaagggcagtgaagaaggcgaaagttgacgccgccaagaacaaggacccggggtacgatcgcacgcaagaggagcttgacgcttacgttgcatcagaagtcaagagacaattcaagcctcgaagtccagaaaagaagattcctatagaccccagtgtcaggaacttcttcaggggtatgtctgcatctgtcaaggaggccatcaagctatcggactatgagcgaacgctgaagaaagcatcttctggaaagtccaaaccagtccctcagcttggagagcaaccaaaccaggagatcgagccgttggtgaccagtaaagaaatgacgatagaacaatttattactgacaccggtctaactacggatcaattgctaggagtcgcaccaatcgaaaaggcgaaagtgaaatacatgtacgaactcggtaaaccgcttgtcaagcctgagctgctgcagtccctacccacacaaatgtacaagttccatcagctgtacatggagatgagcgccaccggtagagagatgatcggagcgacgatcagggacacggacttcttgcaaggagatgacattctctggatcaatttcaggggaatctacgaactataccagctggacgccctcgacgtctctattatgagttgctggattttgtaagtatatcgttcaattataattcttaattactacgtctcctttaattaattaggtccttgtatataagtagactatataaaataaaatactcccttttatcgttgtagaatggagattcaaagggcccgacggcggagggttttcgatactggattcatcgaccctcggagagtaaacgttgcaatgctcgaccaatatccgcaggaaacagaggacaatctcgtccatctcctgaaggcgcagcattacaagacgttcatactgttaccgtacaacacagagttagtttaattttactgtcttcctacataccaaatttcattcccgtacgaacttgctaagtgtttcatatgtaatgcatcccacgcacattgcagattccactgggtgcttttactcttcgacctggaggcctgcaccgtcaacgtatatgactcaatggataaaaaagagtctacgtttgacaaggttttcgaacttatagacaggtaccgtcataagttcctttgttaattaagaaaatcttgttatgttaattgctactacgaatcatagctttaaactccatgtagggcttggtatcggttccgtcatttggtccgcagcaaatggagagaaagacttaggcggaagttcaaatttcctgtgagtacacatgctctacatttatatttctccgattcaaattaatacatacaactgtatattaattagatctcacgccgttaatttgtcatttatttgtagtgcgcaaagcaaaagcagggaactaacttgtgcggctactacgtgtgtgagtattgccactgccttacagaccaaatcatcaccacaagagagctcgatgtacgtacaaataaattcaaaatttcattacgtaacgatttcttgtttaattactaatcaatttcatacattcatatagtttattcgcatgagggataacctgaccacacacaaggaatttatcgcagcggttcaagaacaactcatgggattcatcaacgaagatatccttgatcccaagggtgaattctactacgacggaaacacaattcaccggtccttagcttctgagctagcagcgactactactacgtcgaaatcgtagctagctaggacataaaatggattgtaattaatatatgactacatatgtttctatatgcatgtgtacacattttctataatgtaaatatattttggtcatatatatatctatatacatatgcatttgcataacatatatatgtataaatacatatatattatgcatgtatatacatataatataatatatatatatatatacatatatatatgtatgcatatatatgtattgaaacatatatatgcatggtttttatatatatatatatatatatatatatatatatatatatatatatatatatatatmtatataaaccatgcagcaacagggccatgcaaaaaaaaaaggtcagctcgatctttagtcccggttatttcacccgggactaaagatagcgatctttagtcccggattggtactcccggtttggaaaccgggactaaagggggttacgaaccgggactacaaagggtttctccactagtgtattaatttcatattagttacactacaattacactgtagttacattcgtaagtacagtataactagggtgtaatttttttaaaagaaagattctggtgtagttacactataattacactatagttacattcgtaagtacagtataattagggtgtaatttttttaaaagaaagattttggtgtagttacactataattacactatagttacaagtgtaagtatagtgtaactagggtgtaattttttaaaaaaactttttagtgtagttacactataattatatattagttacacagtacttacatattagttatactataattacactatagttacaaatgtaagtatagtgtaattagggtgtaattttttttaaaaaaagcctttagtatatgtgctgacaagtagttagttttgtggcttggattaacacgccttaatctacactataggcgttgctttttaatttggctagcaagttggccgtttggtggttagaaaaatctgtgtgatttataacaaaaatctgtcgggtgttatatagatagatcttcaaaatttggcagtaaactaaatgtagccacttttttgacaactttaccaaaatttggtaagattaaaaatggcatcaaagtgaacaggcccatagtatatcgtatattgttagggactattaaaaaactgccctaagttctttaagaaaaaaactttaattatagtataattgtaatatgattccactataactattatataacttttatataagtattaaaataatatatacagttttatatctaacttatatagatattacaatatacttacagtgcagatacaatgtaactatatagttacagcgtaatttacaatgtaactatactgtaattacattatactgtaattacattatagttaaacttgcgacagtttttttagcaatttcgtatatatagcatgaaatcgccatggtttaaaacctatctattattagtacccccattatttaattatatgttgtttttactttttttaatcaaatttttttagatttaatcaagtttatagaaaattttagtaacatgtataaaaccaaattagtttcatcagatgaaagatttaatagattttgataatatatatattttgtgttgaaaatgctactatatttttctataaatttggttaaactttaaaaaagttttattaggaaaaaaatcaaaatgacttataatatgaaactgatggagtagaaaaaaggggcaggcctcaaatcctgcaatccaaagaataaa